(19) United States
(12) Patent Application Publication
(10) Pub. No.: US 2024/0115550 A1
(43) Pub. Date: Apr. 11, 2024

(54) TREATMENT OF MENTAL DISORDERS

(71) Applicant: GH Research Ireland Limited, Dublin (IE)

(72) Inventors: Theis TERWEY, Berlin (DE); Conor BURKE, Laytown (IE); Naoise GAFFNEY, Dublin (IE)

(73) Assignee: GH Research Ireland Limited, Dublin (IE)

(21) Appl. No.: 18/373,914

(22) Filed: Sep. 27, 2023

Related U.S. Application Data
(63) Continuation-in-part of application No. PCT/EP2023/057885, filed on Mar. 27, 2023.

(30) Foreign Application Priority Data
Mar. 27, 2022 (EP) 22000084.8
Mar. 27, 2022 (EP) 22000085.5
Jan. 30, 2023 (EP) 23153996.6
Jan. 30, 2023 (EP) 23154014.7
Mar. 27, 2023 (WO) PCT/EP2023/057874

(51) Int. Cl. A61K 31/4045; A61K 9/00; A61P 25/24

(52) U.S. Cl. CPC A61K 31/4045; A61K 9/0019; A61P 25/24

(57) ABSTRACT

5-Methoxy-N,N-dimethyltryptamine (5-MeO-DMT) or a pharmaceutically acceptable salt thereof for use in the treatment of a mental or nervous system disorder in a mother having a child of age 18 months or below, wherein the 5-MeO-DMT or a pharmaceutically acceptable salt thereof is administered via the intravenous, intramuscular or subcutaneous route.

### CROSS-REFERENCE TO RELATED APPLICATIONS

This application is a Continuation-In-Part of PCT/EP2023/057885, filed Mar. 27, 2023, which claims priority to EP 23153996.6, filed Jan. 30, 2023, EP 23154014.7, filed Jan. 30, 2023, EP 22000084.8, filed Mar. 27, 2022, and EP 22000085.5, filed Mar. 27, 2022. This application also claims foreign priority to PCT/EP2023/057874, filed Mar. 27, 2023. The disclosure of each of the applications identified above is expressly incorporated by reference herein in its entirety.

### TECHNICAL FIELD

The present invention relates to 5-methoxy-N,N-dimethyltryptamine (5-MeO-DMT) or a pharmaceutically acceptable salt thereof for use in treating a patient who is a mother, in particular a breastfeeding mother, diagnosed with a mental or nervous system disorder.

The mental disorder is amenable to treatment with 5-MeO-DMT or a pharmaceutically acceptable salt thereof. The treatment also improves maternal functioning.

The invention moreover allows for treating the mental or nervous system disorder in a breastfeeding mother without substantial interruption of breastfeeding.

### BACKGROUND OF THE INVENTION

Mental or nervous system disorders in breastfeeding mothers can lead to a wide range of negative consequences for the affected mother, her infant(s) and her family. For example, women suffering from a mental or nervous system disorder may develop thoughts of self-harm or harming their child and they are at increased risk of suicide.

Mental or nervous system disorders may further lead to disruptions in the interactions between mother and child, exemplified by higher rates of disengaged behaviour and lower rates of visual and vocal communication between mother and child. Evidence also suggests an association between mental or nervous system disorders a mother suffers from and child development, as illustrated by the fact that children of patients suffering from a mental or nervous system disorder may have a greater risk of impaired cognitive development.

Despite these problems, treatment options are rather limited. Quite generally, known treatments for mental or nervous system disorders are often associated with only limited treatment success, in particular in patients suffering not merely from mild symptoms of the disease.

In the case where the patient is a breastfeeding mother, a compounding factor is that for many medicaments, lactating women are advised to discontinue breastfeeding during the period in which they take the medicament and for some time thereafter as the medicament may be excreted in milk and expose the suckling child to a risk.

Moreover, research has shown that breastfeeding mothers may be reluctant to commence pharmacological treatment due to a range of concerns.

In consequence, breastfeeding patients suffering from a mental or nervous system disorder may be confronted with a situation where a decision must be made whether to discontinue breast-feeding or to discontinue/abstain from therapy.

Against this background there is a need for an improved treatment of mental or nervous system disorders, in particular a treatment that not only effectively addresses symptoms of the disorder and leads to a rapid clinical response but also avoids interference with the patient's everyday activities, in particular regarding care of the infant(s). The treatment should improve maternal functioning. There is furthermore a need for a treatment of mental or nervous system disorders that does not require a substantial interruption of breastfeeding.

While there has recently been significant interest in hallucinogens for the treatment of mental disorders, this has so far not led to a treatment of breastfeeding mothers based on such substances. This is due to a general lack of relevant clinical data which would allow drawing conclusions on the clinical utility of hallucinogens as well as due to specific concerns that administration of hallucinogens may not be appropriate for breastfeeding mothers.

Hallucinogens including psychedelics are chemical compounds, some naturally occurring, some synthetic, which are defined by their ability to induce in humans after consumption sensory distortions, such as changes in auditory and visual perception, as well as distortions of mood and cognition. The term hallucinogen encompasses a rather broad group of psychoactive molecules with different modes of action. Some mental disorders have been suggested as in principle being amenable for treatment with psychoactive molecules, like psychedelics.

However, no psychedelic drug has been approved by any regulatory agency. In fact, clinical experience with such molecules is still rather restricted.

One compound already investigated in clinical trials is 5-methoxy-N,N-dimethyltryptamine (5-MeO-DMT). WO 2020/169850 reports on tests in healthy volunteers as well as a clinical trial involving patients suffering from treatment resistant depression (TRD), i.e., a form of major depressive disorder. Patients suffering from PPD were not included in the trial.

Against this background, an aim of the invention is in particular the provision of therapies which are more effective (i.e., a) larger percentage of patients experiencing a clinical response, b) a larger average clinical response, c) an earlier onset of the clinical response, and/or d) a more durable clinical response) than previously described therapies.

A further aim of the current invention is to provide a compound for improved psychoactive therapies and dosing regimens for said therapies which have a better safety profile and/or are better tolerated than previously described therapies. Another aim of the current invention is to provide a compound for improved psychoactive therapies and dosing regimens for said therapies which are more convenient than previously described therapies. Another aim of the current invention is to provide a compound for improved psychoactive therapies and dosing regimens for said therapies which are associated with higher rates of patient compliance (including higher rates of treatment initiation) than previously described therapies. A still further aim of the current invention is to identify specific disease aspects and specific subgroups of disease aspects which benefit from such improved psychoactive therapies.

Still further aims of the invention are to improve maternal functioning in patients suffering from a mental or nervous system disorder, in particular in a breastfeeding mother diagnosed with a mental disorder.

### SUMMARY OF THE INVENTION

The present invention provides 5-Methoxy-N,N-dimethyltryptamine (5-MeO-DMT) or a pharmaceutically acceptable salt thereof for use in treating a mother diagnosed with a mental or nervous system disorder, in particular a breastfeeding mother diagnosed with such a disorder. The disorder may be in particular a disorder characterized by depressive episodes, for example, Major Depressive Disorder (MDD), Persistent Depressive Disorder, Seasonal Affective Disorder and Bipolar Disorder (BD), such as Bipolar I Disorder and Bipolar II Disorder; Anxiety Disorder, for example, Separation Anxiety Disorder, Agoraphobia, Generalised Anxiety Disorder (GAD), Social Anxiety Disorder (SAD), Panic Disorder, Phobia, and Substance/Medication Induced Anxiety Disorder; Somatic Symptom Disorder; Obsessive compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain, Fibromyalgia, and Migraine; Mental and Behavioural Disorders due to Psychoactive Substance Use, for example, Substance Use Disorder (SUD); Psychotic Disorders, for example Schizophrenia; an Eating Disorder; Attention Deficit Hyperactivity Disorder (ADHD); a Personality Disorder, for example Schizotypal Personality Disorder and Borderline Personality Disorder; Autism Spectrum Disorder; Chronic Fatigue Syndrome; a mental disorder or a nervous system disorder associated with HIV, with Post COVID Condition or with Traumatic Brain Injury.

In one aspect, the patient may suffer from sleep disturbance.

The treatment allows for an improvement in maternal functioning.

The patient to be treated is in particular a breastfeeding mother.

The invention also allows for treating a breastfeeding mother without substantial interruption of breastfeeding.

The present invention also provides dose ranges and dosing regimen useful for the treatments indicated above.

In the context of the present invention, 5-MeO-DMT or a pharmaceutically acceptable salt thereof is administered via intravenous, intramuscular or subcutaneous administration.

5-MeO-DMT or a pharmaceutically acceptable salt thereof is administered at a dose or in a dosage regimen that causes the patient to experience a peak psychedelic experience. A dosage of about 1 mg to about 10 mg 5-MeO-DMT or an equimolar amount of a pharmaceutically acceptable salt may be administered.

### DETAILED DESCRIPTION OF THE INVENTION

### Definitions

As used in the context of the present invention, unless otherwise noted, the term “5-MeO-DMT” refers to the free base 5-MeO-DMT. It is contemplated that pharmaceutically acceptable salts of 5-MeO-DMT may also be used. Such salts are in particular acid addition salts, wherein the acid may be selected from, for instance, acetic acid, benzoic acid, citric acid, fumaric acid, hydrobromic acid, hydrochloric acid, hydrofluoric acid, hydroiodic acid, oxalic acid, succinic acid and triflic acid. A preferred example is the hydrobromide salt. The appropriate weight amount of a salt to be administered can be calculated from the weight amount of the free base, assuming that equimolar amounts are used.

As used in the context of the present invention, a “patient” to be treated is a mother having a child, typically of 18 months or younger, in particular of 12 months or younger, who is diagnosed with a mental or nervous system disorder by a licensed professional in accordance with accepted medical practice.

Diagnosis of a mental disorder or a nervous system disorder can, for instance, be in accordance with the Diagnostic and Statistical Manual of Mental Disorders-Fifth Edition (DSM-5) published by the American Psychiatric Association. In some instances, as is apparent from the discussion of specific conditions below, the criteria may be modified or supplemented to better define patients or patient groups particularly benefiting from a treatment according to the invention. The diagnosis will in any event be by a physician or a psychologist. It is not sufficient that the human subject himself considers that he is suffering from the disorder.

As used in the context of the present invention, unless otherwise noted, the terms “treating” and “treatment” shall include the management and care of a patient for the purpose of combating a disease, condition, or disorder and includes the administration of compounds and methods according to the present invention to alleviate the signs and/or symptoms of the disease or eliminate the disease, condition, or disorder.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of the disease, for instance, if the patient suffers from a disorder characterized by depressive episodes, in the current episode of depression.

As used in the context of the present invention, “suicidal ideation” refers to thinking about, considering, or planning for suicide. The presence of suicidal ideation in a patient will be diagnosed by a physician or a psychologist, using established protocols and methods for diagnosing suicidality. It is generally not sufficient that the patient himself considers that he is suffering from suicidal ideation. In some situations, a patient experiencing suicidal ideation will be at imminent risk of committing suicide, or will be considered to have ‘intent to act.’

As used in the context of the present invention, unless otherwise noted, the term “therapeutically effective amount” shall mean the amount of active compound or pharmaceutical ingredient that elicits the biological or clinical response in a human that is being sought by a researcher, medical doctor or other clinician, which includes alleviation of the signs and/or symptoms of the disease, condition or disorder being treated.

“Clinical response” includes, but is not limited to, improvements on rating scales

The severity of a condition as well as changes of the severity can be assessed by the Clinical Global Impression (CGI) rating scales which are measures of symptom severity, treatment response and the efficacy of treatments.

The CGI rating scales were developed to provide a brief, stand-alone assessment of the clinician's view of the patient's global functioning prior to and after a treatment (Busner, J. and Tagrum, S. D., 2007. The Clinical Global Impressions Scale: Applying a Research Tool in Clinical Practice. Psychiatry 2007, 29-37).

The CGI-Severity (CGI-S) is based on one question the clinician has to answer: “Considering your total clinical experience with this particular population, how mentally ill is the patient at this time?” This is rated on the following seven-point scale: 1=normal, not at all ill; 2=borderline mentally ill; 3=mildly ill; 4=moderately ill; 5=markedly ill; 6=severely ill; 7=among the most extremely ill patients.

The CGI-S can be used to assess treatment success by comparing scores before and after treatment.

Alternatively, treatment success can be assessed using the CGI-Improvement (CGI-I), which is similarly simple in its format. After the treatment, the clinician compares the patient's overall clinical condition to the one prior to the treatment (the so-called baseline value). Again, only one query is rated on a seven-point scale: “Compared to the patient's condition at admission to the project [prior to medication initiation], this patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.”

The Patient Global Impression scale (PGI), also known as Subject Global Impression (SGI), is the counterpart to the Clinical Global Impressions scale (CGI). It consists of one item based on the CGI and adapted to the patient. It can measure disease severity (PG IS) or disease improvement (PGI-I).

The severity of a condition as well as changes of the severity can further be assessed rating scales applicable for the particular mental or nervous system disorder the patient suffers from.

Individual items of scales as described herein as well as sub-combinations of individual items may be used to assess specific disease aspects.

Maternal functioning may be assessed using the Barkin Index of Maternal Functioning (BIMF).

When assessing a clinical response at an early timepoint after drug administration (e.g. at 2 hours) based on endpoints which have been developed for a longer recall period (e.g. normally 7 days for the MADRS), a rational modification of such endpoint (e.g. changing the MADRS recall period to 2 hours and carrying forward the sleep item recorded at baseline before drug administration) may be applied.

The considerations outlined apply for early timepoints because, on the one hand, in order to assess a clinical response, the influence of the patients status before the treatment on any score recorded after treatment should be kept as low as possible, whereas on the other hand the sleep item cannot be assessed 2 hours after drug administration.

At later timepoints, for instance, on day 1 or later, typically all items of the relevant scales to assess a clinical response can be assessed, using, if necessary, an adapted recall period, so that it is not necessary to carry forward any pre-treatment score. For instance, if the BIMF is assessed on day 7, a recall period of seven days will be used (instead of the standard recall period of 2 weeks).

As used in the context of the present invention, unless otherwise noted, the term “administration” (or “application”) shall mean the introduction of an amount, which may be a predetermined amount, of active compound or pharmaceutical ingredient into a patient via any route. The active compound is administered by intravenous administration, by intramuscular administration or by subcutaneous administration.

As used in the context of the present invention, unless otherwise noted, the terms “dose” and “dosage” and “dosage amount” shall mean the amount of active compound or pharmaceutical ingredient which is administered to a patient in an individual administration. The term “dosage regimen” (or “dosing regimen”) shall mean a defined sequence of one or more individual administrations.

Mental and Nervous System Disorders

The mental and nervous system disorders to be treated according to the invention have in common that they are associated with one or more symptoms from the symptom clusters discussed below, which include sleep disturbance, cognitive dysfunction, anxiety, psychomotor retardation, social/emotional withdrawal and negative thinking.

In one aspect, the present invention relates to 5-methoxy-N,N-dimethyltryptamine (5-MeO-DMT) or a pharmaceutically acceptable salt thereof for use in treating a patient who is a breastfeeding mother diagnosed with a mental or nervous system disorder.

The mental or nervous system disorder is amenable to treatment with 5-MeO-DMT or a pharmaceutically acceptable salt thereof. It is in particular major depressive disorder, persistent depressive disorder, bipolar disorder, anxiety disorder, posttraumatic stress disorder, body dysmorphic disorder, obsessive-compulsive disorder, eating disorder or psychoactive substance abuse.

In a preferred embodiment, the mental disorder is major depressive disorder.

In another preferred embodiment, the mental disorder is Postpartum depression (PPD), a complex mix of physical, emotional, and behavioral changes that happen in some women after giving birth. PPD is also known as major depressive disorder with peripartum onset. According to DSM-5 (Diagnostic and Statistical Manual of Mental Disorders, 5th Edition) criteria, PPD is diagnosed when major depressive disorder (MDD) symptoms begin during pregnancy or within four weeks of delivery.

In another preferred embodiment, the mental disorder is bipolar disorder, such as bipolar II disorder. The patient diagnosed with bipolar disorder in particular suffers from a current major depressive episode.

Various aspects of bipolar disorder, such as sleep disturbance, psychomotor retardation (reduced energy and activity and reduced motivation), negative thinking (worthlessness; helplessness and hopelessness; guilt), anxiety, cognitive dysfunction (impaired concentration and memory) and social/emotional withdrawal or detachment (anhedonia, emotional withdrawal and affective flattening) can be improved. Further aspects of the disease which can be improved include suicidal ideation and mixed symptoms (psychotic symptoms; irritability; lability; increased motor drive; increased speech; agitation). The improvements that can be achieved are reflected on clinically relevant scales.

Scales to Assess Mental and Nervous System Disorders

Numerous scales have been suggested to assess severity of mental disorders or nervous system disorders. Such scales are based on tests which can be self-administered or administered by a clinician.

Scales for the assessment of mental or nervous system disorders which may be used according to the invention include those known in the art for diagnosis and/or monitoring the mental or nervous system disorders discussed in more detail below.

Treatment outcome is assessed by using one or more indices or scales at one or more time points after completion of a treatment course.

The assessment can be carried out after the acute psychedelic experience has subsided. An appropriate point in time for an early assessment is generally about 2 to 3 hours after the last administration. An early assessment can generally be carried out, for instance, about 2 hours or about 3 hours after the last administration.

An assessment of an effect on sleep disturbance or an effect on a mental or nervous system disorder related to an effect on sleep disturbance can, however, be carried out at the earliest on the day after the treatment (i.e., on day 1) so that the treated patient had the opportunity to sleep for at least one night.

Thus, an assessment at day 1 or on day 1 means an assessment on the day following the administration. The assessment will be carried out not earlier than 12 hours after the last administration and in any event not earlier than one night after the last administration and not later than 36 hours after the last administration. The assessment can be carried out after about 24 hours.

An assessment at day 7 or on day 7 means an assessment on the seventh day following the administration (the day of administration is day 0). Analogous definitions apply for other assessment timings measured in days.

When assessing a clinical response, for instance, using one of the scales to assess severity of a mental disorder or a nervous system disorder, at an early timepoint after drug administration (e.g. at 2 hours) based on endpoints which have been developed for a longer recall period (e.g. normally 7 days for the MADRS), a rational modification of such endpoint (e.g. changing the MADRS recall period to 2 hours and carrying forward the sleep item recorded at baseline before drug administration) may be applied. The same applies with respect to any other scale applied herein to assess treatment effects on a mental or nervous system disorder, unless a recall period is specifically indicated.

The considerations outlined apply for early timepoints because, on the one hand, in order to assess a clinical response, the influence of the patient's status before the treatment on any score recorded after treatment should be kept as low as possible, whereas on the other hand the sleep item cannot be assessed 2 hours after drug administration.

At later timepoints, for instance, on day 1 or later, typically all items of the relevant scales to assess a clinical response can be assessed, using, if required, an adapted recall period, so that it is not necessary to carry forward any pre-treatment score.

The Active Agent

Mental or nervous system disorders are characterized by several aspects which as such present a significant disease burden and deserve appropriate treatment. Thus, there is not only a need for a treatment, in particular by pharmacological intervention, to improve overall disease scores but also to improve specific aspects of the diseases.

The inventors considered that a carefully chosen hallucinogen may lead to an improved treatment and may moreover lead to overall improvements of the disease and of maternal functioning.

The inventors further considered that a carefully chosen hallucinogen may allow continuing breastfeeding without substantial interruption in case of treatment of a breastfeeding mother suffering from a mental or nervous system disorder.

One group of hallucinogens entails compounds which bind to the 5-hydroxytryptamine (5-HT) receptors, which are also referred to as serotonin receptors (described are 7 families 5-HT1 to 5-HT7 with several subtypes). Examples are lysergic acid diethylamide (LSD), psilocybin, and N,N-dimethyltryptamine (DMT). These serotonergic agents are often referred to as “psychedelics”, which emphasizes their predominant ability to induce qualitatively altered states of consciousness such as euphoria, trance, transcendence of time and space, spiritual experiences, dissolution of self-boundaries, or even near-death experiences, while other effects such as sedation, narcosis, or excessive stimulation are only minimal.

Chemically, serotonergic psychedelics are either phenylalkylamines or indoleamines, with the indoleamine class being divided into two subsets, ergolines and tryptamines, the latter being derived from tryptamine.

The various serotonergic psychedelics have different binding affinity and activation potency for various serotonin receptors, particularly 5-HT1A, 5-HT2A, and 5-HT2C, and their activity may also be modulated by interaction with other targets such as monoamine transporters and trace amine-associated receptors.

Recently published clinical studies which have used serotonergic psychedelic drugs such as LSD, psilocybin and DMT (using the shamanic brew Ayahuasca, which contains DMT) in certain mental disorders suggest that those compounds could provide an alternative to the currently available treatments for certain mental disorders. However, there are reports that these compounds can induce mania in patients suffering from depressive symptoms, and this may preclude their clinical use.

For instance, Lake et al. (Lake, C. R., Stirba, A. L., Kinneman, R. E. Jr, Carlson, B., Holloway, H. C., 1981. Mania associated with LSD ingestion. American Journal of Psychiatry. 138(11):1508-9) report about a patient who suffered a manic attack after ingesting LSD or an LSD analogue. The patient experienced acute symptoms of LSD intoxication, which resolved but were followed in about 3 weeks by a typical manic episode of psychotic magnitude. Hendin and Penn (Hendin, H. M., Penn, A. D., 2021. An episode of mania following self-reported ingestion of psilocybin mushrooms in a woman previously not diagnosed with bipolar disorder: A case report. Bipolar Disorders 23(4):1-3) report about an episode of mania following self-reported ingestion of psilocybin mushrooms. Szmulewicz et al. (Szmulewicz, A. G., Valerio, M. P., and Jose M Smith, J. M., 2015. Switch to mania after ayahuasca consumption in a man with bipolar disorder: a case report. International Journal of Bipolar Disorders (2015) 3:4) report on a switch to mania after consumption of ayahuasca, a DMT containing brew, in a man with bipolar disorder.

A further case report is found in Brown, T., Shao, W., Ayub, S., Chong, D., & Cornelius, C. (2017). A Physician's attempt to self-medicate bipolar depression with N, N-dimethyltryptamine (DMT). Journal of Psychoactive Drugs, 49(4), 294-296.

The inventors considered that in order to avoid the induction of mania or hypomania or at least reduce the risk of induction of mania or hypomania, the compound administered must be appropriately chosen and preferably is administered in a particular dosing regimen.

The inventors identified 5-methoxy-N,N-dimethyltryptamine (5-MeO-DMT) as a psychedelic of particular interest for use in therapy. 5-MeO-DMT has a distinct pharmacological profile which differs from that of other psychedelic compounds.

5-MeO-DMT is a potent, fast-acting, naturally occurring serotonin (5-HT) agonist, acting at both the 5-HT1A and the 5-HT2A receptor, with higher affinity for the 5-HT1A receptor subtype compared to other classical psychedelics.

Inhibition constants (Kᵢ values) as further detailed on the example section below for psilocin (the dephosphorylated from of psilocybin which is formed after uptake of psilocybin), DMT and 5-MeO-DMT are 48, 38 and 1.80 nM, respectively, at 5-HT1A receptors located in the hippocampus of post-mortem human brain. Thus, 5-MeO-DMT exhibits high affinity and psilocin and DMT exhibit moderate affinity for 5-HT1A receptors. Inhibition constants (Kᵢ values) for psilocin, DMT and 5-MeO-DMT are 37, 117 and 122 nM, respectively, at 5-HT2A receptors located in the frontal cortex of post-mortem human brain. Therefore, psilocin exhibits moderate/strong affinity and DMT and 5-MeO-DMT exhibit comparatively weak affinity for 5-HT2A receptors.

Relative to the other psychoactive compounds mentioned previously, 5-MeO-DMT displays an enhanced affinity for the 5-HT1A receptor, where it acts as a potent agonist. In the case of psilocin and DMT, there is an increased contribution of 5-HT2A binding, relative to 5-MeO-DMT, with the latter displaying the largest differential affinity for 5-HT1A over 5-HT2A of the three compounds. Therefore, 5-HT1A binding plays a much bigger role in the overall effect of 5-MeO-DMT relative to 5-HT2A binding compared to the other two compounds.

It has been reported that 5-HT1A agonism reduces impulsivity and aggression, whereas 5-HT2A agonism can result in short-term increases in these same traits. Furthermore, the dopamine system has been implicated in contributing to mania, with increased dopamine drive being linked to mania. LSD, psilocybin and DMT all display increased affinity for a variety of dopamine receptors relative to 5-MeO-DMT

Compared to other psychedelics, like LSD, psylocibin or DMT, 5-MeO-DMT can be administered to patients, preferably using dosing schemes as described herein, without a significant risk of inducing mania or hypomania in a patient suffering from a mental or nervous system disorder, including a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; a Psychotic Disorder, such as Schizophrenia; or a personality disorder, such as Schizotypal Personality Disorder. The patient suffering from such a mental or nervous system disorder, treated according to the invention, does not experience treatment-emergent mania or hypomania.

It is also noted that reports of treatment-emergent mania or hypomania related to psychoactive substance use seem to indicate large quantities of the respective compounds (e.g., DMT/ayahuasca, psilocybin, LSD) were used.

The inventors' approach of sequential up-titration of 5-MeO-DMT significantly reduces the risk of excessive dose administration with its potential for attendant adverse events.

Still further, the induction by antidepressants of isolated events of hypomania has been reported in patients suffering from treatment resistant depression (TRD) (Bader, Cynthia D., and David L. Dunner. “Antidepressant-induced hypomania in treatment-resistant depression.” Journal of Psychiatric Practice 13.4 (2007): 233-237). However, the recently concluded clinical trial of 5-MeO-DMT in TRD patients showed no evidence of hypomania induction.

5-MeO-DMT can induce peak experiences, i.e., experiences characterized by an emotional perspective shift, which is described as “loss of ego” which often culminates in an overwhelming sense of “oneness with the universe”, more rapidly than other psychedelics and has a short duration of acute psychedelic effects (5 to 30 minutes after intravenous injection compared with several hours for e.g. oral psilocybin and oral LSD). These characteristics of 5-MeO-DMT are associated with an improved therapeutic profile which can be explained by specific alterations of Resting State Network (RSN) activity under 5-MeO-DMT treatment.

Furthermore, 5-MeO-DMT is a 5-HT7 receptor agonist showing high affinity towards the receptor. The inventors determined, using recombinant human 5-HT7 receptor, [³H]LSD as a radio ligand and serotonin to estimate non-specific binding, a Kᵢ of 2.3 nM.

Thus, besides the 5-HT1A and 5-HT2A receptors discussed above, 5-MeO-DMT also interacts with the 5-HT7 receptor. 5-MeO-DMT act as an agonist on this receptor and shows a high (nanomolar) binding affinity.

The 5-HT7 receptor has a role in neurogenesis, synaptogenesis and dendritic spine formation. It is, among other things, associated with central processes such as learning and memory, with sleep regulation and circadian rhythm and with nociception.

The 5-HT7 receptor is in particular expressed in the spinal cord, raphe nuclei, thalamus, hypothalamus including the suprachiasmatic nucleus, hippocampus, prefrontal cortex, striatel complex, amygdala and in the Purkinje neurons of the cerebellum.

The suprachiasmatic nucleus is the central pacemaker of the circadian timing system. It coordinates circadian rhythms in various brain regions. Disruption of this coordination will result in disease states, in particular disease states involving sleep disturbance. In patients suffering from sleep disturbance resting state functional connectivity analysis reveals alterations in functional connectivity between the suprachiasmatic nucleus and regions within the default mode network.

The expression of the 5-HT7 receptor in the suprachiasmatic nucleus corresponds to the function of the receptor in regulation of sleep/wake cycles. The inventors consider that this allows treatment of patients suffering from sleep disturbance by 5-MeO-DMT which acts on the receptor.

The inventors consider that binding of 5-MeO-DMT to the 5-HT7 receptor as one mediator of the pharmacological effects of 5-MeO-DMT, which involve functional connectivity “resets” of networks and neuroplasticity effects, contributes to the beneficial effects of 5-MeO-DMT in the treatment of patients suffering from sleep disturbance.

The inventors further consider that binding of 5-MeO-DMT to the 5-HT7 receptor as well as to the 5-HT1A receptor as two mediators of effects exerted by 5-MeO-DMT, which include functional connectivity “resets” of networks and neuroplasticity effects, allows achieving beneficial effects also in patients suffering from other symptoms or conditions, such as cognitive dysfunction, anxiety, psychomotor retardation, negative thinking or social/emotional withdrawal. This is supported by the clinical results demonstrated in studies referred to herein.

Another feature of 5-MeO-DMT is its short half-life.

5-MeO-DMT is mainly inactivated through a deamination pathway mediated by monoamine oxidase A, and it is 0-demethylated by cytochrome P450 2D6 (CYP2D6) enzyme.

The inventors investigated pharmacokinetic properties of 5-MeO-DMT and observed rapid absorption and distribution of inhaled 5-MeO-DMT, with maximum concentrations and pharmacological effects observed during and immediately after dosing.

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Uptitration as disclosed herein will not lead to accumulation and thus not to higher plasma concentrations, for instance, 10 minutes, 2 hours, or 3 hours after administration.

The inventors have further determined that 5-MeO-DMT offers various characteristics that renders it an attractive treatment for PPD. In contrast to SSRIs, it is a rapid-acting agent (in a 5-MeO-DMT-TRD trial, 5/8 patients with TRD achieved a remission within 2h after dosing, and 8/8 patients achieved a remission on day 1, with 7/8 patients maintaining their remission at Day 7). Using 5-MeO-DMT to treat PPD patients, not only can a rapid improvement of depressive symptoms be achieved, but also a rapid improvement of maternal functioning. Furthermore, 5-MeO-DMT is administered during a single-day treatment session, with optional infrequent redosing, thus differentiating it from SSRIs, which require a chronic daily dosing regimen associated with low compliance, and in the case of brexanolone, requiring protracted infusions and hospital admission.

The present invention thus also addresses compliance and patient convenience.

Furthermore, the inventors determined that a treatment of PPD with 5-MeO-DMT or a pharmaceutically acceptable salt thereof allows continuing breastfeeding with only a short interruption for the treatment.

According to the invention, isotopic variants of 5-MeO-DMT and pharmaceutically acceptable salts thereof can also be used. When reference is made to the use of 5-MeO-DMT or a pharmaceutically acceptable salt thereof, the use of isotopic variants is also contemplated.

These variants are in particular deuterated forms of 5-MeO-DMT and pharmaceutically acceptable salts of such forms.

Deuterated forms of 5-MeO-DMT are forms having a higher deuterium content than expected based on the natural abundance of this isotope.

Deuterated forms of 5-MeO-DMT are in particular forms wherein deuterium has been introduced at one or more defined hydrogen positions.

Examples of deuterated forms of 5-MeO-DMT include, without limitation, 1-deuterio-2-(5-methoxy-1H-indol-3-yl)-N,N-dimethylethanamine, 1,1-dideuterio-2-(5-methoxy-1H-indol-3-yl)-N,N-dimethylethanamine, 1,1,2,2-tetradeuterio-2-(5-methoxy-1H-indol-3-yl)N,N-dimethylethanamine, and N,N-dimethyl-2-[5-(trideuteriomethoxy)-1H-indol-3-yl]ethanamine.

Further examples include forms of 5-MeO-DMT wherein deuterium has been introduced at one or more hydrogen positions of the N-bound methyl groups. Still further examples include forms of 5-MeO-DMT wherein one or more deuterium atoms replace hydrogen atoms of the indole ring system. It is moreover noted that combinations of the above substitution patterns are also contemplated.

Preparation methods for these compounds are known in the art.

According to the invention, mixtures of deuterated forms of 5-MeO-DMT, mixtures of one or more deuterated form with non-deuterated 5-MeO-DMT, pharmaceutically acceptable salts of deuterated forms of 5-MeO-DMT, mixture of such salts as well as mixtures of salts of deuterated and non-deuterated 5-MeO-DMT can also be used.

Further according to the invention, deuterated 5-MeO-DMT and salts of deuterated 5-MeO-DMT are used in amounts that are equimolar to the amounts of the corresponding non-deuterated forms.

According to the invention, prodrugs of 5-MeO-DMT and pharmaceutically acceptable salts of such prodrugs can also be used. Such prodrugs of 5-MeO-DMT can be metabolically converted to 5-MeO-DMT. Thus, when reference is made to the use of 5-MeO-DMT or a pharmaceutically acceptable salt thereof, this can be replaced by a 5-MeO-DMT prodrug or a salt thereof.

In suitable prodrugs, the hydrogen in position 1 of the indole moiety is substituted by an organic moiety which can be split off after administration.

Examples of suitable organic moieties are —C(O)OR¹, —C(O)R², —CH(R³)OR⁴, —C(O)OCH(R³)OC(O)R⁴, —C(O)OCH(R³)OC(O)OR⁴, —CH(R³)C(O)R⁴, —CH(R³)OC(O)R⁴, —CH(R³)OC(O)OR⁴, wherein each of R¹, R², R³, and R⁴ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently substituted or unsubstituted.

Preferred examples of organic moieties are —CH(R³)OC(O)R⁴ and —C(O)OR¹, wherein R¹, R³, and R⁴ are defined as above.

Prodrugs, especially those of the above structure, can also be used on the form of pharmaceutically acceptable salts.

Specific examples of prodrugs are 5-MeO-DMT carboxy-isopropyl valinate, preferably in salt form, in particular as ditrifluoroacetate (1-(((S)-2-amino-3-methylbutanoyl)oxy)-2-methylpropyl 3-(2-(dimethylamino)ethyl)-5-methoxy-1H-indole-1-carboxylate di-trifluoro-acetate) and 5-MeO-DMT methyl pivalate (3-(2-(dimethylamino)ethyl)-5-methoxy-1H-indol-1-yl)methyl pivalate).

Preparation methods for prodrugs as discussed herein are known in the art.

According to the invention, the Tₘₐₓ value of the metabolite 5-MeO-DMT as measured in male Sprague-Dawley (SD) rats following oral dosing of the prodrug at 10 mg/kg is preferably 1 hour or less, more preferably 0.7 hours or less and in particular 0.5 hours or less.

Further according to the invention, prodrugs of 5-MeO-DMT and salts of prodrugs of 5-MeO-DMT are used in amounts that are equimolar to the amounts of the corresponding non-prodrug forms.

Modes of Administration

The therapeutically effective amount of 5-MeO-DMT is administered by intravenous administration, by intramuscular administration or by subcutaneous administration. Administration via these routes can assure a rapid onset of action. A most preferred route of administration is administration via the intravenous route, i.e. by intravenous injection. 5-MeO-DMT can be employed as a pharmaceutically acceptable salt, preferably the hydrobromide salt, or in the form of a formulation for administration via injection, examples of excipients and vehicles for such formulations being known in the art.

Dosing Regimen

The present invention also provides dose ranges, particular doses as well as dosing regimens (administration schemes) and appropriate routes of administration.

The invention is in part based on the inventors' conclusion that the occurrence of a peak psychedelic experience during the acute phase after administration of 5-MeO-DMT or a pharmaceutically acceptable salt thereof is driving its therapeutic benefit in patients as defined herein, who is diagnosed with a disorder as defined herein, including a treatment-resistant form of this disorder, and including this disorder associated with suicidal ideation, in particular one or more of the aspects defined above, either in a causal relationship or at least as a surrogate behavioral marker for the underlying unknown therapeutic mechanism.

Consequently, achieving peak experiences more rapidly, in a larger proportion of patients and with better reproducibility in an individual patient, compared with previously tested psychedelic agents, dosing regimens and administration routes, will lead to a better therapeutic profile.

Further, the present invention also relies on the short duration of action of 5-MeO-DMT and the absence of relevant tolerance (i.e., the absence of diminished or no psychedelic effects after re-administration), as a basis for enabling a dosing regimen with frequent re-administrations (such as more than once daily, or daily), which are designed to increase the rate of occurrence of peak experiences, thereby increasing the therapeutic benefit. Such repeat administrations within short time also allow an intraindividual dose-optimization which reduces the risk of overdosing, which may otherwise lead to somatic side effects, such as the serotonin syndrome, negative psychic reactions, such as flashbacks of the experience at later timepoints, induction of mania or hypomania or to less meaningful psychedelic experiences with few or no memories of the altered state (so-called “white-outs”). Further, starting with a low dose allows familiarization of the patient with the psychedelic experience in general, and allows preparation for the more intense symptoms to occur at the higher doses, which will positively influence the experience at those higher doses. Also, the prospect of being able to initiate treatment with a low dose will increase patient acceptance of the therapeutic approach and improve overall compliance rates on the patient population level.

Frequent re-administrations of a serotonergic psychedelic with the aim to increase the rate and tailor the reproducibility of peak experiences and to improve the therapeutic effect, reduce the side effects and improve the compliance rates may not be possible with other psychedelics, due to the late onset and long duration of psychedelic effects and due to the rapid development of tolerance (i.e. diminished or no psychedelic effects after re-administration) which can last for several days.

A patient as defined herein, who is diagnosed with a disorder as defined herein, including a treatment-resistant form of this disorder, and including this disorder associated with suicidal ideation, who is treated by administration of 5-MeO-DMT or a pharmaceutically acceptable salt thereof.

In a preferred embodiment, the 5-MeO-DMT is administered as a monotherapy, i.e., the patient does not receive any other treatment for diagnosed disorder.

The dosage amount of 5-MeO-DMT administered to a patient, as defined herein, who is diagnosed with a disorder as defined herein, including a treatment-resistant form of this disorder, and including this disorder associated with suicidal ideation, is in the range of about 1 mg to about 10 mg, or any amount of range therein and is administered in the form of a formulation for administration based on a pharmaceutically acceptable salt of 5-MeO-DMT, such as the hydrobromide salt, which weight amount can be calculated from the stated weight amounts of the 5-MeO-DMT free base, assuming that equimolar amounts are used. Useful specific amounts of 5-MeO-DMT are e.g. about 1 mg, about 2 mg, about 3 mg, about 4 mg, about 5 mg, about 6 mg, about 7 mg, about 8 mg, about 9 mg, and about 10 mg. Note that in this specification, when ranges are set forth, such as “about 1 mg to about 10 mg,” the inventor contemplates all discrete values within that range, some of which are specifically mentioned, but all of which are not—simply for the purpose of brevity.

In preferred embodiments the improved methods for the treatment of a patient, as defined herein, who is diagnosed with a disorder as defined herein, including a treatment-resistant form of this disorder, and including this disorder associated with suicidal ideation, with a therapeutically effective amount of 5-MeO-DMT, comprise the occurrence of a clinical response not later than about 2 hours after administration of 5-MeO-DMT.

In preferred embodiments the improved methods for the treatment of a patient, as defined herein, who is diagnosed with a disorder as defined herein, including a treatment-resistant form of this disorder, and including this disorder associated with suicidal ideation, with a therapeutically effective amount of 5-MeO-DMT, comprise the persistence of a clinical response, including a clinical response which occurred not later than about 2 hours after administration of 5-MeO-DMT, until at least about 6 days after the last administration of 5-MeO-DMT, preferably until at least about 14 days after the last administration of 5-MeO-DMT, more preferably until at least about 28 days after the last administration of 5-MeO-DMT.

In preferred embodiments the improved methods for the treatment of a patient, as defined herein, who is diagnosed with a disorder as defined herein, including a treatment-resistant form of this disorder, and including this disorder associated with suicidal ideation, with a therapeutically effective amount of 5-MeO-DMT comprise the administration of more than a single dose of 5-MeO-DMT.

In a preferred embodiment this more than a single dose of 5-MeO-DMT is administered to a patient in one or more treatment blocks, each block consisting of 2 to 7 administrations, with not less than about 1 hour and not more than about 24 hours between each administration within each treatment block, and not less than about 6 days between the end of one treatment block and the start of the next treatment block.

In an even more preferred embodiment this more than a single dose of 5-MeO-DMT is administered to a patient in one or more treatment blocks, each block consisting of 1 to 3 administrations, with about 24 hours between each administration within each treatment block, and not less than about 6 days between the end of one treatment block and the start of the next treatment block.

In a most preferred embodiment this more than a single dose of 5-MeO-DMT is administered to a patient in one or more treatment blocks, each block consisting of 1 to 3 administrations, with about 1 to 4 hours, preferably 1 to 2 hours, between each administration within each treatment block, and not less than about 6 days between the end of one treatment block and the start of the next treatment block.

In an embodiment the dosage amount of the 5-MeO-DMT administered to an individual patient in each of the administrations and in each of the treatment blocks is constant for that individual patient and is selected from about 1 mg to about 10 mg.

In a preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered.

In an even more preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered or the patient having experienced a peak psychedelic experience or the supervising physician having decided that further dose increases are inappropriate based on observed side effects.

For embodiments where the dosage amount increases for subsequent administrations, the dosage amount for the next administration is determined by adding about 0.25 mg to about 3 mg, preferably about 0.5 mg to about 3 mg to the dosage amount of the prior administration. For example, if the dosage amount of the first administration was 1 mg and the dosage amount increase is 3 mg, unless one of the previously mentioned stopping criteria has been reached, then the dosage amount of the second administration will be 4 mg. Preferably, the dosage amount for the third administration will be 7 mg.

In a preferred embodiment the dosage amount of the 5-MeO-DMT administered to an individual patient in each treatment block is selected from about 1 mg to about 3 mg for the first administration, and then increased, unless the patient has already experienced a peak psychedelic experience within that treatment block or the supervising physician has decided that further dose increases are inappropriate based on observed side effects, to a dosage selected from about 4 mg to about 6 mg for the second administration, and from about 7 mg to about 9 mg for the third administration. Useful specific amounts for the first, second and third administration are e.g. about 2 mg, about 5 mg, and about 8 mg.

In an additional preferred embodiment, the dosage amount of the 5-MeO-DMT administered to an individual patient in each treatment block is selected from about 0.5 mg to about 1.5 mg for the first administration, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration, and from about 2.5 mg to about 3.5 mg for the third administration. Useful specific amounts for the first, second and third administration are e.g. about 1 mg, about 2 mg, and about 3 mg.

In an also preferred embodiment, the dosage amount of the 5-MeO-DMT administered to an individual patient in each treatment block is selected from about 1.5 mg to about 2.5 mg for the first administration, and then increased, unless the patient has already experienced a peak psychedelic experience within that treatment block or the supervising physician has decided that further dose increases are inappropriate based on observed side effects, to a dosage selected from about 3.5 mg to about 4.5 mg for the second administration, and from about 5.5 mg to about 6.5 mg for the third administration. Useful specific amounts for the first, second and third administration are e.g. about 2 mg, about 4 mg, and about 6 mg.

In a further preferred embodiment, the dosage amount of the 5-MeO-DMT administered to an individual patient in each treatment block is selected from about 2.5 mg to about 3.5 mg for the first administration, and then increased, unless the patient has already experienced a peak psychedelic experience within that treatment block or the supervising physician has decided that further dose increases are inappropriate based on observed side effects, to a dosage selected from about 4.5 mg to about 5.5 mg for the second administration, and from about 6.5 mg to about 7.5 mg for the third administration. Useful specific amounts for the first, second and third administration are e.g. about 3 mg, about 5 mg, and about 7 mg.

In a further preferred embodiment, the dosage amount of the 5-MeO-DMT administered to an individual patient in each treatment block is selected from about 2 mg to about 3 mg for the first administration, and then increased, unless the patient has already experienced a peak psychedelic experience within that treatment block or the supervising physician has decided that further dose increases are inappropriate based on observed side effects, to a dosage selected from about 4.5 mg to about 5.5 mg for the second administration, and from about 7 mg to about 8 mg for the third administration. Useful specific amounts for the first, second and third administration are e.g. about 2.5 mg, about 5 mg, and about 7.5 mg.

In a further preferred embodiment the dosage amount of the 5-MeO-DMT administered to an individual patient is selected from about 1 mg to about 2 mg for the first administration of the first treatment block, and then increases with each subsequent administration within that first treatment block until the earlier of 10 mg being reached or all administrations within that treatment block being administered or the patient having experienced a peak psychedelic experience or the supervising physician having decided that further dose increases are inappropriate based on observed side effects, with that highest dosage in that first treatment block being used as the dosage for all subsequent treatment blocks and administrations within those subsequent treatment blocks. For example, if the highest dosage in the first treatment block was 8 mg because the patient experienced a peak psychedelic experience at that dose, then the dosage for all subsequent treatment blocks and administrations within those subsequent treatment blocks will be 8 mg.

In a particularly preferred embodiment the dosage amount of the 5-MeO-DMT administered to an individual patient is selected from about 1 mg to about 3 mg for the first administration of the first treatment block, and then increased, unless the patient has already experienced a peak psychedelic experience within that treatment block or the supervising physician has decided that further dose increases are inappropriate based on observed side effects, to a dosage selected from about 4 mg to about 6 mg for the second administration of the first treatment block, and from about 7 mg to about 9 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 2 mg, about 5 mg, and about 8 mg.

In a particularly preferred embodiment the dosage amount of the 5-MeO-DMT administered to an individual patient is selected from about 0.5 mg to about 1.5 mg for the first administration of the first treatment block, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration of the first treatment block, and from about 2.5 mg to about 3.5 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 1 mg, about 2 mg, and about 3 mg.

In a particularly preferred embodiment the dosage amount of the 5-MeO-DMT administered to an individual patient is selected from about 1 mg to about 3 mg for the first administration of the first treatment block, and then increased, unless the patient has already experienced a peak psychedelic experience within that treatment block or the supervising physician has decided that further dose increases are inappropriate based on observed side effects, to a dosage selected from about 3 mg to about 5 mg for the second administration of the first treatment block, and from about 5 mg to about 7 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 2 mg, about 4 mg, and about 6 mg.

In a particularly preferred embodiment the dosage amount of the 5-MeO-DMT administered to an individual patient is selected from about 2 mg to about 4 mg for the first administration of the first treatment block, and then increased, unless the patient has already experienced a peak psychedelic experience within that treatment block or the supervising physician has decided that further dose increases are inappropriate based on observed side effects, to a dosage selected from about 4 mg to about 6 mg for the second administration of the first treatment block, and from about 6 mg to about 8 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 3 mg, about 5 mg, and about 7 mg.

In a particularly preferred embodiment the dosage amount of the 5-MeO-DMT administered to an individual patient is selected from about 1.5 mg to about 3.5 mg for the first administration of the first treatment block, and then increased, unless the patient has already experienced a peak psychedelic experience within that treatment block or the supervising physician has decided that further dose increases are inappropriate based on observed side effects, to a dosage selected from about 4 mg to about 6 mg for the second administration of the first treatment block, and from about 6.5 mg to about 8.5 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 2.5 mg, about 5 mg, and about 7.5 mg. It is understood that a pharmaceutically acceptable salt of 5-MeO-DMT is preferably used in all of the above dosing regimen, and that the appropriate weight amounts of a salt to be administered can be calculated from the stated weight amounts of the free base, assuming that equimolar amounts are used.

According to the invention, 5-MeO-DMT is preferably not administered together with a MAO inhibitor.

The occurrence of a “peak psychedelic experience” in a patient can be identified through achievement of at least 60% of the maximum possible score in each of the four subscales (mystical, positive mood, transcendence of time and space, and ineffability) of the 30-item revised Mystical Experience Questionnaire (MEQ-30) (as described in Barrett FS, J Psychopharmacol. 2015; 29(11):1182-90).

The occurrence of a “peak psychedelic experience” in a patient can also be identified through achievement of at least 60% of the maximum possible score of the Oceanic Boundlessness (OBN) dimension of the Altered States of Consciousness (ASC) questionnaire (as described in Roseman L et al., Front Pharmacol. 2018; 8:974).

In accordance with the invention, the occurrence of a “peak psychedelic experience” in a patient is preferably identified through achievement of a score of at least 75 in the Peak Experience Scale (PES) Total Score, also referred to as the Peak Psychedelic Experience Questionnaire (PPEQ), which averages answers scored by the patient from 0 to 100 for the following three questions: 1. How intense was the experience; 2. To what extent did you lose control; 3. How profound (i.e. deep and significant) was the experience?Sleep Disturbance There are two fundamental types of sleep: rapid eye movement (REM) sleep and non-REM sleep. Non-REM sleep can be divided into four stages (I-IV). These non-REM stages correspond to an increasing depth of sleep. Non-REM and REM sleep alternate during each of the four to five cycles of normal human sleep each night. During the earlier proportion of the night, non-REM sleep is deeper and occupies a disproportionately large amount of time, particularly within the first cycle of sleep. As the night progresses, non-REM sleep becomes shallow and more of each cycle is allocated to REM sleep.

Normal healthy sleep consists of different phases as outlined above that proceed in successive, tightly regulated order through the night.

Disruption of this tight regulation results in sleep disturbances.

Sleep disturbance refers to conditions, whether idiopathic or occurring in the context of a medical condition such as for example a mental disorder or a nervous system disorder, that affect sleep quality, timing, or duration. It impacts a person's ability to properly function while the person is awake.

Common forms of sleep disturbances encompass disorders of initiating and maintaining sleep (insomnia), disorders of excessive somnolence (hypersomnia), disorders of sleep-wake schedule (circadian rhythm disorders), dysfunctions associated with sleep, sleep stages, or partial arousals (parasomnia), disorders characterized by respiratory disturbance during sleep (sleep-related breathing disorders) and disorders characterized by abnormal movements during sleep (sleep-related movement disorders).

Insomnia is a sleep disturbance where people have difficulty falling or staying asleep.

People with insomnia have difficulty falling asleep; wake up often during the night and have trouble going back to sleep; wake up too early in the morning; have unrefreshing sleep; and/or have at least one daytime problem such as fatigue, sleepiness, problems with mood, concentration, accidents at work or while driving, etc. due to poor sleep.

Hypersomnia is characterized by excessive daytime sleepiness, and/or prolonged nighttime sleep. Sleep drunkenness is also a symptom found in hypersomnia patients. It is a difficulty transitioning from sleep to wake. Individuals experiencing sleep drunkenness report waking with confusion, disorientation, slowness and repeated returns to sleep.

Circadian rhythm disorders are characterized by chronic or recurring sleep disturbances due to alterations of the individual's internal circadian rhythm or due to misalignments between their circadian rhythm and their desired or required work or social schedule.

This dyssynchrony may be transient or persistent. The ensuing clinical picture combines elements of both insomnia and hypersomnia. Sleep periods are usually shortened and disrupted, performance during the desired waking state is impaired, and temporary opportunities to revert to a regular sleep schedule are unsuccessful.

Parasomnia designates various forms of sleep disturbance characterized by abnormal behavioural or physiological activity (such as sleepwalking or nightmares) that people experience prior to falling asleep, while asleep, or during the arousal period between sleep and wakefulness. There are considerable variations in terms of characteristics, severity, and frequency. Parasomnia may compromise the quality of sleep.

Sleep-related breathing disorders are characterized by abnormal and difficult respiration during sleep. Respiration is a complex process that relies heavily on the coordinated action of the muscles of respiration and the (control center in the) brain. One form of a sleep-related breathing disorder is central sleep apnoea. It occurs when the brain stops sending signals that control breathing, for instance, based on an underlying health condition. Central sleep apnoea has a potentially serious impact on sleep and the balance of oxygen and carbon dioxide in the blood. The reduction of airflow leads to intermittent hypoxia which in leads to sleep fragmentation due to microarousals or awakenings. A consequence can be excessive daytime sleepiness.

In sleep-related movement disorders repetitive, relatively simple, usually stereotyped, movements interfere with sleep or its onset. The most common of these are restless leg syndrome (RLS) and periodic limb movement disorder (PLMD).

Not getting the proper amount or quality of sleep may lead to personality changes and may not only exacerbate existing mental illness, but also be a trigger for the development of mental illness. Sleep disturbance may also interfere with cognitive function and lead to memory impairment. A subject who is deprived of sleep may experience difficulty making decisions, irritability, have problems with performance, and may have slower reaction times. Sleep loss can also adversely affect life by contributing to the development of obesity, diabetes, and heart disease.

Treatment of sleep disorders varies depending on the type and underlying cause.

Maintenance of good sleep hygiene, a healthy sleep environment, and a consistent sleep-wake schedule are often considered as first-line treatment. If not successful, treatment also involves pharmacotherapy or psychotherapy.

Available treatments are not successful in all patients, may be associated with side effects and/or require treatment over a long period of time to achieve a relevant treatment effect.

In patients suffering from sleep disturbance in association with a mental disorder or a nervous system disorder known treatments of the mental or nervous system disorder do not necessarily improve the sleep disturbance.

For instance, sleep disturbance is frequently associated with mental disorders, such as depression. However, treatment of depression does not necessarily lead to an improvement of the concomitant sleep disturbances. While most antidepressants have been proven to influence the sleep architecture, some classes of antidepressants improve sleep, but others may cause sleep impairment.

While sleep disturbance may be considered a condition deserving treatment independent of any other condition, disorder or symptom an individual may suffer from, several mental disorders and nervous system disorders are associated with sleep disturbance.

Notably, the relationship between sleep and a mental or nervous system disorder is often bidirectional. Not only can mental or nervous system disorders have a negative impact on a healthy sleep pattern but sleep disturbance can also be a contributing factor to the onset, progression, and prognosis of mental health or nervous system disorders.

The treatment according to the invention reduces or eliminates sleep disturbance and preferably also improves the associated mental disorder or nervous system disorder.

Measuring Sleep Disturbance

Sleep can be assessed by measuring parameters such as sleep duration, sleep architecture, sleep latency, and the frequency and duration of awakenings throughout the night. The quantitative metrics may be measured using objective methods, including polysomnography, actigraphy, and the determination of sleep latency, or by way of self-reported measures (questionnaires).

Polysomnography is a technique requiring that a patient is monitored overnight at a specialized clinic. A variety of functions are measured throughout the night, including eye movements, brain and muscle activity, respiratory effort and airflow, blood oxygen levels, body positioning and movements, snoring, and heart rate.

Another quantitative measurement is actigraphy. An actimetry sensor is worn to measure motor activity, which is recorded continually and used to assess sleep-wake cycles. This technique allows the patient to continue normal routines while the required data are being recorded in natural sleep environment.

Sleep latency can be measured by the multiple sleep latency test (MSLT). This test provides an objective measure to determine how long it takes a person to fall asleep across a multiplicity of test naps. An average sleep latency of approximately 10 minutes is considered to be normal; less than eight minutes is indicative of sleep disturbance (excessive daytime sleepiness). Accompanying analysis of brain activity can assist in the further diagnosis of the sleep disturbance.

Sleep-rating questionnaires capture ratings of components of sleep quality, such as perceptions of sleep depth, rousing difficulties, and restfulness after sleep, in addition to other factors that could affect sleep quality, such as comorbid conditions and medication use. The evaluation of the qualitative aspects of sleep experience is important, as sleep complaints can often persist despite normal values for quantitative measures of sleep.

Questionnaires not only facilitate a quick and accurate assessment of a complex clinical problem, but they are potentially also helpful for tracking a patient's progress.

Various sleep quality indexes are known. The following indexes include examples of questionnaires to assess sleep in general and questionnaires to assess in particular insomnia, hypersomnia, circadian rhythm disorders and parasomnia, respectively. The invention is, however, not limited to the use of a particular index or questionnaire.

Some questionnaires rely on recall periods (recall windows) of several days or even weeks. While this may be appropriate for diagnosing sleep disturbances, it is not always appropriate for assessing treatment effects, in particular rapid onset of effect after treatment. For several of the questionnaires the recall period can be modified so that the scores obtained reflect a period after treatment. Questionnaires specifically discussed herein to assess effects of a treatment on sleep in patients suffering from specific conditions rely on a recall period that does not start earlier than the time point when acute psychedelic experiences have subsided after the last administration. To meet this criterion, the normally applied recall period is modified if necessary.

Sleep quality in general can be assessed, for instance, with the Sleep-50 questionnaire.

The SLEEP-50 questionnaire consists of 50 items designed to screen for a variety of sleep disorders in the general population. The scale consists of nine subscales, reflecting some of the most common disorders and complaints related to sleep and the factors required for diagnosis such as sleep apnoea, insomnia, narcolepsy, restless legs/periodic leg movement disorder, circadian rhythm sleep disorder, sleepwalking, nightmares, factors influencing sleep, and the impact of sleep complaints on daily functioning. For each item, respondents are provided with a scale ranging from 1 (“not at all”) to 4 (“very much”) and are asked to indicate the extent to which the statement has matched their experience over the previous month or another appropriate recall window.

For diagnosing a sleep disorder not only the specific subscale (e.g., insomnia) must exceed a certain cut-off point, but respondents must also meet a cut-off of at least 3 or 4 (“rather much” or “very much”, respectively) on the subscale evaluating the impact of sleep complaints on daily functioning (Spoormaker et al., Initial validation of the SLEEP-50 questionnaire. Behav Sleep Med. 2005; 3(4):227-46).

Treatment success is indicated (i) by a decrease of the score, preferably (ii) by a decrease to below the cut-off value.

A common questionnaire assessing sleep disturbance is the Pittsburgh Sleep Quality Index. Other instruments are the insomnia severity index, the Espie sleep disturbance questionnaire and the Patient Reported Outcomes Measurement Information System (PROMIS®) Sleep Disturbance.

The Pittsburgh Sleep Quality Index (PSQI) assesses overall sleep quality and disturbances. The PSQI is a self-rated questionnaire comprising 19 questions. Respondents are asked to indicate how frequently they have experienced certain sleep difficulties over the past month or another appropriate recall window.

The 19 self-rated questions assess a wide variety of factors relating to sleep quality, including estimates of sleep duration and latency and of the frequency and severity of specific sleep-related problems. These 19 items are grouped into seven component scores: (1) subjective sleep quality; (2) sleep latency; (3) sleep duration; (4) habitual sleep efficiency; (5) sleep disturbances; (6) use of sleeping medication; (7) daytime dysfunction.

Each component is assigned a score of 0 to 3. Higher scores indicate more acute sleep disturbances. Detailed Scoring Instructions for the Pittsburgh Sleep Quality Index can be found in the Appendix of Buysse et al. The Pittsburgh Sleep Quality Index: a new instrument for psychiatric practice and research. Psychiatry Res. 1989 May; 28(2):193-213.

The seven component scores are then summed to yield one global score, with a range of 0-21 points, “0” indicating no difficulty and “21” indicating severe difficulties in all areas. A global score cut-off of 5 distinguishes poor from good sleepers. A global score >5 indicates that a patient is having severe difficulties in at least two areas, or moderate difficulties in more than three areas.

If treatment outcome is assessed using the PSQI, treatment success is indicated (i) by a decrease of the score, preferably (ii) by a decrease to 5 or below.

The insomnia severity index (ISI) is a short questionnaire relating to subjective sleep quality, severity of symptoms, subjective satisfaction with sleep, the degree to which insomnia interferes with daily functioning, how noticeable the respondent feels his or her insomnia is compared to others, and the overall level of distress created by the sleep problem. Individual responses can be scored from 0 (=none) to 4 (=very); a higher total score corresponds to more severe insomnia. A total score of 0-7 indicates “no clinically significant insomnia,” 8-14 means “subthreshold insomnia,” 15-21 is “clinical insomnia (moderate severity),” and 22-28 means “clinical insomnia (severe)” (A. Shahid et al. (eds.), STOP, THAT and One Hundred Other Sleep Scales, Springer Science+Business Media, LLC 2012; The recall window is two weeks. Another appropriate recall window can also be used.

Treatment success is indicated (i) by a decrease of the score, for instance, by >7 points, in particular >8 point; preferably (ii) by a decrease to below the cut-off value for clinically significant insomnia.

The Espie sleep disturbance questionnaire (SDQ) evaluates subjective experiences of insomnia. With ratings on restlessness/agitation, mental overactivity, consequences of insomnia, and lack of sleep readiness, the SDQ is concerned specifically with beliefs about the sources of sleep issues. Respondents use a five-point scale to indicate how often certain statements about insomnia are representative of their experience. 1 means “never true,” while 5 means “very often true.” Higher scores are indicative of more dysfunctional beliefs about the causes and correlates of insomnia (A. Shahid et al., loc. cit.;).

Treatment success is indicated by a decrease of the score.

The Patient-Reported Outcomes Information System (PROMIS)® Sleep Disturbance instrument is a universal measure to evaluate sleep disturbances. The instrument is available as a long form and 4 different short forms (e.g., 4-, 6-, and 8-items) and assesses self-reported perceptions of sleep quality, sleep depth, and any perceived difficulties related to getting and staying asleep over a 7-day period.

Each item on the measure is rated on a 5-point scale. The raw scores on the items are summed to obtain a total raw score. Total raw scores are then converted into a standardized T-score using conversion tables.

Treatment success is indicated by a decrease of the T-score.

Hypersomnia or hypersomnolence can be assessed by the Epworth Sleepiness Scale, the Stanford Sleepiness Scale, or the Idiopathic Hypersomnia Severity scale.

The Epworth Sleepiness Scale (ESS) evaluates overall daytime sleepiness. The questionnaire asks respondents to rate how likely they are to fall asleep in eight different situations representing a moment of relative inactivity, such as a nap in the afternoon or sitting in a car stopped in traffic. Using a scale of 0-3 (with 0 meaning “would never doze” and 3 meaning “high chance of dozing”), respondents rate their likelihood of falling asleep. Scoring ranges from 0-24; the higher the score, the higher the severity of daytime sleepiness. A cut-off score of 10 identifies daytime sleepiness at a potentially clinical level (A. Shahid et al., loc. cit.).

Treatment success is indicated (i) by a decrease of the score, preferably (ii) by a decrease to 10 or below.

The Stanford Sleepiness Scale is a subjective measure of sleepiness, evaluating sleepiness at specific moments in time. Consisting of only one item, the scale requires respondents to select one of seven statements best representing their current level of perceived sleepiness. A scale from 1 (=Feeling active and vital; alert; wide awake) to 7 (=Almost in reverie; sleep onset soon; lost struggle to remain awake) is used to assess the level of sleepiness (A. Shahid et al., loc. cit.;).

Treatment success is indicated by a decrease of the score.

Parasomnias can be evaluated by the Paris Arousal Disorders Severity Scale (PADSS).

The Paris Arousal Disorders Severity Scale (PADSS) is a self-rating scale listing parasomniac behaviours, assessing their frequency and includes an evaluation of consequences (Arnulf et al., A scale for assessing the severity of arousal disorders. Sleep. 2014 Jan. 1; 37(1):127-36).

Treatment success is indicated (i) by a decrease of the score, preferably (ii) by a decrease to below the cut-off value.

A common questionnaire that assesses sleep-related breathing disorders is the Berlin Questionnaire (A. Shahid et al., loc. cit.;). An appropriate recall period can also be chosen.

Treatment success is indicated by a decrease of the score.

A common questionnaire that assesses sleep-related movement disorders is the International Restless Legs Syndrome Study Group Rating Scale. The 10-item questionnaire asks respondents to use Likert-type ratings to indicate how acutely the disorder has affected them over the course of the past week. Questions can be divided into one of two categories: disorder symptoms (nature, intensity, and frequency) and their impact (sleep issues, disturbances in daily functioning, and resultant changes in mood. Each of the ten questions requires respondents to rate their experiences with RLS on a scale from 0 to 4, with 4 representing the most severe and frequent symptoms and 0 representing the least. Total scores can range from 0 to 40. As a brief scale with excellent psychometric qualities, the instrument may be suitable for a variety of research and clinical purposes, including screening and assessment of treatment outcomes. (A. Shahid et al., loc. cit.).

Treatment response can be assessed by a decrease of the score.

Resting State Networks and Sleep Disturbance Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly based on spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system.

RSNs have been shown to be responsible for various aspects of complex brain function, and it has been found that these connectivity networks are compromised in various disease states. Such disease states are associated with altered functional connectivity within a specific resting state network and/or between one or more regions in one or more additional resting state networks.

Altered resting state networks can be found in insomnia, hypersomnia, circadian rhythm disorders, parasomnias, sleep-related breathing disorders and sleep-related movement disorders.

A key network involved in sleep is the default mode network (DMN). Generally, the DMN is deactivated during tasks and activated at rest. It is involved in multiple cognitive processes such as higher cognition, emotion, and interoception. During sleep, its overall activity level decreases. Given the importance of the DMN for sleep physiology, altered activity of the DMN is of particular relevance in the context of sleep disturbance.

Compromised resting state networks can also be found in mental disorders or nervous system disorders as further discussed herein.

Resting state networks involved in sleep disturbance are also affected by mental or nervous system conditions which are a consequence of certain medical health conditions.

In insomnia patients, dysfunctional connectivity is observed within the default mode network (DMN) and within the salience network, which is implicated in the detection and integration of emotional and sensory stimuli. Studies have suggested that these networks contain critical regions integrating emotional and bodily states, and the dysfunctional connectivity within and/or between these networks and other brain areas may underlie the vigilance, subjective distress, and poor sleep continuity of patients.

In hypersomnia patients, the default mode network is affected. For instance, in idiopathic hypersomnia, distinct DMN hubs—the precuneus and medial prefrontal cortex—demonstrate significant changes, and functional connectivity in the DMN correlates with self-reported sleepiness severity.

A study investigating differences between night shift nurses and day work nurses revealed that dysrhythmia of circadian rhythms contributes to resting-state functional changes in the cerebellum, involved in sleep regulation, and cognitive functions such as responsiveness and alertness. Moreover, the functional connectivity of the DMN is fundamentally different in early and late circadian phenotypes. Like other forms of sleep disturbance, circadian rhythm disorders can lead to changes in brain functional connectivity. Changes in resting state brain functional connectivity have been reported in various diseases with circadian rhythm disorders.

While functional brain imaging is technically difficult to perform during a parasomnia event, differences in the precuneus have been observed in disorders of arousal representing a non-REM parasomnia.

The precuneus is involved in the analysis and integration of visual, audio, and somesthetic information and the monitoring of movements. The precuneus is a subregion of the DMN. Thus, in patients with parasomnias the default mode network is affected.

Resting-state fMRI studies in patients suffering from sleep-related breathing disorders, such as central sleep apnoea, indicate significant global and regional connectivity deficits, especially in the default mode network (DMN) and regions involved in the arousal and sensorimotor systems.

Sleep-related movement disorders, such as for example periodic limb movements during sleep are reflected by alterations in the prefrontal motor control pathway, a subregion of the default mode network. Also, activity in the cerebellum and thalamus, with additional activation in the red nuclei and brainstem can be observed.

In many instances, aberrant functional connectivity of resting state networks involved in sleep disturbance are also involved in the conditions listed above. Thus, according to the invention, influencing those networks by a therapy according to the invention will lead to an improvement of the sleep disturbance and, if the patient treated suffers from a mental disorder or a nervous system disorder, also of that disorder.

Treatment of Sleep Disturbance and Mental and Nervous System Disorders

According to the invention, idiopathic sleep disturbance as well as sleep disturbance in patients suffering from mental disorders or nervous system disorders can be treated. In patients suffering from sleep disturbance in association with a mental disorder or a nervous system disorder a treatment of sleep disturbance according to the invention leads to an improvement of the condition with which the sleep disturbance is associated.

In many instances, resting state networks involved in sleep disturbance are also involved in the conditions listed above.

5-MeO-DMT has the ability to disrupt established functional connectivity patterns of resting state networks. This disruption leads to a reset of the pathological ill-connected brain connections as the networks reconnect. New, healthy functional connections are established with persistent effects.

The persistence of the effects can be explained by the neuroplasticity-promoting characteristics of 5-MeO-DMT. 5-MeO-DMT in particular fosters the structural and functional plasticity of synapses, i.e., of sites where neurons connect and communicate with each other. 5-MeO-DMT modulates morphogenesis and maturation of dendritic spines so that the formation of new synaptic connections is initiated. These new connections will be strengthened or weakened or even be eliminated, dependent on activity.

New synapses ultimately formed will in turn influence the activity patterns of the neurons. The inventors conclude that such reciprocal structural and functional modifications contribute to a proper establishment of networks and the persistence of effects after administration of 5-MeO-DMT.

From a biochemical perspective, 5-MeO-DMT interacts, among others, with 5-HT receptors. 5-HT receptors, receptors for the neurotransmitter serotonin or 5-hydroxytryptamine (5-HT), are found throughout the central and the peripheral nervous system. A wide range of physiological and pathological functions are mediated via these receptors.

In the brain, seven types of 5-HT receptors which can be further separated into several subtypes are expressed. The various types and subtypes show distinct spatial distributions. 5-MeO-DMT interacts with several of the 5-HT receptors. These receptors are involved in mediating effects of 5-MeO-DMT on resting state networks and neuronal plasticity.

Besides 5-HT1A and 5-HT2A discussed above, 5-MeO-DMT also interacts with the 5-HT7 receptor. 5-MeO-DMT act as an agonist on this receptor and shows a high (nanomolar) binding affinity.

The 5-HT7 receptor has a role in neurogenesis, synaptogenesis and dendritic spine formation. It is, among other things, associated with central processes such as learning and memory, with sleep regulation and circadian rhythm and with nociception.

The 5-HT7 receptor is in particular expressed in the spinal cord, raphe nuclei, thalamus, hypothalamus including the suprachiasmatic nucleus, hippocampus, prefrontal cortex, striatal complex, amygdala and in the Purkinje neurons of the cerebellum.

The suprachiasmatic nucleus is the central pacemaker of the circadian timing system. It coordinates circadian rhythms in various brain regions. Disruption of this coordination will result in disease states, in particular disease states involving sleep disturbance. In patients suffering from sleep disturbance resting state functional connectivity analysis reveals alterations in functional connectivity between the suprachiasmatic nucleus and regions within the default mode network.

The expression of the 5-HT7 receptor in the suprachiasmatic nucleus corresponds to the function of the receptor in regulation of sleep/wake cycles. The inventors consider that this allows treatment of patients suffering from sleep disturbance by 5-MeO-DMT which acts on the receptor.

The inventors consider that binding of 5-MeO-DMT to the 5-HT7 receptor as one mediator of the pharmacological effects of 5-MeO-DMT, which involve functional connectivity “resets” of networks and neuroplasticity effects, contributes to the beneficial effects of 5-MeO-DMT in the treatment of patients suffering from sleep disturbance.

The inventors further consider that binding of 5-MeO-DMT to the 5-HT7 receptor as well as to the 5-HT1A receptor as two mediators of effects exerted by 5-MeO-DMT, which include functional connectivity “resets” of networks and neuroplasticity effects, allows achieving beneficial effects also in patients suffering from other symptoms or conditions, such as cognitive dysfunction, anxiety, psychomotor retardation, negative thinking or social/emotional withdrawal. This is supported by the clinical results demonstrated in studies referred to herein.

A treatment according to the invention will lead to an improvement of the sleep disturbance and, if the patient treated suffers from a mental disorder or a nervous system disorder, also of that disorder.

Clinical data from studies of patients suffering from Treatment Resistant Depression (TRD) or Postpartum Depression (PPD) confirm that sleep disturbance can successfully be treated by the administration of 5-MeO-DMT.

In the TRD studies, which are described in more detail in the example section below, among others the MADRS item “reduced sleep”, which reflects insomnia, was assessed.

The MADRS item “reduced sleep” represents the experience of reduced duration or depth of sleep compared to the subject's own normal pattern when well. A score of 0 is assigned when the subject sleeps as usual. A score of 2 reflects slight difficulty dropping off to sleep or slightly reduced, light or fitful sleep. A score of 4 means that sleep is reduced or broken by at least two hours. A score of 6 means less than two or three hours sleep.

In the study group receiving the individualized dosing regimen, the aggregated score for the MADRS item “reduced sleep” across all 8 patients was 25 at base line. At day 1 after treatment, the earliest timepoint for assessing an impact of the treatment on sleep, it was reduced to 12 which corresponds to an improvement of 13 points or 52%. At day 7 after treatment, it was reduced to 9 which corresponds to an improvement of 16 points or 64%.

In the 12 mg group, the aggregated score for the MADRS item “reduced sleep” across all 4 patients was 12 at base line. At day 1 after treatment, it was reduced to 10 which corresponds to an improvement of 2 points or 17%. At day 7 after treatment, it was reduced to 6 which corresponds to an improvement of 6 points or 50%.

Thus, the score of the scale item that is of particular relevance to sleep disturbance, “reduced sleep”, is markedly improved. The inventors conclude that 5-MeO-DMT can be used to treat sleep disturbance, in particular patients suffering from a mental disorder or a nervous system disorder.

Cognitive Dysfunction Cognition includes the skills needed for thinking, remembering, paying attention, and solving problems. Loss or decline of these skills leads to cognitive dysfunction, a term used herein to refer to a deficit in, or an impairment of, any domain of cognition. Cognitive dysfunction may be one of the manifestations of a patient's underlying condition.

The DSM-5 defines six key domains of cognitive function, namely complex attention, executive function, learning and memory, language, perceptual-motor function, and social cognition.

Cognitive dysfunction can impact one or more of those domains. In fact, cognitive abilities are highly interrelated, and it is not unusual that more than one domain is affected.

For instance, the domain complex attention has the subdomains sustained attention (commonly referred to as ‘concentration’ or ‘focus’), divided attention, selective attention, and processing speed.

Thus, complex attention evidently encompasses aspects which are critical for a variety of cognitive tasks, such as executive function and learning and memory. Cognitive control or executive function is intrinsically attentional. Also, perception, and decision-making are profoundly influenced by attention abilities.

As a consequence, attention is not only tested for in isolation, but for example, also tested by cognitive control tasks/executive function. If attention is impaired, other types of cognitive abilities will likely also be impaired. Before language can be comprehended, visualspatial relationships perceived, information remembered or problems solved, the stimuli must be attended to.

Cognitive dysfunction, which term herein means an acquired condition and thus represents a decline from a previously attained level of functioning, can be associated with various processes.

In a healthy individual, certain cognitive abilities, such as accumulated knowledge and vocabulary, are maintained upon ageing and can even improve over time. However, even in the absence of any pathological condition, ageing leads to declines in abilities like thinking abstractly, reasoning, and decision-making. These deteriorations are linked to underlying age-related deficits in processing speed, attention, memory, and executive function, which are indicative of cognitive ageing.

Independent of normal ageing, cognitive dysfunction can be associated with a mental disorder or a nervous system disorder or some other medical conditions.

Mental or nervous system disorders as discussed herein lead to, or are associated with, cognitive dysfunction.

Cognitive dysfunction furthermore occurs in disorders showing symptoms characteristic of a neurocognitive disorder that cause clinically significant distress or impairment in social, occupational, or other important areas of functioning but do not meet the full criteria for any aetiology-related disorder.

Cognitive dysfunction may take the form of a neurocognitive disorder.

Mild neurocognitive disorder, also referred to as mild cognitive impairment, is characterized by a modest cognitive decline from a previous level of performance in one or more of the cognitive domains. Affected patients are still able to stay independent and do daily tasks. However, the patient usually functions at a suboptimal level. Everyday tasks become more effortful owing to the engagement of compensatory strategies to maintain independence.

In major neurocognitive disorder, a significant cognitive decline from a previous level of performance in one or more of the cognitive domains is observed. The cognitive deficits interfere with independence in everyday activities.

Measuring Cognitive Dysfunction Cognitive dysfunction can be evaluated by questionnaires or by neuropsychological assessments.

Questionnaires assess the mental status of a patient based on observations made by the patient himself, caregivers or the clinician administering the questionnaire. Questionnaires used to assess whether a patient suffers from a particular mental or nervous system disorder may comprise items related to cognitive function.

A neuropsychological assessment is a process by which a person's cognitive, psychological/emotional and behavioural functioning is comprehensively evaluated. A core part of neuropsychological assessment is the administration of neuropsychological tests for the formal assessment of cognitive function.

Performance in these tests is compared with norms appropriate to the patient's age, educational attainment, and cultural background. Testing often uses a set of performance-based questions, also known as a neuropsychological test battery.

The abilities tested include language processing, visuospatial processing, attention/concentration, verbal learning and memory, visual learning and memory, executive functions, speed of processing, and sensory-perceptual functions.

Common tests that assess cognitive dysfunction are the Montreal Cognitive Assessment (MoCA), the Mini-Mental State Examination (MMSE), the Mini-Cog™, the Screen for Cognitive Impairment in Psychiatry (SCIP), and the MATRICS Consensus Cognitive Battery (MCCB).

The Montreal Cognitive Assessment (MoCA) is a widely used screening assessment for detecting cognitive impairment. It assesses different cognitive domains: short-term memory; visuospatial abilities; executive functions; attention, concentration and working memory; language; orientation to time and space. The total possible score is 30 points; a score of 26 or above is considered normal; a score of 18-25 is considered mild cognitive impairment, a score of 10-17 is considered moderate cognitive impairment and a score less than 10 is considered severe cognitive impairment.

The Mini-Mental State Examination (MMSE) is an 11-question measure that tests five areas of cognitive function: orientation, registration, attention and calculation, recall, and language. The maximum score is 30. The raw score may also need to be corrected for educational attainment and age.

Four cut-off levels are employed herein to classify the severity of cognitive impairment: 24-30 means no cognitive impairment; 19-23 means mild cognitive impairment; 10-18 means moderate cognitive impairment; and 59 means severe cognitive impairment.

Used repeatedly, the MMSE is suitable to measure changes in cognitive status.

The Mini-Cog™ is a short cognitive impairment screening questionnaire. It combines a 3-word recall with a clock drawing test. The clock drawing test assesses many cognitive areas that can be affected, such as executive function, visuospatial abilities, motor programming, and attention. One point is given for each of the three words correctly recalled after performing the clock drawing test; a correctly drawn clock is worth two points. A score of <4 indicates cognitive impairment.

The Screen for Cognitive Impairment in Psychiatry (SCIP) is a well-evaluated screening instrument for the examination of cognitive performance in psychiatric patients.

The SCIP consists of five subscales: verbal learning test-immediate (VLT-1), working memory test (WMT), verbal fluency test (VFT), verbal learning test-delayed (VLT-D) and processing speed test (PST). There are three different test forms to facilitate test repetition and therefore reducing learning effect. Subscale scores are calculated for each of the five tests, and a total score is calculated from the sum of the subscale scores. A total score of less than 70 indicates cognitive dysfunction.

Cognitive dysfunction can also be assessed by the MCCB (MATRICS Consensus Cognitive Battery) or by one or more of the various subtests. The subtests are: Trail Making Test, Part A (testing speed of processing); Brief Assessment of Cognition in Schizophrenia, symbol coding subtest (speed of processing); Hopkins Verbal Learning Test-Revised, immediate recall, three learning trials only (verbal learning); Wechsler Memory Scale, 3rd ed., spatial span subtest (working memory (nonverbal)); Letter-Number Span test (working memory (verbal)); Neuropsychological Assessment Battery, mazes subtest (reasoning and problem solving); Brief Visuospatial Memory Test-Revised (visual learning); Category fluency test, animal naming (speed of processing); Mayer-Salovey-Caruso Emotional Intelligence Test, managing emotions branch (social cognition); and Continuous Performance Test, Identical Pairs version (attention/vigilance).

The test battery is appropriate to measure cognitive change.

Further tests are the Verbal Recognition Memory (VRM) test, the Rapid Visual information Processing (RVP) test, the Spatial Working Memory (SWM) test and the Digit Symbol Substitution Test (DSST).

Mechanisms Underlying Cognitive Dysfunction Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly based on spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system.

RSNs have been shown to be responsible for various aspects of complex brain function, and it has been found that these connectivity networks are compromised in various disease states. Such disease states, which include certain forms of cognitive dysfunction, are associated with altered functional connectivity within a specific resting state network and/or between one or more regions in one or more additional resting state networks.

Resting state fMRI is particularly advantageous when studying populations affected by cognitive dysfunction because it allows for the examination of functional connectivity while removing the demand of a task that may be confounded by potential cognitive or motor impairments.

Cognitive processes are reflected by functional connectivity of certain brain regions within and/or between regions located in different networks.

In particular, certain core networks, also referred to as “higher-order cognitive networks” appear to be crucial for most mental activities.

The frontoparietal control network (FPCN), also referred to as frontoparietal network (FPN), central executive network (CEN) or executive network (EN), is typically associated with executive functions. These functions include keeping and updating relevant information in working memory, inhibiting impulsive responses, and using flexible problem-solving strategies to guide decisions and goal-directed behaviour.

Another core network is the default mode network (DMN). The DMN contains regions in the brain that are most active when the person's attention is not directed to any specific task. The activity of the DMN is related to introspection, episodic memory, memory consolidation, social and self-related cognition, integration of cognitive and emotional processing, and task-unrelated free thoughts of mind wandering.

A third network is the salience network, also referred to as cingulo-opercular network. This network is involved in identifying salient stimuli and events, that is, what other brain networks need to attend to. This network has a central role in governing mental processes and behaviour.

A fourth network is the dorsal attention network (DAN). The DAN is associated with topdown, goal-directed attention processes.

The above networks do not operate independently. In fact, there are numerous connections between them. Cooperation between the networks is crucial for task-specific functions.

Over the course of the lifespan, brain networks undergo functional reorganization with concurrent implications for cognition. In healthy aging, age-related alterations are observed in higher-order cognitive networks.

Patients suffering from cognitive dysfunction show altered functional connectivity within and/or between resting state networks when compared to healthy, age-matched controls. Alterations are observed within and/or between the default mode network, the executive network, the salience network, and the dorsal attention network.

In many instances, resting state networks involved in cognition are affected by mental or nervous system disorders as discussed herein.

Resting state networks involved in cognition are also affected by mental or nervous system conditions which are a consequence of certain medical health conditions as well as in unspecified neurocognitive disorders.

Further, resting state networks involved in cognition are affected by sleep disturbance, for instance, insomnia. In fact, cognitive dysfunction and impairment of sleep are correlated.

Cognitive function is deteriorated in patients suffering from sleep disturbance, and patients suffering from cognitive dysfunction often also suffer from impaired sleep.

Treatment of Cognitive Dysfunction

According to the invention, cognitive dysfunction occurring in a patient suffering from a mental disorder or a nervous system disorder, or a medical health condition leading to an associated mental or nervous system condition, can be treated. Moreover, cognitive dysfunction occurring in a patient suffering from sleep disturbance, for instance, insomnia can be treated.

Cognitive dysfunction in unspecified neurocognitive disorders can likewise be treated.

In patients suffering from cognitive dysfunction in association with another condition as detailed above, a treatment of cognitive dysfunction according to the invention leads to an improvement of the condition with which the cognitive dysfunction is associated.

Treatment according to the invention is by administration of 5-MeO-DMT or a pharmaceutically acceptable salt thereof. 5-MeO-DMT administered to a patient disrupts established functional connectivity patterns within and/or between resting state networks. This disruption leads to a reset of the pathological ill-connected connections as the networks reconnect. New, healthy functional connections are established with persistent effects.

Thus, according to the invention, influencing those networks by a therapy as described herein will lead to an improvement of the cognitive dysfunction and, if the patient treated suffers from a mental disorder or a nervous system disorder, also of that disorder; if the patient treated suffers from a medical health condition leading to an associated mental or nervous system condition, also of the associated mental or nervous system condition; if the patient treated suffers from sleep disturbance, for instance, insomnia, also of the sleep disturbance, for instance, insomnia; if the patient suffers from an unspecified neurocognitive disorder, also of one or more other symptoms of that disorder.

To further support the clinical application of 5-MeO-DMT in patients suffering from cognitive dysfunction, the inventors assessed clinical data relating to the use of 5-MeO-DMT in patients treated because of mental disease and noted particular improvements in cognitive dysfunction which is typically also observed in patients with other disorders.

The data stem from a recently completed clinical trial investigating the use of 5-MeO-DMT in the treatment of patients diagnosed with Treatment Resistant Depression (TRD; see also the examples section below). While TRD is a specific condition, the inventors determined, as discussed in detail below, that certain clinical observations are made in the trial are relevant for devising a treatment for other conditions which are associated with cognitive dysfunction.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Asberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed that particular subscore items, like items related to cognitive dysfunction, are relevant for other conditions in which cognitive dysfunction is based on similarly altered functional connectivity within and/or between the default mode network, the executive control network, the salience network, and the dorsal attention network.

Multiple patients within the recruited cohort displayed significant improvements, a result that confirms the inventors' finding that 5-MeO-DMT is a compound suitable for treating patients presenting with these symptoms.

More in particular, an aspect which can be treated by administration of 5-MeO-DMT, is cognitive dysfunction, in particular concentration difficulties. 5-MeO-DMT can be administered to patients to reduce or eliminate cognitive dysfunction, in particular concentration difficulties, in said patients.

The MADRS item that is of particular relevance to impaired concentration and memory is “concentration difficulties”. This item represents difficulties in collecting one's thoughts mounting to incapacitating lack of concentration and is scored on a scale ranging from 0 to 6. The score is 0 if the patient has no difficulties in concentrating. The score is 2 in case of occasional difficulties in collecting one's thoughts. A score of 4 is assigned in case of difficulties in concentrating and sustaining thought which reduces ability to read or hold a conversation. The score is 6 if the patient is unable to read or converse without great difficulty.

In the study group receiving the individualized dosing regimen, the aggregated score for the MADRS item “concentration difficulties” across all 8 patients was 30 at base line.

After 2 hours, it was reduced to 11 which corresponds to an improvement of 19 points or 63%. At day 1 after treatment, it was reduced to 1 which corresponds to an improvement of 29 points or 97%. At day 7 after treatment, it was reduced to 9 which corresponds to an improvement of 21 points or 70%.

In the 12 mg group, the aggregated score for the MADRS item “concentration difficulties” across all 4 patients was 16 at base line.

After 2 hours, it was reduced to 7 which corresponds to an improvement of 9 points or 56%. At day 1 after treatment, it was reduced to 2 which corresponds to an improvement of 14 points or 88%. At day 7 after treatment, it was reduced to 3 which corresponds to an improvement of 13 points or 81%.

Consequently, according to the invention, the treatment of a patient suffering from cognitive dysfunction with 5-MeO-DMT or a pharmaceutically acceptable salt thereof reduces or eliminates cognitive dysfunction.

More in particular, according to the invention, the treatment of a patient with 5-MeO-DMT or a pharmaceutically acceptable salt thereof, wherein the patient suffers from cognitive dysfunction, which is a deficit in, or an impairment of, one or more cognitive domains selected from complex attention, executive function, learning and memory, language, perceptual-motor function, and social cognition, reduces or eliminates the cognitive dysfunction. For instance, the cognitive dysfunction is reduced or eliminated if it affects the cognitive domain complex attention, such as one or more subdomains of the cognitive domain complex attention selected from sustained attention, divided attention, selective attention, and processing speed, especially sustained attention.

In patients suffering from cognitive dysfunction in association with a mental disorder or a nervous system disorder a treatment of cognitive dysfunction according to the invention leads to an improvement of the condition with which the cognitive dysfunction is associated.

While cognitive dysfunction may be considered a condition deserving treatment independent of any other conditions, disorders or symptoms an individual may suffer from, several mental disorders and disorders of the nervous system are associated with cognitive dysfunction. Notably, the relationship between cognitive dysfunction and mental disorder is bidirectional. Not only can mental disorders have a negative impact on cognitive function, but cognitive dysfunction can also be a contributing factor to the onset, progression and prognosis of mental or nervous system disorders.

In many instances, resting state networks involved in cognitive dysfunction are also involved in the conditions listed above. 5-MeO-DMT has the ability to disrupt established functional connectivity patterns of resting state networks. This disruption leads to a reset of the pathological ill-connected brain connections as the networks reconnect. New, healthy functional connections are established with persistent effects.

Anxiety

Anxiety is sometimes defined as an “apprehensive anticipation of future danger or misfortune accompanied by a feeling of dysphoria or somatic symptoms of tension”.

Anxiety is characterized by an intense, excessive, and persistent worry and fear about a situation that is only subjectively seen as menacing and is often accompanied by muscular tension, restlessness, fatigue, inability to catch one's breath, tightness in the abdominal region, nausea, and problems in concentration.

In anxiety disorders or other mental or nervous system disorders associated with anxiety, the feelings of anxiety are difficult to control and interfere with daily activities.

Anxiety is a core feature of anxiety disorders, including separation anxiety disorder, specific phobia, social anxiety disorder (social phobia), panic disorder, generalized anxiety disorder (GAD), agoraphobia, and substance/medication-induced anxiety disorder.

Anxiety is moreover associated with several other mental and nervous system disorders. Anxiety is also associated with sleep disturbance.

Measuring Anxiety

Several rating scales to assess anxiety are known on the art, and anxiety symptoms are furthermore assessed as part of various rating scales used to assess mental and nervous system disorders.

The Hamilton Anxiety Rating Scale (HAM-A) is designed to assess anxiety symptoms. The scale is clinician-administered. It has 14 items which can be divided into a group of psychic items (1-6 and 14) measuring in particular mental agitation and psychological distress and into a group of somatic items (items 7-13) measuring in particular physical complaints related to anxiety.

The HAM-A items are shown in the table below.

| | | |
|---|---|---|
| 1 | Anxious mood | Worries, anticipation of the worst, fearful anticipation, |
| | | irritability |
| 2 | Tension | Feelings of tension, fatigability, startle response, moved |
| | | to tears easily, trembling, feelings of restlessness, |
| | | inability to relax |
| 3 | Fears | Of dark, of strangers, of being left alone, of animals, of |
| | | traffic, of crowds |
| 4 | Insomnia | Difficulty in falling asleep, broken sleep, unsatisfying |
| | | sleep and fatigue on waking, dreams, nightmares, night |
| | | terrors |
| 5 | Intellectual | Difficulty in concentration, poor memory |
| 6 | Depressed mood | Loss of interest, lack of pleasure in hobbies, depression, |
| | | early waking, diurnal swing |
| 7 | Somatic (muscular) | Pains and aches, twitching, stiffness, myoclonic jerks, |
| | | grinding of teeth, unsteady voice, increased muscular |
| | | tone |
| 8 | Somatic (sensory) | Tinnitus, blurring of vision, hot and cold flushes, feelings |
| | | of weakness, pricking sensation |
| 9 | Cardiovascular | Tachycardia, palpitations, pain in chest, throbbing of |
| | symptoms | vessels, fainting feelings, missing beat |
| 10 | Respiratory symptoms | Pressure or constriction in chest, choking feelings, |
| | | sighing, dyspnea |
| 11 | Gastrointestinal | Difficulty in swallowing, wind abdominal pain, burning |
| | symptoms | sensations, abdominal fullness, nausea, vomiting, |
| | | borborygmi, looseness of bowels, loss of weight, |
| | | constipation |
| 12 | Genitourinary symptoms | Frequency of micturition, urgency of micturition, |
| | | amenorrhea, menorrhagia, development of frigidity, |
| | | premature ejaculation, loss of libido, impotence |
| 13 | Autonomic symptoms | Dry mouth, flushing, pallor, tendency to sweat, giddiness, |
| | | tension headache, raising of hair |
| 14 | Behaviour at interview | Fidgeting, restlessness or pacing, tremor of hands, |
| | | furrowed brow, strained face, sighing or rapid |
| | | respiration, facial pallor, swallowing, etc. |

Each item is rated by the interviewer on a scale from 0 to 4: 0=Not present, 1=Mild, 2=Moderate, 3=Severe, 4=Very severe.

A total score is obtained by summing the 14 items. The total score range is 0-56. Higher scores indicate more anxiety.

A score s 7 is considered to represent no or minimal anxiety; a score of 8-14 mild anxiety; a score of 15-23 moderate anxiety; a score 24 severe anxiety.

The Beck Anxiety Inventory (BAI) is a 21-item self-report questionnaire developed to assess anxiety, with a focus on somatic symptoms. The items are rated on a four-point Likert scale ranging from zero (not at all) to three (severely: I could barely stand it). The total score ranges from 0 to 63.

Subthreshold anxiety as the term is used herein in particular means that the patient has a Hamilton Rating Scale for Anxiety (HAM-A) score of at least 9 but of less than 18 and/or a Beck Anxiety Inventory (BAI) score of at least 11 but of less than 16.

Mechanisms Underlying Anxiety

Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly based on spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system.

RSNs have been shown to be responsible for various aspects of complex brain function, and it has been found that these connectivity networks are compromised in various disease states. Such disease states, which include certain forms of anxiety, are associated with altered functional connectivity within a specific resting state network and/or between one or more regions in one or more additional resting state networks.

Based on such studies, a number of brain regions have been implicated in anxiety and anxiety disorders. Accordingly, anxiety and anxiety disorder pathophysiology involves aberrant connectivity between amygdala-frontal and frontal-striatal regions. Anxiety and anxiety disorders are associated with specific alterations to resting state networks.

Anxiety and anxiety disorders show abnormalities within and/or between default mode network, salience network and sensorimotor network. The resting state balance within and/or between each of these networks differs in the different anxiety disorders.

Treatment of Anxiety

According to the invention, anxiety occurring in a patient suffering from an anxiety disorder, or another mental disorder or nervous system disorder associated with anxiety, can be treated. Moreover, anxiety occurring in a patient suffering from sleep disturbance, for instance, insomnia can be treated.

In patients suffering from anxiety in association with another mental disorder or nervous system disorder or sleep disturbance, for instance, insomnia, a treatment of anxiety according to the invention leads to an improvement of the condition with which the anxiety is associated.

Treatment according to the invention is by administration of 5-MeO-DMT or a pharmaceutically acceptable salt thereof.

5-MeO-DMT administered to a patient disrupts established functional connectivity patterns within and/or between resting state networks. This disruption leads to a reset of the pathological ill-connected connections as the networks reconnect. New, healthy functional connections are established with persistent effects.

Thus, according to the invention, influencing those networks by a therapy as described herein will lead to an improvement of the anxiety and, if the patient treated suffers from another mental disorder or nervous system disorder associated with anxiety, also of that disorder; if the patient treated suffers from sleep disturbance, for instance, insomnia, also of the sleep disturbance, for instance, insomnia.

To further support the clinical application of 5-MeO-DMT in patients suffering from anxiety, the inventors assessed clinical data relating to the use of 5-MeO-DMT in patients treated because of mental disease and noted particular improvements in anxiety which is typically also observed in patients with other disorders.

The data stem from a recently completed clinical trial investigating the use of 5-MeO-DMT in the treatment of patients diagnosed with Treatment Resistant Depression (TRD; see also the examples section below). While TRD is a specific condition, the inventors determined, as discussed in detail below, that certain clinical observations are made in the trial are relevant for devising a treatment for anxiety disorders and other conditions which are associated with anxiety.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Asberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed that particular subscore items, like items related to anxiety, are relevant for other conditions in which anxiety is based on similarly altered functional connectivity within and/or between resting state networks.

Multiple patients within the recruited cohort displayed significant improvements, a result that confirms the inventors' finding that 5-MeO-DMT is a compound suitable for treating patients presenting with these symptoms.

More in particular, an aspect which can be treated by administration of 5-MeO-DMT, is anxiety. 5-MeO-DMT can be administered to patients to reduce or eliminate anxiety in said patients.

The BPRS item that is of particular relevance in this context is “anxiety”. This item relates to reported apprehension, tension, fear, panic or worry. Possible scores are:
1—No anxiety
2—Very Mild. Reports some discomfort due to worry or infrequent worries that occur more than usual for most normal individuals.
3—Mild. Worried frequently but can readily turn attention to other things.
4—Moderate. Worried most of the time and cannot turn attention to other things easily but no impairment in functioning or occasional anxiety with autonomic accompaniment but no impairment in functioning.
5—Moderately Severe. Frequent, but not daily, periods of anxiety with autonomic accompaniment or some areas of functioning are disrupted by anxiety or worry.
6—Severe. Anxiety with autonomic accompaniment daily but not persisting throughout the day or many areas of functioning are disrupted by anxiety or constant worry.
7—Extremely Severe. Anxiety with autonomic accompaniment persisting throughout the day or most areas of functioning are disrupted by anxiety or constant worry.

In the study group receiving the individualized dosing regimen, the aggregated score for the BPRS item “anxiety” across all 8 patients was 37 at base line.

After 3 hours, it was reduced to 19 which corresponds to an improvement of 18 points or 49%. At day 1 after treatment, it was reduced to 16 which corresponds to an improvement of 21 points or 57%. At day 7 after treatment, it was reduced to 17 which corresponds to an improvement of 20 points or 54%.

In the 12 mg group, the aggregated score for the BPRS item “anxiety” across all 4 patients was 25 at base line.

After 3 hours, it was reduced to 11 which corresponds to an improvement of 14 points or 56%. At day 1 after treatment, it was reduced to 6 which corresponds to an improvement of 19 points or 76%. At day 7 after treatment, it was reduced to 6 which corresponds to an improvement of 19 points or 76%.

The inventors conclude that 5-MeO-DMT can be used to treat anxiety in patients, such as patients suffering from an anxiety disorder and patients suffering from a amental or nervous system disorder with associated anxiety.

Consequently, according to the invention, the treatment of a patient suffering from anxiety with 5-MeO-DMT or a pharmaceutically acceptable salt thereof reduces or eliminates anxiety.

The MADRS item “inner tension” represents feelings of ill-defined discomfort, edginess, inner turmoil, mental tension mounting to either panic, dread or anguish. It is rated according to intensity, frequency, duration and the extent of reassurance called for.

A score of 0 is assigned if the patient is placid and there is only fleeting inner tension. A score of 2 is assigned if there are occasional feelings of edginess and ill defined discomfort. The score is 4 if there are continuous feelings of inner tension or intermittent panic which the patient can only master with some difficulty. The score is 6 in case of unrelenting dread or anguish and overwhelming panic.

In the above indicated trial involving TRD patients, in the study group receiving the individualized dosing regimen, the aggregated score for the MADRS item “inner tension” across all 8 patients was 26 at base line. After 2 hours, it was reduced to 11 which corresponds to an improvement of 15 points or 58%. At day 1 aftertreatment, it was reduced to 6 which corresponds to an improvement of 20 points or 77%. At day 7 after treatment, it was reduced to 12 which corresponds to an improvement of 14 points or 54%.

In the 12 mg group, the aggregated score for the MADRS item “inner tension” across all 4 patients was 13 at base line. After 2 hours, it was reduced to 2 which corresponds to an improvement of 11 points or 85%. At day 1 after treatment, it was reduced to 3 which corresponds to an improvement of 10 points or 77%. At day 7 after treatment, it was reduced to 5 which corresponds to an improvement of 8 points or 62%.

These results further support the inventors' conclusion that a treatment according to the invention reduces or eliminates symptoms of anxiety.

A treatment according to the invention leads to a clinical response in a patient suffering from anxiety symptoms which is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from anxiety symptoms, as reflected by at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from anxiety symptoms, as reflected by at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of the anxiety symptoms in a patient suffering from anxiety symptoms is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of the anxiety symptoms in a patient suffering from anxiety symptoms, as reflected by a HAM-A score of 7 or less, occurs not later than about 2 hours after the last administration of 5-MeO-DMT or a pharmaceutically acceptable salt thereof. The remission of the anxiety symptoms in a patient suffering from anxiety symptoms, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

Consequently, according to the invention, the treatment of a patient suffering from anxiety with 5-MeO-DMT or a pharmaceutically acceptable salt thereof reduces or eliminates anxiety.

Psychomotor Retardation Key aspects observed with patients suffering from psychomotor retardation are reduced energy and activity and reduced motivation.

Psychomotor retardation involves a slowing down of thought and a reduction of physical movements in an individual. Psychomotor impairment can cause a visible slowing of physical and emotional reactions.

Psychomotor retardation can be associated with a mental disorder or a nervous system disorder or some other medical conditions.

Mental or nervous system disorders which lead to, or are associated with, psychomotor retardation include disorders characterized by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson's Disease; Chronic Fatigue Syndrome.

Psychomotor retardation can also occur in a patient suffering from sleep disturbance, for instance, insomnia.

Measuring Psychomotor Retardation

Psychomotor retardation can be assessed by measuring various aspects. These may include for instance various types of drawing tasks and tests, such as the trail making test (TMT), the digit symbol substitution test (DSST), or the Gibson Spiral Maze Test (GSM) and others which are known in the art.

In the trail making test (TMT), for instance, subjects must connect 25 circles that contain either numbers (TMT A) or a combination of numbers and letters (TMT B) in ascending order. Task requirements are similar for TMT-B, except that the subject must alternate between numbers and letters (1, A, 2, B, 3, C and so on). The test thus evaluates processing speed (TMT A) or cognitive flexibility (TMT B). The score for each part represents the amount of time required to complete the task.

Another test that involves graphomotor ability is the Gibson Spiral Maze (GSM) assessing psychomotor speed only, is not influenced by cognitive abilities. Subjects who complete the GSM must correctly trace through a spiral maze from a starting point to an end point without touching bordering lines.

The digit symbol substitution test (DSST) measures also psychomotor speed and consists of digit-symbol pairs followed by a list of digits. Under each digit, the subject should write down the corresponding symbol as fast as possible. The score consists in the number of symbols correctly reported in 90 s. A further example for a motor test is the finger tapping test.

Thus, certain tests combine measurements of both motor and cognitive aspects of psychomotor retardation, while further others assess only motor aspects.

Analysis of speech can be a further indicator of psychomotor retardation.

Major scales available to assess and measure include the severity of psychomotor retardation, the Salpetriere Retardation Rating Scale (SRRS) and the Motor Agitation and Retardation Scale (MARS).

The Salpetriere Retardation Rating Scale (SRRS), developed by Widlocher assesses cognitive and motor aspects by fifteen items. The first three measure movement, specifically the quality of stride and slowness of limb, trunk, head, and neck movement. The next three items focus on speech including verbal flow, tone of voice, and length of response. Two items are designed to objectively measure cognitive function. These questions are based on the interview conversation and measure the patient's ability to approach and expand on topics. The further items are subjective and assess rumination, fatigue, level of interest, perception of time, memory, and concentration. The last item of the scale relates to an overall assessment of the patient's psychomotor retardation. The items are scaled from 0 (symptom absence) to 4 (severe) based on the severity of the presenting symptom, for a total score range of 0 to 60.

The Motor Agitation and Retardation Scale (MARS) assesses motor aspects only. It was designed to assess psychomotor disturbances in depressive disorders. Psychomotor disturbances are divided into five major body categories including eyes, face, voice, limbs, and trunk with a total of 19 items on the scale. Items of the eyes category include direction of gaze, amount of blinking, staring, and eye movement. Items associated with the face category include facial expression and facial expressivity. The category of voice has items that include volume, slurring, tone and time for onset. Items under the limbs category include hand, foot, and leg movement, stride, motor slowness, and tension in hands. The trunk category items include posture, immobility, and axial movement. The severity of each item ranges from a 1 to a 4, with 4 being the most severe. Of the 19 items 9 relate to motor agitation and 10 items assess motor retardation. The retardation items include abnormal gait, immobility of trunk/proximal limbs, postural collapse, motor slowness (i.e. the limb and trunk category); lack of facial expressivity, downcast gaze (i.e. the eyes and face category); and reduced voice volume, slurring of speech, delayed speech onset, monotone speech (i.e. the voice category). The MARS scale offers a rapid clinical assessment of motor signs.

Resting State Networks and Psychomotor Retardation Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly based on spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system RSNs have been shown to be responsible for various aspects of complex brain function, and it has been found that these connectivity networks are compromised in various disease states. Such disease states, which include certain forms of psychomotor retardation, are associated with altered functional connectivity within a specific resting state network and/or between one or more regions in one or more additional resting state networks.

For instance, abnormal functional connectivity was reported from somatosensory motor networks (SMN) to visual (VN), dorsal attention (DAN), and default mode networks which correlated with both psychomotor retardation and agitation in depressive disorders.

In many instances, resting state networks involved in psychomotor retardation are affected by mental disorders or nervous system disorders, characterized by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson Disease; Chronic Fatigue Syndrome.

Resting state networks involved in psychomotor retardation are also affected by sleep disturbance, for instance, insomnia. In fact, psychomotor retardation and impairment of sleep are correlated.

Treatment of Psychomotor Retardation and Mental or Nervous System Disorders According to the invention, psychomotor retardation in patients suffering from a mental disorder or a nervous system disorder can be treated. Moreover, psychomotor retardation occurring in a patient suffering from sleep disturbance, for instance, insomnia can be treated.

In patients suffering from psychomotor retardation in association with another condition as detailed above, a treatment of psychomotor retardation according to the invention leads to an improvement of the condition with which the psychomotor retardation is associated.

Treatment according to the invention is by administration of 5-MeO-DMT or a pharmaceutically acceptable salt thereof. 5-MeO-DMT administered to a patient disrupts established functional connectivity patterns within and/or between resting state networks. This disruption leads to a reset of the pathological ill-connected connections as the networks reconnect. New, healthy functional connections are established with persistent effects.

Thus, according to the invention, influencing those resting state networks by a therapy as described herein will lead to an improvement of psychomotor retardation and, if the patient treated suffers from a mental disorder or a nervous system disorder, also of that disorder; if the patient treated suffers from sleep disturbance, for instance, insomnia, also of the sleep disturbance, for instance, insomnia.

To further support the clinical application of 5-MeO-DMT in patients suffering from psychomotor retardation, the inventors assessed clinical data relating to the use of 5-MeO-DMT in patients treated because of mental disease and noted particular improvements in psychomotor retardation which is typically also observed in patients with other disorders.

The data stem from a recently completed clinical trial investigating the use of 5-MeO-DMT in the treatment of patients diagnosed with Treatment Resistant Depression (TRD; see also the examples section below). While TRD is a specific condition, the inventors determined, as discussed in detail below, that certain clinical observations are made in the trial are relevant for devising a treatment for other conditions which are associated with psychomotor retardation.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Asberg Depression Rating Scale (MADRS). While the focus of the trial was on demonstrating treatment efficacy through improvements in overall MADRS score, the inventors focused on the items comprising the various rating scales and noticed that particular subscore items, like items related to psychomotor retardation, are relevant for other conditions in which psychomotor retardation is based on similarly altered functional connectivity within and/or between the somatomotor/sensorimotor network, the visual network, the dorsal attention network and the default mode networks.

Multiple patients within the recruited cohort displayed significant improvements, a result that confirms the inventors' finding that 5-MeO-DMT is a compound suitable for treating patients presenting with these symptoms.

More in particular, an aspect which can be treated by administration of 5-MeO-DMT, is psychomotor retardation. 5-MeO-DMT can be administered to patients to reduce or eliminate psychomotor retardation in said patients.

The MADRS scale item that is of particular relevance to psychomotor retardation is “lassitude”, which represents a difficulty getting started or slowness initiating and performing everyday activities.

A score of 0 means that there is hardly any difficulty in getting started and no sluggishness. A score of 2 is assigned if the patient has difficulties in starting activities. A score of 4 means difficulties in starting simple routine activities which are carried out with effort.

A score of 6 is assigned in the case of complete lassitude, the patient being unable to do anything without help.

In the study group receiving the individualized dosing regimen, the aggregated score for the MADRS item “lassitude” across all 8 patients was 27 at base line.

After 2 hours, it was reduced to 10 which corresponds to an improvement of 17 points or 63%. At day 1 after treatment, it was reduced to 5 which corresponds to an improvement of 22 points or 81%. At day 7 after treatment, it was reduced to 3 which corresponds to an improvement of 24 points or 89%.

In the 12 mg group, the aggregated score for the MADRS item “lassitude” across all 4 patients was 16 at base line. After 2 hours, it was reduced to 10 which corresponds to an improvement of 6 points or 38%. At day 1 after treatment, it was reduced to 0 which corresponds to an improvement of 16 points or 100%. At day 7 after treatment, it was reduced to 3 which corresponds to an improvement of 13 points or 81%.

Thus, the score of the scale item that is of particular relevance to psychomotor retardation, “lassitude”, is markedly improved. The inventors conclude that 5-MeO-DMT can be used to treat psychomotor retardation in patients, in particular patients also suffering from a mental disorder or a nervous system disorder or a sleep disturbance, for instance insomnia.

Consequently, according to the invention, the treatment of a patient suffering from psychomotor retardation with 5-MeO-DMT or a pharmaceutically acceptable salt thereof reduces or eliminates psychomotor retardation.

Social/Emotional Withdrawal or Detachment

Symptoms such as anhedonia; emotional withdrawal and affective flattening are clustered together here as social/emotional withdrawal or detachment. Reduced social engagement is a further aspect associated with social/emotional withdrawal or detachment.

Anhedonia is the inability to experience pleasure. The patient does not suffer from anhedonia if there is subjectively no reduced ability to experience pleasure in usual activities. Anhedonia is mild in the case of slight reduction in pleasure from usually pleasurable activities; moderate in the case of significant reduction in pleasure from usually pleasurable activities or some pleasure from isolated activities retained; or severe in the case of complete inability to experience pleasure.

Anhedonia comprises consummatory (or liking) and anticipatory (or wanting) components. Consummatory pleasure refers to the “in the moment” pleasure experienced by the subject directly engaged in an enjoyable activity, whereas anticipatory pleasure refers to the experience of pleasure related to future activities.

Affective flattening characterises the subjective sense of reduced intensity or range of feelings or emotions. The subject does not show affective flattening if there is no sense of reduced intensity or range of feeling or emotions. It is mild in the case of slight constriction of range of affect, or transient reduction in range or intensity of feelings; moderate in the case of significant constriction of range or intensity of feelings with preservation of some emotions, e.g., inability to cry; and severe in the case of marked and pervasive constriction of range of affect or inability to experience usual emotions.

Emotional withdrawal or detachment is an inability or unwillingness to connect with other people on an emotional level. For example, the BPRS contains an item relating to emotional withdrawal, which is characterised as the deficiency in the subject's ability to relate emotionally during the interview situation. According to the description of this BPRS item, there is no emotional withdrawal if there is no lack of emotional involvement shown by occasional failure to make reciprocal comments, occasionally appearing preoccupied, or smiling in a stilted manner, but spontaneously engages the interviewer most of the time. There is a mild form of emotional withdrawal if there is a lack of emotional involvement shown by noticeable failure to make reciprocal comments, appearing preoccupied, or lacking in warmth, but responds to the interviewer when approached. It is moderate if the emotional contact is not present much of the interview because the subject does not elaborate responses, fails to make eye contact, does not seem to care if the interviewer is listening, or may be preoccupied with psychotic material. It is moderately severe if, in addition, emotional contact is not present most of the interview. Severe forms are present if emotional participation is actively avoided by the subject or if the subject is frequently unresponsive or responds with yes/no answers or responds with only minimal affect. It is extremely severe if the subject consistently avoids emotional participation, is unresponsive or responds with yes/no answers or may leave during the interview or just not respond at all.

Reduced social engagement characterises subjective reports of reduced social and interpersonal engagement or interactions. There is no reduced social engagement if there are no reports of reduced social and interpersonal engagement or interactions. It is mild in the case of slight reduction in social engagement with no impairment in social or interpersonal function; moderate in the case of clear reduction in social engagement with some functional sequelae, e.g., avoiding some social engagements or conversations; and severe in the case of marked reduction in social interaction or avoidance of almost all forms of social contact, e.g., refusing to answer the phone or see friends or family.

Social/emotional withdrawal or detachment can be associated with a mental disorder or a nervous system disorder or some other medical conditions.

Mental or nervous system disorders which lead to, or are associated with, social/emotional withdrawal or detachment include disorders characterized by depressive episodes, for example Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorders, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain and Fibromyalgia; Mental and Behavioural Disorders due to Psychoactive Substance Use, for example Substance Use Disorder (SUD); Psychotic Disorders, for example Schizophrenia; Dementia, for example Alzheimer's Dementia (AD); Dementia with Lewy Bodies (DLB); Vascular Dementia and Fronto-Temporal Dementia (FTD); Parkinson's Disease (PD); Eating Disorders; Autism Spectrum Disorder (ASD); Attention Deficit Hyperactivity Disorder (ADHD); and Personality Disorders, for example Schizotypal Personality Disorder and Borderline Personality Disorder (BPD).

The social/emotional withdrawal or detachment can also occur in a patient suffering from sleep disturbance, for instance, insomnia.

The social/emotional withdrawal or detachment can also occur in a patient suffering from medical health conditions leading to an associated mental or nervous system condition include Traumatic Brain Injury (TBI).

Measuring Social/Emotional Withdrawal or Detachment

Social/emotional withdrawal or detachment (herein often referred to as social/emotional withdrawal) or individual aspects thereof, such as anhedonia, emotional withdrawal and affective flattening, can be evaluated by different instruments, such as questionnaires or scales.

Questionnaires assess the mental status of a patient based on observations made by the patient himself/herself, caregivers or the clinician administering the questionnaire.

Questionnaires used to assess whether a patient suffers from a particular mental or nervous system disorder may comprise items related to social/emotional withdrawal or detachment.

The Snaith-Hamilton Pleasure Scale (SHAPS) is a 14-item scale that measures anhedonia, i.e., the inability to experience pleasure. The items cover the domains of: social interaction, food and drink, sensory experience, and interest/pastimes. A score of 2 or less constitutes a “normal” score, while an “abnormal” score is defined as 3 or more.

Each item has four possible responses: strongly disagree, disagree, agree, or strongly agree. Either of the “disagree” responses score one point, and either of the “agree” responses score 0 points. Thus, the final score ranges from 0 to 14. The SHAPS has adequate construct validity and satisfactory test-retest reliability. High internal consistency has also been reported. The SHAPS has been used for measuring anhedonia in depression, but it is also frequently used to assess anhedonia in other patient groups.

In principle, the SHAPS measures hedonic tone during the last few days with 14 hypothetically formulated items. However, due to the hypothetical nature of the items an appropriate shorter recall period can also be applied for an earlier assessment time point.

Alternatively or additionally, the Dimensional Anhedonia Rating Scale (DARS) measuring interest, motivation, effort and consummatory pleasure across four domains: hobbies, food/drink, social activities and sensory experience can be used for the assessment of anhedonia. It comprises 17 items assessing state anhedonia right now. The DARS is rated on a five-point Likert scale from 0 (not at all) to 4 (very much), higher values indicating less anhedonia. All items are summed up to a total score in the range of 0 to 68. For each of the four hedonic domains, hobbies (four items, sum score 0-16), food/drink (four items, sum score 0-16), social activities (four items, sum score 0-16) and sensory experiences (5 items, sum score 0-20), participants are asked to provide two or three of their own favourite examples.

The Personality Inventory for DSM-5 (PID-5)—Adult is a 220 item self-rated personality trait assessment scale for adults age 18 and older. It assesses 25 personality trait facets including Anhedonia, Anxiousness, Attention Seeking, Callousness, Deceitfulness, Depressivity, Distractibility, Eccentricity, Emotional Lability, Grandiosity, Hostility, Impulsivity, Intimacy Avoidance, Irresponsibility, Manipulativeness, Perceptual Dysregulation, Perseveration, Restricted Affectivity, Rigid Perfectionism, Risk Taking, Separation Insecurity, Submissiveness, Suspiciousness, Unusual Beliefs and Experiences, and Withdrawal, with each trait facet consisting of 4 to 14 items.

The trait facet Anhedonia contains the items 1, 23, 26, 30R, 124, 155R, 157, 189 (reverse scored items are marked with the letter “R”), the trait facet Withdrawal contains the items 10, 20, 75, 82, 136, 146, 147, 161, 182, 186 and the trait facet Intimacy Avoidance contains the items 89, 97R, 108, 120, 145, 203. These three trait facets can be combined to yield the broader trait domain designated Detachment.

The measure is completed by the individual prior to a visit with the clinician. Each item asks the individual to rate how well the item describes him or her generally.

Each item on the measure is rated on a 4-point scale. The response categories for the items are 0=very false or often false; 1=sometimes or somewhat false; 2=sometimes or somewhat true; 3=very true or often true. For items 7, 30, 35, 58, 87, 90, 96, 97, 98, 131, 142, 155, 164, 177, 210, and 215, the items are reverse-coded prior to entering into scale score computations.

The scores on the items within each trait facet should be summed and entered in the appropriate raw facet score box. In addition, the clinician is asked to calculate and use average scores for each facet and domain. The average scores reduce the overall score as well as the scores for each domain to a 4-point scale, which allows the clinician to think of the individual's personality dysfunction relative to observed norms. The average facet score is calculated by dividing the raw facet score by the number of items in the facet (e.g., if all the items within the “Anhedonia” facet are rated as being “sometimes or somewhat true,” then the average facet score would be 16/8=2, indicating moderate anhedonia). An average domain score is calculated by summing and then averaging the 3 facet scores contributing primarily to the specific domain. For example, if the average facet scores on Anhedonia, Intimacy Avoidance and Withdrawal (scales primarily indexing Detachment) are all 2, then the sum of these scores would be 6, and the average domain score would be 6/3=2. Higher average scores indicate greater dysfunction in a specific personality trait facet or domain.

High scores on a facet or domain may indicate significant and problematic areas for the individual receiving care that might warrant further assessment, treatment, and follow-up.

Resting State Networks and Social/Emotional Withdrawal or Detachment

Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly on the basis of spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system.

RSNs have been shown to be responsible for various aspects of complex brain function, and it has been found that these connectivity networks are compromised in various disease states. Such disease states, which include certain forms of social/emotional withdrawal or detachment, are associated with altered functional connectivity within a specific resting state network and/or between one or more regions in one or more additional resting state networks.

Alterations in RSNs are also involved in anhedonia, which is one key aspect of social/emotional withdrawal or detachment. More specifically, anhedonia is associated with visual network hyperconnectivity and expansion of the visual network, dorsal attention network (DAN), and default mode network (DMN). Anhedonia also involves decreased between-network connectivity among the DMN, salience, DAN, somatomotor, and visual networks.

In addition, emotional detachment in adult psychopathy has been associated with structural abnormalities in the dorsal DMN. The dorsal DMN is of particular interest in the development of psychopathy due to the functions associated with it. Specifically, the dorsal DMN, and the regions it connects (the medial prefrontal cortex and posterior cingulate cortex (PCC)), underpin affective, social and moral processing. In adult psychopathy, microstructural abnormalities within the dorsal DMN are linked to the affective and interpersonal differences that define the disorder.

Thus, patients suffering from social/emotional withdrawal or detachment show altered functional connectivity within and/or between RSNs when compared to healthy, age-matched controls. Alterations are observed within and/or between the DMN, the salience, DAN, somatomotor, and visual networks.

In many instances, RSNs involved in social/emotional withdrawal or detachment are affected by mental or nervous system disorders, such as disorders characterized by depressive episodes, for example Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorders, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain and Fibromyalgia; Mental and Behavioural Disorders due to Psychoactive Substance Use, for example Substance Use Disorder (SUD); Psychotic Disorders, for example Schizophrenia; Dementia, for example Alzheimer's Dementia (AD); Dementia with Lewy Bodies (DLB); Vascular Dementia and Fronto-Temporal Dementia (FTD); Parkinson's Disease (PD); Eating Disorders; Autism Spectrum Disorder (ASD); Attention Deficit Hyperactivity Disorder (ADHD); and Personality Disorders, for example Schizotypal Personality Disorder and Borderline Personality Disorder (BPD).

Resting state networks involved in social/emotional withdrawal or detachment are also affected by mental or nervous system conditions which are a consequence of certain medical health conditions, such as Traumatic Brain Injury (TBI).

Resting state networks involved in social/emotional withdrawal or detachment are also affected by sleep disturbance, for instance, insomnia. In fact, social/emotional withdrawal or detachment and impairment of sleep are correlated.

Treatment of Social/Emotional Withdrawal or Detachment and Mental or Nervous System Disorders

According to the invention, social/emotional withdrawal or detachment occurring in a patient suffering from a mental disorder or a nervous system disorder can be treated. Moreover, social/emotional withdrawal or detachment occurring in a patient suffering from sleep disturbance, for instance, insomnia, can be treated.

In patients suffering from social/emotional withdrawal or detachment in association with another condition as detailed above, a treatment of social/emotional withdrawal or detachment according to the invention leads to an improvement of the condition with which the social/emotional withdrawal or detachment is associated.

Treatment according to the invention is by administration of 5-MeO-DMT or a pharmaceutically acceptable salt thereof. 5-MeO-DMT administered to a patient disrupts established functional connectivity patterns within and/or between resting state networks. This disruption leads to a reset of the pathological ill-connected connections as the networks reconnect. New, healthy functional connections are established with persistent effects.

Thus, according to the invention, influencing those networks by a therapy as described herein will lead to an improvement of the social/emotional withdrawal or detachment and, if the patient treated suffers from a mental disorder or a nervous system disorder, also of that disorder; if the patient treated suffers from sleep disturbance, for instance, insomnia, also of the sleep disturbance, for instance, insomnia.

To further support the clinical application of 5-MeO-DMT in patients suffering from social/emotional withdrawal or detachment, the inventors assessed clinical data relating to the use of 5-MeO-DMT in patients treated because of mental disease and noted particular improvements in social/emotional withdrawal or detachment which is typically also observed in patients with other disorders.

The data stem from a recently completed clinical trial investigating the use of 5-MeO-DMT in the treatment of patients diagnosed with Treatment Resistant Depression (TRD; see also the examples section below). While TRD is a specific condition, the inventors determined, as discussed in detail below, that certain clinical observations are made in the trial are relevant for devising a treatment for other conditions which are associated with social/emotional withdrawal or detachment.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Asberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed that particular subscore items, like items related to social/emotional withdrawal or detachment, are relevant for other conditions in which social/emotional withdrawal or detachment is based on similarly altered functional connectivity within and/or between the default mode network, the salience, dorsal attention, somatomotor, and visual networks.

Multiple patients within the recruited cohort displayed significant improvements, a result that confirms the inventors' finding that 5-MeO-DMT is a compound suitable for treating patients presenting with these symptoms.

More in particular, an aspect which can be treated by administration of 5-MeO-DMT, is social/emotional withdrawal or detachment, in particular anhedonia, emotional withdrawal and/or affective flattening. A further treated aspect is reduced social engagement. 5-MeO-DMT can be administered to patients to reduce or eliminate social/emotional withdrawal or detachment, in particular anhedonia, emotional withdrawal and/or affective flattening, in said patients. In addition, reduced social engagement is improved, i.e. it is reduced or eliminated.

The MADRS scale item “inability to feel”, that is of particular relevance to social/emotional withdrawal or detachment, represents the subjective experience of reduced interest in the surroundings, or activities that normally give pleasure. The ability to react with adequate emotion to circumstances or people is reduced.

A score of 0 indicates normal interest in the surroundings and in other people, a score of 2 indicates a reduced ability to enjoy usual interests. A score of 4 is assigned in case of a loss of interest in the surroundings and a loss of feelings for friends and acquaintances. A score of 6 reflects the experience of being emotionally paralysed, inability to feel anger, grief or pleasure and a complete or even painful failure to feel for close relatives and friends.

In the study group receiving the individualized dosing regimen, the aggregated score for the MADRS item “inability to feel” across all 8 patients was 36 at base line. After 2 hours, it was reduced to 12 which corresponds to an improvement of 24 points or 67%. At day 1 after treatment, it was reduced to 2 which corresponds to an improvement of 34 points or 94%. At day 7 after treatment, it was reduced to 6 which corresponds to an improvement of 30 points or 83%.

In the 12 mg group, the aggregated score for the MADRS item “inability to feel” across all 4 patients was 16 at base line. After 2 hours, it was reduced to 9 which corresponds to an improvement of 7 points or 44%. At day 1 after treatment, it was reduced to 1 which corresponds to an improvement of 15 points or 94%. At day 7 after treatment, it was reduced to 1 which corresponds to an improvement of 15 points or 94%.

The BPRS scale items which are of particular relevance to social/emotional withdrawal or detachment are “emotional withdrawal” and “blunted affect”.

The BPRS item “emotional withdrawal” relates to a deficiency in the patient's ability to relate emotionally during the interview situation. Possible scores are:
1—No emotional withdrawal.
2—Very Mild. Lack of emotional involvement shown by occasional failure to make reciprocal comments, occasionally appearing preoccupied, or smiling in a stilted manner, but spontaneously engages the interviewer most of the time.
3—Mild. Lack of emotional involvement shown by noticeable failure to make reciprocal comments, appearing preoccupied, or lacking in warmth, but responds to interviewer when approached.
4—Moderate. Emotional contact not present much of the interview because subject does not elaborate responses, fails to make eye contact, doesn't seem to care if interviewer is listening, or may be preoccupied with psychotic material.
5—Moderately Severe. Same as “4” but emotional contact not present most of the interview.
6—Severe. Actively avoids emotional participation. Frequently unresponsive or responds with yes/no answers (not solely due to persecutory delusions). Responds with only minimal affect.
7—Extremely Severe. Consistently avoids emotional participation. Unresponsive or responds with yes/no answers (not solely due to persecutory delusions). May leave during interview or just not respond at all.

The aggregated score for the BPRS item “emotional withdrawal” was 13 at base line. After 3 hours, it was reduced to 8 which corresponds to an improvement of 5 points or 38%. At day 1 after treatment, it was reduced to 8 which corresponds to an improvement of 5 points or 38%. At day 7 after treatment, it was reduced to 8 which corresponds to an improvement of 5 points or 38%.

In the 12 mg group, the aggregated score for the BPRS item “emotional withdrawal” was 13 at base line. After 3 hours, it was reduced to 11 which corresponds to an improvement of 2 points or 15%. At day 1 after treatment, it was reduced to 8 which corresponds to an improvement of 5 points or 38%. At day 7 after treatment, it was reduced to 6 which corresponds to an improvement of 7 points or 54%.

The BPRS item “blunted affect” relates to a restricted range in emotional expressiveness of face, voice, and gestures as well as a marked indifference or flatness even when discussing distressing topics. Possible scores are:
1—No blunted affect.
2—Very Mild. Emotional range is slightly subdued or reserved but displays appropriate facial expressions and tone of voice that are within normal limits.
3—Mild. Emotional range overall is diminished, subdued, or reserved, without many spontaneous and appropriate emotional responses. Voice tone is slightly monotonous.
4—Moderate. Emotional range is noticeably diminished, patient doesn't show emotion, smile, or react to distressing topics except infrequently. Voice tone is monotonous or there is noticeable decrease in spontaneous movements. Displays of emotion or gestures are usually followed by a return to flattened affect.
5—Moderately Severe. Emotional range very diminished, patient doesn't show emotion, smile or react to distressing topics except minimally, few gestures, facial expression does not change very often. Voice tone is monotonous much of the time.
6—Severe. Very little emotional range or expression. Mechanical in speech and gestures most of the time. Unchanging facial expression. Voice tone is monotonous most of the time.
7—Extremely Severe. Virtually no emotional range or expressiveness, stiff movements. Voice tone is monotonous all of the time.

The aggregated score for the BPRS item “blunted affect” was 15 at base line. After 3 hours, it was reduced to 11 which corresponds to an improvement of 4 points or 27%. At day 1 after treatment, it was reduced to 8 which corresponds to an improvement of 7 points or 47%. At day 7 after treatment, it was reduced to 8 which corresponds to an improvement of 7 points or 47%.

In the 12 mg group, the aggregated score for the BPRS item “blunted affect” was 11 at base line. After 3 hours, it was reduced to 8 which corresponds to an improvement of 3 points or 27%. At day 1 after treatment, it was reduced to 6 which corresponds to an improvement of 5 points or 45%. At day 7 after treatment, it was reduced to 5 which corresponds to an improvement of 6 points or 55%.

Thus, the scores of the scale items that are of particular relevance to social/emotional withdrawal or detachment, i.e., “inability to feel” (MADRS), “emotional withdrawal” (BPRS) and “blunted affect” (BPRS), are markedly improved. The inventors conclude that 5-MeO-DMT can be used to treat social/emotional withdrawal or detachment in patients, in particular in patients also suffering from a mental disorder or a nervous system disorder or a sleep disturbance, for instance insomnia.

Consequently, according to the invention, the treatment of a patient suffering from social/emotional withdrawal or detachment with 5-MeO-DMT or a pharmaceutically acceptable salt thereof reduces or eliminates social/emotional withdrawal or detachment.

Negative Thinking

Symptoms such as pessimism, feelings of worthlessness, feelings of helplessness and hopelessness, and feelings of pathological, excessive or inappropriate guilt are clustered together here as negative thinking.

Feelings of helplessness and hopelessness (also simply referred to as helplessness and hopelessness) characterize the subjective sense of pessimism or gloom regarding the future, inability to cope, or sense of loss of control. There is no helplessness and hopelessness if the patient does not have such feelings. Helplessness and hopelessness are mild in the case of occasional and mild feelings of not being able to cope as usual or pessimism; moderate in the case the patient often feels unable to cope or has significant feelings of helplessness or hopelessness which lift at times; or severe if there are marked and persistent feelings of pessimism, helplessness, or hopelessness.

Feelings of worthlessness (also simply referred to as worthlessness) characterize the subjective sense or thoughts of decreased self-value or self-worth. There is no worthlessness if the patient does not have such feelings. They may be mild, i.e., slight decrease in sense of self-worth; moderate; i.e., some thoughts of worthlessness and decreased self-worth; or severe, i.e., marked, pervasive, or persistent feelings of worthlessness, e.g., feels others better off without them, unable to appreciate positive attributes.

Feelings of guilt (also simply referred to as guilt) characterise the subjective sense of self blame, failure, or remorse for real or imagined past errors. There is no guilt if the patient does not have such feelings. They are mild in the case of slight decrease in self-esteem or increased self-criticism; moderate in the case of significant thoughts of failure, self-criticism, inability to cope, or ruminations regarding past failures and the effect on others; able to recognise as excessive; or severe in the case of marked, pervasive, or persistent guilt, e.g., feelings of deserving punishment; or does not clearly recognise as excessive.

Negative thinking can be associated with a mental disorder or a nervous system disorder or some other medical conditions.

Mental or nervous system disorders which lead to, or are associated with, negative thinking include disorders characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorders due to Psychoactive Substance Use, for example Substance Use Disorder (SUD); Psychotic Disorder, for example Schizophrenia; Dementia, for example Alzheimer's Dementia (AD); Eating Disorders; Attention Deficit Hyperactivity Disorder (ADHD); Personality Disorders, for example Schizotypal Personality Disorder and Borderline Personality Disorder (BPD).

The negative thinking can also occur in a patient suffering from sleep disturbance, for instance, insomnia.

The negative thinking can also occur in a patient suffering from medical health conditions leading to an associated mental or nervous system condition including Traumatic Brain Injury (TBI).

Measuring Negative Thinking

Negative thinking or individual aspects thereof, such as worthlessness, helplessness and hopelessness, and guilt, can be evaluated by different instruments, such as questionnaires or scales.

Questionnaires assess the mental status of a patient based on observations made by the patient himself/herself, caregivers or the clinician administering the questionnaire. Questionnaires used to assess whether a patient suffers from a particular mental or nervous system disorder may comprise items related to negative thinking.

Instruments evaluating relevant aspects of negative thinking include, for example, the State Shame and Guilt Scale (SSGS), the Positive and Negative Affect Schedule-Expanded Form (PANAS-X) or the State Hope Scale (SHS).

The State Shame and Guilt Scale (SSGS) is a self-rating scale of in-the-moment (state) feelings of shame, and guilt experiences. It comprises two subscales, a shame and a guilt subscale. The shame subscale comprises items 1, 3, 5, 7, 9. The guilt subscale comprises items 2, 4, 6, 8, 10. All items are scored in a positive direction and are rated on a 5-point Likert scale. It contains some statements which may or may not describe how the patient is feeling right now. A higher score indicates a more intense feeling of shame or guilt.

The Positive and Negative Affect Schedule-Expanded Form (PANAS-X) is a 60-item, expanded version of the PANAS. The PANAS-X measures 11 specific affects: Fear, Sadness, Guilt, Hostility, Shyness, Fatigue, Surprise, Joviality, Self-Assurance, Attentiveness, and Serenity. The PANAS-X thus provides for mood measurement at two different levels. The basic negative emotion scales are fear, hostility, guilt and sadness, while the scale guilt encompasses six items: guilty, ashamed, blameworthy, angry at self, disgusted with self, dissatisfied with self. Each answer should be scored as 1=very slightly or not at all; 2=a little; 3=moderately; 4=quite a bit; or 5=extremely. However, investigators facing more severe time constraints can select and assess only those scales that are most relevant to their research.

More intense feelings of guilt are reflected by a higher score on the guilt scale.

The PANAS-X is simple and easy to administer. Most subjects complete the entire 60-item schedule in 10 minutes or less. This scale consists of a number of words and phrases that describe different feelings and emotions. While it should be indicated to what extent the patient has felt this way during the past few weeks, it has been found that the trait scores on the PANAS-X scales are stable overtime, including “at the present moment”, “today” and during “the past few days”, indicating that an appropriate shorter recall period can be applied.

The State Hope Scale (SHS) has three agency and three pathways items to which respondents describe themselves in terms of how they are “right now.” The agency subscale score is derived by summing items 2, 4 and 6, relating to the perceived capacity to use one's pathways to reach desired goals; the pathways subscale score is derived by adding the items 1, 3 and 5, relating to thinking that is used to identify possible ways to achieve a goal. The total State Hope Scale score is derived by summing the three agency and the three pathways items. Scores can range from a low of 6 to a high of 48, wherein higher hope is reflected by a higher score on this scale.

Negative thinking or aspects thereof are also reflected in other scales such as HAM-D, the MADRS, the BPRS or the BDRS, wherein relevant items thereof may be commonly applicable for assessing negative thinking or aspects thereof.

Resting State Networks and Negative Thinking

Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly on the basis of spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system.

RSNs have been shown to be responsible for various aspects of complex brain function, and it has been found that these connectivity networks are compromised in various disease states. Such disease states, which include certain forms of negative thinking, are associated with altered functional connectivity within a specific resting state network and/or between one or more regions in one or more additional resting state networks.

Major Depressive Disorder (MDD) is a disorder characterized broadly by a high level of negative emotions, and a lower level of positive emotions. More specifically, lower levels of positive emotions such as hope, meaning higher level of hopelessness, and higher levels of negative emotions such as guilt. MDD has been a focus of study in the field of rs-fMRI indicating that MDD is a disorder characterized by widespread network dysfunction. This dysfunction has been found primarily in networks and areas relating to emotional regulation. These include the Default Mode Network (DMN), salience network, affective network, and the prefrontal cortex. Thus, different aspects of negative thinking can be associated with abnormal resting state networks.

Dysfunctional connectivity in resting state networks has been also reported for patients with repetitive negative thinking (RNT) including an altered connectivity of the left Executive Control Network and the Anterior Salience Network with the ventral Default Mode Network.

Thus, patients suffering from negative thinking show altered functional connectivity within and/or between resting state networks when compared to healthy, age-matched controls. Alterations are observed within and/or between at least the default mode network, the executive control network, the salience network, the affective network, and the prefrontal cortex.

In many instances, RSNs involved in negative thinking are affected by mental or nervous system disorders, such as disorders characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorders due to Psychoactive Substance Use, for example Substance Use Disorder (SUD); Psychotic Disorder, for example Schizophrenia; Dementia, for example Alzheimer's Dementia (AD); Eating Disorders; Attention Deficit Hyperactivity Disorder (ADHD); Personality Disorders, for example Schizotypal Personality Disorder and Borderline Personality Disorder (BPD).

Resting state networks involved in negative thinking are also affected by mental or nervous system conditions which are a consequence of certain medical health conditions, such as Traumatic Brain Injury (TBI).

Resting state networks involved in negative thinking are also affected by sleep disturbance, for instance, insomnia. In fact, negative thinking and impairment of sleep are correlated.

Treatment of Negative Thinking and Mental or Nervous System Disorders

According to the invention, negative thinking occurring in a patient suffering from a mental disorder or a nervous system disorder can be treated. Moreover, negative thinking occurring in a patient suffering from sleep disturbance, for instance, insomnia, can be treated.

In patients suffering from negative thinking in association with another condition as detailed above, a treatment of negative thinking according to the invention leads to an improvement of the condition with which the negative thinking is associated.

Treatment according to the invention is by administration of 5-MeO-DMT or a pharmaceutically acceptable salt thereof.

5-MeO-DMT administered to a patient disrupts established functional connectivity patterns within and/or between resting state networks. This disruption leads to a reset of the pathological ill-connected connections as the networks reconnect. New, healthy functional connections are established with persistent effects.

Thus, according to the invention, influencing those networks by a therapy as described herein will lead to an improvement of the negative thinking and, if the patient treated suffers from a mental disorder or a nervous system disorder, also of that disorder; if the patient treated suffers from sleep disturbance, for instance, insomnia, also of the sleep disturbance, for instance, insomnia.

To further support the clinical application of 5-MeO-DMT in patients suffering from negative thinking, the inventors assessed clinical data relating to the use of 5-MeO-DMT in patients treated because of mental disease and noted particular improvements in negative thinking which is typically also observed in patients with other disorders.

The data stem from a recently completed clinical trial investigating the use of 5-MeO-DMT in the treatment of patients diagnosed with Treatment Resistant Depression (TRD; see also the examples section below). While TRD is a specific condition, the inventors determined, as discussed in detail below, that certain clinical observations are made in the trial are relevant for devising a treatment for other conditions which are associated with negative thinking.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Asberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed that particular subscore items, like items related to negative thinking, are relevant for other conditions in which negative thinking is based on similarly altered functional connectivity within and/or between the default mode network, the executive control network, the salience network, the affective network, and the prefrontal cortex.

Multiple patients within the recruited cohort displayed significant improvements, a result that confirms the inventors' finding that 5-MeO-DMT is a compound suitable for treating patients presenting with these symptoms.

More in particular, an aspect which can be treated by administration of 5-MeO-DMT, is negative thinking, in particular feelings of worthlessness, helplessness and hopelessness, and/or guilt. 5-MeO-DMT can be administered to patients to reduce or eliminate negative thinking, in particular feelings of worthlessness, helplessness and hopelessness, and/or guilt, in said patients.

The MADRS scale item that is of particular relevance to negative thinking is “pessimistic thoughts”, which represents thoughts of guilt, inferiority, self-reproach, sinfulness, remorse and ruin.

A score of 0 is assigned if there are no pessimistic thoughts. The score is 2 in the case of fluctuating ideas of failure, self-reproach or self-depreciation. A score means persistent self-accusations, or definite but still rational ideas of guilt or sin as well as the patient being increasingly pessimistic about the future. A score of 6 is assigned in the case of delusions of ruin, remorse or unredeemable sin and self-accusations which are absurd and unshakable.

In the study group receiving the individualized dosing regimen, the aggregated score for the MADRS item “pessimistic thoughts” across all 8 patients was 28 at base line.

After 2 hours, it was reduced to 7 which corresponds to an improvement of 21 points or 75%. At day 1 after treatment, it was reduced to 4 which corresponds to an improvement of 24 points or 86%. At day 7 after treatment, it was reduced to 3 which corresponds to an improvement of 25 points or 89%.

In the 12 mg group, the aggregated score for the MADRS item “pessimistic thoughts” across all 4 patients was 16 at base line. After 2 hours, it was reduced to 8 which corresponds to an improvement of 8 points or 50%. At day 1 after treatment, it was reduced to 7 which corresponds to an improvement of 9 points or 56%.

At day 7 after treatment, it was reduced to 8 which corresponds to an improvement of 8 points or 50%.

The BPRS item that is of particular relevance to negative thinking is “guilt feelings”. This item relates to over concern or remorse for past behaviour. Possible scores are:
1—No guilt feelings.
2—Very Mild. Concerned about having failed someone or at something but not preoccupied. Can shift thoughts to other matters easily.
3—Mild. Concerned about having failed someone or at something with some preoccupation. Tends to voice guilt to others.
4—Moderate. Disproportionate preoccupation with guilt, having done wrong, injured others by doing or failing to do something, but can readily turn attention to other things.
5—Moderately Severe. Preoccupation with guilt, having failed someone or at something, can turn attention to other things, but only with great effort. Not delusional.
6—Severe; Delusional guilt or unreasonable self-reproach very out of proportion to circumstances. Moderate preoccupation present.
7—Extremely Severe. Delusional guilt or unreasonable self-reproach grossly out of proportion to circumstances. Subject is very preoccupied with guilt and is likely to disclose to others or act on delusions.

In the study group receiving the individualized dosing regimen, the aggregated score for the BPRS item “guilt feelings” across all 8 patients was 34 at base line.

After 3 hours, it was reduced to 14 which corresponds to an improvement of 20 points or 59%. At day 1 after treatment, it was reduced to 11 which corresponds to an improvement of 23 points or 68%. At day 7 after treatment, it was reduced to 10 which corresponds to an improvement of 24 points or 71%.

In the 12 mg group, the aggregated score for the BPRS item “guilt feelings” across all 4 patients was 18 at base line.

After 3 hours, it was reduced to 9 which corresponds to an improvement of 9 points or 50%. At day 1 after treatment, it was reduced to 5 which corresponds to an improvement of 13 points or 72%. At day 7 after treatment, it was reduced to 5 which corresponds to an improvement of 13 points or 72%.

Thus, the score of the MADRS scale item that is of particular relevance to negative thinking, “pessimistic thoughts”, is markedly improved, as is the score of the BPRS item “guilt feelings”. The inventors conclude that 5-MeO-DMT can be used to treat negative thinking in patients, in particular patients also suffering from a mental disorder or a nervous system disorder or a sleep disturbance, for instance insomnia.

Consequently, according to the invention, the treatment of a patient suffering from negative thinking with 5-MeO-DMT or a pharmaceutically acceptable salt thereof reduces or eliminates negative thinking.

Maternal Functioning

In addition to the above, the inventors consider that mental or nervous system disorders as defined herein, in particular disorders involving one or more symptoms selected from sleep disturbance, cognitive dysfunction, anxiety, psychomotor retardation, social/emotional withdrawal and negative thinking compromises maternal functioning. In fact, each of the symptoms listed has as such the potential to compromise maternal functioning (and thus independently deserves treatment).

In particular the first year after childbirth marks a critical window for both mother and child. In most cases, mothers are the primary caregivers and are, therefore, responsible for the majority of the work related to infant care tasks.

Maternal functioning includes aspects of maternal competence relating to interactions with the infant(s) as well as maternal self-care.

Maternal functioning, including the emotional aspect of mothering, is also important for the child's development. In fact, the quality of mother-child interaction in the year after birth affects infant development. High levels of maternal functioning are likely to correlate with positive infant development outcomes. Likewise, impaired functioning in the postpartum period might impede optimal infant development.

The Barkin Index of Maternal Functioning (BIMF) was designed to measure functioning in the year after childbirth. The BIMF is a 20-item self-report measure of functioning. Each item is assigned a score between 0 and 6 so that the maximum total score is 120. The higher the score, the better maternal functioning is rated.

The BIMF identifies the key functional domains of a mother during the postnatal period as: self-care, infant care, mother-child interaction, psychological wellbeing of the mother, social support, management, and adjustment.

A BIMF score of 95 or below is considered herein as representing slightly compromised maternal functioning, score of 80 or below is considered herein as representing compromised maternal functioning, a score of 65 or below is considered herein as representing severely compromised maternal functioning. The invention in particular allows improving maternal functioning in patients having a score of 80 or below before treatment and in patients having a score of even 65 or below.

As already indicated above, the present invention allows treating patients suffering from a mental or nervous system disorder. The treatment does not only lead to reductions in scores assessing the severity of depression, but also improves maternal functioning as discussed in detail below.

To further support the clinical application of 5-MeO-DMT in patients suffering from a mental or nervous system disorder the inventors assessed clinical data relating to the use of 5-MeO-DMT in patients treated because of mental disease and noted particular improvements in disease aspects typically also observed in patients with a mental or nervous system disorder. The inventors in particular noted improvements in various symptoms and combinations of symptoms which the inventors determined to be also associated with maternal functioning.

The data stem from a recently completed clinical trial investigating the use of 5-MeO-DMT in the treatment of patients diagnosed with Treatment Resistant Depression (TRD; see also the examples section below). The results are confirmed by a recent trial in patients suffering from Post Partum Depression (see the example section below).

In the TRD clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery Asberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed that several of the subscore items are of particular relevance for a mental or nervous system disorder patients and are related to maternal functioning.

Multiple patients within the recruited cohort displayed significant improvements in one or more of these subscore items, a result that confirms the inventors' finding that 5-MeO-DMT is a compound suitable for treating a mental or nervous system disorder patients and for improving maternal functioning in those patients.

The specific subscore items in each of the scales are identified in more detail below. The inventors conclude that efficacy in treating one or more of these symptoms will result in significant improvements in overall outcomes in mental or nervous system disorder patients treated using 5-MeO-DMT.

Thus, a treatment according to the invention reduces or eliminates (or improves or eliminates) an aspect of the disease.

If the aspect is assessed on the MADRS scale, there is an improvement by at least one point (reduction) or the patient is in complete remission after the treatment (elimination), i.e., the respective aspect is scored 0.

If the aspect is assessed on the BPRS scale, there is an improvement by at least one point (reduction) or the patient is in complete remission after the treatment (elimination), i.e., the respective aspect is scored 1.

A clinical response may also be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

The inventors further consider that improvements observed in certain MADRS items will translate into improvements in aspects of maternal functioning.

MADRS items of particular relevance are discussed in more detail below.

The MADRS item “inner tension” represents feelings of ill-defined discomfort, edginess, inner turmoil, mental tension mounting to either panic, dread or anguish. It is rated according to intensity, frequency, duration and the extent of reassurance called for.

A score of 0 is assigned if the patient is placid and there is only fleeting inner tension. A score of 2 is assigned if there are occasional feelings of edginess and ill-defined discomfort. The score is 4 if there are continuous feelings of inner tension or intermittent panic which the patient can only master with some difficulty. The score is 6 in case of unrelenting dread or anguish and overwhelming panic.

The inventors have determined that increases in the score of the MADRS item “inner tension” have negative impacts on both aspects of maternal functioning (maternal competence relating to interactions with the infant(s) as well as maternal self-care). Increased scores in the MADRS item “inner tension” impair mother-child interaction as well as psychological well-being of the mother as assessed by the BIMF.

Conversely, improvements regarding this MADRS item lead to improvements in maternal functioning, in particular in the BIMF functional domains mother-child interaction and/or psychological well-being of the mother.

In the above indicated trial involving TRD patients, in the study group receiving the individualized dosing regimen, the aggregated score for the MADRS item “inner tension” across all 8 patients was 26 at base line. After 2 hours, it was reduced to 11 which corresponds to an improvement of 15 points or 58%. At day 1 after treatment, it was reduced to 6 which corresponds to an improvement of 20 points or 77%. At day 7 after treatment, it was reduced to 12 which corresponds to an improvement of 14 points or 54%.

In the 12 mg group, the aggregated score for the MADRS item “inner tension” across all 4 patients was 13 at base line. After 2 hours, it was reduced to 2 which corresponds to an improvement of 11 points or 85%. At day 1 after treatment, it was reduced to 3 which corresponds to an improvement of 10 points or 77%. At day 7 after treatment, it was reduced to 5 which corresponds to an improvement of 8 points or 62%.

The inventors conclude that 5-MeO-DMT can be used to treat a mental or nervous system disorder patients to achieve a reduction or elimination of inner tension.

An improvement in inner tension is reflected by at least an improvement in the score of the MADRS item inner tension about 2 hours; on day 1, for instance, after about 24 hours; on day 7; on day 14; and/or on day 28 after the last administration of 5-MeO-DMT or a pharmaceutically acceptable salt thereof.

An improvement in inner tension as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in inner tension, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in inner tension, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of inner tension achieved by treating a a mental or nervous system disorder patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since inner tension also affects other aspects of a mental or nervous system disorder, the inventors conclude that the observed improvement in the “inner tension” item on the MADRS will additionally contribute to an overall improvement in maternal functioning.

The MADRS item “lassitude” represents a difficulty getting started or slowness initiating and performing everyday activities.

A score of 0 means that there is hardly any difficulty in getting started and no sluggishness. A score of 2 is assigned if the patient has difficulties in starting activities. A score of 4 means difficulties in starting simple routine activities which are carried out with effort.

A score of 6 is assigned in case of complete lassitude, the patient being unable to do anything without help.

The inventors have determined that increases in the score of the MADRS item “lassitude” have negative impacts on both aspects of maternal functioning (maternal competence relating to interactions with the infant(s) as well as maternal self-care). Increased scores in the MADRS item “lassitude” impair infant care, self-care, psychological well-being, management and adjustment.

Conversely, improvements regarding this MADRS item lead to improvements in maternal functioning, in particular in the BIMF functional domains infant care, self-care, psychological well-being, management and/or adjustment.

In the study group receiving the individualized dosing regimen, the aggregated score for the MADRS item “lassitude” across all 8 patients was 27 at base line. After 2 hours, it was reduced to 10 which corresponds to an improvement of 17 points or 63%. At day 1 after treatment, it was reduced to 5 which corresponds to an improvement of 22 points or 81%. At day 7 after treatment, it was reduced to 3 which corresponds to an improvement of 24 points or 89%.

In the 12 mg group, the aggregated score for the MADRS item “lassitude” across all 4 patients was 16 at base line. After 2 hours, it was reduced to 10 which corresponds to an improvement of 6 points or 38%. At day 1 after treatment, it was reduced to 0 which corresponds to an improvement of 16 points or 100%. At day 7 after treatment, it was reduced to 3 which corresponds to an improvement of 13 points or 81%.

The inventors conclude that 5-MeO-DMT can be used to treat mental or nervous system disorder patients to achieve a reduction or elimination of lassitude.

An improvement in lassitude is reflected by at least an improvement in the score of the MADRS item lassitude about 2 hours; on day 1, for instance, after about 24 hours; on day 7; on day 14; and/or on day 28 after the last administration of 5-MeO-DMT or a pharmaceutically acceptable salt thereof.

An improvement in lassitude as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in lassitude, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in lassitude, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of lassitude achieved by treating a mental or nervous system disorder patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof. Since lassitude also affects other aspects of a mental or nervous system disorder, the inventors conclude that the observed improvement in the “lassitude” item on the MADRS will additionally contribute to an overall improvement in maternal functioning.

The MADRS item “inability to feel” represents the subjective experience of reduced interest in the surroundings, or activities that normally give pleasure. The ability to react with adequate emotion to circumstances or people is reduced.

A score of 0 indicates normal interest in the surroundings and in other people, a score of 2 reduced ability to enjoy usual interests. A score of 4 is assigned in case of a loss of interest in the surroundings and a loss of feelings for friends and acquaintances. A score of 6 reflects the experience of being emotionally paralysed, inability to feel anger, grief or pleasure and a complete or even painful failure to feel for close relatives and friends.

The inventors have determined that increases in the score of the MADRS item “inability to feel” have negative impacts on both aspects of maternal functioning (maternal competence relating to interactions with the infant(s) as well as maternal self-care). Increased scores in the MADRS item “inability to feel” impair mother-child interaction and psychological well-being.

Conversely, improvements regarding this MADRS item lead to improvements in maternal functioning, in particular in the BIMF functional domains mother-child interaction and/or psychological well-being.

In the study group receiving the individualized dosing regimen, the aggregated score for the MADRS item “inability to feel” across all 8 patients was 36 at base line. After 2 hours, it was reduced to 12 which corresponds to an improvement of 24 points or 67%. At day 1 after treatment, it was reduced to 2 which corresponds to an improvement of 34 points or 94%. At day 7 after treatment, it was reduced to 6 which corresponds to an improvement of 30 points or 83%.

In the 12 mg group, the aggregated score for the MADRS item “inability to feel” across all 4 patients was 16 at base line. After 2 hours, it was reduced to 9 which corresponds to an improvement of 7 points or 44%. At day 1 after treatment, it was reduced to 1 which corresponds to an improvement of 15 points or 94%. At day 7 after treatment, it was reduced to 1 which corresponds to an improvement of 15 points or 94%.

The inventors conclude that 5-MeO-DMT can be used to treat mental or nervous system disorder patients to achieve a reduction or elimination of inability to feel. An improvement in inability to feel is reflected by at least an improvement in the score of the MADRS item inability to feel about 2 hours; on day 1, for instance, after about 24 hours; on day 7; on day 14; and/or on day 28 after the last administration of 5-MeO-DMT or a pharmaceutically acceptable salt thereof.

An improvement in inability to feel as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in inability to feel, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in inability to feel, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of inability to feel by treating a mental or nervous system disorder patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 7; on day 14; and/or on day 28 after the last administration of 5-MeO-DMT or a pharmaceutically acceptable salt thereof. 13 Since inability to feel also affects other aspects of a mental or nervous system disorder, the inventors conclude that the observed improvement in the “inability to feel” item on the MADRS will additionally contribute to an overall improvement in maternal functioning.

The MADRS item “concentration difficulties” represents difficulties in collecting one's thoughts mounting to incapacitating lack of concentration.

The score is 0 if the patient has no difficulties in concentrating. The score is 2 in case of occasional difficulties in collecting one's thoughts. A score of 4 is assigned in case of difficulties in concentrating and sustaining thought which reduces ability to read or hold a conversation. The score is 6 if the patient is unable to read or converse without great difficulty.

The inventors have determined that increases in the score of the MADRS item “concentration difficulties” have negative impacts on both aspects of maternal functioning (maternal competence relating to interactions with the infant(s) as well as maternal self-care).

Increased scores in the MADRS item “concentration difficulties” impair infant care as well as management.

Conversely, improvements regarding this MADRS item lead to improvements in maternal functioning, in particular in the BIMF functional domains infant care and/or management.

In the study group receiving the individualized dosing regimen, the aggregated score for the MADRS item “concentration difficulties” across all 8 patients was 30 at base line. After 2 hours, it was reduced to 11 which corresponds to an improvement of 19 points or 63%. At day 1 after treatment, it was reduced to 1 which corresponds to an improvement of 29 points or 97%. At day 7 after treatment, it was reduced to 9 which corresponds to an improvement of 21 points or 70%.

In the 12 mg group, the aggregated score for the MADRS item “concentration difficulties” across all 4 patients was 16 at base line. After 2 hours, it was reduced to 7 which corresponds to an improvement of 9 points or 56%. At day 1 after treatment, it was reduced to 2 which corresponds to an improvement of 14 points or 88%. At day 7 after treatment, it was reduced to 3 which corresponds to an improvement of 13 points or 81%.

The inventors conclude that 5-MeO-DMT can be used to treat mental or nervous system disorder patients to achieve a reduction or elimination of concentration difficulties.

An improvement in concentration difficulties is reflected by at least an improvement in the score of the MADRS item concentration difficulties about 2 hours; on day 1, for instance, after about 24 hours; on day 7; on day 14; and/or on day 28 after the last administration of 5-MeO-DMT or a pharmaceutically acceptable salt thereof.

An improvement in concentration difficulties as reflected by a reduction in the Clinical Global Impression-Severity (CGI-S) score, occurs not later than about 2 hours after the last administration of 5-MeO-DMT or a pharmaceutically acceptable salt thereof. Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in concentration difficulties, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in concentration difficulties, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of concentration difficulties by treating a mental or nervous system disorder patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 7; on day 14; and/or on day 28 after the last administration of 5-MeO-DMT or a pharmaceutically acceptable salt thereof.

Since concentration difficulties also affect other aspects of a mental or nervous system disorder, the inventors conclude that the observed improvement in the “concentration difficulties” item on the MADRS will additionally contribute to an overall improvement in maternal functioning.

The MADRS item “pessimistic thoughts” represents thoughts of guilt, inferiority, self-reproach, sinfulness, remorse and ruin.

A score of 0 is assigned if there are no pessimistic thoughts. The score is 2 in case of fluctuating ideas of failure, self-reproach or self-depreciation. A score means persistent self-accusations, or definite but still rational ideas of guilt or sin as well as the patient being increasingly pessimistic about the future. A score of 6 is assigned in case of delusions of ruin, remorse or unredeemable sin and self-accusations which are absurd and unshakable.

The inventors have determined that increases in the score of the MADRS item “pessimistic thoughts” have negative impacts on both aspects of maternal functioning (maternal competence relating to interactions with the infant(s) as well as maternal self-care).

Increased scores in the MADRS item “pessimistic thoughts” impair psychological well-being, social support and management.

Conversely, improvements regarding this MADRS item lead to improvements in maternal functioning, in particular in the BIMF functional domains psychological wellbeing, social support and/or management.

In the study group receiving the individualized dosing regimen, the aggregated score for the MADRS item “pessimistic thoughts” across all 8 patients was 28 at base line. After 2 hours, it was reduced to 7 which corresponds to an improvement of 21 points or 75%. At day 1 after treatment, it was reduced to 4 which corresponds to an improvement of 24 points or 86%. At day 7 after treatment, it was reduced to 3 which corresponds to an improvement of 25 points or 89%.

In the 12 mg group, the aggregated score for the MADRS item “pessimistic thoughts” across all 4 patients was 16 at base line. After 2 hours, it was reduced to 8 which corresponds to an improvement of 8 points or 50%. At day 1 after treatment, it was reduced to 7 which corresponds to an improvement of 9 points or 56%. At day 7 after treatment, it was reduced to 8 which corresponds to an improvement of 8 points or 50%.

The inventors conclude that 5-MeO-DMT can be used to treat mental or nervous system disorder patients to achieve a reduction or elimination of pessimistic thoughts.

An improvement in pessimistic thoughts is reflected by at least an improvement in the score of the MADRS item pessimistic thoughts about 2 hours; on day 1, for instance, after about 24 hours; on day 7; on day 14; and/or on day 28 after the last administration of 5-MeO-DMT or a pharmaceutically acceptable salt thereof.

An improvement in pessimistic thoughts as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in pessimistic thoughts, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in pessimistic thoughts, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of pessimistic thoughts by treating a mental or nervous system disorder patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since pessimistic thoughts also affects other aspects of a mental or nervous system disorder, the inventors conclude that the observed improvement in the “pessimistic thoughts” item on the MADRS will additionally contribute to an overall improvement in maternal functioning.

The MADRS item “reduced sleep” represents the experience of reduced duration or depth of sleep compared to the subject's own normal pattern when well.

A score of 0 is assigned when the subject sleeps as usual. A score of 2 reflects slight difficulty dropping off to sleep or slightly reduced, light or fitful sleep. A score of 4 means that sleep is reduced or broken by at least two hours. A score of 6 means less than two or three hours sleep.

The inventors have determined that increases in the score of the MADRS item “reduced sleep” have negative impacts on both aspects of maternal functioning (maternal competence relating to interactions with the infant(s) as well as maternal self-care). Increased scores in the MADRS item “reduced sleep” impair self-care, psychological well-being and management.

Conversely, improvements regarding this MADRS item lead to improvements in maternal functioning, in particular in the BIMF functional domains self-care, psychological well-being and/or management.

In the study group receiving the individualized dosing regimen, the aggregated score for the MADRS item “reduced sleep” across all 8 patients was 25 at base line. At day 1 after treatment, the earliest timepoint for assessing an impact of the treatment on sleep, it was reduced to 12 which corresponds to an improvement of 13 points or 52%. At day 7 after treatment, it was reduced to 9 which corresponds to an improvement of 16 points or 64%.

In the 12 mg group, the aggregated score for the MADRS item “reduced sleep” across all 4 patients was 12 at base line. At day 1 after treatment, it was reduced to 10 which corresponds to an improvement of 2 points or 17%. At day 7 after treatment, it was reduced to 6 which corresponds to an improvement of 6 points or 50%.

The inventors conclude that 5-MeO-DMT can be used to treat mental or nervous system disorder patients to achieve a reduction or elimination of reduced sleep.

The reduction or elimination of reduced sleep is reflected by at least an improvement in the score of the MADRS item reduced sleep on day 1, for instance, after about 24 hours; on day 7; on day 14; and/or on day 28 after the last administration of 5-MeO-DMT or a pharmaceutically acceptable salt thereof.

An improvement in reduced sleep as reflected by a reduction in the Clinical Global Impression-Severity (CGI-S) score, occurs not later than about 24 hours after the last administration of 5-MeO-DMT or a pharmaceutically acceptable salt thereof.

An improvement in reduced sleep, as reflected by at least a score of “much improved” in the Clinical Global Impression-Improvement (CGI-I) score or the Patient Global Impression-Improvement (PGI-I) score, preferably occurs not later than about 24 hours after the last administration of 5-MeO-DMT or a pharmaceutically acceptable salt thereof.

An improvement in reduced sleep, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of reduced sleep by treating a mental or nervous system disorder patient does not only lead to a reduction in the MADRS total score, but also to an improvement in maternal functioning, as reflected by an increase in the BIMF score. This improvement will be achieved rapidly, namely within about 24 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since reduced sleep also affects other aspects of a mental or nervous system disorder, the inventors conclude that the observed improvement in the “reduced sleep” item on the MADRS will additionally contribute to an overall improvement in maternal functioning.

A further aspect of a mental or nervous system disorder which can be treated by administration of 5-MeO-DMT, is suicidal ideation. 5-MeO-DMT can be administered to mental or nervous system disorder patients to reduce or eliminate suicidal ideation in said patients.

In the above-mentioned clinical studies involving the administration of 5-MeO-DMT, among others the MADRS item “suicidal thoughts” was assessed. “Suicidal thoughts” represents a feeling that life is not worth living, that a natural death would be welcome, having suicidal thoughts, and/or making the preparations for suicide. Suicidal attempts should not in themselves influence the rating for this MADRS item.

A score of 0 means that the patient enjoys life. A score of 2 is assigned if the mental or nervous system disorder patient is weary of life, and/or has only fleeting suicidal thoughts. A score of 4 means the patient feels they would be better off dead, suicidal thoughts are common and suicide is considered as a possible solution but the patient has no specific plans or intention. A score of 6 is assigned in case the patient has explicit plans for suicide and/or is making active preparations.

This MADRS scale item is of particular relevance to suicidal ideation.

The inventors have determined that increases in the score of the MADRS item “suicidal thoughts” have negative impacts on both aspects of maternal functioning (maternal competence relating to interactions with the infant(s) as well as maternal self-care). Increased scores in the MADRS item “suicidal thoughts” impair self-care, psychological well-being and management.

Conversely, improvements regarding this MADRS item lead to improvements in maternal functioning, in particular in the BIMF functional domains self-care, psychological well-being and/or management.

In the study group receiving the individualized dosing regimen, the aggregated score for the MADRS item “suicidal thoughts” across all 8 patients was 11 at base line. After 2 hours, it was reduced to 3 which corresponds to an improvement of 8 points or 73%. At day 1 after treatment, it was reduced to 1 which corresponds to an improvement of 10 points or 91%. At day 7 after treatment, it was reduced to 3 which corresponds to an improvement of 8 points or 73%.

In the 12 mg group, the aggregated score for the MADRS item “suicidal thoughts” across all 4 patients was 8 at base line. After 2 hours, it was reduced to 3 which corresponds to an improvement of 5 points or 63%. At day 1 after treatment, it was reduced to 5 which corresponds to an improvement of 3 points or 38%. At day 7 after treatment, it was reduced to 7 which corresponds to an improvement of 1 point or 13%.

Thus, the score of the scale item that is of particular relevance to suicidal ideation, “suicidal thoughts”, is markedly improved, at least in the individualized dosing regimen patients. The inventors conclude that 5-MeO-DMT can be used to treat suicidal ideation in mental or nervous system disorder patients.

Thus, according to the invention, the treatment of a mental or nervous system disorder patient suffering from suicidal ideation reduces or eliminates the suicidal ideation.

The reduction or elimination of suicidal ideation is reflected by at least an improvement in the score of the MADRS item suicidal thoughts about 2 hours; on day 1, for instance, after about 24 hours, on day 7; on day 14; and/or on day 28 after the last administration of 5-MeO-DMT or a pharmaceutically acceptable salt thereof.

If the patient suffers from suicidal ideation the improvement in suicidal ideation is reflected by a reduction in the Clinical Global Impression-Severity (CGI-S) score about 2 hours; on day 1, for instance, after about 24 hours; on day 7; on day 14; and/or on day 28 after the last administration of 5-MeO-DMT or a pharmaceutically acceptable salt thereof.

An improvement in suicidal ideation as reflected by a reduction in the Clinical Global Impression-Severity (CGI-S) score, occurs not later than about 2 hours after the last administration of 5-MeO-DMT or a pharmaceutically acceptable salt thereof. Alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in suicidal ideation, as assessed by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in suicidal ideation, as assessed by a reduction of the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of suicidal thoughts by treating a mental or nervous system disorder patient does not only lead to a reduction in the MADRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since suicidal thoughts also affects other aspects of a mental or nervous system disorder, the inventors conclude that the observed improvement in the “suicidal thoughts” item on the MADRS will additionally contribute to an overall improvement in maternal functioning.

The BPRS item “emotional withdrawal” relates to a deficiency in the patient's ability to relate emotionally during the interview situation. Possible scores are:
1—No emotional withdrawal.
2—Very Mild. Lack of emotional involvement shown by occasional failure to make reciprocal comments, occasionally appearing preoccupied, or smiling in a stilted manner, but spontaneously engages the interviewer most of the time.
3—Mild. Lack of emotional involvement shown by noticeable failure to make reciprocal comments, appearing preoccupied, or lacking in warmth, but responds to interviewer when approached.
4—Moderate. Emotional contact not present much of the interview because subject does not elaborate responses, fails to make eye contact, doesn't seem to care if interviewer is listening, or may be preoccupied with psychotic material.
5—Moderately Severe. Same as “4” but emotional contact not present most of the interview.
6—Severe. Actively avoids emotional participation. Frequently unresponsive or responds with yes/no answers (not solely due to persecutory delusions). Responds with only minimal affect.
7—Extremely Severe. Consistently avoids emotional participation. Unresponsive or responds with yes/no answers (not solely due to persecutory delusions). May leave during interview or just not respond at all.

The inventors have determined that increases in the score of the BPRS item “emotional withdrawal” have negative impacts on both aspects of maternal functioning (maternal competence relating to interactions with the infant(s) as well as maternal self-care). Increased scores in the BPRS item “emotional withdrawal” impair psychological well-being, mother-child interaction and social support.

Conversely, improvements regarding this BPRS item lead to improvements in maternal functioning, in particular in the BIMF functional domains psychological well-being, mother-child interaction and/or social support.

In the study group receiving the individualized dosing regimen, aggregated score for the BPRS item “emotional withdrawal” was 13 at base line. After 3 hours, it was reduced to 8 which corresponds to an improvement of 5 points or 38%. At day 1 after treatment, it was reduced to 8 which corresponds to an improvement of 5 points or 38%. At day 7 after treatment, it was reduced to 8 which corresponds to an improvement of 5 points or 38%.

In the 12 mg group, the aggregated score for the BPRS item “emotional withdrawal” was 13 at base line. After 3 hours, it was reduced to 11 which corresponds to an improvement of 2 points or 15%. At day 1 after treatment, it was reduced to 8 which corresponds to an improvement of 5 points or 38%. At day 7 after treatment, it was reduced to 6 which corresponds to an improvement of 7 points or 54%.

The inventors conclude that 5-MeO-DMT can be used to treat mental or nervous system disorder patients to achieve a reduction or elimination of emotional withdrawal.

The reduction or elimination of emotional withdrawal is reflected by at least an improvement in the score of the BPRS item emotional withdrawal about 2 hours; on day 1, for instance, after about 24 hours; on day 7; on day 14; and/or on day 28 after the last administration of 5-MeO-DMT or a pharmaceutically acceptable salt thereof.

An improvement in emotional withdrawal as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in emotional withdrawal, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in emotional withdrawal, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of emotional withdrawal by treating a mental or nervous system disorder patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof. Since emotional withdrawal also affects other aspects of a mental or nervous system disorder, the inventors conclude that the observed improvement in the “emotional withdrawal” item on the BPRS will additionally contribute to an overall improvement in maternal functioning.

The BPRS item “blunted affect” relates to a restricted range in emotional expressiveness of face, voice, and gestures as well as a marked indifference or flatness even when discussing distressing topics. Possible scores are:
1—No blunted affect.
2—Very Mild. Emotional range is slightly subdued or reserved but displays appropriate facial expressions and tone of voice that are within normal limits.
3—Mild. Emotional range overall is diminished, subdued, or reserved, without many spontaneous and appropriate emotional responses. Voice tone is slightly monotonous.
4—Moderate. Emotional range is noticeably diminished, patient doesn't show emotion, smile, or react to distressing topics except infrequently. Voice tone is monotonous or there is noticeable decrease in spontaneous movements. Displays of emotion or gestures are usually followed by a return to flattened affect.
5—Moderately Severe. Emotional range very diminished, patient doesn't show emotion, smile or react to distressing topics except minimally, few gestures, facial expression does not change very often. Voice tone is monotonous much of the time.
6—Severe. Very little emotional range or expression. Mechanical in speech and gestures most of the time. Unchanging facial expression. Voice tone is monotonous most of the time.
7—Extremely Severe. Virtually no emotional range or expressiveness, stiff movements. Voice tone is monotonous all of the time.

The inventors have determined that increases in the score of the BPRS item “blunted affect” have negative impacts on both aspects of maternal functioning (maternal competence relating to interactions with the infant(s) as well as maternal self-care). Increased scores in the BPRS item “blunted affect” impair psychological well-being and mother-child interaction.

Conversely, improvements regarding this BPRS item lead to improvements in maternal functioning, in particular in the BIMF functional domains psychological well-being and/or mother-child interaction.

The aggregated score for the BPRS item “blunted affect” was 15 at base line. After 3 hours, it was reduced to 11 which corresponds to an improvement of 4 points or 27%. At day 1 after treatment, it was reduced to 8 which corresponds to an improvement of 7 points or 47%. At day 7 after treatment, it was reduced to 8 which corresponds to an improvement of 7 points or 47%.

In the 12 mg group, the aggregated score for the BPRS item “blunted affect” was 11 at base line. After 3 hours, it was reduced to 8 which corresponds to an improvement of 3 points or 27%. At day 1 after treatment, it was reduced to 6 which corresponds to an improvement of 5 points or 45%. At day 7 after treatment, it was reduced to 5 which corresponds to an improvement of 6 points or 55%.

The inventors conclude that 5-MeO-DMT can be used to treat mental or nervous system disorder patients to achieve a reduction or elimination of blunted affect.

The reduction or elimination of blunted affect is reflected by at least an improvement in the score of the BPRS item blunted affect about 2 hours; on day 1, for instance, after about 24 hours; on day 7; on day 14; and/or on day 28 after the last administration of 5-MeO-DMT or a pharmaceutically acceptable salt thereof.

An improvement in blunted affect as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in blunted affect, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in blunted affect, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of blunted affect by treating a mental or nervous system disorder patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since blunted affect also affects other aspects of a mental or nervous system disorder, the inventors conclude that the observed improvement in the “blunted affect” item on the BPRS will additionally contribute to an overall improvement in maternal functioning.

The BPRS item “guilt feelings” relates to over concern or remorse for past behavior.

Possible scores are:
1—No guilt feelings.
2—Very Mild. Concerned about having failed someone or at something but not preoccupied. Can shift thoughts to other matters easily.
3—Mild. Concerned about having failed someone or at something with some preoccupation. Tends to voice guilt to others.
4—Moderate. Disproportionate preoccupation with guilt, having done wrong, injured others by doing or failing to do something, but can readily turn attention to other things.
5—Moderately Severe. Preoccupation with guilt, having failed someone or at something, can turn attention to other things, but only with great effort. Not delusional.
6—Severe. Delusional guilt or unreasonable self-reproach very out of proportion to circumstances. Moderate preoccupation present.
7—Extremely Severe. Delusional guilt or unreasonable self-reproach grossly out of proportion to circumstances. Subject is very preoccupied with guilt and is likely to disclose to others or act on delusions.

The inventors have determined that increases in the score of the BPRS item “guilt feelings” have negative impacts on both aspects of maternal functioning (maternal competence relating to interactions with the infant(s) as well as maternal self-care). Increased scores in the BPRS item “guilt feelings” impair self-care, mother-child interaction, psychological wellbeing and management.

Conversely, improvements regarding this BPRS item lead to improvements in maternal functioning, in particular in the BIMF functional domains self-care, mother-child interaction, psychological wellbeing and/or management.

In the study group receiving the individualized dosing regimen, the aggregated score for the BPRS item “guilt feelings” across all 8 patients was 34 at base line. After 3 hours, it was reduced to 14 which corresponds to an improvement of 20 points or 59%. At day 1 after treatment, it was reduced to 11 which corresponds to an improvement of 23 points or 68%. At day 7 after treatment, it was reduced to 10 which corresponds to an improvement of 24 points or 71%.

In the 12 mg group, the aggregated score for the BPRS item “guilt feelings” across all 4 patients was 18 at base line. After 3 hours, it was reduced to 9 which corresponds to an improvement of 9 points or 50%. At day 1 after treatment, it was reduced to 5 which corresponds to an improvement of 13 points or 72%. At day 7 after treatment, it was reduced to 5 which corresponds to an improvement of 13 points or 72%.

The inventors conclude that 5-MeO-DMT can be used to treat mental or nervous system disorder patients to achieve a reduction or elimination of guilt feelings.

The reduction or elimination of guilt feelings is reflected by at least an improvement in the score of the BPRS item guilt feelings about 2 hours; on day 1, for instance, after about 24 hours; on day 7; on day 14; and/or on day 28 after the last administration of 5-MeO-DMT or a pharmaceutically acceptable salt thereof.

An improvement in guilt feelings as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in guilt feelings, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in guilt feelings, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of guilt feelings by treating a mental or nervous system disorder patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since guilt feelings also affects other aspects of a mental or nervous system disorder, the inventors conclude that the observed improvement in the “guilt feelings” item on the BPRS will additionally contribute to an overall improvement in maternal functioning.

The BPRS item “anxiety” relates to reported apprehension, tension, fear, panic or worry.

Possible scores are:
1—No anxiety.
2—Very Mild. Reports some discomfort due to worry or infrequent worries that occur more than usual for most normal individuals.
3—Mild. Worried frequently but can readily turn attention to other things.
4—Moderate. Worried most of the time and cannot turn attention to other things easily but no impairment in functioning or occasional anxiety with autonomic accompaniment but no impairment in functioning.
5—Moderately Severe. Frequent, but not daily, periods of anxiety with autonomic accompaniment or some areas of functioning are disrupted by anxiety or worry.
6—Severe. Anxiety with autonomic accompaniment daily but not persisting throughout the day or many areas of functioning are disrupted by anxiety or constant worry.
7—Extremely Severe. Anxiety with autonomic accompaniment persisting throughout the day or most areas of functioning are disrupted by anxiety or constant worry.

The inventors have determined that increases in the score of the BPRS item “anxiety” have negative impacts on both aspects of maternal functioning (maternal competence relating to interactions with the infant(s) as well as maternal self-care). Increased scores in the BPRS item “anxiety” impair psychological wellbeing, social support and management.

Conversely, improvements regarding this BPRS item lead to improvements in maternal functioning, in particular in the BIMF functional domains psychological wellbeing, social support and/or management.

In the study group receiving the individualized dosing regimen, the aggregated score for the BPRS item “anxiety” across all 8 patients was 37 at base line. After 3 hours, it was reduced to 19 which corresponds to an improvement of 18 points or 49%. At day 1 after treatment, it was reduced to 16 which corresponds to an improvement of 21 points or 57%. At day 7 after treatment, it was reduced to 17 which corresponds to an improvement of 20 points or 54%.

In the 12 mg group, the aggregated score for the BPRS item “anxiety” across all 4 patients was 25 at base line. After 3 hours, it was reduced to 11 which corresponds to an improvement of 14 points or 56%. At day 1 after treatment, it was reduced to 6 which corresponds to an improvement of 19 points or 76%. At day 7 after treatment, it was reduced to 6 which corresponds to an improvement of 19 points or 76%.

The inventors conclude that 5-MeO-DMT can be used to treat mental or nervous system disorder patients to achieve a reduction or elimination of anxiety.

The reduction or elimination of anxiety is reflected by at least an improvement in the score of the BPRS item anxiety about 2 hours; on day 1, for instance, after about 24 hours; on day 7; on day 14; and/or on day 28 after the last administration of 5-MeO-DMT or a pharmaceutically acceptable salt thereof.

An improvement in anxiety as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in anxiety, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in anxiety, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of anxiety by treating a mental or nervous system disorder patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since anxiety also affects other aspects of a mental or nervous system disorder, the inventors conclude that the observed improvement in the “anxiety” item on the BPRS will additionally contribute to an overall improvement in maternal functioning.

The BPRS item “tension” relates to observable physical and motor manifestations of tension, “nervousness,” and agitation. Possible scores are
1—No tension.
2—Very Mild. More fidgety than most but within normal range. A few transient signs of tension, e.g., picking at fingernails, foot wagging, scratching scalp several times, or finger tapping.
3—Mild. Same as “2,” but with more frequent or exaggerated signs of tension.
4—Moderate. Many and frequent signs of motor tension with one or more signs sometimes occurring simultaneously, e.g., wagging one's foot while wringing hands together. There are times when no signs of tension are present.
5—Moderately Severe. Many and frequent signs of motor tension with one or more signs often occurring simultaneously. There are still rare times when no signs of tension are present.
6—Severe. Same as “5,” but signs of tension are continuous.
7—Extremely Severe. Multiple motor manifestations of tension are continuously present, e.g., continuous pacing and hand wringing.

The inventors have determined that increases in the score of the BPRS item “tension” have negative impacts on both aspects of maternal functioning (maternal competence relating to interactions with the infant(s) as well as maternal self-care). Increased scores in the BPRS item “tension” impair mother-child interaction and psychological well-being.

Conversely, improvements regarding this BPRS item lead to improvements in maternal functioning, in particular in the BIMF functional domains mother-child interaction and/or psychological well-being.

In the study group receiving the individualized dosing regimen, the aggregated score for the BPRS item “tension” across all 8 patients was 16 at base line. After 3 hours, it was reduced to 11 which corresponds to an improvement of 5 points or 31%. At day 1 after treatment, it was reduced to 11 which corresponds to an improvement of 5 points or 31%. At day 7 after treatment, it was reduced to 10 which corresponds to an improvement of 6 points or 38%.

In the 12 mg group, the aggregated score for the BPRS item “tension” across all 4 patients was 14 at base line. After 3 hours, it was reduced to 9 which corresponds to an improvement of 5 points or 36%. At day 1 after treatment, it was reduced to 6 which corresponds to an improvement of 8 points or 57%. At day 7 after treatment, it was reduced to 6 which corresponds to an improvement of 8 points or 57%.

The inventors conclude that 5-MeO-DMT can be used to treat mental or nervous system disorder patients to achieve a reduction or elimination of tension.

The reduction or elimination of tension is reflected by at least an improvement in the score of the BPRS item tension about 2 hours; on day 1, for instance, after about 24 hours; on day 7; on day 14; and/or on day 28 after the last administration of 5-MeO-DMT or a pharmaceutically acceptable salt thereof.

An improvement in tension as reflected by a reduction in the Clinical Global Impression-Severity (CGI-S) score, occurs not later than about 2 hours after the last administration of 5-MeO-DMT or a pharmaceutically acceptable salt thereof.

Additionally or alternatively, a reduction in the Clinical Global Impression-Severity (CGI-S) score, occurs on day 1, for instance, about 24 hours after the last administration of 5-MeO-DMT or a pharmaceutically acceptable salt thereof.

An improvement in tension, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in tension, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

The inventors furthermore conclude that a reduction or elimination of tension by treating a mental or nervous system disorder patient does not only lead to a reduction in the BPRS total score, but also to an improvement in maternal functioning, as reflected by an increase in the BIMF score. This improvement will be achieved rapidly, namely within about 2 hours, and an increased BIMF score will also be observed on day 1, for instance, after about 24 hours; on day 7; on day 14; and/or on day 28 after the last administration of 5-MeO-DMT or a pharmaceutically acceptable salt thereof.

Since tension also affects other aspects of a mental or nervous system disorder, the inventors conclude that the observed improvement in the “tension” item on the BPRS will additionally contribute to an overall improvement in maternal functioning.

Improvements in one or more aspects of a mental or nervous system disorder will also lead to overall improvements. Preferably, treatment leads to a remission.

A remission of depressive symptoms may be reflected by a MADRS score equal to or less than 10 and occurs not later than about 2 hours; occurs on day 1, for instance, after about 24 hours; on day 7; on day 14 and/or on day 28 after the last administration of 5-MeO-DMT or a pharmaceutically acceptable salt thereof.

Further alternatively or in addition, a remission of depressive symptoms may be reflected by a HAM-D score equal to or less than 7 and occurs not later than about 2 hours; occurs on day 1, for instance, after about 24 hours; on day 7; on day 14 and/or on day 28 after the last administration of 5-MeO-DMT or a pharmaceutically acceptable salt thereof.

It follows from the above that treatment of mental or nervous system disorder patients with 5-MeO-DMT or a pharmaceutically acceptable salt thereof does not only lead to a reduction of the MADRS score, including in particular the subscores as detailed above, but also to improvements in the domains of the BIMF scale. The reduction of the MADRS score as well as improvements in maternal functioning are confirmed by clinical data as discussed in the example section below.

Improvements in maternal functioning include improvements in the functional domain of self-care. For instance, improvements in the MADRS items lassitude and/or reduced sleep lead to an increase in the BIMF scale scores reflecting self-care. The improvement of the cumulative score of the BIMF scale items reflecting self-care is preferably at least 10%, more preferably at least 20%.

Improvements in maternal functioning include improvements in the functional domain of infant care. For instance, improvements in the MADRS items lassitude and/or concentration difficulties lead to an increase in the BIMF scale scores reflecting infant care. The improvement of the cumulative score of the BIMF scale items reflecting self-care is preferably at least 15%, more preferably at least 25%.

Improvements in maternal functioning include improvements in the functional domain of mother-child interaction. For instance, improvements in the MADRS items inability to feel and inner tension lead to an increase in the BIMF scale scores reflecting mother-child interaction. The improvement of the cumulative score of the BIMF scale items reflecting mother-child interaction is preferably at least 5%, more preferably at least 15% Improvements in maternal functioning include improvements in the functional domain of psychological well-being. For instance, improvements in the MADRS items lassitude, pessimistic thoughts, inability to feel, inner tension and/or reduced sleep lead to an increase in the BIMF scale scores reflecting psychological well-being. The improvement of the cumulative score of the BIMF scale items reflecting psychological well-being is preferably at least 25%, more preferably at least 35%.

Improvements in maternal functioning include improvements in the functional domain of social support. For instance, improvements in the MADRS item pessimistic thoughts leads to an increase in the BIMF scale scores reflecting social support. The improvement of the cumulative score of the BIMF scale items reflecting social support is preferably at least 10%, more preferably at least 20% Improvements in maternal functioning include improvements in the functional domain of management. For instance, improvements in the MADRS items lassitude, pessimistic thoughts and/or concentration difficulties lead to an increase in the BIMF scale scores reflecting management. The improvement of the cumulative score of the BIMF scale items reflecting management is preferably at least 20%, more preferably at least 30% Improvements in maternal functioning include improvements in the functional domain of adjustment. For instance, improvements in the MADRS item lassitude leads to an increase in the BIMF scale scores reflecting adjustment. The improvement of the cumulative score of the BIMF scale items reflecting adjustment is preferably at least 5%, more preferably at least 15% The improvement in maternal functioning relates to one or more, in particular two or more functional domains according to the Barkin Index of Maternal Functioning (BIMF) selected from self-care, infant care, mother-child interaction, psychological wellbeing of the mother, social support, management, and adjustment.

The BIMF total score is improved by 10% or more, preferably by 20% or more.

Breastfeeding

Breastfeeding, as the term is used herein, is the process of feeding human breast milk to a child. Breastfeeding includes feeding milk directly from the breast, as well as feeding the child with breast milk previously pumped and then bottle fed.

As indicated above, for many medications, breastfeeding patients may be confronted with a situation where a decision must be made whether to discontinue breastfeeding or to discontinue/abstain from therapy.

In case the decision is taken to discontinue breast-feeding in order to receive a treatment, this decision will have a negative impact on maternal functioning and in particular compromise the functional domains mother-child interaction and psychological well-being.

The present invention also addresses the need for treating a mental or nervous system disorder in a breastfeeding mother without substantial interruption of breastfeeding.

According to the invention, breastfeeding can be resumed shortly after the treatment.

The inventors have investigated pharmacokinetic properties and metabolization of 5-MeO-DMT in an effort to determine from which point in time onwards after administration of 5-MeO-DMT or of a pharmaceutically acceptable salt breastfeeding is possible without exposing the suckling child to any relevant risk.

Moreover, breast milk was obtained from a breastfeeding patient treated with 5-MeO-DMT for PPD. As described in more detail in the example section, a breastfeeding patient suffering from PPD received a dose of 6 mg 5-MeO-DMT and, after 1 hour, a further dose of 12 mg 5-MeO-DMT.

Breast milk samples as well as serum und urine samples were analysed at several points in time after the last administration of 5-MeO-DMT for presence of 5-MeO-DMT, bufotenine (a primary metabolite of 5-MeO-DMT) and 5-MIAA (a final metabolite of 5-MeO-DMT).

As regards the administered compound, 5-MeO-DMT, itself, absorption and distribution are rapid, with maximum concentrations and pharmacological effects observed during and immediately after dosing, for example, by inhalation or by injection.

Plasma protein binding is low (13-23%).

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Uptitration as disclosed herein will not lead to accumulation and thus not to higher plasma concentrations of 5-MeO-DMT, for instance, 10 minutes, 2 hours, or 3 hours after administration. The rapid decline of the plasma concentration has as a consequence that there is only a short period of time during which 5-MeO-DMT can enter into breast milk. Consequently, 5-MeO-DMT will be found in breast milk only during a short period of time.

The patient data obtained confirm the fast decrease of the 5-MeO-DMT concentration in breast milk (see Example 12). Measurement after 24 hours did not detect any 5-MeO-DMT.

When determining the potential impact of maternal drug administration on a breastfed infant, it is standard practice to calculate the Relative Infant Dose (RID) (Bennett, P. N., and L. J. Notarianni. “Risk from drugs in breast milk: an analysis by relative dose.” Br J Clin Pharmacol 42.5 (1996): P673-4). The RID is the dosage (in μg/kg/day) the infant is exposed to by the intake of breast milk, divided by the dosage (in μg/kg/day) the mother receives.

Using estimates for daily intake of breast milk and the number of feeds as well as the measured breast milk concentrations the exposure of the infant can be determined.

For example, a published estimate for daily intake of breast milk as a function of infant weight is 150 ml/kg/day. To model the exposure, it is assumed that an infant of 5 kg is fed three times over 24 hours, receiving 250 ml breast milk each time. For the first feed, a 5-MeO-DMT concentration of 2167.0 μg/ml (the value determined at 1 hour) is assumed, for the second feed of 560.6 μg/ml (the value determined at 2.5 hours), and for the third feed of 42.1 μg/ml (the value determined at 8.5 hours). On this basis, the total exposure of the infant (Daily Infant Dosage, DID) is 692425 μg 5-MeO-DMT/day (0.000692425 mg/day), which value corresponds to 0.000138485 mg/kg/day.

As regards the maternal 5-MeO-DMT exposure, the total dose is 18 mg (6 mg as the first dose and 12 mg as the second dose according to the uptitration scheme applied).

Since the amount actually delivered to the patient may be lower than the indicated dose, a calculation of the RID is also carried out assuming that the actually delivered amount is only 50% of the indicated dose so that any a potential underestimation of the infant exposure is avoided.

Assuming a standard maternal weight of 60 kg, RIDs of 0.092% (based on 9 mg as the maternal exposure) to 0.046% (based on 18 mg as the maternal exposure) are obtained. The accepted threshold for “low risk” RID is 10%. It is clear from the calculated value that the 5-MeO-DMT RID is significantly below this threshold.

It is moreover noted that a range of 0.046% to 0.092% is a conservative estimate. It assumes constant 5-MeO-DMT concentrations for the period until the next feed (despite the rapid drop in actual concentrations) and ignores the fact that there are likely to be more than three feeds per day, with each smaller volume feed exposing the infant to decreasing concentrations of 5-MeO-DMT over time.

Furthermore, for the estimate it is assumed that no breast milk is expressed and discarded.

Thus, the actual RID will be lower than the estimate.

Metabolites of 5-MeO-DMT which may occur in humans were identified to assess the potential relevance of such metabolites. In an in vitro metabolism identification study of human hepatocytes, 5-MeO-DMT free base was incubated for up to 120 minutes at 10 μM. Compounds identified and their relative proportions are shown in Table 1 below:

TABLE 1

| Compound | Relative Proportion |
|---|---|
| Bufotenine (5-OH-DMT) | 5% |
| 5-MeO-DMT | 7% |
| 5-MeO-DMT-N-oxide | 1% |
| 5-Methoxyindole-3-ethanol (5-methoxy- | 25% |
| tryptophol) | |
| 5-Methoxyindole acetic acid (5-MIAA) | 61% |

It is noted that subsequent assays repeatedly failed to detect the presence of 5-methoxytryptophol but reproducibly indicated the presence of 5-MIAA as the predominant metabolite. 5-Methoxytryptophol will not play any significant role in vivo.

Metabolites as listed in the above table are formed via three different pathways.

The two most significant metabolites, 5-methoxyindole acetic acid and 5-methoxyindole-3-ethanol, are formed via oxidative deamination. This involves enzymatic removal of the N-methyl groups and oxidation so that an acetaldehyde is formed:

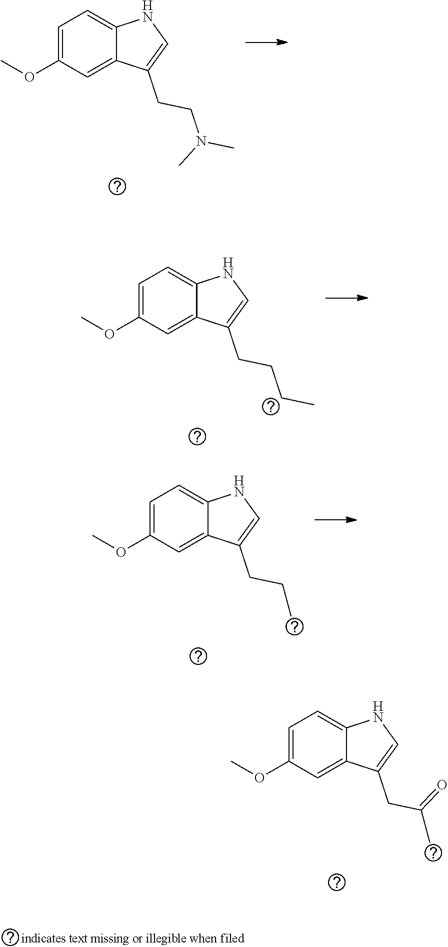

The indicated reaction is catalysed by monoamine oxidase A (MAO-A).

The secondary amine, the primary amine and the aldehyde were not identified which indicates that they are not present at any time in a significant concentration.

The aldehyde intermediate metabolite undergoes 2 separate biotransformations in human liver hepatocytes. It is either oxidised to 5-methoxyindole acetic acid (5-MIAA) or reduced to 5-methoxyindole-3-ethanol.

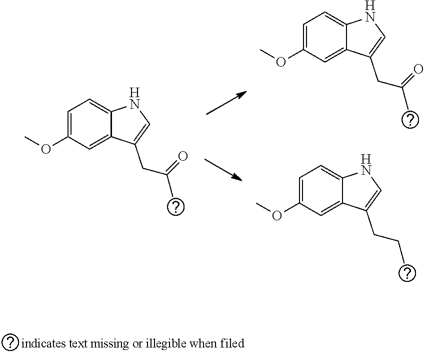

Both resulting metabolites are endogenous substances and are formed in the human body, for instance, during synthesis and metabolism of melatonin and serotonin (see e.g. Biochemistry of the Pineal. Chapter 3. in Melatonin and the Mammalian Pineal Gland. Arendt J (Ed.) Chapman & Hall, 1995; Slominski R and Slominski A T. Synthesis and Metabolism of Melatonin in the Skin and Retinal Pigment Epithelium. Chapter 3. in Melatonin in the Promotion of Health. Watson RR (Ed.) CRC Press 2012).

Since the predominant pathway of 5-MeO-DMT metabolization rapidly leads to metabolites that are also part of endogenous metabolic pathways, the inventors determined that the oxidative deamination of 5-MeO-DMT does not involve metabolites that would require imposing a limitation regarding breastfeeding.

Furthermore, as described in detail in the example section, incubation of 5-methoxytryptophol with human hepatocytes indicates a high metabolic turnover, with complete disappearance of the compound in 24 h. At 1 μM test concentration, in vitro intrinsic clearance for 5-methoxytryptophol was 16.2 μl/min/million cells (half-life 142 min).

Thus, the plasma concentration of 5-methoxytryptophol, should it be formed at all, would rapidly decrease and reach endogenous levels.

5-MIAA has been identified as the major human metabolite.

Incubation of 5-MIAA with human hepatocytes indicates a low metabolic turnover with, remaining 5-MIAA concentrations after 72h being 75-82%. 5-MIAA is considered to be a final metabolite of 5-MeO-DMT.

5-MIAA shows relatively low plasma binding of ˜50% (mean fraction unbound (Fu); see the example section). It remains in circulation subject to renal clearance.

If the endogenous formation of 5-MIAA is disregarded and if it is assumed that 5-MIAA is instantaneously formed from 5-MeO-DMT after administration of a single dose of that compound, a standard glomerular filtration rate of 90-120 ml/min would suggest that 5-MIAA is removed from circulation for urinary excretion in approximately 1-2 hours.

For example, the 5-MIAA urine concentration determined after 2.5 hours of 12 980 501 μg/ml (about 12.98 mg/I) demonstrates that most of the 5-MIAA formed will be rapidly excreted.

In consequence, the plasma concentration of 5-MIAA will rapidly decrease.

For more precise estimates of the development of the plasma concentration of 5-MIAA, several factors must be taken into consideration, including patient size and the increase in blood volume that occurs during pregnancy. It is understood that there is inter-individual variation of the glomerular filtration rate and of the rate of metabolization of 5-MeO-DMT.

Moreover, while the formation of 5-MIAA is rapid, pharmacokinetic data for healthy volunteers as well as for PPD patients indicate that small amounts of 5-MeO-DMT (corresponding to less than 10% Cmax) may still be present after about 1 hour. In consequence, 5-MIAA will in fact be formed over some period of time after administration of 5-MeO-DMT.

Still further, while in case of administration of more than one dosage of 5-MeO-DMT no accumulation of 5-MeO-DMT will occur if the interval between the administrations is at least about 1 hour, since there are still measurable amounts of 5-MIAA present in serum at 2.5 hours, there will be some accumulation of this metabolite if the subject is dosed 1 hour apart.

5-MIAA is a weak acid, which will be present in plasma in ionized form, which reduces the tendency of the compound to enter into breast milk.

It can nevertheless be assumed that some 5-MIAA will enter into breast milk, in particular during the limited time period of relatively high 5-MIAA plasma concentrations.

Further information regarding the concentration time profile follows from the measured breast milk concentrations of 5-MIAA outlined in Example 12. Using these data, estimates for the exposure to 5-MIAA of the infant can be calculated.

For the first feed of 250 ml, a 5-MIAA concentration of 13945.2 μg/ml (the value determined at 1 hour) is assumed, for the second feed of 13240.9 μg/ml (the value determined at 2.5 hours), and for the third feed of 359.4 μg/ml (the value determined at 8.5 hours). On this basis, the total exposure of the infant (DID) is 0.00688638 mg 5-MIAA/day, which value corresponds to 0.00137728 mg/kg/day.

It is noted that the 5-MIAA concentration in breast milk at 24 hours is nearly 400-fold lower than the concentration at one hour so that there will be no relevant exposure after 1 day.

The maternal exposure to 5-MIAA can be estimated based on the amount of 5-MeO-DMT administered and the proportion of 5-MeO-DMT converted into 5-MIAA.

In the metabolization experiment described above, the mixture contains about 60% 5-MIAA after 2 hours. Since metabolization is not complete, the actual proportion of 5-MeO-DMT converted into 5-MIAA will be higher. It can be assumed that more than 60% up to close to 100% of 5-MeO-DMT will be converted into 5-MIAA as the final metabolite, which is then excreted.

Taking the molecular weights into account (218.29 g/mol for 5-MeO-DMT and 205.21 g/mol for 5-MIAA), a total dose of 18 mg 5-MeO-DMT leads to between 10.15 mg 5-MIAA (60% conversion) and 16.92 mg 5-MIAA (100% conversion). Assuming as above that the delivered amount of 5-MeO-DMT is only 9 mg, between 5.08 mg 5-MIAA (60% conversion) and 8.46 mg 5-MIAA (100% conversion) is formed.

Thus, the maternal exposure will be between 0.085 mg/kg/day and 0.282 mg/kg/day. This leads to an estimate for the RID of between 0.49% and 1.62%.

As indicated, the accepted threshold for “low risk” RID is 10%, and it is clear from the calculated values, which represent conservative estimates, that the 5-MIAA RID is significantly below this threshold.

Moreover, the risk profile for 5-MIAA is low considering not just the sub-threshold daily RID value but the fact that the compound is endogenously formed as a metabolite of certain naturally occurring tryptophane derivatives, such as serotonin.

Finally, levels of bufotenine, a primary metabolite of 5-MeO-DMT, were assessed in urine, serum, and breast milk as described in Example 12. Notably, bufotenine was not detected in serum and breastmilk at any time point, and it was only detected in urine at the 2.5-hour timepoint (32.3 μg/ml). This data further demonstrates that bufotenine does not add to the risk profile for 5-MeO-DMT.

The above calculations are based on an uptitration regimen involving doses of 6 mg and 12 mg 5-MeO-DMT. The conclusions reached are valid as well for single doses up to 12 mg, which will lead to lower exposure to 5-MeO-DMT as well as its metabolites.

Extrapolation to higher doses, assuming a linear relationship between dose increment and increase in breast milk concentration and thus infant exposure leads to the conclusion that the RID for 5-MeO-DMT as well as for 5-MIAA will still be significantly below 10% for a breastfeeding mother treated with a higher dose of 5-MeO-DMT, for instance, 18 mg or 25 mg as single dose or as final dose of an uptitration scheme.

This holds true even if the reservoir effect for 5-MIAA in breastmilk is considered.

The linear extrapolation is justified based on the observed linear pharmacokinetic profile for 5-MeO-DMT.

Finally, the single-day administration must be taken into account when considering risk to the infant, as it eliminates the need to consider cumulative infant exposures over an extended period of time, as is the case for chronic treatment regimes.

A further metabolite identified, bufotenine, is the result of O-demethylation, which is catalysed by CYP2D6. The metabolized formed is then subject to glucuronidation, which is catalysed by UGT:

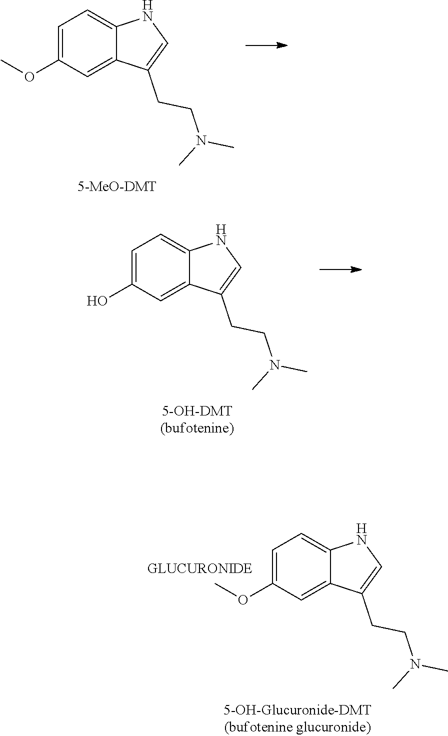

As part of a pharmacokinetic study, it was determined that bufotenine is barely detected in human serum. In no case is it detected 15 minutes after administration of 5-MeO-DMT. A small amount was detected in urine at 2.5 hours (Example 12).

Bufotenine glucuronide cannot bind to receptors and does not exert any effect. Moreover, its concentration is so low that it was not detected in the hepatocyte assay.

Bufotenine glucuronide is further converted to 5-hydroxyindole acetic acid:

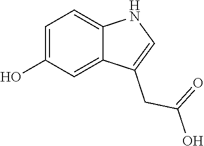 [SMILES: O=C(O)Cc1c[nH]c2ccc(O)cc12]

5-hydroxyindole acetic acid is an endogenous substance, for instance, it occurs in the metabolism of melatonin and serotonin (references as above).

Since the O-demethylation pathway of 5-MeO-DMT only plays a minor role and leads to a primary metabolite, bufotenine, which is rapidly cleared from the plasma and the further metabolization leads to compounds present only in very low concentration and ultimately to a metabolite that is also part of endogenous metabolic pathways, the inventors determined that the O-demethylation of 5-MeO-DMT does not involve metabolites that would require imposing a limitation regarding breastfeeding.

The third metabolic pathway involves N-oxidation:

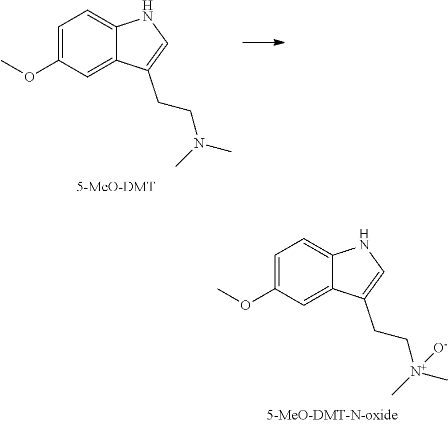

In silico modelling of the metabolite formed, 5-MeO-DMT-N-oxide was deemed to be non-genotoxic in line with the negative in vitro genotoxicity assessment of the parent molecule. The compound is water soluble and subject to rapid excretion, as confirmed by observations in the rat (Sitaram, B. R., Lockett, L., Blackman, G. L., McLeod, W. R., 1987. Urinary excretion of 5-methoxy-N,N-dimethyltryptamine, N,N-dimethyltryptamine and their N-oxides in the rat. Biochemical Pharmacology 36: 2235-2231). Since the pathway of 5-MeO-DMT metabolization involving N-oxidation plays only a minor role and leads to a low proportion of a metabolite without apparent toxicity which is rapidly excreted, the inventors determined that the N-oxidation of 5-MeO-DMT does not involve metabolites that would require imposing a limitation regarding breastfeeding.

Based on the above, the inventors have determined that breast feeding can be resumed shortly after the treatment with 5-MeO-DMT or a pharmaceutically acceptable salt thereof.

It is safe for a breastfeeding patient to only temporarily cease breastfeeding. Breastfeeding will not normally be possible during the actual treatment, i.e., during administration of 5-MeO-DMT or the pharmaceutically acceptable salt thereof and during the subsequent period of psychedelic experience. However, once the immediate effects of the administration are over, a breastfeeding patient may safely resume breastfeeding.

Early resumption of breastfeeding constitutes a significant advantage for lactating patients, their wellbeing and that of their infant.

The treated mother may be advised to temporarily cease breastfeeding, for instance, for a certain time period or until a certain event.

Ceasing breastfeeding means that the infant is neither directly fed from the breast nor fed with breastmilk pumped when breastfeeding is discouraged. Bottle feeding breastmilk obtained beforehand is, however, possible. It is the timepoint at which the breastmilk is expressed (and not when the child is fed) that is determinative.

Breastfeeding can be resumed immediately after the indicated time period or event.

For instance, the mother is advised to temporarily cease breastfeeding only for the period of the actual treatment, for instance, until the Clinical Assessment of Discharge Readiness (CADR) indicates discharge readiness.

The Clinical Assessment of Discharge Readiness (CADR) is carried out to determine that there are no clinical obstacles preventing the patient from returning home.

Discharge readiness according to the CADR requires finding that any adverse events are resolved or, if not resolved, are not preventing discharge; that the patient is fully orientated; that the patient has no hallucinations or perception distortions; that the patient is alert (responds readily to name spoken in normal tone; (Modified Observer's Assessment of Alertness/Sedation scored as 5); that the vital signs are without clinically significant changes compared to baseline; and that the patient is discharge ready in the opinion of the treating physician.

The CADR may be administered about 1 hour after administration of the last dose. Alternatively, a licensed professional may perform their own discharge readiness assessment on the basis of relevant factors such as patient vital signs and/or alertness/sedation.

The patient may be advised not to recommence breastfeeding before the later of discharge and 6 hours after the last dose; preferably the later of discharge and 3 hours after the last dose; more preferably the later of discharge and 2 hours after the last dose; in particular the later of discharge and 1 hour after the last dose.

It is also possible to wait longer until resuming breastfeeding, for instance, until the concentrations of 5-MeO-DMT and/or its metabolites in a breastmilk sample fall below certain thresholds.

For instance, breastfeeding may be temporarily ceased until the 5-MeO-DMT concentration in a breastmilk sample falls below 2000 μg/ml, 500 μg/ml or 75 μg/ml and/or until the 5-MIAA concentration in breastmilk falls below 14000 μg/ml, 2000 μg/ml or 75 μg/ml.

Alternatively, breastmilk may be pumped and discarded until the concentrations of 5-MeO-DMT and/or 5-MIAA fall below the indicated levels.

Further, breastfeeding may be temporarily ceased for a fixed period, for instance, based on clinical experience regarding concentrations of 5-MeO-DMT and/or its metabolites in breastmilk. In one example, the patient is advised to cease breastfeeding until 48 hours after receiving the last dose of 5-MeO-DMT or a pharmaceutically acceptable salt thereof. The patient is preferably advised to discontinue breastfeeding until 24 hours after the last administration of 5-MeO-DMT or a pharmaceutically acceptable salt thereof, more preferably until 12 hours after the last administration of 5-MeO-DMT or a pharmaceutically acceptable salt thereof. Still more preferably, breastfeeding has to be interrupted for only 6 hours, even more preferably, for only 3 hours, in particular for only 2 hours and most preferably for only 1 hours after the last administration of 5-MeO-DMT or a pharmaceutically acceptable salt thereof.

This short interruption and the corresponding possibility to resume breastfeeding shortly after treatment contributes to treatment success and in particular to maternal functioning and well-being and development of the infant(s).

It is desired that the concentration of 5-MeO-DMT and/or 5-MIAA in breast milk is as low as possible, in order to avoid any relevant risk on the suckling child. Any relevant risk on the suckling child can be avoided, if any expressed breast milk is discarded as long as the concentrations of 5-MeO-DMT and/or 5-MIAA in breast milk exceed predetermined thresholds or breastfeeding is only resumed when the 5-MeO-DMT concentration and/or the 5-MIAA concentration in breast milk is below a predetermined threshold. In a preferred embodiment, the threshold for 5-MeO-DMT in breast milk is as low as possible. In a further preferred embodiment, the threshold for 5-MIAA in breast milk is as low as possible. In a most preferred embodiment, the threshold for both, 5-MeO-DMT and 5-MIAA, in breast milk is as low as possible.

In the following different preferred thresholds for 5-MeO-DMT and/or 5-MIAA in expressed breast milk are provided:

| | |
|---|---|
| Threshold for 5-MeO-DMT in | Threshold for 5-MIAA in |
| expressed breast milk | expressed breast milk |

| | | | |
|---|---|---|---|
| 2000 | pg | 14000 | pg |
| 2000 | pg | 2000 | pg |
| 2000 | pg | 75 | pg |
| 500 | pg | 14000 | pg |
| 500 | pg | 2000 | pg |
| 500 | pg | 75 | pg |
| 75 | pg | 14000 | pg |
| 75 | pg | 2000 | pg |
| 75 | pg | 75 | pg |

The Delivered Infant Dose (DID) for 5-MeO-DMT and/or 5-MIAA

The Delivered Infant Dose (DID) for 5-MeO-DMT and/or 5-MIAA should be kept as low as possible. In a preferred embodiment, the DID for 5-MeO-DMT is kept as low as possible. In a further preferred embodiment, the DID for 5-MIAA is kept as low as possible. In a most preferred embodiment, the DID for both, 5-MeO-DMT and 5-MIAA, is kept as low as possible.

Relevant DID for 5-MeO-DMT and/or 5-MIAA of the invention are as follows:

| DID for 5-MeO-DMT per kg | DID for 5-MIAA per kg |
|---|---|
| infant weight for the first | infant weight for the first |
| 24 hours of feeding after | 24 hours of feeding after |
| resumption of breastfeeding | resumption of breastfeeding |
| 1 μg/kg/day or below | 4 μg/kg/day or below |
| 1 μg/kg/day or below | 2 μg/kg/day or below |
| 1 μg/kg/day or below | 1 μg/kg/day or below |
| 0.4 μg/kg/day or below | 4 μg/kg/day or below |
| 0.4 μg/kg/day or below | 2 μg/kg/day or below |
| 0.4 μg/kg/day or below | 1 μg/kg/day or below |
| 0.2 μg/kg/day or below | 4 μg/kg/day or below |
| 0.2 μg/kg/day or below | 2 μg/kg/day or below |
| 0.2 μg/kg/day or below | 1 μg/kg/day or below |

In order to achieve the above DID, breastfeeding needs adaption. This adaptation is selected from an appropriate time point for resumption of breastfeeding, an appropriate number of feeds during the first 24 hours after resumption of breastfeeding, by pumping and discarding breastmilk for an appropriate period of time or by a combination of these measures.

The Relative Infant Dose (RID) for 5-MeO-DMT and/or 5-MIAA

The Relative Infant Dose (RID) for 5-MeO-DMT and/or 5-MIAA should be as low as possible. In a preferred embodiment, the RID for 5-MeO-DMT is kept as low as possible. In a further preferred embodiment, the RID for 5-MIAA is kept as low as possible. In a most preferred embodiment, the RID for both, 5-MeO-DMT and 5-MIAA, is kept as low as possible.

Relevant RID for 5-MeO-DMT and/or 5-MIAA of the invention are as follows:

| RID for 5-MeO-DMT for the | RID for 5-MIAA for the |
|---|---|
| first 24 hours of feeding after | first 24 hours of feeding after |
| resumption of breastfeeding | resumption of breastfeeding |
| 10% or below | 4% or below |
| 10% or below | 2% or below |
| 10% or below | 1% or below |
| 5% or below | 4% or below |
| 5% or below | 2% or below |
| 5% or below | 1% or below |
| 1% or below | 4% or below |
| 1% or below | 2% or below |
| 1% or below | 1% or below |

In order to achieve the above RID, breastfeeding needs adaption. This adaptation is selected from an appropriate time point for resumption of breastfeeding, an appropriate number of feeds during the first 24 hours after resumption of breastfeeding, by pumping and discarding breastmilk for an appropriate period of time or by a combination of these measures.

Combination of the Delivered Infant Dose (DID) and the Relative Infant Dose (RID) for 5-MeO-DMT and/or 5-MIAA

It has been described that the DID and the RID for 5-MeO-DMT and/or 5-MIAA are relevant aspects of the present invention. While each individual aspect is relevant as such, in a preferred embodiment of the invention both aspects, the DID for 5-MeO-DMT and/or 5-MIAA and the RID for 5-MeO-DMT and/or 5-MIAA, should be considered in combination. Relevant DID and relevant RID that can be combined are provided in the present invention.

In one embodiment, the DID for 5-MeO-DMT and the RID for 5-MeO-DMT are combined. In a preferred embodiment, the DID and the RID for 5-MeO-DMT is kept as low as possible. Relevant DID and relevant RID that can be combined are provided in the present invention.

In a further embodiment, the DID for 5-MIAA and the RID for 5-MIAA are combined. In a preferred embodiment, the DID and the RID for 5-MIAA is kept as low as possible. Relevant DID and relevant RID that can be combined are provided in the present invention.

In a further embodiment, the DID for 5-MeO-DMT and the RID for 5-MIAA are combined. In a preferred embodiment, the DID for 5-MeO-DMT and the RID for 5-MIAA is kept as low as possible. Relevant DID and relevant RID that can be combined are provided in the present invention.

In a further embodiment, the RID for 5-MeO-DMT and the DID for 5-MIAA are combined. In a preferred embodiment, the RID for 5-MeO-DMT and the DID for 5-MIAA is kept as low as possible. Relevant DID and relevant RID that can be combined are provided in the present invention.

In a more preferred embodiment, the DID for 5-MeO-DMT and 5-MIAA and the RID for 5-MeO-DMT and 5-MIAA are combined. In a preferred embodiment, the DID for 5-MeO-DMT and 5-MIAA and the RID for 5-MeO-DMT and 5-MIAA is kept as low as possible. Relevant DID and relevant RID that can be combined are provided in the present invention.

Treatment of Mental or Nervous System Conditions

Disorders Characterized by Depressive Episodes

There are several disorders which are characterized by depressive episodes.

A depressive episode is a period of depressed mood and/or loss of pleasure in most activities.

For instance, according to DSM-V, a Major Depressive Episode is characterized by five or more symptoms that have been present during the same 2-week period and represent a change from previous functioning; at least one of the symptoms being either (1) depressed mood or (2) loss of interest or pleasure.

The patient suffering from a Disorder Characterized by Depressive Episodes may suffer from a treatment resistant form of the disorder.

Disorders Characterized by Depressive Episodes involve one or more of sleep disturbance, cognitive dysfunction, anxiety, psychomotor retardation, social/emotional withdrawal and negative thinking.

Severity and treatment success can be assessed, for instance, by the Montgomery-Asberg Depression Rating Scale (MADRS) or the Hamilton Rating Scale for Depression (HAM-D).

In patients suffering from a disorder characterized by depressive episodes altered functional connectivity is observed within and/or between several brain regions implicated in processing, regulation, emotional memory; cognitive processes related to rumination; impaired concentration and physiological arousal.

Treating a patient suffering from a Disorder Characterized by Depressive Episodes, including a treatment resistant form of the disorder, with 5-MeO-DMT or a pharmaceutically acceptable salt thereof leads to an improvement of the Disorder Characterized by Depressive Episodes.

An improvement of the Disorder Characterized by Depressive Episodes, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of the Disorder Characterized by Depressive Episodes in a patient, as reflected by a reduction in the CGI-S score, occurs not later than about 2 hours after the last administration of 5-MeO-DMT or a pharmaceutically acceptable salt thereof. An improvement of the Disorder Characterized by Depressive Episodes, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

An improvement of the Disorder Characterized by Depressive Episodes, as reflected by a reduction in the MADRS score, is observed on day 1, for instance after about 24 hours; on day 7; on day 14; and/or on day 28 after the last administration of 5-MeO-DMT or a pharmaceutically acceptable salt thereof.

An improvement of the Disorder Characterized by Depressive Episodes in a patient, as reflected by a reduction in the MADRS score, occurs not later than about 2 hours after the last administration of 5-MeO-DMT or a pharmaceutically acceptable salt thereof. An improvement of the Disorder Characterized by Depressive Episodes, as reflected by a reduction in the MADRS score, preferably persists until at least 6 days; in particular until at least 14 days; more preferably until at least 28 days after the last administration of 5-MeO-DMT or a pharmaceutically acceptable salt thereof.

An improvement of the Disorder Characterized by Depressive Episodes, as reflected by a reduction in the HAM-D score, is observed on day 1, for instance after about 24 hours; on day 7; on day 14; and/or on day 28 after the last administration of 5-MeO-DMT or a pharmaceutically acceptable salt thereof.

An improvement of the Disorder Characterized by Depressive Episodes in a patient, as reflected by a reduction in the HAM-D score, occurs not later than about 2 hours after the last administration of 5-MeO-DMT or a pharmaceutically acceptable salt thereof. An improvement of the Disorder Characterized by Depressive Episodes, as reflected by a reduction in the HAM-D score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from a Disorder Characterized by Depressive Episodes is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from a Disorder Characterized by Depressive Episodes, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Major Depressive Disorder (MDD) is a mood disorder that causes a persistent feeling of sadness and loss of interest. It affects how a person feels, thinks, and behaves and can lead to a variety of emotional and physical problems.

The patient may suffer from moderate or severe MDD as indicated by a Montgomery-Åsberg Depression Rating Scale (MADRS) score of 20 or more or by a Hamilton Depression Rating Scale (HAM-D) score of 17 or more. It is further considered that the patient may suffers from severe major depressive disorder as indicated by a Montgomery-Asberg Depression Rating Scale (MADRS) score of 35 or more or by a Hamilton Depression Rating Scale (HAM-D) score of 25 or more.

The patient suffering from MDD may suffer from a treatment resistant form of the disorder (TRD).

MDD involves one or more of sleep disturbance, cognitive dysfunction, anxiety, psychomotor retardation, social/emotional withdrawal and negative thinking.

Severity and treatment success can be assessed, for instance, by the Montgomery-Asberg Depression Rating Scale (MADRS) or the Hamilton Rating Scale for Depression (HAM-D).

In patients suffering from MDD dysfunctional connectivity and regulation within and/or between multiple resting-state networks including the DMN, salience network, executive control network and limbic network is observed. Functional connectivity differs significantly from that observed in healthy controls.

Treating a patient suffering from MDD, including a treatment resistant form of the disorder, with 5-MeO-DMT or a pharmaceutically acceptable salt thereof leads to an improvement of the MDD.

An improvement of MDD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of MDD in a patient, as reflected by a reduction in the CGI-S score, occurs not later than about 2 hours after the last administration of 5-MeO-DMT or a pharmaceutically acceptable salt thereof. An improvement of MDD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

An improvement of MDD, as reflected by a reduction in the MADRS score, is observed on day 1, for instance after about 24 hours; on day 7; on day 14; and/or on day 28 after the last administration of 5-MeO-DMT or a pharmaceutically acceptable salt thereof.

An improvement of MDD in a patient, as reflected by a reduction in the MADRS score, occurs not later than about 2 hours after the last administration of 5-MeO-DMT or a pharmaceutically acceptable salt thereof. An improvement of MDD, as reflected by a reduction in the MADRS score, preferably persists until at least 6 days; in particular until at least 14 days; more preferably until at least 28 days after the last administration of 5-MeO-DMT or a pharmaceutically acceptable salt thereof.

An improvement of MDD, as reflected by a reduction in the HAM-D score, is observed on day 1, for instance after about 24 hours; on day 7; on day 14; and/or on day 28 after the last administration of 5-MeO-DMT or a pharmaceutically acceptable salt thereof.

An improvement of MDD in a patient, as reflected by a reduction in the HAM-D score, occurs not later than about 2 hours after the last administration of 5-MeO-DMT or a pharmaceutically acceptable salt thereof. An improvement of MDD, as reflected by a reduction in the HAM-D score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from MDD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from MDD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Persistent Depressive Disorder

Persistent Depressive Disorder, also referred to as dysthymia, is a chronic form of depression. It is diagnosed if depression is present for most of the day for the majority of days over at least a two-year period. Any symptom-free period is less than 2 months.

While depressed, two or more of the following must be present: 1. Hopelessness; 2. Energy low or fatigue; 3. Self-esteem is low; 4. Sleep decreased (insomnia) or increased (hypersomnia): 5. Appetite poor, or overeating; 6. Difficulty making decisions or poor concentration.

The patient suffering from Persistent Depressive Disorder may suffer from a treatment resistant form of the disorder.

Persistent Depressive Disorder involves one or more of sleep disturbance, cognitive dysfunction, anxiety, psychomotor retardation, social/emotional withdrawal and negative thinking.

Severity and treatment success can be assessed, for instance, by the Montgomery-Asberg Depression Rating Scale (MADRS) or the Hamilton Rating Scale for Depression (HAM-D).

In patients suffering from Persistent Depressive Disorder altered functional connectivity is observed within and/or between several brain regions implicated in processing, regulation, emotional memory; cognitive processes related to rumination; impaired concentration and physiological arousal. Dysfunctional connectivity is observed within and/or between the DMN, salience network, executive control network and limbic network. Functional connectivity differs significantly from that observed in healthy controls.

Treating a patient suffering from Persistent Depressive Disorder, including a treatment resistant form of the disorder, with 5-MeO-DMT or a pharmaceutically acceptable salt thereof leads to an improvement of the Persistent Depressive Disorder.

An improvement of Persistent Depressive Disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Persistent Depressive Disorder in a patient, as reflected by a reduction in the CGI-S score, occurs not later than about 2 hours after the last administration of 5-MeO-DMT or a pharmaceutically acceptable salt thereof. An improvement of Persistent Depressive Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

An improvement of Persistent Depressive Disorder, as reflected by a reduction in the MADRS score, is observed on day 1, for instance after about 24 hours; on day 7; on day 14; and/or on day 28 after the last administration of 5-MeO-DMT or a pharmaceutically acceptable salt thereof.

An improvement of Persistent Depressive Disorder in a patient, as reflected by a reduction in the MADRS score, occurs not later than about 2 hours after the last administration of 5-MeO-DMT or a pharmaceutically acceptable salt thereof. An improvement of Persistent Depressive Disorder, as reflected by a reduction in the MADRS score, preferably persists until at least 6 days; in particular until at least 14 days; more preferably until at least 28 days after the last administration of 5-MeO-DMT or a pharmaceutically acceptable salt thereof.

An improvement of Persistent Depressive Disorder, as reflected by a reduction in the HAM-D score, is observed on day 1, for instance after about 24 hours; on day 7; on day 14; and/or on day 28 after the last administration of 5-MeO-DMT or a pharmaceutically acceptable salt thereof.

An improvement of Persistent Depressive Disorder in a patient, as reflected by a reduction in the HAM-D score, occurs not later than about 2 hours after the last administration of 5-MeO-DMT or a pharmaceutically acceptable salt thereof. An improvement of Persistent Depressive Disorder, as reflected by a reduction in the HAM-D score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Persistent Depressive Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Persistent Depressive Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Seasonal Affective Disorder

Seasonal Affective Disorder is a mood disorder with seasonal pattern, with symptoms often beginning in autumn and remitting in spring. Many people experience sadness, hopelessness, a loss of interest in activities, tiredness, and social withdrawal.

The patient suffering from Seasonal Affective Disorder may suffer from a treatment resistant form of the disorder.

Seasonal Affective Disorder involves one or more of sleep disturbance, cognitive dysfunction, anxiety, psychomotor retardation, social/emotional withdrawal and negative thinking.

Severity and treatment success can be assessed, for instance, by the Montgomery-Asberg Depression Rating Scale (MADRS) or the Hamilton Rating Scale for Depression (HAM-D).

In patients suffering from Seasonal Affective Disorder altered functional connectivity is observed within and/or between several brain regions implicated in processing, regulation, emotional memory; cognitive processes related to rumination; impaired concentration and physiological arousal. Dysfunctional connectivity is observed within and/or between the DMN, salience network, executive control network and limbic network. Functional connectivity differs significantly from that observed in healthy controls.

Treating a patient suffering from Seasonal Affective Disorder, including a treatment resistant form of the disorder, with 5-MeO-DMT or a pharmaceutically acceptable salt thereof leads to an improvement of the Seasonal Affective Disorder.

An improvement of Seasonal Affective Disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Seasonal Affective Disorder in a patient, as reflected by a reduction in the CGI-S score, occurs not later than about 2 hours after the last administration of 5-MeO-DMT or a pharmaceutically acceptable salt thereof. An improvement of Seasonal Affective Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

An improvement of Seasonal Affective Disorder, as reflected by a reduction in the MADRS score, is observed on day 1, for instance after about 24 hours; on day 7; on day 14; and/or on day 28 after the last administration of 5-MeO-DMT or a pharmaceutically acceptable salt thereof.

An improvement of Seasonal Affective Disorder in a patient, as reflected by a reduction in the MADRS score, occurs not later than about 2 hours after the last administration of 5-MeO-DMT or a pharmaceutically acceptable salt thereof. An improvement of Seasonal Affective Disorder, as reflected by a reduction in the MADRS score, preferably persists until at least 6 days; in particular until at least 14 days; more preferably until at least 28 days after the last administration of 5-MeO-DMT or a pharmaceutically acceptable salt thereof.

An improvement of Seasonal Affective Disorder, as reflected by a reduction in the HAM-D score, is observed on day 1, for instance after about 24 hours; on day 7; on day 14; and/or on day 28 after the last administration of 5-MeO-DMT or a pharmaceutically acceptable salt thereof.

An improvement of Seasonal Affective Disorder in a patient, as reflected by a reduction in the HAM-D score, occurs not later than about 2 hours after the last administration of 5-MeO-DMT or a pharmaceutically acceptable salt thereof. An improvement of Seasonal Affective Disorder, as reflected by a reduction in the HAM-D score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Seasonal Affective Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Seasonal Affective Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Bipolar Disorder

Bipolar Disorder (BD) is a mental health condition characterized by extreme mood swings that include emotional lows (major depressive episodes) and highs (manic or hypomanic episodes). BD is a recurrent chronic disorder that affects more than 1% of the world's population irrespective of ethnic origin or socioeconomic status.

The patient suffering from BD may suffer from a treatment resistant form of the disorder.

BD is classified as Bipolar I Disorder if there has been at least one manic episode, with or without depressive episodes. It is classified as Bipolar II Disorder if there has been at least one hypomanic episode (but no full manic episodes) and one major depressive episode. If these symptoms are due to drugs or medical problems, they are not diagnosed as Bipolar Disorder.

The patient suffering from BD, whether diagnosed with Bipolar II Disorder or with Bipolar I Disorder, in particular suffers from a current major depressive episode. The severity of a current major depressive episode may be assessed using the Montgomery-Åsberg Depression Rating Scale (MADRS). The patient may have a total score of equal to or greater than 19, such as greater or equal than 24, in particular greater or equal than 37.

Alternatively or in addition, the patient may have a Bipolar Depression Rating Scale (BDRS) total score of 19, such as greater or equal than 24, in particular greater or equal than 37.

BD involves one or more of sleep disturbance, cognitive dysfunction, anxiety, psychomotor retardation, social/emotional withdrawal and negative thinking.

Severity and treatment success can be assessed, for instance, by the Montgomery-Åsberg Depression Rating Scale (MADRS), the Hamilton Rating Scale for Depression (HAM-D) or the Bipolar Depression Rating Scale (BDRS).

The BDRS is designed to measure the severity of depressive symptoms in bipolar depression. The BDRS is validated for clinical use by trained raters. Based on a clinical interview, the BDRS items rate the severity of depressive and/or mixed symptoms expressed by patients currently and during the past few days. If there is a discordance between symptoms currently and the last few days, the rating should reflect current symptoms. The scale contains 20 questions and the maximum score possible is 60. Higher scores indicate greater severity.

The questions address depressed mood; sleep disturbance; appetite disturbance; reduced social engagement; reduced energy and activity; reduced motivation; impaired concentration and memory; anxiety; anhedonia; affective flattening; feelings of worthlessness; feelings of helplessness and hopelessness; suicidal ideation; feelings of guilt; psychotic symptoms; irritability; lability; increased motor drive; increased speech; agitation.

Each of these aspects is assessed and assigned a score of 0, 1, 2 or 3.

Patients suffering from BD show characteristic aberrant intrinsic organization and interconnectivity of resting state networks. In comparison with healthy controls, resting state functional magnetic resonance imaging studies demonstrated functional connectivity alterations of specific regions within and/or between the default mode network, the salience network, and the central executive network. In particular functional connectivity alterations within the default mode network are also observed in patients suffering from sleep disturbance.

Treating a patient suffering from BD, including a treatment resistant form of the disorder, with 5-MeO-DMT or a pharmaceutically acceptable salt thereof leads to an improvement of the BD.

An improvement of BD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of BD in a patient, as reflected by a reduction in the CGI-S score, occurs not later than about 2 hours after the last administration of 5-MeO-DMT or a pharmaceutically acceptable salt thereof. An improvement of BD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

An improvement of BD, as reflected by a reduction in the MADRS score, is observed on day 1, for instance after about 24 hours; on day 7; on day 14; and/or on day 28 after the last administration of 5-MeO-DMT or a pharmaceutically acceptable salt thereof.

An improvement of BD in a patient, as reflected by a reduction in the MADRS score, occurs not later than about 2 hours after the last administration of 5-MeO-DMT or a pharmaceutically acceptable salt thereof. An improvement of BD, as reflected by a reduction in the MADRS score, preferably persists until at least 6 days; in particular until at least 14 days; more preferably until at least 28 days after the last administration of 5-MeO-DMT or a pharmaceutically acceptable salt thereof.

An improvement of BD, as reflected by a reduction in the HAM-D score, is observed on day 1, for instance after about 24 hours; on day 7; on day 14; and/or on day 28 after the last administration of 5-MeO-DMT or a pharmaceutically acceptable salt thereof.

An improvement of BD in a patient, as reflected by a reduction in the HAM-D score, occurs not later than about 2 hours after the last administration of 5-MeO-DMT or a pharmaceutically acceptable salt thereof. An improvement of BD, as reflected by a reduction in the HAM-D score, preferably persists until at least 6 days; in particular until at least 14 days; more preferably until at least 28 days after the last administration of 5-MeO-DMT or a pharmaceutically acceptable salt thereof.

An improvement of BD, as reflected by a reduction in the BDRS score, is observed on day 1, for instance after about 24 hours; on day 7; on day 14; and/or on day 28 after the last administration of 5-MeO-DMT or a pharmaceutically acceptable salt thereof.

An improvement of BD in a patient, as reflected by a reduction in the BDRS score, occurs not later than about 2 hours after the last administration of 5-MeO-DMT or a pharmaceutically acceptable salt thereof. An improvement of BD, as reflected by a reduction in the BDRS score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from BD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from BD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Anxiety Disorders

An Anxiety Disorder is a type of mental health condition. Symptoms include feelings of nervousness, panic and fear as well as sweating and a rapid heartbeat. Anxiety involves a complex cognitive, affective, physiological, and behavioural response system associated with preparation for anticipated events or circumstances perceived as threatening.

An Anxiety Disorder also involves one or more of sleep disturbance, cognitive dysfunction, social/emotional withdrawal and negative thinking.

The patient suffering from an Anxiety Disorder may suffer from a treatment resistant form of the disorder.

The severity of an Anxiety Disorder can be assessed by using the Beck Anxiety Inventory (BAI). It can also be assessed by using the Hamilton Anxiety Rating Scale (HAM-A); the Psychic Anxiety subscale of the HAM-A (sum of items 1-6 and 14); the Anxious Mood item of the HAM-A (item 1); the Brief Psychiatric Rating Scale (BPRS) item “anxiety”.

Anxiety Disorders are moreover associated with suicidal ideation.

Suicidal ideation may be assessed using the Columbia Suicide Severity Rating Scale (C-SSRS).

Anxiety disorders are studied by functional magnetic resonance imaging. In general, all anxiety disorders show abnormalities in the default mode network (DMN). Further networks affected by these disorders are the salience network (SN) and the sensorimotor network (SMN). The resting state balance within and/or between each of these networks, e.g., SMN and SN, relative to the DMN may be abnormal in the different anxiety disorders.

Treating a patient suffering from an Anxiety Disorder, including a treatment resistant form for the Disorder, with 5-MeO-DMT or a pharmaceutically acceptable salt thereof reduces or eliminates the anxiety.

The reduction or elimination of anxiety in a patient suffering from an Anxiety Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from an Anxiety Disorder occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from an Anxiety Disorder, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from an Anxiety Disorder, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from an Anxiety Disorder, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such an Anxiety Disorder, including a treatment resistant form of the Disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of anxiety in a patient suffering from such an Anxiety Disorder, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such an Anxiety Disorder, including a treatment resistant form of the Disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from an Anxiety Disorder, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from an Anxiety Disorder, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from an Anxiety Disorder, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from an Anxiety Disorder, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from an Anxiety Disorder, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from an Anxiety Disorder, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from an Anxiety Disorder, including a treatment resistant form of the Disorder, with 5-MeO-DMT or a pharmaceutically acceptable salt thereof reduces or eliminates suicidal ideation.

The reduction or elimination of suicidal ideation in a patient suffering from an Anxiety Disorder, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from an Anxiety Disorder, including a treatment resistant form of the Disorder, occurs not later than about 2 hours after the last administration of 5-MeO-DMT or a pharmaceutically acceptable salt thereof. The reduction or elimination of suicidal ideation preferably persists until at least 6 days; in particular until at least 14 days; more preferably until at least 28 days after the last administration of 5-MeO-DMT or a pharmaceutically acceptable salt thereof.

The above applies to Anxiety Disorders generally, including the specific conditions discussed in detail below as well as anxiety disorder due to a medical condition, which occurs when a medical condition causes extreme fear, anxiety, or panic; other specified anxiety disorder, which may be diagnosed if a patients has most but not all of the criteria for an anxiety disorder; and unspecified anxiety disorder; which is often diagnosed if a patient experiences anxiety or panic but there is a lack of information to make a complete diagnosis of another anxiety disorder.

The improvement of maternal functioning in a patient suffering from an Anxiety Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from an Anxiety Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Separation Anxiety Disorder is characterized by an excessive anxiety regarding separation from home and/or from someone to whom the patient has a strong emotional attachment.

Situations of separation cause the patient significant distress, and they may have difficulty going to school or work due to the separation. Patients suffering from Separation Anxiety Disorder may also have excessive anxiety about unwelcome events happening to important people in their lives, such as family members. Separation Anxiety Disorder also involves one or more of sleep disturbance, cognitive dysfunction, social/emotional withdrawal and negative thinking.

A patient suffering from Separation Anxiety Disorder may suffer from a treatment resistant form of the disorder.

The severity of Separation Anxiety Disorder can be assessed by using the Beck Anxiety Inventory (BAI). It can also be assessed by using the Hamilton Anxiety Rating Scale (HAM-A); the Psychic Anxiety subscale of the HAM-A (sum of items 1-6 and 14); the Anxious Mood item of the HAM-A (item 1); the Brief Psychiatric Rating Scale (BPRS) item “anxiety”.

Separation Anxiety Disorder is moreover associated with suicidal ideation.

Suicidal ideation may be assessed using the Columbia Suicide Severity Rating Scale (C-SSRS).

Separation Anxiety Disorder disorders are studied by functional magnetic resonance imaging. In general, all anxiety disorders show abnormalities in the default mode network (DMN). Further networks affected by these disorders are the salience network (SN) and the sensorimotor network (SMN). The resting state balance within and/or between each of these networks, e.g., SMN and SN, relative to the DMN may be abnormal in the different anxiety disorders.

Treating a patient suffering from Separation Anxiety Disorder, including a treatment resistant form for the Disorder, with 5-MeO-DMT or a pharmaceutically acceptable salt thereof reduces or eliminates the anxiety.

The reduction or elimination of anxiety in a patient suffering from Separation Anxiety Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Separation Anxiety Disorder occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from Separation Anxiety Disorder, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from Separation Anxiety Disorder, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from Separation Anxiety Disorder, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such Separation Anxiety Disorder, including a treatment resistant form of the Disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of anxiety in a patient suffering from such Separation Anxiety Disorder, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such Separation Anxiety Disorder, including a treatment resistant form of the disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Separation Anxiety Disorder, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Separation Anxiety Disorder, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Separation Anxiety Disorder, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Separation Anxiety Disorder, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Separation Anxiety Disorder, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Separation Anxiety Disorder, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from Separation Anxiety Disorder, including a treatment resistant form of the Disorder, with 5-MeO-DMT or a pharmaceutically acceptable salt thereof reduces or eliminates suicidal ideation.

The reduction or elimination of suicidal ideation in a patient suffering from Separation Anxiety Disorder, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from Separation Anxiety Disorder, including a treatment resistant form of the Disorder, occurs not later than about 2 hours after the last administration of 5-MeO-DMT or a pharmaceutically acceptable salt thereof. The reduction or elimination of suicidal ideation preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Separation Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Separation Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Agoraphobia is a fear of situations or places that may cause feelings of panic, entrapment, helplessness, or embarrassment.

A patient suffering from Agoraphobia may have difficulty leaving the house. The thought of leaving the house may cause considerable anxiety to the point of avoidance. Fears of crowds, traveling, elevators, movie theatres, malls, etc., might cause significant challenges.

Patients with Agoraphobia may also have recurrent panic attacks.

Agoraphobia also involves one or more of sleep disturbance, cognitive dysfunction, social/emotional withdrawal and negative thinking.

A patient suffering from Agoraphobia may suffer from a treatment resistant form of the disorder.

The severity of Agoraphobia can be assessed by using the Beck Anxiety Inventory (BAI). It can also be assessed by using the Hamilton Anxiety Rating Scale (HAM-A); the Psychic Anxiety subscale of the HAM-A (sum of items 1-6 and 14); the Anxious Mood item of the HAM-A (item 1); the Brief Psychiatric Rating Scale (BPRS) item “anxiety”.

Agoraphobia is moreover associated with suicidal ideation.

Suicidal ideation may be assessed using the Columbia Suicide Severity Rating Scale (C-SSRS).

Functional magnetic resonance imaging in individuals suffering from Agoraphobia reveals alterations in functional connectivity within and/or between resting-state networks involved in Anxiety.

Treating a patient suffering from Agoraphobia, including a treatment resistant form for the Disorder, with 5-MeO-DMT or a pharmaceutically acceptable salt thereof reduces or eliminates the anxiety.

The reduction or elimination of anxiety in a patient suffering from Agoraphobia is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Agoraphobia occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from Agoraphobia, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from Agoraphobia, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from Agoraphobia, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such Agoraphobia, including a treatment resistant form of the Disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of anxiety in a patient suffering from such Agoraphobia, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such Agoraphobia, including a treatment resistant form of the Disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Agoraphobia, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Agoraphobia, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Agoraphobia, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Agoraphobia, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Agoraphobia, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Agoraphobia, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from Agoraphobia, including a treatment resistant form of the Disorder, with 5-MeO-DMT or a pharmaceutically acceptable salt thereof reduces or eliminates suicidal ideation.

The reduction or elimination of suicidal ideation in a patient suffering from Agoraphobia, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from Agoraphobia, including a treatment resistant form of the Disorder, occurs not later than about 2 hours after the last administration of 5-MeO-DMT or a pharmaceutically acceptable salt thereof.

The reduction or elimination of suicidal ideation preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Agoraphobia is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Agoraphobia, as reflected by an improvement in the BIMF total score, occurs not later than about 6 days after the last administration of 5-MeO-DMT or a pharmaceutically acceptable salt thereof.

The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Generalized Anxiety Disorder (GAD) is characterized by persistent and excessive, difficult to control worry about a wide range of situations and issues. Patients suffering from GAD may anticipate disaster and may be overly concerned about money, health, family, work, or other issues.

GAD is diagnosed when an individual experiences persistent worry about everyday challenges out of proportion to the perceived threat. Patients with GAD usually experience excessive fear that can last months to years.

GAD interferes with social, occupational, or other important areas of functioning.

GAD also involves one or more of sleep disturbance, cognitive dysfunction, social/emotional withdrawal and negative thinking.

The patient suffering from GAD may suffer from a treatment resistant form of the disorder.

The severity of GAD can be assessed by using the Beck Anxiety Inventory (BAI). It can also be assessed by using the Hamilton Anxiety Rating Scale (HAM-A); the Psychic Anxiety subscale of the HAM-A (sum of items 1-6 and 14); the Anxious Mood item of the HAM-A (item 1); the Brief Psychiatric Rating Scale (BPRS) item “anxiety”.

GAD is moreover associated with suicidal ideation.

Suicidal ideation may be assessed using the Columbia Suicide Severity Rating Scale (C-SSRS).

Patients with GAD show also altered functional connectivity, especially within the default mode network.

Treating a patient suffering from GAD, including a treatment resistant form for the Disorder, with 5-MeO-DMT or a pharmaceutically acceptable salt thereof reduces or eliminates the anxiety.

The reduction or elimination of anxiety in a patient suffering from GAD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from GAD occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from GAD, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from GAD, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from GAD, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such GAD, including a treatment resistant form of the Disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of anxiety in a patient suffering from such GAD, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such GAD, including a treatment resistant form of the Disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from GAD, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from GAD, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from GAD, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from GAD, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from GAD, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from GAD, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from GAD, including a treatment resistant form of the Disorder, with 5-MeO-DMT or a pharmaceutically acceptable salt thereof reduces or eliminates suicidal ideation.

The reduction or elimination of suicidal ideation in a patient suffering from GAD, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from GAD, including a treatment resistant form of the Disorder, occurs not later than about 2 hours after the last administration of 5-MeO-DMT or a pharmaceutically acceptable salt thereof. The reduction or elimination of suicidal ideation preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from GAD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from GAD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Social Anxiety Disorder (SAD), also called social phobia, is one of the most common types of anxiety.

SAD is characterized by intense anxiety or fear of being judged, negatively evaluated, or rejected in a social or performance situation. This often leads to avoidance of the social situation and can cause impairments in school, work, or relationships.

SAD also involves one or more of sleep disturbance, cognitive dysfunction, social/emotional withdrawal and negative thinking.

The patient suffering from SAD may suffer from a treatment resistant form of the disorder.

The severity of SAD can be assessed by using the Beck Anxiety Inventory (BAI). It can also be assessed by using the Hamilton Anxiety Rating Scale (HAM-A); the Psychic Anxiety subscale of the HAM-A (sum of items 1-6 and 14); the Anxious Mood item of the HAM-A (item 1); the Brief Psychiatric Rating Scale (BPRS) item “anxiety”.

SAD is moreover associated with suicidal ideation.

Suicidal ideation may be assessed using the Columbia Suicide Severity Rating Scale (C-SSRS).

In SAD patients, altered functional connectivity within and/or between resting state networks, such as the default mode network and salience network, is observed.

Treating a patient suffering from SAD, including a treatment resistant form for the Disorder, with 5-MeO-DMT or a pharmaceutically acceptable salt thereof reduces or eliminates the anxiety.

The reduction or elimination of anxiety in a patient suffering from SAD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from SAD occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from SAD, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from SAD, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from SAD, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such SAD, including a treatment resistant form of the disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof. A remission of anxiety in a patient suffering from such SAD, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such SAD, including a treatment resistant form of the Disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from SAD, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from SAD, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from SAD, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from SAD, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from SAD, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from SAD, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from SAD, including a treatment resistant form of the Disorder, with 5-MeO-DMT or a pharmaceutically acceptable salt thereof reduces or eliminates suicidal ideation.

The reduction or elimination of suicidal ideation in a patient suffering from SAD, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from SAD, including a treatment resistant form of the Disorder, occurs not later than about 2 hours after the last administration of 5-MeO-DMT or a pharmaceutically acceptable salt thereof. The reduction or elimination of suicidal ideation preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from SAD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from SAD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Anxiety is a core feature of Panic Disorder. Patients suffering from Panic Disorder experience spontaneous panic attacks with an abrupt onset of intense fear or discomfort that reaches a peak within minutes.

The panic attacks feature many somaticized symptoms of anxiety including sweating, trembling, shaking, headache, palpitations, shortness of breath, chest pain, abdominal pain, and nausea.

Furthermore, the disorder can often feature anxiety of future panic attacks. Patients may be very preoccupied with the fear of a recurring attack.

Panic Disorder also involves one or more of sleep disturbance, cognitive dysfunction, social/emotional withdrawal and negative thinking.

The patient suffering from Panic Disorder may suffer from a treatment resistant form of the disorder.

The severity of Panic Disorder can be assessed by using the Beck Anxiety Inventory (BAI). It can also be assessed by using the Hamilton Anxiety Rating Scale (HAM-A); the Psychic Anxiety subscale of the HAM-A (sum of items 1-6 and 14); the Anxious Mood item of the HAM-A (item 1); the Brief Psychiatric Rating Scale (BPRS) item “anxiety”.

Panic Disorder is moreover associated with suicidal ideation.

Suicidal ideation may be assessed using the Columbia Suicide Severity Rating Scale (C-SSRS).

Patients with panic disorder show altered functional connectivity within and/or between the default mode network and sensorimotor network.

Treating a patient suffering from Panic Disorder, including a treatment resistant form for the Disorder, with 5-MeO-DMT or a pharmaceutically acceptable salt thereof reduces or eliminates the anxiety.

The reduction or elimination of anxiety in a patient suffering from Panic Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Panic Disorder occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from Panic Disorder, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from Panic Disorder, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from Panic Disorder, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such Panic Disorder, including a treatment resistant form of the disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of anxiety in a patient suffering from such Panic Disorder, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such Panic Disorder, including a treatment resistant form of the Disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Panic Disorder, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Panic Disorder, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Panic Disorder, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Panic Disorder, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Panic Disorder, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Panic Disorder, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from Panic Disorder, including a treatment resistant form of the Disorder, with 5-MeO-DMT or a pharmaceutically acceptable salt thereof reduces or eliminates suicidal ideation.

The reduction or elimination of suicidal ideation in a patient suffering from Panic Disorder, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from Panic Disorder, including a treatment resistant form of the Disorder, occurs not later than about 2 hours after the last administration of 5-MeO-DMT or a pharmaceutically acceptable salt thereof.

The reduction or elimination of suicidal ideation preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Panic Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Panic Disorder, as reflected by an improvement in the BIMF total score, occurs not later than about 6 days after the last administration of 5-MeO-DMT or a pharmaceutically acceptable salt thereof.

The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

A Phobia is an anxiety disorder defined by a persistent and excessive fear of an object or situation. A patient suffering from phobia experiences extreme anxiety when anticipating exposure or being exposed to a feared stimulus. There are animal type (spiders, snakes, dogs) phobias, natural environment type (tornadoes, heights, water, fire) phobias, blood injection type (needles, medical procedures) phobias, situational type (flying on an airplane, enclosed spaces) phobias and other type phobias (phobias that do not fit into one the previous categories).

Phobias typically result in a rapid onset of fear and are usually present for more than six months. Patients make great efforts to avoid the feared stimulus. Fear and avoidance cause significant distress and/or impairment in occupational, academic, or social functioning.

Phobia also involves one or more of sleep disturbance, cognitive dysfunction, social/emotional withdrawal and negative thinking.

The patient suffering from Phobia may suffer from a treatment resistant form of the disorder.

The severity of Phobia can be assessed by using the Beck Anxiety Inventory (BAI). It can also be assessed by using the Hamilton Anxiety Rating Scale (HAM-A); the Psychic

Anxiety subscale of the HAM-A (sum of items 1-6 and 14); the Anxious Mood item of the HAM-A (item 1); the Brief Psychiatric Rating Scale (BPRS) item “anxiety”.

Phobia is moreover associated with suicidal ideation.

Suicidal ideation may be assessed using the Columbia Suicide Severity Rating Scale (C-SSRS).

In individuals with specific Phobia, the amygdala, the anterior cingulate cortex (ACC) and insular cortex all appear to be hyperresponsive to phobia-related stimuli. For instance, functional neuroimaging studies identified the dorsal part of the anterior cingulate cortex (dACC), which is part of the salience network, to be hyperresponsive to phobia-related stimuli or the anticipation of such stimuli.

Treating a patient suffering from Phobia, including a treatment resistant form for the Disorder, with 5-MeO-DMT or a pharmaceutically acceptable salt thereof reduces or eliminates the anxiety.

The reduction or elimination of anxiety in a patient suffering from Phobia is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Phobia occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from Phobia, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from Phobia, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from Phobia, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such Phobia, including a treatment resistant form of the Disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of anxiety in a patient suffering from such Phobia, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such Phobia, including a treatment resistant form of the Disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Phobia, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Phobia, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Phobia, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Phobia, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Phobia, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Phobia, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from Phobia, including a treatment resistant form of the Disorder, with 5-MeO-DMT or a pharmaceutically acceptable salt thereof reduces or eliminates suicidal ideation.

The reduction or elimination of suicidal ideation in a patient suffering from Phobia, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from Phobia, including a treatment resistant form of the Disorder, occurs not later than about 2 hours after the last administration of 5-MeO-DMT or a pharmaceutically acceptable salt thereof. The reduction or elimination of suicidal ideation preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Phobia is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Phobia, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Substance/Medication-Induced Anxiety Disorder is an anxiety disorder in which anxiety or panic occurs after using alcohol, a drug of abuse, or a medication or after a toxin exposure. Substance/Medication-Induced Anxiety Disorder leads to prominent symptoms of panic or anxiety and can occur during the intoxication or withdrawal phases of using a substance or medication.

The disturbance causes clinically significant distress or impairment in social, occupational, or other important areas of functioning.

While taking substances or medication or within a short time thereafter, individuals suffering from Substance/Medication-Induced Anxiety Disorder may feel nervous and worried, experience symptoms of negative thinking, may have trouble concentrating or remembering things, may have fear of losing control or insanity or death, may lose weight due to gastrointestinal problems, may have chills, hot flashes, sweating, shaking, numbness, or a pounding heartbeat, trouble breathing, trouble swallowing, or chest pain.

Substance/Medication-Induced Anxiety Disorder also involves one or more of sleep disturbance, cognitive dysfunction, social/emotional withdrawal and negative thinking.

The severity of Substance/Medication-Induced Anxiety Disorder can be assessed by using the Beck Anxiety Inventory (BAI). It can also be assessed by using the Hamilton Anxiety Rating Scale (HAM-A); the Psychic Anxiety subscale of the HAM-A (sum of items 1-6 and 14); the Anxious Mood item of the HAM-A (item 1); the Brief Psychiatric Rating Scale (BPRS) item “anxiety”.

Substance/Medication-Induced Anxiety Disorder is moreover associated with suicidal ideation.

Suicidal ideation may be assessed using the Columbia Suicide Severity Rating Scale (C-SSRS).

Functional magnetic resonance imaging in individuals suffering from Substance/medication-induced Anxiety Disorder reveals alterations in functional connectivity within and/or between resting-state networks involved in anxiety.

Treating a patient suffering from Substance/Medication-Induced Anxiety Disorder, including a treatment resistant form for the Disorder, with 5-MeO-DMT or a pharmaceutically acceptable salt thereof reduces or eliminates the anxiety.

The reduction or elimination of anxiety in a patient suffering from Substance/Medication-Induced Anxiety Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Substance/Medication-Induced Anxiety Disorder occurs not later than about 2 hours after the last administration of 5-MeO-DMT or a pharmaceutically acceptable salt thereof. The reduction or elimination of anxiety preferably persists until at least 6 days; in particular until at least 14 days; more preferably until at least 28 days after the last administration of 5-MeO-DMT or a pharmaceutically acceptable salt thereof.

A clinical response in a patient suffering from Substance/Medication-Induced Anxiety Disorder, including a treatment resistant form of the Disorder, is reflected by an at least 50% decrease of the HAM-A score, compared to the respective score prior to treatment, about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A clinical response in a patient suffering from Substance/Medication-Induced Anxiety Disorder, including a treatment resistant form of the Disorder, as reflected by an at least 50% decrease of the HAM-A score, compared to the respective score prior to treatment, occurs not later than about 2 hours after the last administration of 5-MeO-DMT or a pharmaceutically acceptable salt thereof. The clinical response in a patient suffering from Substance/Medication-Induced Anxiety Disorder, including a treatment resistant form of the Disorder, as reflected by an at least 50% decrease of the HAM-A score, preferably persists until at least 6 days; in particular until at least 14 days; more preferably until at least 28 days after the last administration of 5-MeO-DMT or a pharmaceutically acceptable salt thereof.

A remission of anxiety in a patient suffering from such Substance/Medication-Induced Anxiety Disorder, including a treatment resistant form of the Disorder, is reflected by a HAM-A score of 7 or less about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A remission of anxiety in a patient suffering from such Substance/Medication-Induced Anxiety Disorder, including a treatment resistant form of the Disorder, as reflected by a HAM-A score of 7 or less, occurs not later than about 2 hours after the last administration of 5-MeO-DMT or a pharmaceutically acceptable salt thereof. The remission of anxiety in a patient suffering from such Substance/Medication-Induced Anxiety Disorder, including a treatment resistant form of the Disorder, as reflected by a HAM-A score of 7 or less, preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Substance/Medication-Induced Anxiety Disorder, including a treatment resistant form of the Disorder, as reflected by a decrease of the score of the Psychic Anxiety subscale of the HAM-A (sum of items 1-6 and 14), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Substance/Medication-Induced Anxiety Disorder, including a treatment resistant form of the Disorder, as reflected by a decrease of the score of the Psychic Anxiety subscale of the HAM-A (sum of items 1-6 and 14) score, occurs not later than about 2 hours after the last administration of 5-MeO-DMT or a pharmaceutically acceptable salt thereof. The reduction or elimination of anxiety, as reflected by a decrease of the score of the Psychic Anxiety subscale of the HAM-A (sum of items 1-6 and 14), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Substance/Medication-Induced Anxiety Disorder, including a treatment resistant form of the Disorder, as reflected by a decrease of the score of the Anxious Mood item of the HAM-A (item 1), is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Substance/Medication-Induced Anxiety Disorder, including a treatment resistant form of the Disorder, as reflected by a decrease of the score of the Anxious Mood item of the HAM-A (item 1), occurs not later than about 2 hours after the last administration of 5-MeO-DMT or a pharmaceutically acceptable salt thereof. The reduction or elimination of anxiety, as reflected by a decrease of the score of the Anxious Mood item of the HAM-A (item 1), preferably persists until at least 6 days; in particular until at least 14 days; more preferably until at least 28 days after the last administration of 5-MeO-DMT or a pharmaceutically acceptable salt thereof.

The reduction or elimination of anxiety in a patient suffering from Substance/Medication-Induced Anxiety Disorder, including a treatment resistant form of the Disorder, as reflected by a decrease of the score of the BPRS item “anxiety”, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of anxiety in a patient suffering from Substance/Medication-Induced Anxiety Disorder, including a treatment resistant form of the Disorder, as reflected by a decrease of the score of the BPRS item “anxiety”, occurs not later than about 2 hours after the last administration of 5-MeO-DMT or a pharmaceutically acceptable salt thereof. The reduction or elimination of anxiety, as reflected by a decrease of the score of the BPRS item “anxiety”, preferably persists until at least 6 days; in particular until at least 14 days; more preferably until at least 28 days after the last administration of 5-MeO-DMT or a pharmaceutically acceptable salt thereof.

Treating a patient suffering from Substance/Medication-Induced Anxiety Disorder, including a treatment resistant form of the Disorder, with 5-MeO-DMT or a pharmaceutically acceptable salt thereof reduces or eliminates suicidal ideation.

The reduction or elimination of suicidal ideation in a patient suffering from Substance/Medication-Induced Anxiety Disorder, including a treatment resistant form of the Disorder, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of suicidal ideation in a patient suffering from Substance/Medication-Induced Anxiety Disorder, including a treatment resistant form of the Disorder, occurs not later than about 2 hours after the last administration of 5-MeO-DMT or a pharmaceutically acceptable salt thereof. The reduction or elimination of suicidal ideation preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Substance/Medication-Induced Anxiety Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Substance/Medication-Induced Anxiety Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Somatic Symptom Disorder

Somatic Symptom Disorder is a mental disorder diagnosed when a person has a significant focus on physical symptoms, such as pain, weakness, or shortness of breath to a level that results in major distress and/or problems functioning and/or cause disruption in daily life. Feelings and behaviours related to the illness are excessive or out of proportion.

Health-related quality of life is often impaired, both physically and mentally. In severe Somatic Symptom Disorder, the impairment is marked, and when persistent, the disorder can lead to invalidism.

Somatic Symptom Disorder involves one or more of sleep disturbance, cognitive dysfunction, and anxiety.

Brain functional connectivity analysis reveals alterations within and/or between resting state networks in patients with Somatic Symptom Disorder in comparison to healthy controls. Alterations are identified within and/or between default mode network (DMN), salience network, dorsal attention network (DAN) and sensorimotor network. Somatic Symptom Disorder may be associated with alterations of sensory-discriminative processing of somatic symptoms, which is influenced by affective processing.

Treating a patient suffering from Somatic Symptom Disorder, including a treatment resistant form of the disorder, with 5-MeO-DMT or a pharmaceutically acceptable salt thereof leads to an improvement of the Somatic Symptom Disorder.

An improvement of Somatic Symptom Disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Somatic Symptom Disorder in a patient, as reflected by a reduction in the CGI-S score, occurs not later than about 2 hours after the last administration of 5-MeO-DMT or a pharmaceutically acceptable salt thereof. An improvement of Somatic Symptom Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Somatic Symptom Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Somatic Symptom Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Obsessive Compulsive and Related Disorders Obsessive Compulsive Disorder (OCD) is a mental illness that causes repeated unwanted thoughts or sensations (obsessions) or the urge to do something over and over again (compulsions). Patients may suffer from both obsessions and compulsions.

The patient suffering from OCD may suffer from a treatment resistant form of the disorder.

OCD involves one or more of sleep disturbance, cognitive dysfunction, anxiety, social/emotional withdrawal and negative thinking.

Neuroimaging studies using functional magnetic resonance imaging of patients suffering from OCD show functional connectivity alterations within and/or between frontoparietal network, salience network, and default mode network. Altered functional connectivity in affected networks, in particular the default mode network, is also associated with sleep disturbances.

Treating a patient suffering from OCD, including a treatment resistant form of the disorder, with 5-MeO-DMT or a pharmaceutically acceptable salt thereof leads to an improvement of the OCD.

An improvement of OCD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of OCD in a patient, as reflected by a reduction in the CGI-S score, occurs not later than about 2 hours after the last administration of 5-MeO-DMT or a pharmaceutically acceptable salt thereof. An improvement of OCD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from OCD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from OCD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Patients suffering from Body Dysmorphic Disorder (BDD) misperceive defects in their appearance, disrupting their ability to function in their daily lives, with disturbing preoccupations, ritualistic behaviours, and emotional distress.

The patient suffering from BDD may suffer from a treatment resistant form of the disorder.

BDD involves one or more of sleep disturbance, cognitive dysfunction, anxiety, social/emotional withdrawal and negative thinking.

Functional magnetic resonance imaging of patients suffering from BDD reveals alterations within and/or between certain brain areas located in the default mode network, the dorsal attention network and the salience network. Altered functional connectivity in affected networks, in particular the default mode network, is also associated with sleep disturbances.

Treating a patient suffering from BDD, including a treatment resistant form of the disorder, with 5-MeO-DMT or a pharmaceutically acceptable salt thereof leads to an improvement of the BDD.

An improvement of BDD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of BDD in a patient, as reflected by a reduction in the CGI-S score, occurs not later than about 2 hours after the last administration of 5-MeO-DMT or a pharmaceutically acceptable salt thereof. An improvement of BDD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from BDD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from BDD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Post-Traumatic Stress Disorder (PTSD)

Post-Traumatic Stress Disorder (PTSD) is a mental health condition that can develop based on a terrifying event—either experienced or witnessed by the patient. Symptoms may include flashbacks, nightmares and severe anxiety, as well as uncontrollable thoughts about the event.

The patient suffering from PTSD may suffer from a treatment resistant form of the disorder.

PTSD involves one or more of sleep disturbance, cognitive dysfunction, anxiety, social/emotional withdrawal and negative thinking.

Analysis of resting state functional magnetic resonance imaging in patients suffering from PTSD reveals alterations within and/or between regions located in the default mode network and salience network.

Treating a patient suffering from PTSD, including a treatment resistant form of the disorder, with 5-MeO-DMT or a pharmaceutically acceptable salt thereof leads to an improvement of the PTSD.

An improvement of PTSD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of PTSD in a patient, as reflected by a reduction in the CGI-S score, occurs not later than about 2 hours after the last administration of 5-MeO-DMT or a pharmaceutically acceptable salt thereof. An improvement of PTSD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from PTSD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from PTSD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Pain Disorders

Sleep disturbance occurs in patients suffering from pain and has been associated with Chronic Pain.

Chronic Pain, also referred to as persistent pain, is long standing pain that persists beyond the usual recovery period, for instance, after an injury or operation, despite medication or treatment. Patients may also suffer from Chronic Pain without any apparent cause, such as a history of an injury or operation.

Chronic Pain involves one or more of sleep disturbance, cognitive dysfunction, anxiety, social/emotional withdrawal and negative thinking.

Chronic Pain patients display brain alterations regarding brain function and structure. These changes are related to the persistence of pain, long after the initial nociceptive input has disappeared. Resting state functional magnetic resonance imaging reveals alterations in distinct regions within and/or between the default mode network, the somatomotor/sensorimotor network, and the salience network.

Treating a patient suffering from Chronic Pain, including a treatment resistant form of the disorder, with 5-MeO-DMT or a pharmaceutically acceptable salt thereof leads to an improvement of the Chronic Pain.

An improvement of Chronic Pain, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Chronic Pain in a patient, as reflected by a reduction in the CGI-S score, occurs not later than about 2 hours after the last administration of 5-MeO-DMT or a pharmaceutically acceptable salt thereof. An improvement of Chronic Pain, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Chronic Pain is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Chronic Pain, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Fibromyalgia is a chronic disorder that is characterized by widespread musculoskeletal pain throughout the body or at multiple sites, accompanied by fatigue, sleep disturbances, memory and mood issues. Patients may also encounter muscle and joint stiffness, tenderness to touch, numbness or tingling in the arms and legs, problems with concentrating, thinking clearly, and memory (sometimes called “fibro fog”), heightened sensitivity to light, noise, odors, and temperature, or digestive issues, such as bloating or constipation.

Studies show that Fibromyalgia patients have a heightened sensitivity to pain, so they feel pain when others do not.

Fibromyalgia involves one or more of sleep disturbance, cognitive dysfunction, anxiety, social/emotional withdrawal and negative thinking.

Brain imaging studies and other research have uncovered evidence of altered signaling in neural pathways that transmit and receive pain in people with Fibromyalgia. These changes may also contribute to the fatigue, sleep disturbances, and cognitive problems that many people with the disorder experience.

Resting-state functional magnetic resonance imaging of patients with Fibromyalgia shows altered functional connectivity within and/or between the DMN and executive attention network and between the DMN and the insular cortex, a brain region known to process evoked pain.

Treating a patient suffering from Firbomyalgia, including a treatment resistant form of the disorder, with 5-MeO-DMT or a pharmaceutically acceptable salt thereof leads to an improvement of the Firbomyalgia.

An improvement of Firbomyalgia, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Firbomyalgia in a patient, as reflected by a reduction in the CGI-S score, occurs not later than about 2 hours after the last administration of 5-MeO-DMT or a pharmaceutically acceptable salt thereof. An improvement of Firbomyalgia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Firbomyalgia is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Firbomyalgia, as reflected by an improvement in the BIMF total score, occurs not later than about 6 days after the last administration of 5-MeO-DMT or a pharmaceutically acceptable salt thereof.

The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Migraine is a headache that can cause severe throbbing pain or a pulsing sensation, usually on one side of the head. Migraine is often accompanied by nausea, vomiting, and extreme sensitivity to light and sound. Migraine attacks can last for hours to days, and the pain can be so severe that it interferes with daily activities.

In some patients, a symptom known as an aura occurs before or with the headache. This symptom can include visual disturbances, such as flashes of light or blind spots, or other disturbances, such as tingling on one side of the face or in an arm or leg and difficulty speaking.

Migraine involves one or more of sleep disturbance, cognitive dysfunction, anxiety, social/emotional withdrawal and negative thinking.

Headache disorders are associated with atypical functional connectivity of regions associated with pain processing as well as atypical functional connectivity within and/or between multiple core resting state networks, including the salience and the default mode network.

In patients suffering from migraine, resting state network analysis shows differences to healthy controls. Studies during the migraine attacks reveal marked abnormalities in networks relevant for mediating cognitive, attentional, somatosensory and emotional components of pain.

Treating a patient suffering from Migraine, including a treatment resistant form of the disorder, with 5-MeO-DMT or a pharmaceutically acceptable salt thereof leads to an improvement of the Migraine.

An improvement of Migraine, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Migraine in a patient, as reflected by a reduction in the CGI-S score, occurs not later than about 2 hours after the last administration of 5-MeO-DMT or a pharmaceutically acceptable salt thereof. An improvement of Migraine, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Migraine is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Migraine, as reflected by an improvement in the BIMF total score, occurs not later than about 6 days after the last administration of 5-MeO-DMT or a pharmaceutically acceptable salt thereof.

The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Mental and Behavioural Disorders due to Psychoactive Substance Use

Sleep disturbance occurs in patients suffering from certain mental and behavioural disorders due to psychoactive substance use.

A Substance Use Disorder (SUD) is a mental disorder that affects a person's behaviour, leading to a person's inability to control their use of substances such as legal or illegal drugs, alcohol, or medications. Symptoms can range from moderate to severe, with addiction being the most severe form of SUDs.

Substance Use Disorder involves one or more of sleep disturbance, cognitive dysfunction, anxiety, social/emotional withdrawal, negative thinking, and psychomotor retardation.

Resting state functional connectivity (rsFC) was found to be altered not only in patients with sleep disturbances, but also in patients with Substance Use Disorders. In particular, deficits in cognitive control are associated with altered connectivity within and/or between resting state networks, such as the default mode network, the salience network, the central executive network, the limbic network and the reward network.

Treating a patient suffering from Substance Use Disorder, including a treatment resistant form of the disorder, with 5-MeO-DMT or a pharmaceutically acceptable salt thereof leads to an improvement of the Substance Use Disorder.

An improvement of Substance Use Disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Substance Use Disorder in a patient, as reflected by a reduction in the CGI-S score, occurs not later than about 2 hours after the last administration of 5-MeO-DMT or a pharmaceutically acceptable salt thereof. An improvement of Substance Use Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Substance Use Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Substance Use Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Psychotic Disorders

Psychotic Disorders are severe mental disorders that cause abnormal thinking and perceptions. Psychotic disorders are characterised by significant impairments in reality testing and alterations in behaviour manifest in positive symptoms such as persistent delusions, persistent hallucinations, disorganised thinking (typically manifest as disorganised speech), grossly disorganised behaviour, and experiences of passivity and control, negative symptoms such as blunted or flat affect and avolition, and psychomotor disturbances.

The patient suffering from a Psychotic Disorder may suffer from a treatment resistant form of the disorder.

Psychotic Disorders involve one or more of sleep disturbance, cognitive dysfunction, anxiety, social/emotional withdrawal, negative thinking, and psychomotor retardation.

Brain imaging of patients suffering from psychosis by functional magnetic resonance imaging of brain resting state networks, reveals profound alterations in distinct regions within and/or between the central executive network, the default mode network and the salience network. Even in patient populations at risk for psychosis, alterations in resting state networks can be identified.

Treating a patient suffering from a Psychotic Disorder, including a treatment resistant form of the disorder, with 5-MeO-DMT or a pharmaceutically acceptable salt thereof leads to an improvement of the a Psychotic Disorder.

An improvement of a Psychotic Disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of a Psychotic Disorder in a patient, as reflected by a reduction in the CGI-S score, occurs not later than about 2 hours after the last administration of 5-MeO-DMT or a pharmaceutically acceptable salt thereof. An improvement of a Psychotic Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from a Psychotic Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from a Psychotic Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Schizophrenia

Schizophrenia is a severe mental health condition characterised by disturbances in multiple mental modalities, including thinking, perception, self-experience, cognition, volition, affect and behaviour. Psychomotor disturbances, including catatonia, may be present.

The patient suffering from Schizophrenia may suffer from a treatment resistant form of the disorder.

Schizophrenia involves one or more of sleep disturbance, cognitive dysfunction, anxiety, social/emotional withdrawal, negative thinking, and psychomotor retardation.

Abnormal resting state functional connectivity, particularly within and/or between the default mode network, the frontoparietal network and the salience network, is reported in individuals with schizophrenia. In individuals at high risk for psychosis and in those diagnosed with schizophrenia, several studies have found associations between sleep disturbances and symptom severity.

Treating a patient suffering from Schizophrenia, including a treatment resistant form of the disorder, with 5-MeO-DMT or a pharmaceutically acceptable salt thereof leads to an improvement of the Schizophrenia.

An improvement of Schizophrenia, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Schizophrenia in a patient, as reflected by a reduction in the CGI-S score, occurs not later than about 2 hours after the last administration of 5-MeO-DMT or a pharmaceutically acceptable salt thereof. An improvement of Schizophrenia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Schizophrenia is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Schizophrenia, as reflected by an improvement in the BIMF total score, occurs not later than about 6 days after the last administration of 5-MeO-DMT or a pharmaceutically acceptable salt thereof.

The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Eating Disorders

An Eating Disorder is a mental disorder characterised by abnormal eating behaviours that negatively affect a person's physical or mental health.

The patient suffering from an Eating Disorder may suffer from a treatment resistant form of the disorder.

Eating Disorders involve one or more of sleep disturbance, cognitive dysfunction, anxiety, social/emotional withdrawal, and negative thinking.

In patients suffering from Eating Disorders, results from functional network connectivity studies indicate disrupted resting-state connectivity within and/or between executive networks, the default-mode network and the salience network.

Treating a patient suffering from an Eating Disorder, including a treatment resistant form of the disorder, with 5-MeO-DMT or a pharmaceutically acceptable salt thereof leads to an improvement of the an Eating Disorder.

An improvement of an Eating Disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of an Eating Disorder in a patient, as reflected by a reduction in the CGI-S score, occurs not later than about 2 hours after the last administration of 5-MeO-DMT or a pharmaceutically acceptable salt thereof. An improvement of an Eating Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from an Eating Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from an Eating Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Attention Deficit Hyperactivity Disorder (ADHD)

Attention Deficit Hyperactivity Disorder (ADHD) is a condition that affects a patient's behaviour. A patient suffering from ADHD can seem restless, may have trouble concentrating and may act on impulse.

ADHD involves one or more of sleep disturbance, cognitive dysfunction, anxiety, social/emotional withdrawal, and negative thinking.

Resting state network analysis in patients suffering from ADHD reveals dysfunctional connectivity across multiple brain resting state networks, in particular dysfunctional connectivity within and/or between distinct regions of the default mode network, the dorsal attention network and the salience network.

Treating a patient suffering from ADHD, including a treatment resistant form of the disorder, with 5-MeO-DMT or a pharmaceutically acceptable salt thereof leads to an improvement of the ADHD.

An improvement of ADHD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of ADHD in a patient, as reflected by a reduction in the CGI-S score, occurs not later than about 2 hours after the last administration of 5-MeO-DMT or a pharmaceutically acceptable salt thereof. An improvement of ADHD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from ADHD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from ADHD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Personality Disorders

Schizotypal Personality Disorder is a mental health condition marked by a consistent pattern of intense discomfort with relationships and social interactions. Patients suffering from Schizotypal Personality Disorder have unusual thoughts, speech, and behaviours, which hinder their ability to form and maintain relationships.

The patient suffering from Schizotypal Personality Disorder may suffer from a treatment resistant form of the disorder.

Schizotypal Personality Disorder involves one or more of sleep disturbance, cognitive dysfunction, anxiety, social/emotional withdrawal, and negative thinking.

In patients with Schizotypal Personality Disorder, DMN functional connectivity, particularly that involving cognitive or emotional regulation, is altered. Neuroimaging analysis by fMRI further reveals alterations within and/or between frontoparietal network and dorsal attention network.

Treating a patient suffering from Schizotypal Personality Disorder, including a treatment resistant form of the disorder, with 5-MeO-DMT or a pharmaceutically acceptable salt thereof leads to an improvement of the Schizotypal Personality Disorder.

An improvement of Schizotypal Personality Disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Schizotypal Personality Disorder in a patient, as reflected by a reduction in the CGI-S score, occurs not later than about 2 hours after the last administration of 5-MeO-DMT or a pharmaceutically acceptable salt thereof. An improvement of Schizotypal Personality Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Schizotypal Personality Disorder is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Schizotypal Personality Disorder, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Persistent mood instability, impulsivity, identity disturbance and interpersonal dysfunction are characteristic traits for Borderline Personality Disorder (BPD).

The patient suffering from BPD may suffer from a treatment resistant form of the disorder.

BPD involves one or more of sleep disturbance, cognitive dysfunction, anxiety, social/emotional withdrawal, and negative thinking.

Patients with Borderline Personality Disorder show abnormal connectivity patterns in resting-state networks. Analysis of functional connectivity of brain resting state networks using functional magnetic resonance imaging reveals alterations within and/or between various networks, such as for example the default mode network and salience network.

Treating a patient suffering from BPD, including a treatment resistant form of the disorder, with 5-MeO-DMT or a pharmaceutically acceptable salt thereof leads to an improvement of the BPD.

An improvement of BPD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of BPD in a patient, as reflected by a reduction in the CGI-S score, occurs not later than about 2 hours after the last administration of 5-MeO-DMT or a pharmaceutically acceptable salt thereof. An improvement of BPD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from BPD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from BPD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Autism Spectrum Disorder (ASD)

Patients suffering from an Autism Spectrum Disorder (ASD), including autism, Asperger's syndrome and atypical autism, have persistent deficits in the ability to initiate and to sustain reciprocal social interaction and social communication. The condition is moreover characterized by a range of restricted, repetitive, and inflexible patterns of behaviour, interests or activities that are clearly atypical or excessive for the individual's age and sociocultural context.

The onset of the disorder is during the developmental period, but symptoms may not become fully manifest until later. Deficits are sufficiently severe to affect a patient's ability to function in personal, family, social, educational, occupational or other areas.

ASD involves one or more of sleep disturbance, cognitive dysfunction, anxiety, and social/emotional withdrawal.

ASD as a disorder of brain network connectivity. Neuroimaging studies indicate that ASD is related to the anomalous responses in certain brain areas, significant alteration of brain networks, including altered functional connectivity within and/or between the default mode network (DMN) and the sensory processing network, and disturbed neural synchronization between brain areas.

Treating a patient suffering from ASD, including a treatment resistant form of the disorder, with 5-MeO-DMT or a pharmaceutically acceptable salt thereof leads to an improvement of the ASD.

An improvement of ASD, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of ASD in a patient, as reflected by a reduction in the CGI-S score, occurs not later than about 2 hours after the last administration of 5-MeO-DMT or a pharmaceutically acceptable salt thereof. An improvement of ASD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from ASD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from ASD, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Chronic Fatigue Syndrome Fatigue describes a feeling of exhaustion, lethargy, and decreased energy. Fatigue is experienced as a weakening or depletion of one's physical or mental resources. Even though considered as normal following a period of exertion, mental or physical, fatigue may occur in the absence of such exertion as a symptom of health conditions.

Chronic fatigue exacerbated by activity is moreover a prominent symptom in Chronic Fatigue Syndrome. In this disorder, severe fatigue is accompanied by neurocognitive, autonomic, and immunological symptoms.

A variety of abnormalities in normal sleep patterns, which may act as perpetuating factors, have been reported in Chronic Fatigue Syndrome patients.

In the early stages of illness, Chronic Fatigue Syndrome patients tend to complain of hypersomnia. This is often followed by a general decrease in sleep efficiency once the illness enters a more chronic stage. Even if patients had many hours of sleep, they tend not to feel refreshed when they wake up.

Compensatory behaviour for the non-restorative night-sleep can lead to alterations in circadian rhythm. Also, vivid dreams, periodic limb movements during sleep and restless legs syndrome are frequently reported.

Chronic Fatigue Syndrome involves one or more of sleep disturbance, cognitive dysfunction, and anxiety.

Patients suffering from Chronic Fatigue Syndrome show abnormal resting state functional connectivity, significantly correlated with the severity of their chronic fatigue. Networks involved are also involved in sleep regulation.

Treating a patient suffering from Chronic Fatigue Syndrome, including a treatment resistant form of the disorder, with 5-MeO-DMT or a pharmaceutically acceptable salt thereof leads to an improvement of the Chronic Fatigue Syndrome.

An improvement of Chronic Fatigue Syndrome, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Chronic Fatigue Syndrome in a patient, as reflected by a reduction in the CGI-S score, occurs not later than about 2 hours after the last administration of 5-MeO-DMT or a pharmaceutically acceptable salt thereof. An improvement of Chronic Fatigue Syndrome, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Chronic Fatigue Syndrome is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Chronic Fatigue Syndrome, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Medical Health Conditions Leading to an Associated Mental or Nervous System Condition

Patients with Traumatic Brain Injury

A traumatic brain injury (TBI) is an injury to the brain caused by an external force.

Traumatic brain injuries are characterized as primary or secondary brain injuries. A primary brain injury refers to the structural damage created during the time of impact from contact, acceleration-deceleration, and/or rotational forces. A secondary brain injury refers to the damage sustained from the subsequent cellular processes that occur from the primary injury (ie, hypoxia and/or raised intracranial pressure).

TBI leads to one or more of sleep disturbance, cognitive dysfunction, anxiety, social/emotional withdrawal and negative thinking.

Resting state functional connectivity analysis reveals alterations in overall functional connectivity within and/or between resting state networks, such as the DMN, the frontoparietal network, the executive network, and the sensory motor network. For instance, highly connected regions that can be found within the DMN are particularly susceptible to alterations in functional connectivity following traumatic brain injury.

Treating a patient suffering from TBI, including a treatment resistant form of the disorder, with 5-MeO-DMT or a pharmaceutically acceptable salt thereof leads to an improvement of the TBI.

An improvement of TBI, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of TBI in a patient, as reflected by a reduction in the CGI-S score, occurs not later than about 2 hours after the last administration of 5-MeO-DMT or a pharmaceutically acceptable salt thereof. An improvement of TBI, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from TBI is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from TBI, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Patients with HIV Infection

HIV disease is caused by infection with human immunodeficiency virus type-1 (HIV-1). Despite combined antiretroviral therapy, HIV-infected patients develop symptoms of nervous system conditions.

HIV leads to one or more of sleep disturbance, cognitive dysfunction, anxiety, social/emotional withdrawal and negative thinking.

Resting state functional connectivity analysis of patients suffering from neurocognitive disorder due to HIV infection, shows that functional connectivity is impaired within and/or between certain regions of the default mode network, the salience network, and the executive network.

Treating a patient suffering from HIV, including a treatment resistant form of the disorder, with 5-MeO-DMT or a pharmaceutically acceptable salt thereof leads to an improvement of the HIV.

An improvement of HIV, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of HIV in a patient, as reflected by a reduction in the CGI-S score, occurs not later than about 2 hours after the last administration of 5-MeO-DMT or a pharmaceutically acceptable salt thereof. An improvement of HIV, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from HIV is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from HIV, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Patients with Post COVID Condition Coronavirus disease 2019 (COVID-19) is a contagious disease caused by the severe acute respiratory syndrome coronavirus 2 (SARS-CoV-2).

Post COVID Condition (sometimes referred to as “Long COVID”) is a multisystemic condition comprising often severe symptoms that follow a SARS-CoV-2 infection. It is an often-debilitating illness that occurs in at least 10% of the infections.

The World Health Organization (WHO) defines this condition as a symptom complex which occurs within 3 months after infection, lasts at least 2 months, can fluctuate and for which there is no explanatory explanation there is not attributed to alternative diagnoses.

Post COVID Condition can include a wide range of ongoing health problems; these conditions can last weeks, months, or years and can sometimes result in disability.

Individuals suffering from Post COVID Condition most commonly report typical symptoms such as fatigue, shortness of breath and cognitive disorders such as for example difficulty thinking or concentrating (sometimes referred to as “brain fog”). However, patients may also experience respiratory and heart symptoms, neurological symptoms, for example sleep problems (for example insomnia), and digestive symptoms.

Post COVID Condition patients often experience lingering symptoms, such as anxiety, even after surviving a mild case of the disease. Anxiety in Post COVID Condition can be either a direct result of the SARS-CoV-2 infection or caused by an acute SARS-CoV-2 infection-related stay in hospital, in particular a stay in an intensive care unit.

Individuals suffering from Post COVID Condition induced anxiety often have fears about health and recovery, have stress about being off work and the impact on finances and worry about family and friends also getting ill. Moreover, Post COVID Condition induced anxiety leads to trouble sleeping, difficulty concentrating, difficulty remembering things, changes in mood, flashbacks, breathing dysfunction, chest pain and racing thoughts.

Post COVID Condition involves one or more of sleep disturbance, cognitive dysfunction, and anxiety.

Functional magnetic resonance imaging of patients suffering from Post COVID Condition reveals alterations within and/or between resting state networks. Most commonly, the default mode network is affected.

Treating a patient suffering from Post COVID Condition, including a treatment resistant form of the disorder, with 5-MeO-DMT or a pharmaceutically acceptable salt thereof leads to an improvement of the Post COVID Condition.

An improvement of Post COVID Condition, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.

An improvement of Post COVID Condition in a patient, as reflected by a reduction in the CGI-S score, occurs not later than about 2 hours after the last administration of 5-MeO-DMT or a pharmaceutically acceptable salt thereof. An improvement of Post COVID Condition, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

The improvement of maternal functioning in a patient suffering from Post COVID Condition is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from Post COVID Condition, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Idiopathic Sleep Disturbance

Treating a patient suffering from idiopathic sleep disturbance with 5-MeO-DMT or a pharmaceutically acceptable salt thereof reduces or eliminates the idiopathic sleep disturbance.

The reduction or elimination of idiopathic sleep disturbance is observed on day 1, for instance after about 24 hours; on day 7; on day 14; and/or on day 28 after the last administration of 5-MeO-DMT or a pharmaceutically acceptable salt thereof.

The reduction or elimination of idiopathic sleep disturbance preferably occurs not later than about 24 hours after the last administration of 5-MeO-DMT or a pharmaceutically acceptable salt thereof. The reduction or elimination of idiopathic sleep disturbance preferably persists until at least 6 days; in particular until at least 14 days; more preferably until at least 28 days after the last administration of 5-MeO-DMT or a pharmaceutically acceptable salt thereof.

In an embodiment, the reduction or elimination of idiopathic sleep disturbance is observed on day 7 after the last administration of 5-MeO-DMT or a pharmaceutically acceptable salt thereof and preferably persists until at least 14 days, more preferably until at least 28 days.

In cases of idiopathic sleep disturbance, a clinical response may be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

An improvement of idiopathic sleep disturbance as reflected by a reduction in the CGI-S score is observed on day 1, for instance after about 24 hours; on day 7; on day 14; and/or on day 28 after the last administration of 5-MeO-DMT or a pharmaceutically acceptable salt thereof.

An improvement of idiopathic sleep disturbance as reflected by a reduction in the CGI-S score, occurs not later than about 24 hours after the last administration of 5-MeO-DMT or a pharmaceutically acceptable salt thereof.

An improvement in idiopathic sleep disturbance as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in idiopathic sleep disturbance as reflected by a reduction in the CGI-S score is observed on day 7 after the last administration of 5-MeO-DMT or a pharmaceutically acceptable salt thereof and preferably persists until at least 14 days, more preferably until at least 28 days.

In cases of idiopathic sleep disturbance, an improvement in sleep disturbance, as reflected by at least a score of “much improved” in the Clinical Global Impression-Improvement (CGI-I) score or the Patient Global Impression-Improvement (PGI-I) score, is observed on day 1, for instance after about 24 hours; on day 7; on day 14; and/or on day 28 after the last administration of 5-MeO-DMT or a pharmaceutically acceptable salt thereof.

An improvement in idiopathic sleep disturbance, as reflected by at least a score of “much improved” in the CGI-I score or the PGI-I score, preferably occurs not later than about 24 hours after the last administration of 5-MeO-DMT or a pharmaceutically acceptable salt thereof.

The improvement in sleep disturbance, as reflected by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

In an embodiment, an improvement in idiopathic sleep disturbance as reflected by at least a score of “much improved” in the CGI-I score or the PGI-I score is observed on day 7 after the last administration of 5-MeO-DMT or a pharmaceutically acceptable salt thereof and preferably persists until at least 14 days, more preferably until at least 28 days.

Improvements in cases of idiopathic sleep disturbance may also be assessed by any other scale reflecting changes in sleep quality or quantity, as indicated above, for instance the Pittsburgh Sleep Quality Index (PSQI).

If treatment outcome is assessed using the PSQI, treatment success is indicated (i) by a decrease of the global score, preferably (ii) by a decrease to 5 or below. The recall period applied does not start earlier than the time point when acute psychedelic experiences have subsided after the last administration.

An improvement in idiopathic sleep disturbance, as reflected by a decrease of the PSQI global score, in particular by a decrease to 5 or below, preferably occurs not later than about 24 hours after the last administration of 5-MeO-DMT or a pharmaceutically acceptable salt thereof.

Such an improvement in idiopathic sleep disturbance as reflected by a decrease of the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

An improvement of idiopathic sleep disturbance as reflected by a reduction in the PSQI global score, in particular by a decrease to 5 or below, is observed on day 1, for instance after about 24 hours; on day 7; on day 14; and/or on day 28 after the last administration of 5-MeO-DMT or a pharmaceutically acceptable salt thereof.

In an embodiment, an improvement of idiopathic sleep disturbance as reflected by a reduction in the PSQI global score, in particular by a decrease to 5 or below, is observed on day 7 and preferably persists until day 14, more preferably until day 28.

The improvement of maternal functioning in a patient suffering from sleep disturbance is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from sleep disturbance, as reflected by an improvement in the BIMF total score, occurs not later than about 6 days after the last administration of 5-MeO-DMT or a pharmaceutically acceptable salt thereof. The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Further Treatment Aspects

Specific aspects of the treatment of major depressive disorder are summarized below.
1. 5-Methoxy-N,N-dimethyltryptamine (5-MeO-DMT) or a pharmaceutically acceptable salt thereof for use in treating a patient who is diagnosed with major depressive disorder by a licensed professional in accordance with accepted medical practice, wherein the 5-MeO-DMT is administered via the intravenous, intramuscular or subcutaneous route, and wherein the patient is a breastfeeding mother who is advised to discontinue breastfeeding until 48 hours, such as 24 hours, preferably 6 hours, more preferably 3 hours, most preferably 2 hours after the last administration of 5-MeO-DMT or a pharmaceutically acceptable salt thereof.
2. 5-MeO-DMT or a pharmaceutically acceptable salt thereof for use as in aspect 1, wherein the disorder is diagnosed in accordance with the Diagnostic and Statistical Manual of Mental Disorders-Fifth Edition (DSM-5) published by the American Psychiatric Association.
3. 5-MeO-DMT or a pharmaceutically acceptable salt thereof for use as in aspects 1 or 2, wherein the patient suffers from moderate or severe major depressive disorder as indicated by a Montgomery-Åsberg Depression Rating Scale (MADRS) score of 20 or more or by a 17-item Hamilton Depression Rating Scale (HAM-D) score of 17 or more.
4. 5-MeO-DMT or a pharmaceutically acceptable salt thereof for use as in aspect 3, wherein the patient suffers from severe major depressive disorder as indicated by a MADRS score of 35 or more or by a HAM-D score of 25 or more.
5. 5-MeO-DMT or a pharmaceutically acceptable salt thereof for use as in aspects 1 to 4, wherein the patient is diagnosed with a treatment-resistant form of major depressive disorder.
6. 5-MeO-DMT or a pharmaceutically acceptable salt thereof for use as in aspects 1 to 5, wherein the patient suffers in addition from suicidal ideation.
7. 5-MeO-DMT or a pharmaceutically acceptable salt thereof for use as in aspect 6, wherein the patient suffers from suicidal ideation with intent to act.
8. 5-MeO-DMT or a pharmaceutically acceptable salt thereof for use as in aspects 1 to 7, wherein the patient is at imminent risk for suicide.
9. 5-MeO-DMT or a pharmaceutically acceptable salt thereof for use as in aspects 1 to 8, wherein the 5-MeO-DMT or salt thereof is administered at a dose or in a dosage regimen that causes the patient to experience a peak psychedelic experience.
10. 5-MeO-DMT or a pharmaceutically acceptable salt thereof for use as in aspects 1 to 9, wherein a dosage of about 1 mg to about 10 mg 5-MeO-DMT is administered, or wherein equimolar amounts of the pharmaceutically acceptable salt are administered instead of 5-MeO-DMT.
11. 5-MeO-DMT or a pharmaceutically acceptable salt thereof for use as in aspects 1 to 9, wherein a dosage of about 2 mg; or of about 5 mg; or of about 8 mg is administered, or wherein equimolar amounts of the pharmaceutically acceptable salt are administered instead of 5-MeO-DMT.
12. 5-MeO-DMT or a pharmaceutically acceptable salt thereof for use as in aspects 1 to 10, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.
13. 5-MeO-DMT or a pharmaceutically acceptable salt thereof for use as in aspects 1 to 10 or 12, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.
14. 5-MeO-DMT or a pharmaceutically acceptable salt thereof for use as in aspect 13, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.
15. 5-MeO-DMT or a pharmaceutically acceptable salt thereof for use as in aspects 12 to 14, wherein the interval between two administrations is not less than 1 hour and not more than 24 hours, such as about 2 to 4 hours.
16. 5-MeO-DMT or a pharmaceutically acceptable salt thereof for use as in aspects 9 to 15, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Psychedelic Experience Questionnaire (PPEQ) Total Score of at least 75.
17. 5-MeO-DMT or a pharmaceutically acceptable salt thereof for use as in aspect 16, wherein the occurrence of a peak psychedelic experience is identified through achievement of a Peak Psychedelic Experience Questionnaire (PPEQ) Total Score of at least 75.
18. 5-MeO-DMT or a pharmaceutically acceptable salt thereof for use as in any of the prior aspects, wherein the 5-MeO-DMT or a pharmaceutically acceptable salt thereof is administered via intravenous injection.
19. 5-MeO-DMT or a pharmaceutically acceptable salt thereof for use as in aspects 1 to 18, wherein a clinical response, as reflected by a reduction in the Clinical Global Impression-Severity (CGI-S) score, occurs not later than about 2 hours after the last administration of 5-MeO-DMT or a pharmaceutically acceptable salt thereof.
20. 5-MeO-DMT or a pharmaceutically acceptable salt thereof for use as in aspects 1 to 19, wherein a clinical response, as reflected by a reduction in the Clinical Global Impression-Severity (CGI-S) score, persists until at least 6 days after the last administration of 5-MeO-DMT or a pharmaceutically acceptable salt thereof.
21. 5-MeO-DMT or a pharmaceutically acceptable salt thereof for use as in aspects 1 to 20, wherein a clinical response, as reflected by a reduction in the Clinical Global Impression-Severity (CGI-S) score, persists until at least 14 days after the last administration of 5-MeO-DMT or a pharmaceutically acceptable salt thereof.
22. 5-MeO-DMT or a pharmaceutically acceptable salt thereof for use as in aspects 1 to 21, wherein a clinical response, as reflected by a reduction in the Clinical Global Impression-Severity (CGI-S) score, persists until at least 28 days after the last administration of 5-MeO-DMT or a pharmaceutically acceptable salt thereof.
23. 5-MeO-DMT or a pharmaceutically acceptable salt thereof for use as in aspects 1 to 22, wherein a clinical response, as assessed by at least 50% improvement of the MADRS or HAM-D score, compared to the respective score prior to treatment, occurs not later than about 2 hours after the last administration of 5-MeO-DMT or a pharmaceutically acceptable salt thereof.
24. 5-MeO-DMT or a pharmaceutically acceptable salt thereof for use as in aspects 1 to 23, wherein a remission of depressive symptoms, as assessed by a MADRS score equal to or less than 10, or a HAM-D score equal to or less than 7, occurs not later than about 2 hours after the last administration of 5-MeO-DMT or a pharmaceutically acceptable salt thereof.
25. 5-MeO-DMT or a pharmaceutically acceptable salt thereof for use as in aspects 1 to 24, wherein the clinical response, as assessed by at least 50% improvement of the MADRS or HAM-D score, compared to the respective score prior to treatment, persists until at least 6 days after the last administration of 5-MeO-DMT or a pharmaceutically acceptable salt thereof.
26. 5-MeO-DMT or a pharmaceutically acceptable salt thereof for use as in aspects 1 to 25, wherein there is a clinical response, as assessed by at least 75% improvement of the MADRS or HAM-D score, compared to the respective score prior to treatment, on day 7 after the last administration of 5-MeO-DMT or a pharmaceutically acceptable salt thereof.
27. 5-MeO-DMT or a pharmaceutically acceptable salt thereof for use as in aspects 1 to 26, wherein the patient is in remission of depressive symptoms, as assessed by a MADRS score equal to or less than 10, or a HAM-D score equal to or less than 7, on day 7 after the last administration of 5-MeO-DMT or a pharmaceutically acceptable salt thereof.
28. 5-MeO-DMT or a pharmaceutically acceptable salt thereof for use as in aspects 1 to 27, wherein the clinical response, as assessed by at least 50% improvement of the MADRS or HAM-D score, compared to the respective score prior to treatment, persists until at least 14 days after the last administration of 5-MeO-DMT or a pharmaceutically acceptable salt thereof.
29. 5-MeO-DMT or a pharmaceutically acceptable salt thereof for use as in aspects 1 to 28, wherein there is a clinical response, as assessed by at least 75% improvement of the MADRS or HAM-D score, compared to the respective score prior to treatment, on day 14 after the last administration of 5-MeO-DMT or a pharmaceutically acceptable salt thereof.
30. 5-MeO-DMT or a pharmaceutically acceptable salt thereof for use as in aspects 1 to 29, wherein the patient is in remission of depressive symptoms, as assessed by a MADRS score equal to or less than 10, or a HAM-D score equal to or less than 7, on day 14 after the last administration of 5-MeO-DMT or a pharmaceutically acceptable salt thereof.
31. 5-MeO-DMT or a pharmaceutically acceptable salt thereof for use as in aspects 1 to 30, wherein the clinical response, as assessed by at least 50% improvement of the MADRS or HAM-D score, compared to the respective score prior to treatment, persists until at least 28 days after the last administration of 5-MeO-DMT or a pharmaceutically acceptable salt thereof.
32. 5-MeO-DMT or a pharmaceutically acceptable salt thereof for use as in aspects 1 to 31, wherein there is a clinical response, as assessed by at least 75% improvement of the MADRS or HAM-D score, compared to the respective score prior to treatment, on day 28 after the last administration of 5-MeO-DMT or a pharmaceutically acceptable salt thereof.
33. 5-MeO-DMT or a pharmaceutically acceptable salt thereof for use as in aspects 1 to 32, wherein the patient is in remission of depressive symptoms, as assessed by a MADRS score equal to or less than 10, or a HAM-D score equal to or less than 7, on day 28 after the last administration of 5-MeO-DMT or a pharmaceutically acceptable salt thereof.
34. 5-MeO-DMT or a pharmaceutically acceptable salt thereof for use as in aspects 1 to 33, wherein the patient is a lactating woman who is advised to discontinue breastfeeding until 48 hours after the last administration of 5-MeO-DMT or a pharmaceutically acceptable salt thereof.
35. 5-MeO-DMT or a pharmaceutically acceptable salt thereof for use as in aspects 1 to 33, wherein the patient is a lactating woman who is advised to discontinue breastfeeding until 24 hours after the last administration of 5-MeO-DMT or a pharmaceutically acceptable salt thereof.
36. 5-MeO-DMT or a pharmaceutically acceptable salt thereof for use as in aspects 1 to 33, wherein the patient is a lactating woman who is advised to discontinue breastfeeding until 6 hours after the last administration of 5-MeO-DMT or a pharmaceutically acceptable salt thereof.
37. 5-MeO-DMT or a pharmaceutically acceptable salt thereof for use in any of claims 1 to 33, wherein the patient is a breastfeeding mother who is advised to discontinue breastfeeding until 3 hours after the last administration of 5-MeO-DMT or a pharmaceutically acceptable salt thereof.
38. 5-MeO-DMT or a pharmaceutically acceptable salt thereof for use as in aspects 1 to 33, wherein the patient is a lactating woman who is advised to discontinue breastfeeding until 2 hours after the last administration of 5-MeO-DMT or a pharmaceutically acceptable salt thereof.
39. 5-MeO-DMT or a pharmaceutically acceptable salt thereof for use as in aspects 1 to 38, wherein the treatment improves maternal functioning.
40. 5-MeO-DMT or a pharmaceutically acceptable salt thereof for use as in aspect 39, wherein the improvement relates to one or more, in particular two or more functional domains according to the Barkin Index of Maternal Functioning (BIMF) selected from self-care, infant care, mother-child interaction, psychological well-being of the mother, social support, management, and adjustment.
41. 5-MeO-DMT or a pharmaceutically acceptable salt thereof for use as in aspect 39 or 40, wherein the BIMF score is improved by 10% or more, preferably by 20% or more.

Specific aspects of the treatment of bipolar disorder are listed below.
1. 5-Methoxy-N,N-dimethyltryptamine (5-MeO-DMT) or a pharmaceutically acceptable salt thereof for use in treating a patient who is diagnosed with bipolar disorder, wherein the 5-MeO-DMT is administered via the intravenous, intramuscular or subcutaneous route, and wherein the patient is a breastfeeding mother who is advised to discontinue breastfeeding until 48 hours, such as 24 hours, preferably 6 hours, more preferably 3 hours, most preferably 2 hours after the last administration of 5-MeO-DMT or a pharmaceutically acceptable salt thereof.
2. 5-MeO-DMT or a pharmaceutically acceptable salt thereof for use as in aspect 1, wherein the patient is diagnosed with bipolar II disorder.
3. 5-MeO-DMT or a pharmaceutically acceptable salt thereof for use as in aspect 1, wherein the patient is diagnosed with bipolar I disorder.
4. 5-MeO-DMT or a pharmaceutically acceptable salt thereof for use as in aspects 1 to 3, wherein the patient suffers from a current major depressive episode.
5. 5-MeO-DMT or a pharmaceutically acceptable salt thereof for use as in aspect 4, wherein the patient has a Montgomery-Åsberg Depression Rating Scale (MADRS) total score of equal to or greater than 19, such as equal to or greater than 24, in particular equal to or greater than 37.
6. 5-MeO-DMT or a pharmaceutically acceptable salt thereof for use as in aspect 4 or aspect 5, wherein the patient has a Bipolar Depression Rating Scale (BDRS) total score of equal to or greater than 19, such as equal to or greater than 24, in particular equal to or greater than 37.
7. 5-MeO-DMT or a pharmaceutically acceptable salt thereof for use as in aspects 1 to 6, wherein the patient had no adequate improvement after at least two adequate courses of therapy.
8. 5-MeO-DMT or a pharmaceutically acceptable salt thereof for use as in aspects 1 to 6, wherein the patient had no adequate improvement after at least two adequate courses of therapy, wherein at least one of the two courses was a pharmacotherapy.
9. 5-MeO-DMT or a pharmaceutically acceptable salt thereof for use as in aspects 1 to 6, wherein the patient had no adequate improvement after at least two adequate courses of pharmacotherapy.
10. 5-MeO-DMT or a pharmaceutically acceptable salt thereof for use as in aspects 1 to 9, wherein the patient has a Young Mania Rating Scale (YMRS) total score less than or equal to 8.
11. 5-MeO-DMT or a pharmaceutically acceptable salt thereof for use as in aspects 1 to 10, wherein the 5-MeO-DMT or salt thereof is administered at a dose or in a dosage regimen that causes the patient to experience a peak psychedelic experience.
12. 5-MeO-DMT or a pharmaceutically acceptable salt thereof for use as in aspects 1 to 11, wherein a dosage of about 1 mg to about 10 mg 5-MeO-DMT is administered, or wherein equimolar amounts of the pharmaceutically acceptable salt are administered instead of 5-MeO-DMT.
13. 5-MeO-DMT or a pharmaceutically acceptable salt thereof for use as in aspects 1 to 11, wherein a dosage of about 2 mg; or of about 5 mg; or of about 8 mg is administered, or wherein equimolar amounts of the pharmaceutically acceptable salt are administered instead of 5-MeO-DMT.
14. 5-MeO-DMT or a pharmaceutically acceptable salt thereof for use as in aspects 1 to 11, wherein a dosage of about 1 mg; or of about 2 mg; or of about 3 mg is administered, or wherein equimolar amounts of the pharmaceutically acceptable salt are administered instead of 5-MeO-DMT.
15. 5-MeO-DMT or a pharmaceutically acceptable salt thereof for use as in aspects 1 to 12, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.
16. 5-MeO-DMT or a pharmaceutically acceptable salt thereof for use as in aspects 1 to 12 or 15, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.
17. 5-MeO-DMT or a pharmaceutically acceptable salt thereof for use as in aspect 16, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.
18. 5-MeO-DMT or a pharmaceutically acceptable salt thereof for use as in aspects 1 to 12 or 15, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.
19. 5-MeO-DMT or a pharmaceutically acceptable salt thereof for use as in aspect 18, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.
20. 5-MeO-DMT or a pharmaceutically acceptable salt thereof for use as in aspects 15 to 19, wherein the interval between two administrations is not less than 1 hour and not more than 24 hours, such as about 1 to 4 hours, preferably 1 to 2 hours.
21. 5-MeO-DMT or a pharmaceutically acceptable salt thereof for use as in aspects 11 to 20, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.
22. 5-MeO-DMT or a pharmaceutically acceptable salt thereof for use as in aspect 21, wherein the occurrence of a peak psychedelic experience is identified through achievement of a Peak Experience Scale (PES) Total Score of at least 75.
23. 5-MeO-DMT or a pharmaceutically acceptable salt thereof for use as in any of the prior aspects, wherein the 5-MeO-DMT or a pharmaceutically acceptable salt thereof is administered via intravenous injection.
24. 5-MeO-DMT or a pharmaceutically acceptable salt thereof for use as in aspects 1 to 23, wherein a clinical response, as reflected by a reduction in the Clinical Global Impression-Severity (CGI-S) score, occurs not later than about 2 hours after the last administration of 5-MeO-DMT or a pharmaceutically acceptable salt thereof.
25. 5-MeO-DMT or a pharmaceutically acceptable salt thereof for use as in aspects 1 to 24, wherein a clinical response, as reflected by a reduction in the Clinical Global Impression-Severity (CGI-S) score, is observed on day 1, for instance, about 24 hours after the last administration of 5-MeO-DMT or a pharmaceutically acceptable salt thereof.
26. 5-MeO-DMT or a pharmaceutically acceptable salt thereof for use as in aspects 1 or 25, wherein a clinical response, as reflected by a reduction in the Clinical Global Impression-Severity (CGI-S) score, persists until at least 6 days after the last administration of 5-MeO-DMT or a pharmaceutically acceptable salt thereof.
27. 5-MeO-DMT or a pharmaceutically acceptable salt thereof for use as in aspects 1 to 26, wherein a clinical response, as reflected by a reduction in the Clinical Global Impression-Severity (CGI-S) score, persists until at least 14 days after the last administration of 5-MeO-DMT or a pharmaceutically acceptable salt thereof.
28. 5-MeO-DMT or a pharmaceutically acceptable salt thereof for use as in aspects 1 to 27, wherein a clinical response, as reflected by a reduction in the Clinical Global Impression-Severity (CGI-S) score, persists until at least 28 days after the last administration of 5-MeO-DMT or a pharmaceutically acceptable salt thereof.
29. 5-MeO-DMT or a pharmaceutically acceptable salt thereof for use as in aspects 1 to 28, wherein a clinical response, as reflected by at least 50% improvement of the BDRS; the MADRS or the HAM-D score, compared to the respective score prior to treatment, occurs not later than about 2 hours after the last administration of 5-MeO-DMT or a pharmaceutically acceptable salt thereof.
30. 5-MeO-DMT or a pharmaceutically acceptable salt thereof for use as in aspects 1 to 29, wherein a remission of depressive symptoms, as reflected by a BDRS score equal to or less than 10; a MADRS score equal to or less than 10, or a HAM-D score equal to or less than 7, occurs not later than about 2 hours after the last administration of 5-MeO-DMT or a pharmaceutically acceptable salt thereof.
31. 5-MeO-DMT or a pharmaceutically acceptable salt thereof for use as in aspects 1 to 30, wherein a clinical response, as reflected by at least 50% improvement of the BDRS; the MADRS or the HAM-D score, compared to the respective score prior to treatment, is observed on day 1, for instance, about 24 hours after the last administration of 5-MeO-DMT or a pharmaceutically acceptable salt thereof.
32. 5-MeO-DMT or a pharmaceutically acceptable salt thereof for use as in aspects 1 to 31, wherein a remission of depressive symptoms, as reflected by a BDRS score equal to or less than 10; a MADRS score equal to or less than 10, or a HAM-D score equal to or less than 7, is observed on day 1, for instance, about 24 hours after the last administration of 5-MeO-DMT or a pharmaceutically acceptable salt thereof.
33. 5-MeO-DMT or a pharmaceutically acceptable salt thereof for use as in aspects 1 to 32, wherein a clinical response, as reflected by at least 50% improvement of the BDRS; the MADRS or the HAM-D score, compared to the respective score prior to treatment, persists until at least 6 days after the last administration of 5-MeO-DMT or a pharmaceutically acceptable salt thereof.
34. 5-MeO-DMT or a pharmaceutically acceptable salt thereof for use as in aspects 1 to 33, wherein there is a clinical response, as reflected by at least 75% improvement of the BDRS; the MADRS or the HAM-D score, compared to the respective score prior to treatment, on day 7 after the last administration of 5-MeO-DMT or a pharmaceutically acceptable salt thereof.
35. 5-MeO-DMT or a pharmaceutically acceptable salt thereof for use as in aspects 1 to 34, wherein the patient is in remission of depressive symptoms, as reflected by a BDRS score equal to or less than 10; a MADRS score equal to or less than 10, or a HAM-D score equal to or less than 7, on day 7 after the last administration of 5-MeO-DMT or a pharmaceutically acceptable salt thereof.
36. 5-MeO-DMT or a pharmaceutically acceptable salt thereof for use as in aspects 1 to 35, wherein a clinical response, as reflected by at least 50% improvement of the BDRS; the MADRS or the HAM-D score, compared to the respective score prior to treatment, persists until at least 14 days after the last administration of 5-MeO-DMT or a pharmaceutically acceptable salt thereof.
37. 5-MeO-DMT or a pharmaceutically acceptable salt thereof for use as in aspects 1 to 36, wherein there is a clinical response, as reflected by at least 75% improvement of the BDRS; the MADRS or the HAM-D score, compared to the respective score prior to treatment, on day 14 after the last administration of 5-MeO-DMT or a pharmaceutically acceptable salt thereof.
38. 5-MeO-DMT or a pharmaceutically acceptable salt thereof for use as in aspects 1 to 37, wherein the patient is in remission of depressive symptoms, as reflected by a BDRS score equal to or less than 10; a MADRS score equal to or less than 10, or a HAM-D score equal to or less than 7, on day 14 after the last administration of 5-MeO-DMT or a pharmaceutically acceptable salt thereof.
39. 5-MeO-DMT or a pharmaceutically acceptable salt thereof for use as in aspects 1 to 38, wherein a clinical response, as reflected by at least 50% improvement of the BDRS; the MADRS or the HAM-D score, compared to the respective score prior to treatment, persists until at least 28 days after the last administration of 5-MeO-DMT or a pharmaceutically acceptable salt thereof.
40. 5-MeO-DMT or a pharmaceutically acceptable salt thereof for use as in aspects 1 to 39, wherein there is a clinical response, as reflected by at least 75% improvement of the BDRS; the MADRS or the HAM-D score, compared to the respective score prior to treatment, on day 28 after the last administration of 5-MeO-DMT or a pharmaceutically acceptable salt thereof.
41. 5-MeO-DMT or a pharmaceutically acceptable salt thereof for use as in aspects 1 to 40, wherein the patient is in remission of depressive symptoms, as reflected by a BDRS score equal to or less than 10; a MADRS score equal to or less than 10, or a HAM-D score equal to or less than 7, on day 28 after the last administration of 5-MeO-DMT or a pharmaceutically acceptable salt thereof.
42. 5-MeO-DMT or a pharmaceutically acceptable salt thereof for use as in aspects 1 to 41, wherein the patient does not experience treatment-emergent mania or hypomania.
43. 5-MeO-DMT or a pharmaceutically acceptable salt thereof for use as in aspects 1 to 42, wherein the patient has a Young Mania Rating Scale (YMRS) total score less than or equal to 15, preferably less than or equal to 12, as assessed about 2 hours after the last administration of 5-MeO-DMT or a pharmaceutically acceptable salt thereof.
44. 5-MeO-DMT or a pharmaceutically acceptable salt thereof for use as in aspects 1 to 43, wherein the patient has a Young Mania Rating Scale (YMRS) total score less than or equal to 15, preferably less than or equal to 12, as assessed on day 1, for instance, about 24 hours after the last administration of 5-MeO-DMT or a pharmaceutically acceptable salt thereof.
45. 5-MeO-DMT or a pharmaceutically acceptable salt thereof for use as in aspects 1 to 44, wherein the patient has a Young Mania Rating Scale (YMRS) total score less than or equal to 15, preferably less than or equal to 12, as assessed on day 7 after the last administration of 5-MeO-DMT or a pharmaceutically acceptable salt thereof.
46. 5-MeO-DMT or a pharmaceutically acceptable salt thereof for use as in aspects 1 to 45, wherein the patient has a Young Mania Rating Scale (YMRS) total score less than or equal to 15, preferably less than or equal to 12, as assessed on day 14 after the last administration of 5-MeO-DMT or a pharmaceutically acceptable salt thereof.
47. 5-MeO-DMT or a pharmaceutically acceptable salt thereof for use as in aspects 1 to 46, wherein the patient has a Young Mania Rating Scale (YMRS) total score less than or equal to 15, preferably less than or equal to 12, as assessed on day 28 after the last administration of 5-MeO-DMT or a pharmaceutically acceptable salt thereof.
48. 5-MeO-DMT or a pharmaceutically acceptable salt thereof for use as in aspects 1 to 47, wherein the treatment leads to an improvement in at least one of sleep disturbance, psychomotor retardation, negative thinking, anxiety, cognitive dysfunction, and social/emotional withdrawal or detachment.
49. 5-MeO-DMT or a pharmaceutically acceptable salt thereof for use as in aspects 1 to 48, wherein the patient suffers from sleep disturbance and the treatment reduces or eliminates the sleep disturbance.
50. 5-MeO-DMT or a pharmaceutically acceptable salt thereof for use as in aspect 49, wherein the patient suffers from insomnia and the treatment reduces or eliminates insomnia.
51. 5-MeO-DMT or a pharmaceutically acceptable salt thereof for use as in aspect 49, wherein the patient suffers from hypersomnia and the treatment reduces or eliminates hypersomnia.
52. 5-MeO-DMT or a pharmaceutically acceptable salt thereof for use as in aspects 49 to 51, wherein the reduction or elimination of sleep disturbance is reflected by an improvement at least in the score of the BDRS item sleep disturbance on day 1, for instance, about 24 hours after the last administration of 5-MeO-DMT or a pharmaceutically acceptable salt thereof.
53. 5-MeO-DMT or a pharmaceutically acceptable salt thereof for use as in aspects 49 to 52, wherein the reduction or elimination of sleep disturbance is reflected by an improvement at least in the score of the BDRS item sleep disturbance on day 7 after the last administration of 5-MeO-DMT or a pharmaceutically acceptable salt thereof.
54. 5-MeO-DMT or a pharmaceutically acceptable salt thereof for use as in aspects 49 to 53, wherein the reduction or elimination of sleep disturbance is reflected by an improvement at least in the score of the BDRS item sleep disturbance on day 14 after the last administration of 5-MeO-DMT or a pharmaceutically acceptable salt thereof.
55. 5-MeO-DMT or a pharmaceutically acceptable salt thereof for use as in aspects 49 to 54, wherein the reduction or elimination of sleep disturbance is reflected by an improvement at least in the score of the BDRS item sleep disturbance on day 28 after the last administration of 5-MeO-DMT or a pharmaceutically acceptable salt thereof.
56. 5-MeO-DMT or a pharmaceutically acceptable salt thereof for use as in aspect 49, wherein the reduction or elimination of sleep disturbance as reflected by an improvement in the score of the BDRS item sleep disturbance occurs not later than about 24 hours after the last administration of 5-MeO-DMT or a pharmaceutically acceptable salt thereof.
57. 5-MeO-DMT or a pharmaceutically acceptable salt thereof for use as in aspect 56, wherein the reduction or elimination of sleep disturbance as reflected by an improvement in the score of the BDRS item sleep disturbance persists until at least 6 days after the last administration of 5-MeO-DMT or a pharmaceutically acceptable salt thereof.
58. 5-MeO-DMT or a pharmaceutically acceptable salt thereof for use as in aspect 56, wherein the reduction or elimination of sleep disturbance as reflected by an improvement in the score of the BDRS item sleep disturbance persists until at least 14 days after the last administration of 5-MeO-DMT or a pharmaceutically acceptable salt thereof.
59. 5-MeO-DMT or a pharmaceutically acceptable salt thereof for use as in aspect 56, wherein the reduction or elimination of sleep disturbance as reflected by an improvement in the score of the BDRS item sleep disturbance persists until at least 28 days after the last administration of 5-MeO-DMT or a pharmaceutically acceptable salt thereof.
60. 5-MeO-DMT or a pharmaceutically acceptable salt thereof for use as in aspect 49, wherein the sleep disturbance is reduced sleep and the reduction or elimination of sleep disturbance is reflected by an improvement at least in the score of the MADRS item reduced sleep on day 1, for instance, about 24 hours after the last administration of 5-MeO-DMT or a pharmaceutically acceptable salt thereof.
61. 5-MeO-DMT or a pharmaceutically acceptable salt thereof for use as in aspects 49 or 60, wherein the sleep disturbance is reduced sleep and the reduction or elimination of sleep disturbance is reflected by an improvement at least in the score of the MADRS item reduced sleep on day 7 after the last administration of 5-MeO-DMT or a pharmaceutically acceptable salt thereof.
62. 5-MeO-DMT or a pharmaceutically acceptable salt thereof for use as in aspects 49, 60 or 61, wherein the sleep disturbance is reduced sleep and the reduction or elimination of sleep disturbance is reflected by an improvement at least in the score of the MADRS item reduced sleep on day 14 after the last administration of 5-MeO-DMT or a pharmaceutically acceptable salt thereof.
63. 5-MeO-DMT or a pharmaceutically acceptable salt thereof for use as in aspects 49 or 60 to 62, wherein the sleep disturbance is reduced sleep and the reduction or elimination of sleep disturbance is reflected by an improvement at least in the score of the MADRS item reduced sleep on day 28 after the last administration of 5-MeO-DMT or a pharmaceutically acceptable salt thereof.
64. 5-MeO-DMT or a pharmaceutically acceptable salt thereof for use as in aspect 49, wherein the sleep disturbance is reduced sleep and the reduction or elimination of sleep disturbance as reflected by an improvement in the score of the MADRS item reduced sleep occurs not later than about 24 hours after the last administration of 5-MeO-DMT or a pharmaceutically acceptable salt thereof.
65. 5-MeO-DMT or a pharmaceutically acceptable salt thereof for use as in aspect 64, wherein the sleep disturbance is reduced sleep and the reduction or elimination of sleep disturbance as reflected by an improvement in the score of the MADRS item reduced sleep persists until at least 6 days after the last administration of 5-MeO-DMT or a pharmaceutically acceptable salt thereof.
66. 5-MeO-DMT or a pharmaceutically acceptable salt thereof for use as in aspect 64, wherein the sleep disturbance is reduced sleep and the reduction or elimination of sleep disturbance as reflected by an improvement in the score of the MADRS item reduced sleep persists until at least 14 days after the last administration of 5-MeO-DMT or a pharmaceutically acceptable salt thereof.
67. 5-MeO-DMT or a pharmaceutically acceptable salt thereof for use as in aspect 64, wherein the sleep disturbance is reduced sleep and the reduction or elimination of sleep disturbance as reflected by an improvement in the score of the MADRS item reduced sleep persists until at least 28 days after the last administration of 5-MeO-DMT or a pharmaceutically acceptable salt thereof.
68. 5-MeO-DMT or a pharmaceutically acceptable salt thereof for use as in aspect 49, wherein the patient suffers from sleep disturbance and an improvement in sleep disturbance, is reflected by a reduction in the Clinical Global Impression-Severity (CGI-S) score on day 1, for instance, about 24 hours after the last administration of 5-MeO-DMT or a pharmaceutically acceptable salt thereof.
69. 5-MeO-DMT or a pharmaceutically acceptable salt thereof for use as in aspects 49 or 68, wherein the patient suffers from sleep disturbance and an improvement in sleep disturbance is reflected by a reduction in the Clinical Global Impression-Severity (CGI-S) score on day 7 after the last administration of 5-MeO-DMT or a pharmaceutically acceptable salt thereof.
70. 5-MeO-DMT or a pharmaceutically acceptable salt thereof for use as in aspects 49, 68 or 69, wherein the patient suffers from sleep disturbance and an improvement in sleep disturbance is reflected by a reduction in the Clinical Global Impression-Severity (CGI-S) score on day 14 after the last administration of 5-MeO-DMT or a pharmaceutically acceptable salt thereof.
71. 5-MeO-DMT or a pharmaceutically acceptable salt thereof for use as in aspects 49 or 68 to 70, wherein the patient suffers from sleep disturbance and an improvement in sleep disturbance is reflected by a reduction in the Clinical Global Impression-Severity (CGI-S) score on day 28 after the last administration of 5-MeO-DMT or a pharmaceutically acceptable salt thereof.
72. 5-MeO-DMT or a pharmaceutically acceptable salt thereof for use as in aspects 1 to 48, wherein the patient suffers from sleep disturbance and an improvement in sleep disturbance, as reflected by a reduction in the Clinical Global Impression-Severity (CGI-S) score, occurs not later than about 24 hours after the last administration of 5-MeO-DMT or a pharmaceutically acceptable salt thereof.
73. 5-MeO-DMT or a pharmaceutically acceptable salt thereof for use as in aspect 72, wherein the improvement in sleep disturbance, as reflected by a reduction in the Clinical Global Impression-Severity (CGI-S) score, persists until at least 6 days after the last administration of 5-MeO-DMT or a pharmaceutically acceptable salt thereof.
74. 5-MeO-DMT or a pharmaceutically acceptable salt thereof for use as in aspect 72, wherein the improvement in sleep disturbance, as reflected by a reduction in the Clinical Global Impression-Severity (CGI-S) score, persists until at least 14 days after the last administration of 5-MeO-DMT or a pharmaceutically acceptable salt thereof.
75. 5-MeO-DMT or a pharmaceutically acceptable salt thereof for use as in aspect 72, wherein the improvement in sleep disturbance, as reflected by a reduction in the Clinical Global Impression-Severity (CGI-S) score, persists until at least 28 days after the last administration of 5-MeO-DMT or a pharmaceutically acceptable salt thereof.
76. 5-MeO-DMT or a pharmaceutically acceptable salt thereof for use as in aspects 1 to 48, wherein the patient suffers from sleep disturbance and the reduction or elimination of sleep disturbance is reflected by an improvement in the Pittsburgh Sleep Quality Index (PSQI) global score on day 1, for instance, about 24 hours after the last administration of 5-MeO-DMT or a pharmaceutically acceptable salt thereof, wherein the recall period spans from the time point when acute psychedelic experiences have subsided after the last administration to the assessment time point.
77. 5-MeO-DMT or a pharmaceutically acceptable salt thereof for use as in aspects 1 to 48 or 76, wherein the patient suffers from sleep disturbance and the reduction or elimination of sleep disturbance is reflected by an improvement in the Pittsburgh Sleep Quality Index (PSQI) global score on day 7 after the last administration of 5-MeO-DMT or a pharmaceutically acceptable salt thereof, wherein the recall period spans from the time point when acute psychedelic experiences have subsided after the last administration to the assessment time point.
78. 5-MeO-DMT or a pharmaceutically acceptable salt thereof for use as in aspects 1 to 48, 76 or 77, wherein the patient suffers from sleep disturbance and the reduction or elimination of sleep disturbance is reflected by an improvement in the Pittsburgh Sleep Quality Index (PSQI) global score on day 14 after the last administration of 5-MeO-DMT or a pharmaceutically acceptable salt thereof, wherein the recall period spans from the time point when acute psychedelic experiences have subsided after the last administration to the assessment time point.
79. 5-MeO-DMT or a pharmaceutically acceptable salt thereof for use as in aspects 1 to 48 or 76 to 78, wherein the patient suffers from sleep disturbance and the reduction or elimination of sleep disturbance is reflected by an improvement in the Pittsburgh Sleep Quality Index (PSQI) global score on day 28 after the last administration of 5-MeO-DMT or a pharmaceutically acceptable salt thereof, wherein the recall period spans from the time point when acute psychedelic experiences have subsided after the last administration to the assessment time point.
80. 5-MeO-DMT or a pharmaceutically acceptable salt thereof for use as in aspect 49, wherein the patient suffers from sleep disturbance and the reduction or elimination of sleep disturbance as reflected by an improvement in the Pittsburgh Sleep Quality Index (PSQI) global score occurs not later than about 24 hours after the last administration of 5-MeO-DMT or a pharmaceutically acceptable salt thereof, wherein the recall period spans from the time point when acute psychedelic experiences have subsided after the last administration to the assessment time point.
81. 5-MeO-DMT or a pharmaceutically acceptable salt thereof for use as in aspect 80, wherein the patient suffers from sleep disturbance and the reduction or elimination of sleep disturbance as reflected by an improvement in the Pittsburgh Sleep Quality Index (PSQI) global score persists until at least 6 days after the last administration of 5-MeO-DMT or a pharmaceutically acceptable salt thereof, wherein the recall period spans from the time point when acute psychedelic experiences have subsided after the last administration to the assessment time point.
82. 5-MeO-DMT or a pharmaceutically acceptable salt thereof for use as in aspect 80, wherein the patient suffers from sleep disturbance and the reduction or elimination of sleep disturbance as reflected by an improvement in the Pittsburgh Sleep Quality Index (PSQI) global score persists until at least 14 days after the last administration of 5-MeO-DMT or a pharmaceutically acceptable salt thereof, wherein the recall period spans from the time point when acute psychedelic experiences have subsided after the last administration to the assessment time point.
83. 5-MeO-DMT or a pharmaceutically acceptable salt thereof for use as in aspect 80, wherein the patient suffers from sleep disturbance and the reduction or elimination of sleep disturbance as reflected by an improvement in the Pittsburgh Sleep Quality Index (PSQI) global score persists until at least 28 days after the last administration of 5-MeO-DMT or a pharmaceutically acceptable salt thereof, wherein the recall period spans from the time point when acute psychedelic experiences have subsided after the last administration to the assessment time point.
84. 5-MeO-DMT or a pharmaceutically acceptable salt thereof for use as in aspects 1 to 83, wherein the patient suffers from psychomotor retardation and the treatment reduces or eliminates the psychomotor retardation.
85. 5-MeO-DMT or a pharmaceutically acceptable salt thereof for use as in aspect 84, wherein the treatment improves or eliminates reduced energy and activity and/or reduced motivation.
86. 5-MeO-DMT or a pharmaceutically acceptable salt thereof for use as in aspects 84 or 85, wherein the reduction or elimination of psychomotor retardation is reflected by an improvement at least in the score of the BDRS items reduced energy and activity and/or reduced motivation about 2 hours after the last administration of 5-MeO-DMT or a pharmaceutically acceptable salt thereof.
87. 5-MeO-DMT or a pharmaceutically acceptable salt thereof for use as in aspects 84 to 86, wherein the reduction or elimination of psychomotor retardation is reflected by an improvement at least in the score of the BDRS items reduced energy and activity and/or reduced motivation on day 1, for instance, about 24 hours after the last administration of 5-MeO-DMT or a pharmaceutically acceptable salt thereof.
88. 5-MeO-DMT or a pharmaceutically acceptable salt thereof for use as in aspects 84 to 87, wherein the reduction or elimination of psychomotor retardation is reflected by an improvement at least in the score of the BDRS items reduced energy and activity and/or reduced motivation on day 7 after the last administration of 5-MeO-DMT or a pharmaceutically acceptable salt thereof.
89. 5-MeO-DMT or a pharmaceutically acceptable salt thereof for use as in aspects 84 to 88, wherein the reduction or elimination of psychomotor retardation is reflected by an improvement at least in the score of the BDRS items reduced energy and activity and/or reduced motivation on day 14 after the last administration of 5-MeO-DMT or a pharmaceutically acceptable salt thereof.
90. 5-MeO-DMT or a pharmaceutically acceptable salt thereof for use as in aspects 84 to 89, wherein the reduction or elimination of psychomotor retardation is reflected by an improvement at least in the score of the BDRS items reduced energy and activity and/or reduced motivation on day 28 after the last administration of 5-MeO-DMT or a pharmaceutically acceptable salt thereof.
91. 5-MeO-DMT or a pharmaceutically acceptable salt thereof for use as in aspects 84 or 85, wherein the reduction or elimination of psychomotor retardation as reflected by an improvement in the score of the BDRS items reduced energy and activity and/or reduced motivation occurs not later than about 2 hours after the last administration of 5-MeO-DMT or a pharmaceutically acceptable salt thereof.
92. 5-MeO-DMT or a pharmaceutically acceptable salt thereof for use as in aspect 91, wherein the reduction or elimination of psychomotor retardation as reflected by an improvement in the score of the BDRS items reduced energy and activity and/or reduced motivation persists until at least 6 days after the last administration of 5-MeO-DMT or a pharmaceutically acceptable salt thereof.
93. 5-MeO-DMT or a pharmaceutically acceptable salt thereof for use as in aspect 91, wherein the reduction or elimination of psychomotor retardation as reflected by an improvement in the score of the BDRS items reduced energy and activity and/or reduced motivation persists until at least 14 days after the last administration of 5-MeO-DMT or a pharmaceutically acceptable salt thereof.
94. 5-MeO-DMT or a pharmaceutically acceptable salt thereof for use as in aspect 91, wherein the reduction or elimination of psychomotor retardation as reflected by an improvement in the score of the BDRS items reduced energy and activity and/or reduced motivation persists until at least 28 days after the last administration of 5-MeO-DMT or a pharmaceutically acceptable salt thereof.
95. 5-MeO-DMT or a pharmaceutically acceptable salt thereof for use as in aspect 84, wherein the reduction or elimination of psychomotor retardation is reflected by an improvement at least in the score of the MADRS item lassitude about 2 hours after the last administration of 5-MeO-DMT or a pharmaceutically acceptable salt thereof.
96. 5-MeO-DMT or a pharmaceutically acceptable salt thereof for use as in aspects 84 or 95, wherein the reduction or elimination of psychomotor retardation is reflected by an improvement at least in the score of the MADRS item lassitude on day 1, for instance, about 24 hours after the last administration of 5-MeO-DMT or a pharmaceutically acceptable salt thereof.
97. 5-MeO-DMT or a pharmaceutically acceptable salt thereof for use as in aspects 84, 95 or 96, wherein the reduction or elimination of psychomotor retardation is reflected by an improvement at least in the score of the MADRS item lassitude on day 7 after the last administration of 5-MeO-DMT or a pharmaceutically acceptable salt thereof.
98. 5-MeO-DMT or a pharmaceutically acceptable salt thereof for use as in aspects 84 or 95 to 97, wherein the reduction or elimination of psychomotor retardation is reflected by an improvement at least in the score of the MADRS item lassitude on day 14 after the last administration of 5-MeO-DMT or a pharmaceutically acceptable salt thereof.
99. 5-MeO-DMT or a pharmaceutically acceptable salt thereof for use as in aspects 84 or 95 to 98, wherein the reduction or elimination of psychomotor retardation is reflected by an improvement at least in the score of the MADRS item lassitude on day 28 after the last administration of 5-MeO-DMT or a pharmaceutically acceptable salt thereof.
100. 5-MeO-DMT or a pharmaceutically acceptable salt thereof for use as in aspect 84, wherein the reduction or elimination of psychomotor retardation as reflected by an improvement in the score of the MADRS item lassitude occurs not later than about 2 hours after the last administration of 5-MeO-DMT or a pharmaceutically acceptable salt thereof.
101. 5-MeO-DMT or a pharmaceutically acceptable salt thereof for use as in aspect 100, wherein the reduction or elimination of psychomotor retardation as reflected by an improvement in the score of the MADRS item lassitude persists until at least 6 days after the last administration of 5-MeO-DMT or a pharmaceutically acceptable salt thereof.
102. 5-MeO-DMT or a pharmaceutically acceptable salt thereof for use as in aspect 100, wherein the reduction or elimination of psychomotor retardation as reflected by an improvement in the score of the MADRS item lassitude persists until at least 14 days after the last administration of 5-MeO-DMT or a pharmaceutically acceptable salt thereof.
103. 5-MeO-DMT or a pharmaceutically acceptable salt thereof for use as in aspect 100, wherein the reduction or elimination of psychomotor retardation as reflected by an improvement in the score of the MADRS item lassitude persists until at least 28 days after the last administration of 5-MeO-DMT or a pharmaceutically acceptable salt thereof.
104. 5-MeO-DMT or a pharmaceutically acceptable salt thereof for use as in aspect 84, wherein the patient suffers from psychomotor retardation and an improvement in psychomotor retardation is reflected by a reduction in the Clinical Global Impression-Severity (CGI-S) score about 2 hours after the last administration of 5-MeO-DMT or a pharmaceutically acceptable salt thereof.
105. 5-MeO-DMT or a pharmaceutically acceptable salt thereof for use as in aspects 84 or 104, wherein the patient suffers from psychomotor retardation and an improvement in psychomotor retardation is reflected by a reduction in the Clinical Global Impression-Severity (CGI-S) score on day 1, for instance, about 24 hours after the last administration of 5-MeO-DMT or a pharmaceutically acceptable salt thereof.
106. 5-MeO-DMT or a pharmaceutically acceptable salt thereof for use as in aspects 84, 104 or 105, wherein the patient suffers from psychomotor retardation and an improvement in psychomotor retardation is reflected by a reduction in the Clinical Global Impression-Severity (CGI-S) score on day 7 after the last administration of 5-MeO-DMT or a pharmaceutically acceptable salt thereof.
107. 5-MeO-DMT or a pharmaceutically acceptable salt thereof for use as in aspects 84 or 104 to 106, wherein the patient suffers from psychomotor retardation and an improvement in psychomotor retardation is reflected by a reduction in the Clinical Global Impression-Severity (CGI-S) score on day 14 after the last administration of 5-MeO-DMT or a pharmaceutically acceptable salt thereof.
108. 5-MeO-DMT or a pharmaceutically acceptable salt thereof for use as in aspects 84 or 104 to 107, wherein the patient suffers from psychomotor retardation and an improvement in psychomotor retardation is reflected by a reduction in the Clinical Global Impression-Severity (CGI-S) score on day 28 after the last administration of 5-MeO-DMT or a pharmaceutically acceptable salt thereof.
109. 5-MeO-DMT or a pharmaceutically acceptable salt thereof for use as in aspects 1 to 83, wherein the patient suffers from psychomotor retardation and an improvement in psychomotor retardation, as reflected by a reduction in the Clinical Global Impression-Severity (CGI-S) score, occurs not later than about 2 hours after the last administration of 5-MeO-DMT or a pharmaceutically acceptable salt thereof.
110. 5-MeO-DMT or a pharmaceutically acceptable salt thereof for use as in aspect 109, wherein the improvement in psychomotor retardation, as reflected by a reduction in the Clinical Global Impression-Severity (CGI-S) score, persists until at least 6 days after the last administration of 5-MeO-DMT or a pharmaceutically acceptable salt thereof.
111. 5-MeO-DMT or a pharmaceutically acceptable salt thereof for use as in aspect 109, wherein the improvement in psychomotor retardation, as reflected by a reduction in the Clinical Global Impression-Severity (CGI-S) score, persists until at least 14 days after the last administration of 5-MeO-DMT or a pharmaceutically acceptable salt thereof.
112. 5-MeO-DMT or a pharmaceutically acceptable salt thereof for use as in aspect 109, wherein the improvement in psychomotor retardation, as reflected by a reduction in the Clinical Global Impression-Severity (CGI-S) score, persists until at least 28 days after the last administration of 5-MeO-DMT or a pharmaceutically acceptable salt thereof.
113. 5-MeO-DMT or a pharmaceutically acceptable salt thereof for use as in aspects 1 to 112, wherein the patient suffers from negative thinking and the treatment reduces or eliminates the negative thinking.
114. 5-MeO-DMT or a pharmaceutically acceptable salt thereof for use as in aspect 113, wherein the treatment reduces or eliminates feelings of worthlessness; helplessness and hopelessness; and/or guilt.
115. 5-MeO-DMT or a pharmaceutically acceptable salt thereof for use as in aspects 113 or 114, wherein the reduction or elimination of negative thinking is reflected by an improvement at least in the scores of the BDRS items worthlessness; helplessness and hopelessness; and/or guilt about 2 hours after the last administration of 5-MeO-DMT or a pharmaceutically acceptable salt thereof.
116. 5-MeO-DMT or a pharmaceutically acceptable salt thereof for use as in aspects 113 to 115, wherein the reduction or elimination of negative thinking is reflected by an improvement at least in the scores of the BDRS items worthlessness; helplessness and hopelessness; and/or guilt on day 1, for instance, about 24 hours after the last administration of 5-MeO-DMT or a pharmaceutically acceptable salt thereof.
117. 5-MeO-DMT or a pharmaceutically acceptable salt thereof for use as in aspects 113 to 116, wherein the reduction or elimination of negative thinking is reflected by an improvement at least in the scores of the BDRS items worthlessness; helplessness and hopelessness; and/or guilt on day 7 after the last administration of 5-MeO-DMT or a pharmaceutically acceptable salt thereof.
118. 5-MeO-DMT or a pharmaceutically acceptable salt thereof for use as in aspects 113 to 117, wherein the reduction or elimination of negative thinking is reflected by an improvement at least in the scores of the BDRS items worthlessness; helplessness and hopelessness; and/or guilt on day 14 after the last administration of 5-MeO-DMT or a pharmaceutically acceptable salt thereof.
119. 5-MeO-DMT or a pharmaceutically acceptable salt thereof for use as in aspects 113 to 118, wherein the reduction or elimination of negative thinking is reflected by an improvement at least in the score of the BDRS items worthlessness; helplessness and hopelessness; and/or guilt on day 28 after the last administration of 5-MeO-DMT or a pharmaceutically acceptable salt thereof.
120. 5-MeO-DMT or a pharmaceutically acceptable salt thereof for use as in aspects 113 or 114, wherein the reduction or elimination of negative thinking as reflected by an improvement in the scores of the BDRS items worthlessness; helplessness and hopelessness; and/or guilt occurs not later than about 2 hours after the last administration of 5-MeO-DMT or a pharmaceutically acceptable salt thereof.
121. 5-MeO-DMT or a pharmaceutically acceptable salt thereof for use as in aspect 120, wherein the reduction or elimination of negative thinking as reflected by an improvement in the scores of the BDRS items worthlessness; helplessness and hopelessness; and/or guilt persists until at least 6 days after the last administration of 5-MeO-DMT or a pharmaceutically acceptable salt thereof.
122. 5-MeO-DMT or a pharmaceutically acceptable salt thereof for use as in aspect 120, wherein the reduction or elimination of negative thinking as reflected by an improvement in the scores of the BDRS items worthlessness; helplessness and hopelessness; and/or guilt persists until at least 14 days after the last administration of 5-MeO-DMT or a pharmaceutically acceptable salt thereof.
123. 5-MeO-DMT or a pharmaceutically acceptable salt thereof for use as in aspect 120, wherein the reduction or elimination of negative thinking as reflected by an improvement in the scores of the BDRS items worthlessness; helplessness and hopelessness; and/or guilt persists until at least 28 days after the last administration of 5-MeO-DMT or a pharmaceutically acceptable salt thereof.
124. 5-MeO-DMT or a pharmaceutically acceptable salt thereof for use as in aspects 113 or 114, wherein the reduction or elimination of negative thinking is reflected by an improvement at least in the score of the MADRS item pessimistic thoughts about 2 hours after the last administration of 5-MeO-DMT or a pharmaceutically acceptable salt thereof.
125. 5-MeO-DMT or a pharmaceutically acceptable salt thereof for use as in aspects 113, 114 or 124, wherein the reduction or elimination of negative thinking is reflected by an improvement at least in the score of the MADRS item pessimistic thoughts on day 1, for instance, about 24 hours after the last administration of 5-MeO-DMT or a pharmaceutically acceptable salt thereof.
126. 5-MeO-DMT or a pharmaceutically acceptable salt thereof for use as in aspects 113, 114, 124 or 125, wherein the reduction or elimination of negative thinking is reflected by an improvement at least in the score of the MADRS item pessimistic thoughts on day 7 after the last administration of 5-MeO-DMT or a pharmaceutically acceptable salt thereof.
127. 5-MeO-DMT or a pharmaceutically acceptable salt thereof for use as in aspects 113, 114 or 124 to 126, wherein the reduction or elimination of negative thinking is reflected by an improvement at least in the score of the MADRS item pessimistic thoughts on day 14 after the last administration of 5-MeO-DMT or a pharmaceutically acceptable salt thereof.
128. 5-MeO-DMT or a pharmaceutically acceptable salt thereof for use as in aspects 113, 114 or 124 to 127, wherein the reduction or elimination of negative thinking is reflected by an improvement at least in the score of the MADRS item pessimistic thoughts on day 28 after the last administration of 5-MeO-DMT or a pharmaceutically acceptable salt thereof.
129. 5-MeO-DMT or a pharmaceutically acceptable salt thereof for use as in aspects 113 or 114, wherein the reduction or elimination of negative thinking as reflected by an improvement in the score of the MADRS item pessimistic thoughts occurs not later than about 2 hours after the last administration of 5-MeO-DMT or a pharmaceutically acceptable salt thereof.
130. 5-MeO-DMT or a pharmaceutically acceptable salt thereof for use as in aspect 129, wherein the reduction or elimination of negative thinking as reflected by an improvement in the score of the MADRS item pessimistic thoughts persists until at least 6 days after the last administration of 5-MeO-DMT or a pharmaceutically acceptable salt thereof.
131. 5-MeO-DMT or a pharmaceutically acceptable salt thereof for use as in aspect 129, wherein the reduction or elimination of negative thinking as reflected by an improvement in the score of the MADRS item pessimistic thoughts persists until at least 14 days after the last administration of 5-MeO-DMT or a pharmaceutically acceptable salt thereof.
132. 5-MeO-DMT or a pharmaceutically acceptable salt thereof for use as in aspect 129, wherein the reduction or elimination of negative thinking as reflected by an improvement in the score of the MADRS item pessimistic thoughts persists until at least 28 days after the last administration of 5-MeO-DMT or a pharmaceutically acceptable salt thereof.
133. 5-MeO-DMT or a pharmaceutically acceptable salt thereof for use as in aspect 113 or 114, wherein the reduction or elimination of negative thinking is reflected by an improvement at least in the score of the BPRS item guilt feelings about 2 hours after the last administration of 5-MeO-DMT or a pharmaceutically acceptable salt thereof.
134. 5-MeO-DMT or a pharmaceutically acceptable salt thereof for use as in aspects 113, 114 or 133, wherein the reduction or elimination of negative thinking is reflected by an improvement at least in the score of the BPRS item guilt feelings on day 1, for instance, about 24 hours after the last administration of 5-MeO-DMT or a pharmaceutically acceptable salt thereof.
135. 5-MeO-DMT or a pharmaceutically acceptable salt thereof for use as in aspects 113, 114, 133 or 134, wherein the reduction or elimination of negative thinking is reflected by an improvement at least in the score of the BPRS item guilt feelings on day 7 after the last administration of 5-MeO-DMT or a pharmaceutically acceptable salt thereof.
136. 5-MeO-DMT or a pharmaceutically acceptable salt thereof for use as in aspects 113, 114 or 133 to 135, wherein the reduction or elimination of negative thinking is reflected by an improvement at least in the score of the BPRS item guilt feelings on day 14 after the last administration of 5-MeO-DMT or a pharmaceutically acceptable salt thereof.
137. 5-MeO-DMT or a pharmaceutically acceptable salt thereof for use as in aspects 113, 114 or 133 to 136 wherein the reduction or elimination of negative thinking is reflected by an improvement at least in the score of the BPRS item guilt feelings on day 28 after the last administration of 5-MeO-DMT or a pharmaceutically acceptable salt thereof.
138. 5-MeO-DMT or a pharmaceutically acceptable salt thereof for use as in aspects 113 or 114, wherein the reduction or elimination of negative thinking as reflected by an improvement in the score of the BPRS item guilt feelings occurs not later than about 2 hours after the last administration of 5-MeO-DMT or a pharmaceutically acceptable salt thereof.
139. 5-MeO-DMT or a pharmaceutically acceptable salt thereof for use as in aspect 138, wherein the reduction or elimination of negative thinking as reflected by an improvement in the score of the BPRS item guilt feelings persists until at least 6 days after the last administration of 5-MeO-DMT or a pharmaceutically acceptable salt thereof.
140. 5-MeO-DMT or a pharmaceutically acceptable salt thereof for use as in aspect 138, wherein the reduction or elimination of negative thinking as reflected by an improvement in the score of the BPRS item guilt feelings persists until at least 14 days after the last administration of 5-MeO-DMT or a pharmaceutically acceptable salt thereof.
141. 5-MeO-DMT or a pharmaceutically acceptable salt thereof for use as in aspect 138, wherein the reduction or elimination of negative thinking as reflected by an improvement in the score of the BPRS item guilt feelings persists until at least 28 days after the last administration of 5-MeO-DMT or a pharmaceutically acceptable salt thereof.
142. 5-MeO-DMT or a pharmaceutically acceptable salt thereof for use as in aspects 113 or 114, wherein the patient suffers from negative thinking and an improvement in negative thinking is reflected by a reduction in the Clinical Global Impression-Severity (CGI-S) score about 2 hours after the last administration of 5-MeO-DMT or a pharmaceutically acceptable salt thereof.
143. 5-MeO-DMT or a pharmaceutically acceptable salt thereof for use as in aspects 113, 114 or 142, wherein the patient suffers from negative thinking and an improvement in negative thinking is reflected by a reduction in the Clinical Global Impression-Severity (CGI-S) score on day 1, for instance, about 24 hours after the last administration of 5-MeO-DMT or a pharmaceutically acceptable salt thereof.
144. 5-MeO-DMT or a pharmaceutically acceptable salt thereof for use as in aspects 113, 114, 142 or 143, wherein the patient suffers from negative thinking and an improvement in negative thinking is reflected by a reduction in the Clinical Global Impression-Severity (CGI-S) score on day 7 after the last administration of 5-MeO-DMT or a pharmaceutically acceptable salt thereof.
145. 5-MeO-DMT or a pharmaceutically acceptable salt thereof for use as in aspects 113, 114 or 142 to 144, wherein the patient suffers from negative thinking and an improvement in negative thinking is reflected by a reduction in the Clinical Global Impression-Severity (CGI-S) score on day 14 after the last administration of 5-MeO-DMT or a pharmaceutically acceptable salt thereof.
146. 5-MeO-DMT or a pharmaceutically acceptable salt thereof for use as in aspects 113, 114 or 142 to 145, wherein the patient suffers from negative thinking and an improvement in negative thinking is reflected by a reduction in the Clinical Global Impression-Severity (CGI-S) score on day 28 after the last administration of 5-MeO-DMT or a pharmaceutically acceptable salt thereof.
147. 5-MeO-DMT or a pharmaceutically acceptable salt thereof for use as in aspects 1 to 112, wherein the patient suffers from negative thinking and an improvement in negative thinking, as reflected by a reduction in the Clinical Global Impression-Severity (CGI-S) score, occurs not later than about 2 hours after the last administration of 5-MeO-DMT or a pharmaceutically acceptable salt thereof.
148. 5-MeO-DMT or a pharmaceutically acceptable salt thereof for use as in aspect 147, wherein the improvement in negative thinking, as reflected by a reduction in the Clinical Global Impression-Severity (CGI-S) score, persists until at least 6 days after the last administration of 5-MeO-DMT or a pharmaceutically acceptable salt thereof.
149. 5-MeO-DMT or a pharmaceutically acceptable salt thereof for use as in aspect 147, wherein the improvement in negative thinking, as reflected by a reduction in the Clinical Global Impression-Severity (CGI-S) score, persists until at least 14 days after the last administration of 5-MeO-DMT or a pharmaceutically acceptable salt thereof.
150. 5-MeO-DMT or a pharmaceutically acceptable salt thereof for use as in aspect 147, wherein the improvement in negative thinking, as reflected by a reduction in the Clinical Global Impression-Severity (CGI-S) score, persists until at least 28 days after the last administration of 5-MeO-DMT or a pharmaceutically acceptable salt thereof.
151. 5-MeO-DMT or a pharmaceutically acceptable salt thereof for use as in aspects 1 to 150, wherein the patient suffers from anxiety and the treatment reduces or eliminates the anxiety. 152. 5-MeO-DMT or a pharmaceutically acceptable salt thereof for use as in aspect 151, wherein the reduction or elimination of anxiety is reflected by an improvement at least in the score of the BDRS item anxiety 2 hours after the last administration of 5-MeO-DMT or a pharmaceutically acceptable salt thereof.
153. 5-MeO-DMT or a pharmaceutically acceptable salt thereof for use as in aspects 151 or 152, wherein the reduction or elimination of anxiety is reflected by an improvement at least in the score of the BDRS item anxiety on day 1, for instance, about 24 hours after the last administration of 5-MeO-DMT or a pharmaceutically acceptable salt thereof.
154. 5-MeO-DMT or a pharmaceutically acceptable salt thereof for use as in aspects 151 to 153, wherein the reduction or elimination of anxiety is reflected by an improvement at least in the score of the BDRS item anxiety on day 7 after the last administration of 5-MeO-DMT or a pharmaceutically acceptable salt thereof.
155. 5-MeO-DMT or a pharmaceutically acceptable salt thereof for use as in aspects 151 to 154, wherein the reduction or elimination of anxiety is reflected by an improvement at least in the score of the BDRS item anxiety on day 14 after the last administration of 5-MeO-DMT or a pharmaceutically acceptable salt thereof.
156. 5-MeO-DMT or a pharmaceutically acceptable salt thereof for use as in aspects 151 to 155, wherein the reduction or elimination of anxiety is reflected by an improvement at least in the score of the BDRS item anxiety on day 28 after the last administration of 5-MeO-DMT or a pharmaceutically acceptable salt thereof.
157. 5-MeO-DMT or a pharmaceutically acceptable salt thereof for use as in aspect 151, wherein the reduction or elimination of anxiety as reflected by an improvement in the score of the BDRS item anxiety occurs not later than about 2 hours after the last administration of 5-MeO-DMT or a pharmaceutically acceptable salt thereof.
158. 5-MeO-DMT or a pharmaceutically acceptable salt thereof for use as in aspect 157, wherein the reduction or elimination of anxiety as reflected by an improvement in the score of the BDRS item anxiety persists until at least 6 days after the last administration of 5-MeO-DMT or a pharmaceutically acceptable salt thereof.
159. 5-MeO-DMT or a pharmaceutically acceptable salt thereof for use as in aspect 157, wherein the reduction or elimination of anxiety as reflected by an improvement in the score of the BDRS item anxiety persists until at least 14 days after the last administration of 5-MeO-DMT or a pharmaceutically acceptable salt thereof.
160. 5-MeO-DMT or a pharmaceutically acceptable salt thereof for use as in aspect 157, wherein the reduction or elimination of anxiety as reflected by an improvement in the score of the BDRS item anxiety persists until at least 28 days after the last administration of 5-MeO-DMT or a pharmaceutically acceptable salt thereof.
161. 5-MeO-DMT or a pharmaceutically acceptable salt thereof for use as in aspect 151, wherein the reduction or elimination of anxiety is reflected by an improvement at least in the score of the BPRS item anxiety about 2 hours after the last administration of 5-MeO-DMT or a pharmaceutically acceptable salt thereof.
162. 5-MeO-DMT or a pharmaceutically acceptable salt thereof for use as in aspects 151 or 161, wherein the reduction or elimination of anxiety is reflected by an improvement at least in the score of the BPRS item anxiety on day 1 after the last administration of 5-MeO-DMT or a pharmaceutically acceptable salt thereof.
163. 5-MeO-DMT or a pharmaceutically acceptable salt thereof for use as in aspects 151, 161 or 162, wherein the reduction or elimination of anxiety is reflected by an improvement at least in the score of the BPRS item anxiety on day 7 after the last administration of 5-MeO-DMT or a pharmaceutically acceptable salt thereof.
164. 5-MeO-DMT or a pharmaceutically acceptable salt thereof for use as in aspects 151 or 161 to 163, wherein the reduction or elimination of anxiety is reflected by an improvement at least in the score of the BPRS item anxiety on day 14 after the last administration of 5-MeO-DMT or a pharmaceutically acceptable salt thereof.
165. 5-MeO-DMT or a pharmaceutically acceptable salt thereof for use as in aspects 151 or 161 to 164, wherein the reduction or elimination of anxiety is reflected by an improvement at least in the score of the BPRS item anxiety on day 28 after the last administration of 5-MeO-DMT or a pharmaceutically acceptable salt thereof.
166. 5-MeO-DMT or a pharmaceutically acceptable salt thereof for use as in aspect 151, wherein the reduction or elimination of anxiety as reflected by an improvement in the score of the BPRS item anxiety occurs not later than about 2 hours after the last administration of 5-MeO-DMT or a pharmaceutically acceptable salt thereof.
167. 5-MeO-DMT or a pharmaceutically acceptable salt thereof for use as in aspect 166, wherein the reduction or elimination of anxiety as reflected by an improvement in the score of the BPRS item anxiety persists until at least 6 days after the last administration of 5-MeO-DMT or a pharmaceutically acceptable salt thereof.
168. 5-MeO-DMT or a pharmaceutically acceptable salt thereof for use as in aspect 166, wherein the reduction or elimination of anxiety as reflected by an improvement in the score of the BPRS item anxiety persists until at least 14 days after the last administration of 5-MeO-DMT or a pharmaceutically acceptable salt thereof.
169. 5-MeO-DMT or a pharmaceutically acceptable salt thereof for use as in aspect 166, wherein the reduction or elimination of anxiety as reflected by an improvement in the score of the BPRS item anxiety persists until at least 28 days after the last administration of 5-MeO-DMT or a pharmaceutically acceptable salt thereof.
170. 5-MeO-DMT or a pharmaceutically acceptable salt thereof for use as in aspect 151, wherein the patient suffers from anxiety and an improvement in anxiety is reflected by a reduction in the Clinical Global Impression-Severity (CGI-S) score about 2 hours after the last administration of 5-MeO-DMT or a pharmaceutically acceptable salt thereof.
171. 5-MeO-DMT or a pharmaceutically acceptable salt thereof for use as in aspects 151 or 170, wherein the patient suffers from anxiety and an improvement in anxiety is reflected by a reduction in the Clinical Global Impression-Severity (CGI-S) score on day 1, for instance, about 24 hours after the last administration of 5-MeO-DMT or a pharmaceutically acceptable salt thereof.
172. 5-MeO-DMT or a pharmaceutically acceptable salt thereof for use as in aspects 151, 170 or 171, wherein the patient suffers from anxiety and an improvement in anxiety is reflected by a reduction in the Clinical Global Impression-Severity (CGI-S) score on day 7 after the last administration of 5-MeO-DMT or a pharmaceutically acceptable salt thereof.
173. 5-MeO-DMT or a pharmaceutically acceptable salt thereof for use as in aspects 151 or 170 to 172, wherein the patient suffers from anxiety and an improvement in anxiety is reflected by a reduction in the Clinical Global Impression-Severity (CGI-S) score on day 14 after the last administration of 5-MeO-DMT or a pharmaceutically acceptable salt thereof.
174. 5-MeO-DMT or a pharmaceutically acceptable salt thereof for use as in aspects 151 or 170 to 173, wherein the patient suffers from anxiety and an improvement in anxiety is reflected by a reduction in the Clinical Global Impression-Severity (CGI-S) score on day 28 after the last administration of 5-MeO-DMT or a pharmaceutically acceptable salt thereof.
175. 5-MeO-DMT or a pharmaceutically acceptable salt thereof for use as in aspects 1 to 150, wherein the patient suffers from anxiety and an improvement in anxiety, as reflected by a reduction in the Clinical Global Impression-Severity (CGI-S) score, occurs not later than about 2 hours after the last administration of 5-MeO-DMT or a pharmaceutically acceptable salt thereof.
176. 5-MeO-DMT or a pharmaceutically acceptable salt thereof for use as in aspect 175, wherein the improvement in anxiety, as reflected by a reduction in the Clinical Global Impression-Severity (CGI-S) score, persists until at least 6 days after the last administration of 5-MeO-DMT or a pharmaceutically acceptable salt thereof.
177. 5-MeO-DMT or a pharmaceutically acceptable salt thereof for use as in aspect 175, wherein the improvement in anxiety, as reflected by a reduction in the Clinical Global Impression-Severity (CGI-S) score, persists until at least 14 days after the last administration of 5-MeO-DMT or a pharmaceutically acceptable salt thereof.
178. 5-MeO-DMT or a pharmaceutically acceptable salt thereof for use as in aspect 175, wherein the improvement in anxiety, as reflected by a reduction in the Clinical Global Impression-Severity (CGI-S) score, persists until at least 28 days after the last administration of 5-MeO-DMT or a pharmaceutically acceptable salt thereof.
179. 5-MeO-DMT or a pharmaceutically acceptable salt thereof for use as in aspects 1 to 178, wherein the patient suffers from cognitive dysfunction and the treatment reduces or eliminates the cognitive dysfunction.
180. 5-MeO-DMT or a pharmaceutically acceptable salt thereof for use as in aspect 179, wherein the reduction or elimination of cognitive dysfunction is reflected by an improvement at least in the score of the BDRS item impaired concentration and memory about 2 hours after the last administration of 5-MeO-DMT or a pharmaceutically acceptable salt thereof.
181. 5-MeO-DMT or a pharmaceutically acceptable salt thereof for use as in aspects 179 or 180, wherein the reduction or elimination of cognitive dysfunction is reflected by an improvement at least in the score of the BDRS item impaired concentration and memory on day 1, for instance, about 24 hours after the last administration of 5-MeO-DMT or a pharmaceutically acceptable salt thereof.
182. 5-MeO-DMT or a pharmaceutically acceptable salt thereof for use as in aspects 179 to 181, wherein the reduction or elimination of cognitive dysfunction is reflected by an improvement at least in the score of the BDRS item impaired concentration and memory on day 7 after the last administration of 5-MeO-DMT or a pharmaceutically acceptable salt thereof.
183. 5-MeO-DMT or a pharmaceutically acceptable salt thereof for use as in aspects 179 to 182, wherein the reduction or elimination of cognitive dysfunction is reflected by an improvement at least in the score of the BDRS item impaired concentration and memory on day 14 after the last administration of 5-MeO-DMT or a pharmaceutically acceptable salt thereof.
184. 5-MeO-DMT or a pharmaceutically acceptable salt thereof for use as in aspects 179 to 183, wherein the reduction or elimination of cognitive dysfunction is reflected by an improvement at least in the score of the BDRS item impaired concentration and memory on day 28 after the last administration of 5-MeO-DMT or a pharmaceutically acceptable salt thereof.
185. 5-MeO-DMT or a pharmaceutically acceptable salt thereof for use as in aspect 179, wherein the reduction or elimination of cognitive dysfunction as reflected by an improvement in the score of the BDRS item impaired concentration and memory occurs not later than about 2 hours after the last administration of 5-MeO-DMT or a pharmaceutically acceptable salt thereof.
186. 5-MeO-DMT or a pharmaceutically acceptable salt thereof for use as in aspect 185, wherein the reduction or elimination of cognitive dysfunction as reflected by an improvement in the score of the BDRS item impaired concentration and memory persists until at least 6 days after the last administration of 5-MeO-DMT or a pharmaceutically acceptable salt thereof.
187. 5-MeO-DMT or a pharmaceutically acceptable salt thereof for use as in aspect 185, wherein the reduction or elimination of cognitive dysfunction as reflected by an improvement in the score of the BDRS item impaired concentration and memory persists until at least 14 days after the last administration of 5-MeO-DMT or a pharmaceutically acceptable salt thereof.
188. 5-MeO-DMT or a pharmaceutically acceptable salt thereof for use as in aspect 185, wherein the reduction or elimination of cognitive dysfunction as reflected by an improvement in the score of the BDRS item impaired concentration and memory persists until at least 28 days after the last administration of 5-MeO-DMT or a pharmaceutically acceptable salt thereof.
189. 5-MeO-DMT or a pharmaceutically acceptable salt thereof for use as in aspect 179, wherein the reduction or elimination of cognitive dysfunction is reflected by an improvement at least in the score of the MADRS item concentration difficulties about 2 hours after the last administration of 5-MeO-DMT or a pharmaceutically acceptable salt thereof.
190. 5-MeO-DMT or a pharmaceutically acceptable salt thereof for use as in aspects 179 or 189, wherein the reduction or elimination of cognitive dysfunction is reflected by an improvement at least in the score of the MADRS item concentration difficulties on day 1, for instance, about 24 hours after the last administration of 5-MeO-DMT or a pharmaceutically acceptable salt thereof.
191. 5-MeO-DMT or a pharmaceutically acceptable salt thereof for use as in aspects 179, 189 or 190, wherein the reduction or elimination of cognitive dysfunction is reflected by an improvement at least in the score of the MADRS item concentration difficulties on day 7 after the last administration of 5-MeO-DMT or a pharmaceutically acceptable salt thereof.
192. 5-MeO-DMT or a pharmaceutically acceptable salt thereof for use as in aspects 179 or 189 to 191, wherein the reduction or elimination of cognitive dysfunction is reflected by an improvement at least in the score of the MADRS item concentration difficulties on day 14 after the last administration of 5-MeO-DMT or a pharmaceutically acceptable salt thereof.
193. 5-MeO-DMT or a pharmaceutically acceptable salt thereof for use as in aspects 179 or 189 to 192, wherein the reduction or elimination of cognitive dysfunction is reflected by an improvement at least in the score of the MADRS item concentration difficulties on day 28 after the last administration of 5-MeO-DMT or a pharmaceutically acceptable salt thereof.
194. 5-MeO-DMT or a pharmaceutically acceptable salt thereof for use as in aspect 179, wherein the reduction or elimination of cognitive dysfunction as reflected by an improvement in the score of the MADRS item concentration difficulties occurs not later than about 2 hours after the last administration of 5-MeO-DMT or a pharmaceutically acceptable salt thereof.
195. 5-MeO-DMT or a pharmaceutically acceptable salt thereof for use as in aspect 194, wherein the reduction or elimination of cognitive dysfunction as reflected by an improvement in the score of the MADRS item concentration difficulties persists until at least 6 days after the last administration of 5-MeO-DMT or a pharmaceutically acceptable salt thereof.
196. 5-MeO-DMT or a pharmaceutically acceptable salt thereof for use as in aspect 194, wherein the reduction or elimination of cognitive dysfunction as reflected by an improvement in the score of the MADRS item concentration difficulties persists until at least 14 days after the last administration of 5-MeO-DMT or a pharmaceutically acceptable salt thereof.
197. 5-MeO-DMT or a pharmaceutically acceptable salt thereof for use as in aspect 194, wherein the reduction or elimination of cognitive dysfunction as reflected by an improvement in the score of the MADRS item concentration difficulties persists until at least 28 days after the last administration of 5-MeO-DMT or a pharmaceutically acceptable salt thereof.
198. 5-MeO-DMT or a pharmaceutically acceptable salt thereof for use as in aspect 179, wherein the patient suffers from cognitive dysfunction and an improvement in cognitive dysfunction is reflected by a reduction in the Clinical Global Impression-Severity (CGI-S) score about 2 hours after the last administration of 5-MeO-DMT or a pharmaceutically acceptable salt thereof.
199. 5-MeO-DMT or a pharmaceutically acceptable salt thereof for use as in aspects 179 or 198, wherein the patient suffers from cognitive dysfunction and an improvement in cognitive dysfunction is reflected by a reduction in the Clinical Global Impression-Severity (CGI-S) score on day 1, for instance, about 24 hours after the last administration of 5-MeO-DMT or a pharmaceutically acceptable salt thereof.
200. 5-MeO-DMT or a pharmaceutically acceptable salt thereof for use as in aspects 179, 198 or 199, wherein the patient suffers from cognitive dysfunction and an improvement in cognitive dysfunction is reflected by a reduction in the Clinical Global Impression-Severity (CGI-S) score on day 7 after the last administration of 5-MeO-DMT or a pharmaceutically acceptable salt thereof.
201. 5-MeO-DMT or a pharmaceutically acceptable salt thereof for use as in aspects 179 or 198 to 200, wherein the patient suffers from cognitive dysfunction and an improvement in cognitive dysfunction is reflected by a reduction in the Clinical Global Impression-Severity (CGI-S) score on day 14 after the last administration of 5-MeO-DMT or a pharmaceutically acceptable salt thereof.
202. 5-MeO-DMT or a pharmaceutically acceptable salt thereof for use as in aspects 179 or 198 to 201, wherein the patient suffers from cognitive dysfunction and an improvement in cognitive dysfunction is reflected by a reduction in the Clinical Global Impression-Severity (CGI-S) score on day 28 after the last administration of 5-MeO-DMT or a pharmaceutically acceptable salt thereof.
203. 5-MeO-DMT or a pharmaceutically acceptable salt thereof for use as in aspects 1 to 178, wherein the patient suffers from cognitive dysfunction and an improvement in cognitive dysfunction, as reflected by a reduction in the Clinical Global Impression-Severity (CGI-S) score, occurs not later than about 2 hours after the last administration of 5-MeO-DMT or a pharmaceutically acceptable salt thereof.
204. 5-MeO-DMT or a pharmaceutically acceptable salt thereof for use as in aspect 203, wherein the improvement in cognitive dysfunction, as reflected by a reduction in the Clinical Global Impression-Severity (CGI-S) score, persists until at least 6 days after the last administration of 5-MeO-DMT or a pharmaceutically acceptable salt thereof.
205. 5-MeO-DMT or a pharmaceutically acceptable salt thereof for use as in aspect 203, wherein the improvement in cognitive dysfunction, as reflected by a reduction in the Clinical Global Impression-Severity (CGI-S) score, persists until at least 14 days after the last administration of 5-MeO-DMT or a pharmaceutically acceptable salt thereof.
206. 5-MeO-DMT or a pharmaceutically acceptable salt thereof for use as in aspect 203, wherein the improvement in cognitive dysfunction, as reflected by a reduction in the Clinical Global Impression-Severity (CGI-S) score, persists until at least 28 days after the last administration of 5-MeO-DMT or a pharmaceutically acceptable salt thereof.
207. 5-MeO-DMT or a pharmaceutically acceptable salt thereof for use as in aspects 1 to 206, wherein the patient suffers from social/emotional withdrawal or detachment and the treatment reduces or eliminates the social/emotional withdrawal or detachment.
208. 5-MeO-DMT or a pharmaceutically acceptable salt thereof for use as in aspect 207, wherein the treatment reduces or eliminates at least one of anhedonia, emotional withdrawal and affective flattening.
209. 5-MeO-DMT or a pharmaceutically acceptable salt thereof for use as in aspects 207 or 208, wherein the reduction or elimination of social/emotional withdrawal or detachment is reflected by an improvement at least in the scores of the BDRS items anhedonia, emotional withdrawal and/or affective flattening about 2 hours after the last administration of 5-MeO-DMT or a pharmaceutically acceptable salt thereof.
210. 5-MeO-DMT or a pharmaceutically acceptable salt thereof for use as in aspects 207 to 209, wherein the reduction or elimination of social/emotional withdrawal or detachment is reflected by an improvement at least in the scores of the BDRS items anhedonia, emotional withdrawal and/or affective flattening on day 1, for instance, about 24 hours after the last administration of 5-MeO-DMT or a pharmaceutically acceptable salt thereof.
211. 5-MeO-DMT or a pharmaceutically acceptable salt thereof for use as in aspects 207 to 210, wherein the reduction or elimination of social/emotional withdrawal or detachment is reflected by an improvement at least in the score of the BDRS items anhedonia, emotional withdrawal and/or affective flattening on day 7 after the last administration of 5-MeO-DMT or a pharmaceutically acceptable salt thereof.
212. 5-MeO-DMT or a pharmaceutically acceptable salt thereof for use as in aspects 207 to 211, wherein the reduction or elimination of social/emotional withdrawal or detachment is reflected by an improvement at least in the scores of the BDRS items anhedonia, emotional withdrawal and/or affective flattening on day 14 after the last administration of 5-MeO-DMT or a pharmaceutically acceptable salt thereof.
213. 5-MeO-DMT or a pharmaceutically acceptable salt thereof for use as in aspects 207 to 212, wherein the reduction or elimination of social/emotional withdrawal or detachment is reflected by an improvement at least in the scores of the BDRS items anhedonia, emotional withdrawal and/or affective flattening on day 28 after the last administration of 5-MeO-DMT or a pharmaceutically acceptable salt thereof.
214. 5-MeO-DMT or a pharmaceutically acceptable salt thereof for use as in aspects 207 or 208, wherein the reduction or elimination of social/emotional withdrawal or detachment as reflected by an improvement in the scores of the BDRS items anhedonia, emotional withdrawal and/or affective flattening occurs not later than about 2 hours after the last administration of 5-MeO-DMT or a pharmaceutically acceptable salt thereof.
215. 5-MeO-DMT or a pharmaceutically acceptable salt thereof for use as in aspect 214, wherein the reduction or elimination of social/emotional withdrawal or detachment as reflected by an improvement in the scores of the BDRS items anhedonia, emotional withdrawal and/or affective flattening persists until at least 6 days after the last administration of 5-MeO-DMT or a pharmaceutically acceptable salt thereof.
216. 5-MeO-DMT or a pharmaceutically acceptable salt thereof for use as in aspect 214, wherein the reduction or elimination of social/emotional withdrawal or detachment as reflected by an improvement in the scores of the BDRS items anhedonia, emotional withdrawal and/or affective flattening persists until at least 14 days after the last administration of 5-MeO-DMT or a pharmaceutically acceptable salt thereof.
217. 5-MeO-DMT or a pharmaceutically acceptable salt thereof for use as in aspect 214, wherein the reduction or elimination of social/emotional withdrawal or detachment as reflected by an improvement in the scores of the BDRS items anhedonia, emotional withdrawal and/or affective flattening persists until at least 28 days after the last administration of 5-MeO-DMT or a pharmaceutically acceptable salt thereof.
218. 5-MeO-DMT or a pharmaceutically acceptable salt thereof for use as in aspects 207 or 208, wherein the reduction or elimination of social/emotional withdrawal or detachment is reflected by an improvement at least in the score of the MADRS item inability to feel about 2 hours after the last administration of 5-MeO-DMT or a pharmaceutically acceptable salt thereof.
219. 5-MeO-DMT or a pharmaceutically acceptable salt thereof for use as in aspects 207, 208 or 218, wherein the reduction or elimination of social/emotional withdrawal or detachment is reflected by an improvement at least in the score of the MADRS item inability to feel on day 1, for instance, about 24 hours days after the last administration of 5-MeO-DMT or a pharmaceutically acceptable salt thereof.
220. 5-MeO-DMT or a pharmaceutically acceptable salt thereof for use as in aspects 207, 208, 218 or 219, wherein the reduction or elimination of social/emotional withdrawal or detachment is reflected by an improvement at least in the score of the MADRS item inability to feel on day 7 days after the last administration of 5-MeO-DMT or a pharmaceutically acceptable salt thereof.
221. 5-MeO-DMT or a pharmaceutically acceptable salt thereof for use as in aspects 207, 208 or 218 to 220, wherein the reduction or elimination of social/emotional withdrawal or detachment is reflected by an improvement at least in the score of the MADRS item inability to feel on day 14 days after the last administration of 5-MeO-DMT or a pharmaceutically acceptable salt thereof.
222. 5-MeO-DMT or a pharmaceutically acceptable salt thereof for use as in aspects 207, 208 or 218 to 221, wherein the reduction or elimination of social/emotional withdrawal or detachment is reflected by an improvement at least in the score of the MADRS item inability to feel on day 28 after the last administration of 5-MeO-DMT or a pharmaceutically acceptable salt thereof.
223. 5-MeO-DMT or a pharmaceutically acceptable salt thereof for use as in aspects 207 or 208, wherein the reduction or elimination of social/emotional withdrawal or detachment as reflected by an improvement in the score of the MADRS item inability to feel occurs not later than about 2 hours after the last administration of 5-MeO-DMT or a pharmaceutically acceptable salt thereof.
224. 5-MeO-DMT or a pharmaceutically acceptable salt thereof for use as in aspect 223, wherein the reduction or elimination of social/emotional withdrawal or detachment as reflected by an improvement in the score of the MADRS item inability to feel persists until at least 6 days after the last administration of 5-MeO-DMT or a pharmaceutically acceptable salt thereof.
225. 5-MeO-DMT or a pharmaceutically acceptable salt thereof for use as in aspect 223, wherein the reduction or elimination of social/emotional withdrawal or detachment as reflected by an improvement in the score of the MADRS item inability to feel persists until at least 14 days after the last administration of 5-MeO-DMT or a pharmaceutically acceptable salt thereof.
226. 5-MeO-DMT or a pharmaceutically acceptable salt thereof for use as in aspect 223, wherein the reduction or elimination of social/emotional withdrawal or detachment as reflected by an improvement in the score of the MADRS item inability to feel persists until at least 28 days after the last administration of 5-MeO-DMT or a pharmaceutically acceptable salt thereof.
227. 5-MeO-DMT or a pharmaceutically acceptable salt thereof for use as in aspects 207 or 208, wherein the reduction or elimination of social/emotional withdrawal or detachment is reflected by an improvement at least in the score of the BPRS item emotional withdrawal about 2 hours after the last administration of 5-MeO-DMT or a pharmaceutically acceptable salt thereof.
228. 5-MeO-DMT or a pharmaceutically acceptable salt thereof for use as in aspects 207, 208 or 227, wherein the reduction or elimination of social/emotional withdrawal or detachment is reflected by an improvement at least in the score of the BPRS item emotional withdrawal on day 1, for instance, about 24 hours after the last administration of 5-MeO-DMT or a pharmaceutically acceptable salt thereof.
229. 5-MeO-DMT or a pharmaceutically acceptable salt thereof for use as in aspects 207, 208, 227 or 228, wherein the reduction or elimination of social/emotional withdrawal or detachment is reflected by an improvement at least in the score of the BPRS item emotional withdrawal on day 7 after the last administration of 5-MeO-DMT or a pharmaceutically acceptable salt thereof.
230. 5-MeO-DMT or a pharmaceutically acceptable salt thereof for use as in aspects 207, 208 or 227 to 229, wherein the reduction or elimination of social/emotional withdrawal or detachment is reflected by an improvement at least in the score of the BPRS item emotional withdrawal on day 14 after the last administration of 5-MeO-DMT or a pharmaceutically acceptable salt thereof.
231. 5-MeO-DMT or a pharmaceutically acceptable salt thereof for use as in aspects 207, 208 or 227 to 230, wherein the reduction or elimination of social/emotional withdrawal or detachment is reflected by an improvement at least in the score of the BPRS item emotional withdrawal on day 28 after the last administration of 5-MeO-DMT or a pharmaceutically acceptable salt thereof.
232. 5-MeO-DMT or a pharmaceutically acceptable salt thereof for use as in aspects 207 or 208, wherein the reduction or elimination of social/emotional withdrawal or detachment as reflected by an improvement in the score of the BPRS item emotional withdrawal occurs not later than about 2 hours after the last administration of 5-MeO-DMT or a pharmaceutically acceptable salt thereof.
233. 5-MeO-DMT or a pharmaceutically acceptable salt thereof for use as in aspect 232, wherein the reduction or elimination of social/emotional withdrawal or detachment as reflected by an improvement in the score of the BPRS item emotional withdrawal persists until at least 6 days after the last administration of 5-MeO-DMT or a pharmaceutically acceptable salt thereof.
234. 5-MeO-DMT or a pharmaceutically acceptable salt thereof for use as in aspect 232, wherein the reduction or elimination of social/emotional withdrawal or detachment as reflected by an improvement in the score of the BPRS item emotional withdrawal persists until at least 14 days after the last administration of 5-MeO-DMT or a pharmaceutically acceptable salt thereof.
235. 5-MeO-DMT or a pharmaceutically acceptable salt thereof for use as in aspect 232, wherein the reduction or elimination of social/emotional withdrawal or detachment as reflected by an improvement in the score of the BPRS item emotional withdrawal persists until at least 28 days after the last administration of 5-MeO-DMT or a pharmaceutically acceptable salt thereof.
236. 5-MeO-DMT or a pharmaceutically acceptable salt thereof for use as in aspects 207 or 208, wherein the reduction or elimination of social/emotional withdrawal or detachment is reflected by an improvement at least in the score of the BPRS item blunted affect about 2 hours after the last administration of 5-MeO-DMT or a pharmaceutically acceptable salt thereof.
237. 5-MeO-DMT or a pharmaceutically acceptable salt thereof for use as in aspects 207, 208 or 236, wherein the reduction or elimination of social/emotional withdrawal or detachment is reflected by an improvement at least in the score of the BPRS item blunted affect on day 1, for instance, about 24 hours after the last administration of 5-MeO-DMT or a pharmaceutically acceptable salt thereof.
238. 5-MeO-DMT or a pharmaceutically acceptable salt thereof for use as in aspects 207, 208, 236 or 237, wherein the reduction or elimination of social/emotional withdrawal or detachment is reflected by an improvement at least in the score of the BPRS item blunted affect on day 7 after the last administration of 5-MeO-DMT or a pharmaceutically acceptable salt thereof.
239. 5-MeO-DMT or a pharmaceutically acceptable salt thereof for use as in aspects 207, 208 or 236 to 238, wherein the reduction or elimination of social/emotional withdrawal or detachment is reflected by an improvement at least in the score of the BPRS item blunted affect on day 14 after the last administration of 5-MeO-DMT or a pharmaceutically acceptable salt thereof.
240. 5-MeO-DMT or a pharmaceutically acceptable salt thereof for use as in aspects 207, 208 or 236 to 239, wherein the reduction or elimination of social/emotional withdrawal or detachment is reflected by an improvement at least in the score of the BPRS item blunted affect on day 28 after the last administration of 5-MeO-DMT or a pharmaceutically acceptable salt thereof.
241. 5-MeO-DMT or a pharmaceutically acceptable salt thereof for use as in aspects 207 or 208, wherein the reduction or elimination of social/emotional withdrawal or detachment as reflected by an improvement in the score of the BPRS item blunted affect occurs not later than about 2 hours after the last administration of 5-MeO-DMT or a pharmaceutically acceptable salt thereof.
242. 5-MeO-DMT or a pharmaceutically acceptable salt thereof for use as in aspect 241, wherein the reduction or elimination of social/emotional withdrawal or detachment as reflected by an improvement in the score of the BPRS item blunted affect persists until at least 6 days after the last administration of 5-MeO-DMT or a pharmaceutically acceptable salt thereof.
243. 5-MeO-DMT or a pharmaceutically acceptable salt thereof for use as in aspect 241, wherein the reduction or elimination of social/emotional withdrawal or detachment as reflected by an improvement in the score of the BPRS item blunted affect persists until at least 14 days after the last administration of 5-MeO-DMT or a pharmaceutically acceptable salt thereof.
244. 5-MeO-DMT or a pharmaceutically acceptable salt thereof for use as in aspect 241, wherein the reduction or elimination of social/emotional withdrawal or detachment as reflected by an improvement in the score of the BPRS item blunted affect persists until at least 28 days after the last administration of 5-MeO-DMT or a pharmaceutically acceptable salt thereof.
245. 5-MeO-DMT or a pharmaceutically acceptable salt thereof for use as in aspects 207 or 208, wherein the patient suffers from social/emotional withdrawal or detachment and an improvement in social/emotional withdrawal or detachment is reflected by a reduction in the Clinical Global Impression-Severity (CGI-S) score about 2 hours after the last administration of 5-MeO-DMT or a pharmaceutically acceptable salt thereof.
246. 5-MeO-DMT or a pharmaceutically acceptable salt thereof for use as in aspects 207, 208 or 245, wherein the patient suffers from social/emotional withdrawal or detachment and an improvement in social/emotional withdrawal or detachment is reflected by a reduction in the Clinical Global Impression-Severity (CGI-S) score on day 1, for instance, about 24 hours after the last administration of 5-MeO-DMT or a pharmaceutically acceptable salt thereof.
247. 5-MeO-DMT or a pharmaceutically acceptable salt thereof for use as in aspects 207, 208, 245 or 246, wherein the patient suffers from social/emotional withdrawal or detachment and an improvement in social/emotional withdrawal or detachment is reflected by a reduction in the Clinical Global Impression-Severity (CGI-S) score on day 7 after the last administration of 5-MeO-DMT or a pharmaceutically acceptable salt thereof.
248. 5-MeO-DMT or a pharmaceutically acceptable salt thereof for use as in aspects 207, 208 or 245 to 247, wherein the patient suffers from social/emotional withdrawal or detachment and an improvement in social/emotional withdrawal or detachment is reflected by a reduction in the Clinical Global Impression-Severity (CGI-S) score on day 14 after the last administration of 5-MeO-DMT or a pharmaceutically acceptable salt thereof.
249. 5-MeO-DMT or a pharmaceutically acceptable salt thereof for use as in aspects 207, 208 or 245 to 248, wherein the patient suffers from social/emotional withdrawal or detachment and an improvement in social/emotional withdrawal or detachment is reflected by a reduction in the Clinical Global Impression-Severity (CGI-S) score on day 28 after the last administration of 5-MeO-DMT or a pharmaceutically acceptable salt thereof.
250. 5-MeO-DMT or a pharmaceutically acceptable salt thereof for use as in aspects 1 to 206, wherein the patient suffers from social/emotional withdrawal or detachment and an improvement at least in social/emotional withdrawal or detachment, as reflected by a reduction in the Clinical Global Impression-Severity (CGI-S) score, occurs not later than about 2 hours after the last administration of 5-MeO-DMT or a pharmaceutically acceptable salt thereof.
251. 5-MeO-DMT or a pharmaceutically acceptable salt thereof for use as in aspect 250, wherein the improvement in social/emotional withdrawal or detachment, as reflected by a reduction at least in the Clinical Global Impression-Severity (CGI-S) score, persists until at least 6 days after the last administration of 5-MeO-DMT or a pharmaceutically acceptable salt thereof.
252. 5-MeO-DMT or a pharmaceutically acceptable salt thereof for use as in aspect 251, wherein the improvement in social/emotional withdrawal or detachment, as reflected by a reduction at least in the Clinical Global Impression-Severity (CGI-S) score, persists until at least 14 days after the last administration of 5-MeO-DMT or a pharmaceutically acceptable salt thereof.
253. 5-MeO-DMT or a pharmaceutically acceptable salt thereof for use as in aspect 251, wherein the improvement in social/emotional withdrawal or detachment, as reflected by a reduction at least in the Clinical Global Impression-Severity (CGI-S) score, persists until at least 28 days after the last administration of 5-MeO-DMT or a pharmaceutically acceptable salt thereof.
254. 5-MeO-DMT or a pharmaceutically acceptable salt thereof for use as in aspects 1 to 253, wherein the treatment reduces or eliminates suicidal ideation.
255. 5-MeO-DMT or a pharmaceutically acceptable salt thereof for use as in aspect 254, wherein the reduction or elimination of suicidal ideation is reflected by an improvement at least in the score of the BDRS item suicidal ideation about 2 hours after the last administration of 5-MeO-DMT or a pharmaceutically acceptable salt thereof.
256. 5-MeO-DMT or a pharmaceutically acceptable salt thereof for use as in aspects 254 or 255, wherein the reduction or elimination of suicidal ideation is reflected by an improvement at least in the score of the BDRS item suicidal ideation on day 1, for instance, about 24 hours after the last administration of 5-MeO-DMT or a pharmaceutically acceptable salt thereof.
257. 5-MeO-DMT or a pharmaceutically acceptable salt thereof for use as in aspects 254 to 256, wherein the reduction or elimination of suicidal ideation is reflected by an improvement at least in the score of the BDRS item suicidal ideation on day 7 after the last administration of 5-MeO-DMT or a pharmaceutically acceptable salt thereof.
258. 5-MeO-DMT or a pharmaceutically acceptable salt thereof for use as in aspects 254 to 257, wherein the reduction or elimination of suicidal ideation is reflected by an improvement at least in the score of the BDRS item suicidal ideation on day 14 after the last administration of 5-MeO-DMT or a pharmaceutically acceptable salt thereof.
259. 5-MeO-DMT or a pharmaceutically acceptable salt thereof for use as in aspects 254 to 258, wherein the reduction or elimination of suicidal ideation is reflected by an improvement at least in the score of the BDRS item suicidal ideation on day 28 after the last administration of 5-MeO-DMT or a pharmaceutically acceptable salt thereof.
260. 5-MeO-DMT or a pharmaceutically acceptable salt thereof for use as in aspect 254, wherein the reduction or elimination of suicidal ideation as reflected by an improvement in the score of the BDRS item suicidal ideation occurs not later than about 2 hours after the last administration of 5-MeO-DMT or a pharmaceutically acceptable salt thereof.
261. 5-MeO-DMT or a pharmaceutically acceptable salt thereof for use as in aspect 260, wherein the reduction or elimination of suicidal ideation as reflected by an improvement in the score of the BDRS item suicidal ideation persists until at least 6 days after the last administration of 5-MeO-DMT or a pharmaceutically acceptable salt thereof.
262. 5-MeO-DMT or a pharmaceutically acceptable salt thereof for use as in aspect 260, wherein the reduction or elimination of suicidal ideation as reflected by an improvement in the score of the BDRS item suicidal ideation persists until at least 14 days after the last administration of 5-MeO-DMT or a pharmaceutically acceptable salt thereof.
263. 5-MeO-DMT or a pharmaceutically acceptable salt thereof for use as in aspect 260, wherein the reduction or elimination of suicidal ideation as reflected by an improvement in the score of the BDRS item suicidal ideation persists until at least 28 days after the last administration of 5-MeO-DMT or a pharmaceutically acceptable salt thereof.
264. 5-MeO-DMT or a pharmaceutically acceptable salt thereof for use as in aspect 254, wherein the reduction or elimination of suicidal ideation is reflected by an improvement at least in the score of the MADRS item suicidal thoughts about 2 hours after the last administration of 5-MeO-DMT or a pharmaceutically acceptable salt thereof.
265. 5-MeO-DMT or a pharmaceutically acceptable salt thereof for use as in aspects 254 or 264, wherein the reduction or elimination of suicidal ideation is reflected by an improvement at least in the score of the MADRS item suicidal thoughts on day 1, for instance, about 24 hours after the last administration of 5-MeO-DMT or a pharmaceutically acceptable salt thereof.
266. 5-MeO-DMT or a pharmaceutically acceptable salt thereof for use as in aspects 254, 264 or 265, wherein the reduction or elimination of suicidal ideation is reflected by an improvement at least in the score of the MADRS item suicidal thoughts on day 7 after the last administration of 5-MeO-DMT or a pharmaceutically acceptable salt thereof.
267. 5-MeO-DMT or a pharmaceutically acceptable salt thereof for use as in aspects 254 or 264 to 266, wherein the reduction or elimination of suicidal ideation is reflected by an improvement at least in the score of the MADRS item suicidal thoughts on day 14 after the last administration of 5-MeO-DMT or a pharmaceutically acceptable salt thereof.
268. 5-MeO-DMT or a pharmaceutically acceptable salt thereof for use as in aspects 254 or 264 to 267, wherein the reduction or elimination of suicidal ideation is reflected by an improvement at least in the score of the MADRS item suicidal thoughts on day 28 after the last administration of 5-MeO-DMT or a pharmaceutically acceptable salt thereof.
269. 5-MeO-DMT or a pharmaceutically acceptable salt thereof for use as in aspect 254, wherein the reduction or elimination of suicidal ideation as reflected by an improvement in the score of the MADRS item suicidal thoughts occurs not later than about 2 hours after the last administration of 5-MeO-DMT or a pharmaceutically acceptable salt thereof.
270. 5-MeO-DMT or a pharmaceutically acceptable salt thereof for use as in aspect 269, wherein the reduction or elimination of suicidal ideation as reflected by an improvement in the score of the MADRS item suicidal thoughts persists until at least 6 days after the last administration of 5-MeO-DMT or a pharmaceutically acceptable salt thereof.
271. 5-MeO-DMT or a pharmaceutically acceptable salt thereof for use as in aspect 269, wherein the reduction or elimination of suicidal ideation as reflected by an improvement in the score of the MADRS item suicidal thoughts persists until at least 14 days after the last administration of 5-MeO-DMT or a pharmaceutically acceptable salt thereof.
272. 5-MeO-DMT or a pharmaceutically acceptable salt thereof for use as in aspect 269, wherein the reduction or elimination of suicidal ideation as reflected by an improvement in the score of the MADRS item suicidal thoughts persists until at least 28 days after the last administration of 5-MeO-DMT or a pharmaceutically acceptable salt thereof.
273. 5-MeO-DMT or a pharmaceutically acceptable salt thereof for use as in aspect 254, wherein the patient suffers from suicidal ideation and an improvement in suicidal ideation is reflected by a reduction in the Clinical Global Impression-Severity (CGI-S) score about 2 hours after the last administration of 5-MeO-DMT or a pharmaceutically acceptable salt thereof.
274. 5-MeO-DMT or a pharmaceutically acceptable salt thereof for use as in aspects 254 or 273, wherein the patient suffers from suicidal ideation and an improvement in suicidal ideation is reflected by a reduction in the Clinical Global Impression-Severity (CGI-S) score on day 1, for instance, about 24 hours after the last administration of 5-MeO-DMT or a pharmaceutically acceptable salt thereof.
275. 5-MeO-DMT or a pharmaceutically acceptable salt thereof for use as in aspects 254, 273 or 274, wherein the patient suffers from suicidal ideation and an improvement in suicidal ideation is reflected by a reduction in the Clinical Global Impression-Severity (CGI-S) score on day 7 after the last administration of 5-MeO-DMT or a pharmaceutically acceptable salt thereof.
276. 5-MeO-DMT or a pharmaceutically acceptable salt thereof for use as in aspects 254 or 273 to 275, wherein the patient suffers from suicidal ideation and an improvement in suicidal ideation is reflected by a reduction in the Clinical Global Impression-Severity (CGI-S) score on day 14 after the last administration of 5-MeO-DMT or a pharmaceutically acceptable salt thereof.
277. 5-MeO-DMT or a pharmaceutically acceptable salt thereof for use as in aspects 254 or 273 to 276, wherein the patient suffers from suicidal ideation and an improvement in suicidal ideation is reflected by a reduction in the Clinical Global Impression-Severity (CGI-S) score on day 28 after the last administration of 5-MeO-DMT or a pharmaceutically acceptable salt thereof.
278. 5-MeO-DMT or a pharmaceutically acceptable salt thereof for use as in aspects 1 to 253, wherein the patient suffers from suicidal ideation and an improvement in suicidal ideation, as reflected by a reduction in the Clinical Global Impression-Severity (CGI-S) score, occurs not later than about 2 hours after the last administration of 5-MeO-DMT or a pharmaceutically acceptable salt thereof.
279. 5-MeO-DMT or a pharmaceutically acceptable salt thereof for use as in aspect 278, wherein the improvement in suicidal ideation, as reflected by a reduction in the Clinical Global Impression-Severity (CGI-S) score, persists until at least 6 days after the last administration of 5-MeO-DMT or a pharmaceutically acceptable salt thereof.
280. 5-MeO-DMT or a pharmaceutically acceptable salt thereof for use as in aspect 278, wherein the improvement in suicidal ideation, as reflected by a reduction in the Clinical Global Impression-Severity (CGI-S) score, persists until at least 14 days after the last administration of 5-MeO-DMT or a pharmaceutically acceptable salt thereof.
281. 5-MeO-DMT or a pharmaceutically acceptable salt thereof for use as in aspect 278, wherein the improvement in suicidal ideation, as reflected by a reduction in the Clinical Global Impression-Severity (CGI-S) score, persists until at least 28 days after the last administration of 5-MeO-DMT or a pharmaceutically acceptable salt thereof.
282. 5-MeO-DMT or a pharmaceutically acceptable salt thereof for use as in aspects 1 to 281, wherein the treatment reduces or eliminates at least one of psychotic symptoms; irritability; lability; increased motor drive; increased speech; agitation.
283. 5-MeO-DMT or a pharmaceutically acceptable salt thereof for use as in aspects 1 to 282, wherein the patient is a lactating woman who is advised to discontinue breastfeeding until 48 hours after the last administration of 5-MeO-DMT or a pharmaceutically acceptable salt thereof.
284. 5-MeO-DMT or a pharmaceutically acceptable salt thereof for use as in aspects 1 to 282, wherein the patient is a lactating woman who is advised to discontinue breastfeeding until 24 hours after the last administration of 5-MeO-DMT or a pharmaceutically acceptable salt thereof.
285. 5-MeO-DMT or a pharmaceutically acceptable salt thereof for use as in aspects 1 to 282, wherein the patient is a lactating woman who is advised to discontinue breastfeeding until 6 hours after the last administration of 5-MeO-DMT or a pharmaceutically acceptable salt thereof.
286. 5-MeO-DMT or a pharmaceutically acceptable salt thereof for use in any of aspects 1 to 282, wherein the patient is a breastfeeding mother who is advised to discontinue breastfeeding until 3 hours after the last administration of 5-MeO-DMT or a pharmaceutically acceptable salt thereof.
287. 5-MeO-DMT or a pharmaceutically acceptable salt thereof for use as in aspects 1 to 282, wherein the patient is a lactating woman who is advised to discontinue breastfeeding until 2 hours after the last administration of 5-MeO-DMT or a pharmaceutically acceptable salt thereof.
288. 5-MeO-DMT or a pharmaceutically acceptable salt thereof for use as in aspects 1 to 287, wherein the treatment improves maternal functioning.
289. 5-MeO-DMT or a pharmaceutically acceptable salt thereof for use as in aspect 288, wherein the improvement relates to one or more, in particular two or more functional domains according to the Barkin Index of Maternal Functioning (BIMF) selected from self-care, infant care, mother-child interaction, psychological wellbeing of the mother, social support, management, and adjustment.
290. 5-MeO-DMT or a pharmaceutically acceptable salt thereof for use as in aspect 288 or 289, wherein the BIMF score is improved by 10% or more, preferably by 20% or more.

Further Specific Points
1. 5-Methoxy-N,N-dimethyltryptamine (5-MeO-DMT) or a pharmaceutically acceptable salt thereof for use in the treatment of a mental or nervous system disorder in a mother having a child of age 18 months or below, wherein the 5-MeO-DMT is administered via the intravenous, intramuscular or subcutaneous route.
2. 5-MeO-DMT or a pharmaceutically acceptable salt thereof for use as in point 1, wherein the mental or nervous system disorder involves one or more symptoms selected from sleep disturbance, cognitive dysfunction, anxiety, psychomotor retardation, social/emotional withdrawal and negative thinking.
3. 5-MeO-DMT or a pharmaceutically acceptable salt thereof for use as in points 1 or 2, wherein the patient suffers from a treatment resistant form of the disorder.
4. 5-MeO-DMT or a pharmaceutically acceptable salt thereof for use as in point 3, wherein an improvement of the disorder, as reflected by a reduction in the CGI-S score, is observed after about 2 hours; on day 1, for instance after about 24 hours; on day 7; on day 14; and/or on day 28 after the last administration of 5-MeO-DMT or a pharmaceutically acceptable salt thereof.
5. 5-MeO-DMT or a pharmaceutically acceptable salt thereof for use as in point 3, wherein an improvement of the disorder, as reflected by a reduction in the CGI-S score, occurs not later than about 2 hours after the last administration of 5-MeO-DMT or a pharmaceutically acceptable salt thereof.
6. 5-MeO-DMT or a pharmaceutically acceptable salt thereof for use as in points 3 or 5, wherein an improvement of the disorder, as reflected by a reduction in the CGI-S score, persists until at least 6 days; in particular until at least 14 days; more preferably until at least 28 days after the last administration of 5-MeO-DMT or a pharmaceutically acceptable salt thereof.
7. 5-MeO-DMT or a pharmaceutically acceptable salt thereof for use as in points 1 to 6, wherein the patient suffers in addition from slightly compromised, compromised or severely compromised maternal functioning.
8. 5-MeO-DMT or a pharmaceutically acceptable salt thereof for use as in point 7, wherein the patient has a Barkin Index of Maternal Functioning (BIMF) score of 95 or below such as 80 or below, in particular 65 or below.
9. 5-MeO-DMT or a pharmaceutically acceptable salt thereof for use as in points 1 to 8, wherein the treatment improves maternal functioning.
10. 5-MeO-DMT or a pharmaceutically acceptable salt thereof for use as in points 1 to 9, wherein the improvement relates to one or more, in particular two or more functional domains according to the Barkin Index of Maternal Functioning (BIMF) selected from self-care, infant care, mother-child interaction, psychological wellbeing of the mother, social support, management, and adjustment.
11. 5-MeO-DMT or a pharmaceutically acceptable salt thereof for use as in point 8 to 10, wherein the BIMF score is improved by 10% or more, preferably by 20% or more.
12. 5-MeO-DMT or a pharmaceutically acceptable salt thereof for use as in points 1 to 11, wherein the 5-MeO-DMT or salt thereof is administered at a dose or in a dosage regimen that causes the patient to experience a peak psychedelic experience.
13. 5-MeO-DMT or a pharmaceutically acceptable salt thereof for use as in points 1 to 12, wherein a dosage of about 1 mg to about 10 mg 5-MeO-DMT is administered, or wherein equimolar amounts of the pharmaceutically acceptable salt are administered instead of 5-MeO-DMT.
14. 5-MeO-DMT or a pharmaceutically acceptable salt thereof for use as in points 1 to 12, wherein a dosage of about 2 mg; or of about 5 mg; or of about 8 mg is administered, or wherein equimolar amounts of the pharmaceutically acceptable salt are administered instead of 5-MeO-DMT.
15. 5-MeO-DMT or a pharmaceutically acceptable salt thereof for use as in points 1 to 12, wherein a dosage of about 1 mg; or of about 2 mg; or of about 3 mg is administered, or wherein equimolar amounts of the pharmaceutically acceptable salt are administered instead of 5-MeO-DMT.
16. 5-MeO-DMT or a pharmaceutically acceptable salt thereof for use as in points 1 to 13, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.
17. 5-MeO-DMT or a pharmaceutically acceptable salt thereof for use as in points 1 to 13 or 16, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.
18. 5-MeO-DMT or a pharmaceutically acceptable salt thereof for use as in point 17, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.
19. 5-MeO-DMT or a pharmaceutically acceptable salt thereof for use as in points 1 to 13 or 16, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.
20. 5-MeO-DMT or a pharmaceutically acceptable salt thereof for use as in point 19, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.
21. 5-MeO-DMT or a pharmaceutically acceptable salt thereof for use as in points 16 to 20, wherein the interval between two administrations is not less than 1 hour and not more than 24 hours, such as about 1 to 4 hours, preferably about 1 to 2 hours.
22. 5-MeO-DMT or a pharmaceutically acceptable salt thereof for use as in points 12 to 21, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.
23. 5-MeO-DMT or a pharmaceutically acceptable salt thereof for use as in point 22, wherein the occurrence of a peak psychedelic experience is identified through achievement of a Peak Experience Scale (PES) Total Score of at least 75.
24. 5-MeO-DMT or a pharmaceutically acceptable salt thereof for use as in points 1 to 23, wherein the 5-MeO-DMT or a pharmaceutically acceptable salt thereof is administered via intravenous injection.
25. 5-MeO-DMT for use as in points 1 to 24, wherein the disorder is a disorder characterized by depressive episodes, for example, Major Depressive Disorder (MDD), Persistent Depressive Disorder, Seasonal Affective Disorder and Bipolar Disorder (BD), such as Bipolar I Disorder and Bipolar II Disorder; Anxiety Disorder, for example, Separation Anxiety Disorder, Agoraphobia, Generalised Anxiety Disorder (GAD), Social Anxiety Disorder (SAD), Panic Disorder, Phobia, and Substance/Medication Induced Anxiety Disorder; Somatic Symptom Disorder; Obsessive compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain, Fibromyalgia, and Migraine; Mental and Behavioural Disorders due to Psychoactive Substance Use, for example, Substance Use Disorder (SUD); Psychotic Disorders, for example Schizophrenia; an Eating Disorder; Attention Deficit Hyperactivity Disorder (ADHD); a Personality Disorder, for example Schizotypal Personality Disorder and Borderline Personality Disorder; Autism Spectrum Disorder; Chronic Fatigue Syndrome; a mental disorder or a nervous system disorder associated with HIV, with Post COVID Condition or with Traumatic Brain Injury.
26. 5-MeO-DMT for use as in points 1 to 25, wherein the patient suffers from sleep disturbance.
27. 5-Methoxy-N,N-dimethyltryptamine (5-MeO-DMT) or a pharmaceutically acceptable salt thereof for use in any of points 1 to 26, wherein the patient is a breastfeeding mother who is advised to discontinue breastfeeding until 48 hours after the last administration of 5-MeO-DMT or a pharmaceutically acceptable salt thereof.
28. 5-Methoxy-N,N-dimethyltryptamine (5-MeO-DMT) or a pharmaceutically acceptable salt thereof for use in any of points 1 to 26, wherein the patient is a breastfeeding mother who is advised to discontinue breastfeeding until 24 hours after the last administration of 5-MeO-DMT or a pharmaceutically acceptable salt thereof.
29. 5-Methoxy-N,N-dimethyltryptamine (5-MeO-DMT) or a pharmaceutically acceptable salt thereof for use in any of points 1 to 26, wherein the patient is a breastfeeding mother who is advised to discontinue breastfeeding until 6 hours after the last administration of 5-MeO-DMT or a pharmaceutically acceptable salt thereof.
30. 5-Methoxy-N,N-dimethyltryptamine (5-MeO-DMT) or a pharmaceutically acceptable salt thereof for use in any of points 1 to 26, wherein the patient is a breastfeeding mother who is advised to discontinue breastfeeding until 3 hours after the last administration of 5-MeO-DMT or a pharmaceutically acceptable salt thereof.
31. 5-Methoxy-N,N-dimethyltryptamine (5-MeO-DMT) or a pharmaceutically acceptable salt thereof for use in any of points 1 to 26, wherein the patient is a breastfeeding mother who is advised to discontinue breastfeeding until 2 hours after the last administration of 5-MeO-DMT or a pharmaceutically acceptable salt thereof.

Further Specific Items
1. 5-Methoxy-N,N-dimethyltryptamine (5-MeO-DMT) or a pharmaceutically acceptable salt thereof for use in the treatment of a mental or nervous system disorder in a breastfeeding mother,
wherein a dosage of about 1 mg to about 10 mg 5-MeO-DMT or of an equimolar amount of the pharmaceutically acceptable salt is administered as a single dose or as the highest dose in an uptitration scheme involving intervals between administrations of at least about 1 hour,
wherein the 5-MeO-DMT or the pharmaceutically acceptable salt thereof is administered via the intravenous, intramuscular or subcutaneous route,
wherein the patient is advised to temporarily cease breastfeeding.
2. 5-Methoxy-N,N-dimethyltryptamine (5-MeO-DMT) or a pharmaceutically acceptable salt thereof for use as in item 1, wherein the patient is advised to temporarily cease breastfeeding from 1 hour or more prior to receiving the first dose and not to resume breastfeeding until at least 24 hours post last dose.
3. 5-Methoxy-N,N-dimethyltryptamine (5-MeO-DMT) or a pharmaceutically acceptable salt thereof for use as in item 1, wherein the patient is advised to temporarily cease breastfeeding from 1 hour or more prior to receiving the first dose and not to resume breastfeeding until at least 12 hours post last dose.
4. 5-Methoxy-N,N-dimethyltryptamine (5-MeO-DMT) or a pharmaceutically acceptable salt thereof for use as in item 1, wherein the patient is advised to temporarily cease breastfeeding from 1 hour or more prior to receiving the first dose and not to resume breastfeeding until at least 6 hours post last dose.
5. 5-Methoxy-N,N-dimethyltryptamine (5-MeO-DMT) or a pharmaceutically acceptable salt thereof for use as in item 1, wherein the patient is advised to temporarily cease breastfeeding from 1 hour or more prior to receiving the first dose and not to resume breastfeeding until at least 2 hours post last dose.
6. 5-Methoxy-N,N-dimethyltryptamine (5-MeO-DMT) or a pharmaceutically acceptable salt thereof for use as in item 1, wherein the patient is advised to temporarily cease breastfeeding from 1 hour or more prior to receiving the first dose and not to resume breastfeeding until at least 1 hours post last dose.
7. 5-Methoxy-N,N-dimethyltryptamine (5-MeO-DMT) or a pharmaceutically acceptable salt thereof for use as in item 1, wherein the patient is advised to temporarily cease breastfeeding from just prior to receiving the first dose and not to resume breastfeeding until at least 24 hours post last dose.
8. 5-Methoxy-N,N-dimethyltryptamine (5-MeO-DMT) or a pharmaceutically acceptable salt thereof for use as in item 1, wherein the patient is advised to temporarily cease breastfeeding from just prior to receiving the first dose and not to resume breastfeeding until at least 12 hours post last dose.
9. 5-Methoxy-N,N-dimethyltryptamine (5-MeO-DMT) or a pharmaceutically acceptable salt thereof for use as in item 1, wherein the patient is advised to temporarily cease breastfeeding from just prior to receiving the first dose and not to resume breastfeeding until at least 6 hours post last dose.
10. 5-Methoxy-N,N-dimethyltryptamine (5-MeO-DMT) or a pharmaceutically acceptable salt thereof for use as in item 1, wherein the patient is advised to temporarily cease breastfeeding from just prior to receiving the first dose and not to resume breastfeeding until at least 2 hours post last dose.
11. 5-Methoxy-N,N-dimethyltryptamine (5-MeO-DMT) or a pharmaceutically acceptable salt thereof for use as in item 1, wherein the patient is advised to temporarily cease breastfeeding from just prior to receiving the first dose and not to resume breastfeeding until at least 1 hours post last dose.
12. 5-Methoxy-N,N-dimethyltryptamine (5-MeO-DMT) or a pharmaceutically acceptable salt thereof for use as in item 1, wherein the patient is advised to temporarily cease breastfeeding only for the period of the actual treatment.
13. 5-Methoxy-N,N-dimethyltryptamine (5-MeO-DMT) or a pharmaceutically acceptable salt thereof for use as in items 1 to 12, wherein the patient is advised to temporarily cease breastfeeding from just prior to receiving the first dose until discharge readiness.
14. 5-Methoxy-N,N-dimethyltryptamine (5-MeO-DMT) or a pharmaceutically acceptable salt thereof for use as in item 13, wherein the determination of discharge readiness occurs at about 1 hour after the last dose.
15. 5-Methoxy-N,N-dimethyltryptamine (5-MeO-DMT) or a pharmaceutically acceptable salt thereof for use as in item 13 or 14, wherein the patient is advised not to recommence breastfeeding before the later of discharge and 6 hours after the last dose.
16. 5-Methoxy-N,N-dimethyltryptamine (5-MeO-DMT) or a pharmaceutically acceptable salt thereof for use as in items 1 to 15, wherein the patient is advised not to recommence breastfeeding before the later of discharge and 3 hours after the last dose.
17. 5-Methoxy-N,N-dimethyltryptamine (5-MeO-DMT) or a pharmaceutically acceptable salt thereof for use as in items 1 to 15, wherein the patient is advised not to recommence breastfeeding before the later of discharge and 2 hours after the last dose.
18. 5-Methoxy-N,N-dimethyltryptamine (5-MeO-DMT) or a pharmaceutically acceptable salt thereof for use as in items 1 to 15, wherein the patient is advised not to recommence breastfeeding before the later of discharge and 1 hours after the last dose.
19. 5-Methoxy-N,N-dimethyltryptamine (5-MeO-DMT) or a pharmaceutically acceptable salt thereof for use as in items 1 to 18, wherein the patient is advised to temporarily cease breastfeeding from just prior to receiving the first dose until at least 24 hours post last dose and to discard all breast milk expressed during the 24 hour period.
20. 5-Methoxy-N,N-dimethyltryptamine (5-MeO-DMT) or a pharmaceutically acceptable salt thereof for use as in items 1 to 18, wherein the patient is advised to temporarily cease breastfeeding from just prior to receiving the first dose until at least 2.5 hours post last dose and to pump and discard breast milk at 2.5 hours post last dose.
21. 5-Methoxy-N,N-dimethyltryptamine (5-MeO-DMT) or a pharmaceutically acceptable salt thereof for use as in items 1 to 20, wherein the patient is advised to pump and discard breast milk at 24 hours post last dose prior to reinitiating breastfeeding.
22. 5-Methoxy-N,N-dimethyltryptamine (5-MeO-DMT) or a pharmaceutically acceptable salt thereof for use as in items 1 to 21, wherein the patient is advised to breastfeed the child not more than 2 times during the first 12 hours post last dose.
23. 5-Methoxy-N,N-dimethyltryptamine (5-MeO-DMT) or a pharmaceutically acceptable salt thereof for use as in items 1 to 22, wherein any expressed breast milk is discarded as long as the concentrations of 5-MeO-DMT and/or 5-MIAA in breast milk exceed predetermined thresholds.
24. 5-Methoxy-N,N-dimethyltryptamine (5-MeO-DMT) or a pharmaceutically acceptable salt thereof for use as in item 23, wherein the threshold for 5-MeO-DMT is 2000 pg.
25. 5-Methoxy-N,N-dimethyltryptamine (5-MeO-DMT) or a pharmaceutically acceptable salt thereof for use as in item 23, wherein the threshold for 5-MeO-DMT is 500 pg.
26. 5-Methoxy-N,N-dimethyltryptamine (5-MeO-DMT) or a pharmaceutically acceptable salt thereof for use as in item 23, wherein the threshold for 5-MeO-DMT is 75 pg.
27. 5-Methoxy-N,N-dimethyltryptamine (5-MeO-DMT) or a pharmaceutically acceptable salt thereof for use as in items 23 to 26, wherein the threshold for 5-MIAA is 14000 pg.
28. 5-Methoxy-N,N-dimethyltryptamine (5-MeO-DMT) or a pharmaceutically acceptable salt thereof for use as in items 23 to 26, wherein the threshold for 5-MIAA is 2000 pg.
29. 5-Methoxy-N,N-dimethyltryptamine (5-MeO-DMT) or a pharmaceutically acceptable salt thereof for use as in items 23 to 26, wherein the threshold for 5-MIAA is 75 pg.
30. 5-Methoxy-N,N-dimethyltryptamine (5-MeO-DMT) or a pharmaceutically acceptable salt thereof for use as in items 1 to 22, breastfeeding is resumed when the 5-MeO-DMT concentration and/or the 5-MIAA concentration in breast milk is below a predetermined threshold.
31. 5-Methoxy-N,N-dimethyltryptamine (5-MeO-DMT) or a pharmaceutically acceptable salt thereof for use as in item 30, wherein the threshold for 5-MeO-DMT is 2000 pg.
32. 5-Methoxy-N,N-dimethyltryptamine (5-MeO-DMT) or a pharmaceutically acceptable salt thereof for use as in item 30, wherein the threshold for 5-MeO-DMT is 500 pg.
33. 5-Methoxy-N,N-dimethyltryptamine (5-MeO-DMT) or a pharmaceutically acceptable salt thereof for use as in item 30, wherein the threshold for 5-MeO-DMT is 75 pg.
34. 5-Methoxy-N,N-dimethyltryptamine (5-MeO-DMT) or a pharmaceutically acceptable salt thereof for use as in any one of items 30 to 33, wherein the threshold for 5-MIAA is 14000 pg.
35. 5-Methoxy-N,N-dimethyltryptamine (5-MeO-DMT) or a pharmaceutically acceptable salt thereof for use as in any one of items 30 to 33, wherein the threshold for 5-MIAA is 2000 pg.
36. 5-Methoxy-N,N-dimethyltryptamine (5-MeO-DMT) or a pharmaceutically acceptable salt thereof for use as in any one of items 30 to 33, wherein the threshold for 5-MIAA is 75 pg.
37. 5-Methoxy-N,N-dimethyltryptamine (5-MeO-DMT) or a pharmaceutically acceptable salt thereof for use as in items 1 to 36, wherein breastfeeding is adapted such that the DID of 5-MeO-DMT per kg infant weight is 1 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
38. 5-Methoxy-N,N-dimethyltryptamine (5-MeO-DMT) or a pharmaceutically acceptable salt thereof for use as in items 1 to 36, wherein breastfeeding is adapted such that the DID of 5-MeO-DMT per kg infant weight is 0.4 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
39. 5-Methoxy-N,N-dimethyltryptamine (5-MeO-DMT) or a pharmaceutically acceptable salt thereof for use as in items 1 to 36, wherein breastfeeding is adapted such that the DID of 5-MeO-DMT per kg infant weight is 0.2 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
40. 5-Methoxy-N,N-dimethyltryptamine (5-MeO-DMT) or a pharmaceutically acceptable salt thereof for use as in items 1 to 39, wherein breastfeeding is adapted such that the RID of 5-MeO-DMT is 10% or below, for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
41. 5-Methoxy-N,N-dimethyltryptamine (5-MeO-DMT) or a pharmaceutically acceptable salt thereof for use as in items 1 to 39, wherein breastfeeding is adapted such that the RID of 5-MeO-DMT is 5% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
42. 5-Methoxy-N,N-dimethyltryptamine (5-MeO-DMT) or a pharmaceutically acceptable salt thereof for use as in items 1 to 39, wherein breastfeeding is adapted such that the RID of 5-MeO-DMT is 1% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
43. 5-Methoxy-N,N-dimethyltryptamine (5-MeO-DMT) or a pharmaceutically acceptable salt thereof for use as in items 1 to 42, wherein breastfeeding is adapted such that the DID of the terminal metabolite 5-MIAA per kg infant weight is 4 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
44. 5-Methoxy-N,N-dimethyltryptamine (5-MeO-DMT) or a pharmaceutically acceptable salt thereof for use as in items 1 to 42, wherein breastfeeding is adapted such that the DID of the terminal metabolite 5-MIAA per kg infant weight is 2 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
45. 5-Methoxy-N,N-dimethyltryptamine (5-MeO-DMT) or a pharmaceutically acceptable salt thereof for use as in items 1 to 42, wherein breastfeeding is adapted such that the DID of the terminal metabolite 5-MIAA per kg infant weight is 1 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
46. 5-Methoxy-N,N-dimethyltryptamine (5-MeO-DMT) or a pharmaceutically acceptable salt thereof for use as in items 1 to 45, wherein breastfeeding is adapted such that the RID of the terminal metabolite 5-MIAA is 4% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
47. 5-Methoxy-N,N-dimethyltryptamine (5-MeO-DMT) or a pharmaceutically acceptable salt thereof for use as in items 1 to 45, wherein breastfeeding is adapted such that the RID of the terminal metabolite 5-MIAA is 2% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
48. 5-Methoxy-N,N-dimethyltryptamine (5-MeO-DMT) or a pharmaceutically acceptable salt thereof for use as in items 1 to 45, wherein breastfeeding is adapted such that the RID of the terminal metabolite 5-MIAA is 1% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.
49. 5-MeO-DMT or a pharmaceutically acceptable salt thereof for use as in items 1 to 48, wherein the patient suffers from PPD.
50. 5-MeO-DMT or a pharmaceutically acceptable salt thereof for use as in item 49, wherein a remission of depressive symptoms, as assessed by a MADRS score equal to or less than 10 occurs not later than about 2 hours after the last administration of 5-MeO-DMT or a pharmaceutically acceptable salt thereof.
51. 5-MeO-DMT or a pharmaceutically acceptable salt thereof for use as in item 50, wherein a remission of depressive symptoms, as assessed by a HAM-D score equal to or less than 7, occurs not later than about 2 hours after the last administration of 5-MeO-DMT or a pharmaceutically acceptable salt thereof.
52. 5-MeO-DMT or a pharmaceutically acceptable salt thereof for use as in items 1 to 51, wherein maternal functioning is improved which is reflected in improvements in the functional domains according to the Barkin Index of Maternal Functioning (BIMF) of mother-child interaction and psychological well-being.
53. 5-MeO-DMT or a pharmaceutically acceptable salt thereof for use as in item 52, wherein the improvement of the cumulative score of the BIMF scale items reflecting psychological well-being is at least 25% and the improvement of the cumulative score of the BIMF scale items reflecting mother-child interaction is at least 5%.
54. 5-MeO-DMT or a pharmaceutically acceptable salt thereof for use as in item 52 or 53, wherein improvements in the MADRS items lassitude, pessimistic thoughts, inability to feel, inner tension and/or reduced sleep lead to an increase in the BIMF scale scores reflecting psychological well-being and improvements in the MADRS items inability to feel and inner tension lead to an increase in the BIMF scale scores reflecting mother-child interaction.
55. 5-MeO-DMT or a pharmaceutically acceptable salt thereof for use as in any of items 1 to 54, wherein the patient suffers from a mental or nervous system disorders involving one or more symptoms selected from sleep disturbance and anxiety, wherein each of these symptoms compromises maternal functioning.
56. 5-MeO-DMT or a pharmaceutically acceptable salt thereof for use as in item 55, wherein the patient suffers from a mental or nervous system disorders involving anxiety symptoms and wherein a treatment reduces or eliminates symptoms of anxiety, in particular of inner tension.
57. 5-MeO-DMT or a pharmaceutically acceptable salt thereof for use as in item 56, wherein a treatment leads to a clinical response which is reflected by an at least 50% decrease of the HAM-A score, compared to the respective score prior to treatment, about 2 hours after the last administration of 5-MeO-DMT or a pharmaceutically acceptable salt thereof after the last administration of 5-MeO-DMT or a pharmaceutically acceptable salt thereof.
58. 5-MeO-DMT or a pharmaceutically acceptable salt thereof for use as in item 56 or 57, wherein a treatment leads to a clinical response which is reflected by an at least 50% decrease of the HAM-A score, compared to the respective score prior to treatment, on day 1, for instance after about 24 hours after the last administration of 5-MeO-DMT or a pharmaceutically acceptable salt thereof.
59. 5-MeO-DMT or a pharmaceutically acceptable salt thereof for use as in items 56 to 58, wherein a treatment leads to a clinical response which is reflected by an at least 50% decrease of the HAM-A score, compared to the respective score prior to treatment, on day 7 after the last administration of 5-MeO-DMT or a pharmaceutically acceptable salt thereof.
60. 5-MeO-DMT or a pharmaceutically acceptable salt thereof for use as in items 56 to 59, wherein a treatment leads to a clinical response which is reflected by an at least 50% decrease of the HAM-A score, compared to the respective score prior to treatment, on day 14 after the last administration of 5-MeO-DMT or a pharmaceutically acceptable salt thereof.
61. 5-MeO-DMT or a pharmaceutically acceptable salt thereof for use as in items 56 to 60, wherein a treatment leads to a clinical response which is reflected by an at least 50% decrease of the HAM-A score, compared to the respective score prior to treatment, on day 28 after the last administration of 5-MeO-DMT or a pharmaceutically acceptable salt thereof.
62. 5-MeO-DMT or a pharmaceutically acceptable salt thereof for use as in items 55 to 61, wherein the patient suffers from a mental or nervous system disorders involving sleep disturbance symptoms and wherein a treatment reduces or eliminates symptoms of sleep disturbance.
63. 5-MeO-DMT or a pharmaceutically acceptable salt thereof for use as in item 62, wherein the patient suffers from sleep disturbance as reflected by a Pittsburgh Sleep Quality Index (PSQI) global score of >5.
64. 5-MeO-DMT or a pharmaceutically acceptable salt thereof for use as in item 62 or 63, wherein the treatment reduces or eliminates sleep disturbance and the reduction or elimination of sleep disturbance is reflected by an improvement in the Pittsburgh Sleep Quality Index (PSQI) global score on day 1, for instance, about 24 hours after the last administration of 5-MeO-DMT or a pharmaceutically acceptable salt thereof, wherein the recall period spans from the time point when acute psychedelic experiences have subsided after the last administration to the assessment time point.
65. 5-MeO-DMT or a pharmaceutically acceptable salt thereof for use as in items 62 to 64, wherein the treatment success is indicated by a decrease in the PSQI global score.
66. 5-MeO-DMT or a pharmaceutically acceptable salt thereof for use as in items 62 to 65, wherein the treatment success is indicated by a decrease in at least four of the seven component scores of the PSQI.
67. 5-MeO-DMT or a pharmaceutically acceptable salt thereof for use as in items 62 to 66, wherein the treatment success is indicated by a decrease in the PSQI global score to 5 or below.
68. 5-MeO-DMT or a pharmaceutically acceptable salt thereof for use as in items 62 to 67, wherein the sleep disturbance is insomnia.
69. 5-MeO-DMT or a pharmaceutically acceptable salt thereof for use as in items 1 to 68, wherein the 5-MeO-DMT or salt thereof is administered at a dose or in a dosage regimen that causes the patient to experience a peak psychedelic experience.
70. 5-MeO-DMT or a pharmaceutically acceptable salt thereof for use as in items 1 to 69, wherein a dosage of about 2 mg; or of about 5 mg; or of about 8 mg is administered, or wherein equimolar amounts of the pharmaceutically acceptable salt are administered instead of 5-MeO-DMT.
71. 5-MeO-DMT or a pharmaceutically acceptable salt thereof for use as in items 1 to 69, wherein a dosage of about 1 mg; or of about 2 mg; or of about 3 mg is administered, or wherein equimolar amounts of the pharmaceutically acceptable salt are administered instead of 5-MeO-DMT.
72. 5-MeO-DMT or a pharmaceutically acceptable salt thereof for use as in items 1 to 69, wherein a dosage of about 2 mg; or of about 4 mg; or of about 6 mg is administered, or wherein equimolar amounts of the pharmaceutically acceptable salt are administered instead of 5-MeO-DMT.
73. 5-MeO-DMT or a pharmaceutically acceptable salt thereof for use as in items 1 to 69, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.
74. 5-MeO-DMT or a pharmaceutically acceptable salt thereof for use as in items 1 to 69 or 73, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.
75. 5-MeO-DMT or a pharmaceutically acceptable salt thereof for use as in item 74, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.
76. 5-MeO-DMT or a pharmaceutically acceptable salt thereof for use as in items 1 to 69 or 73, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.
77. 5-MeO-DMT or a pharmaceutically acceptable salt thereof for use as in item 76, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.
78. 5-MeO-DMT or a pharmaceutically acceptable salt thereof for use as in items 1 to 69 or 73, wherein the 5-MeO-DMT is administered in a dosage from about 2 mg to about 3 mg for a first administration, and then increased, unless the patient has already experienced a peak psychedelic experience, to a dosage from about 4 mg to about 5 mg for a second administration, and then increased, unless the patient has already experienced a peak psychedelic experience, to a dosage from about 6 mg to about 7.5 mg for a third administration, or wherein equimolar amounts of the pharmaceutically acceptable salt are administered instead of 5-MeO-DMT.
79. 5-MeO-DMT or a pharmaceutically acceptable salt thereof for use as in item 78, wherein the first dosage of 5-MeO-DMT is about 2 mg, the second dosage of 5-MeO-DMT is about 4 mg, and the third dosage of 5-MeO-DMT is about 6 mg; or wherein equimolar amounts of the pharmaceutically acceptable salt are administered instead of 5-MeO-DMT.
80. 5-MeO-DMT or a pharmaceutically acceptable salt thereof for use as in items 73 to 79, wherein the interval between two administrations is not less than 1 hour and not more than 24 hours, such as about 1 to 4 hours, preferably 1 to 2 hours.
81. 5-Methoxy-N,N-dimethyltryptamine (5-MeO-DMT) or a pharmaceutically acceptable salt thereof for use as in items 1 to 69, wherein a dosage of about 1 mg to about 5 mg 5-MeO-DMT or of an equimolar amount of the pharmaceutically acceptable salt is administered as a single dose or as the highest dose in an uptitration scheme involving intervals between administrations of at least about 1 hour, wherein the 5-MeO-DMT or the pharmaceutically acceptable salt thereof is administered via the intravenous, intramuscular or subcutaneous route, wherein the patient is advised to temporarily cease breastfeeding.
82. 5-MeO-DMT or a pharmaceutically acceptable salt thereof for use as in item 81, wherein a dosage of about 1 mg; or of about 5 mg is administered, or wherein equimolar amounts of the pharmaceutically acceptable salt are administered instead of 5-MeO-DMT.
83. 5-MeO-DMT or a pharmaceutically acceptable salt thereof for use as in item 82, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.
84. 5-MeO-DMT or a pharmaceutically acceptable salt thereof for use as in item 83, wherein the interval between two administrations is not less than 1 hour and not more than 24 hours, such as about 1 to 4 hours, preferably 1 to 2 hours.
85. 5-MeO-DMT or a pharmaceutically acceptable salt thereof for use as in items 69 to 84, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.
86. 5-MeO-DMT or a pharmaceutically acceptable salt thereof for use as in item 85, wherein the occurrence of a peak psychedelic experience is identified through achievement of a Peak Experience Scale (PES) Total Score of at least 75.
87. 5-MeO-DMT or a pharmaceutically acceptable salt thereof for use as in items 1 to 86, wherein the 5-MeO-DMT or a pharmaceutically acceptable salt thereof is administered via intravenous injection.

### EXAMPLES

The following Examples are listed to aid understanding of the invention and are not intended and should not be construed to limit in any way the invention set forth in the claims which follow thereafter.

### Example 1-5-MeO-DMT Aerosol Generation and Administration

Step 1: A stock solution of 5-MeO-DMT free base in 100% ethanol is prepared in a volumetric flask, so that the target dosage of 5-MeO-DMT free base to be administered via inhalation to the volunteer or patient is contained in a solution volume of 200 μl. Typical target dosages are from 1 mg to 25 mg 5-MeO-DMT. E.g. for a target dosage of 18 mg 5-MeO-DMT, 90 mg of 5-MeO-DMT will be dissolved in 100% ethanol for a final solution volume of 1 ml. Aliquots of the stock solution can then be stored in vials until further use.

Step 2: 200 μl of the solution is transferred to a dosing capsule containing the drip pad (Storz & Bickel, Germany), and then the dosing capsule is closed with its lid.

Step 3: The dosing capsule filled with the 5-MeO-DMT ethanol solution is transferred to the filling chamber of a first Volcano Medic Vaporizer, which has been pre-heated with the temperature set at 55° C. Then the airflow of the vaporizer is switched on for 60 seconds at the pre-set rate of about 12 1/min. The heated air will flow through the dosing capsule, allowing the ethanol to evaporate, with the target dosage of 5-MeO-DMT being left in the capsule, as a thin layer covering the stainless-steel wire mesh. Accurate preparation of the dosing capsule can be confirmed by demonstrating that the final weight increase of the capsule compared to the weight of the empty capsule is about equal to the target dosage of 5-MeO-DMT.

Step 4: The prepared dosing capsule is removed from the filling chamber. It is then transferred to the filling chamber of a second Volcano Medic Vaporizer, which has been pre-heated with the temperature set at 210° C. and the airflow on for at least 5 minutes and then turned off immediately prior to transfer. An inhalation balloon with a valve (Storz & Bickel, Germany) is mounted on the socket of the filling chamber, the filling chamber is closed tightly and immediately afterwards the airflow is switched on for exactly 15 seconds at the pre-set flow rate of about 12 1/min, and then turned off. This will allow the full dose of 5-MeO-DMT to aerosolize and be distributed in approximately 3 liters of air in the inhalation balloon. Accurate aerosolization of the 5-MeO-DMT can be confirmed by demonstrating that the capsule weight has returned to about its initial weight.

Step 5: The balloon is then disconnected from the filling chamber, which automatically closes the valve. After attachment of the mouthpiece to the balloon, the aerosol is ready for immediate administration to the volunteer or patient.

Step 6: To prepare for the administration, the patient is asked to initially perform 1-2 deep inhalations with full exhalations, ending this sequence with a deep exhalation. Then, with the mouthpiece firmly held against the lips, the full and complete volume of the inhalation balloon is inhaled in one inhalation, holding the breath for 10 (±2.5) seconds, followed by a normal exhalation. After completing the inhalation procedure, the patient will be instructed to lie down.

Further details regarding the administration of 5-MeO-DMT by inhalation are disclosed in Example 1 of WO 2020/169850 A1, the contents of which is incorporated herein by reference.

### Example 2—Preparation of 5-MeO-DMT in High Purity

5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to 12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC as described in WO 2020/169850 A1. The purity was found to be 99.74% area.

The results from the analysis further indicate that the level of individual impurities was below 0.10% area. Solvent analysis of sample indicated an MTBE level of 17 ppm.

### Example 3—Preparation of 5-MeO-DMT Hydrobromide Salt

5-MeO-DMT HBr was prepared on a 100 mg scale.

5-MeO-DMT free base was combined with isopropyl acetate (10 vols), and the resulting solution of 5-MeO-DMT was heated to 50° C. HBr was charged (1M in ethanol, 1 eq) in one single aliquot. The mixture was held at temperature and equilibrated for 3 hours.

After 1 hour, a suspension had formed. The suspension was finally cooled to room temperature and equilibrated for 18 hours. Solids were isolated by filtration and dried in vacuo at 40° C. for 18 hours.

An off-white crystalline material was obtained.

The salt has a melting point of 174° C. and is characterized by an X-ray diffraction pattern comprising peaks at 14.5° 2θ±0.2° 2θ; 16.7° 2θ±0.2° 2θ; 17.0° 2θ±0.2° 2θ; 20.6° 2θ±0.2° 2θ; 20.7° 2θ±0.2° 2θ; 21.4° 2θ±0.2° 2θ; 24.2° 2θ±0.2° 2θ; 24.8° 2θ±0.2° 2θ; 25.3° 2θ±0.2° 2θ; 27.4° 2θ±0.2° 2θ; measured using Cu Kα radiation.

### Example 4—Determination of Inhibition Constants for Central 5-HT1A and 5-HT2A Receptors in Post-Mortem Human Brain Membrane Preparations

In this study, the affinity of three psychedelic test compounds (psilocin, DMT and 5-MeO-DMT) for 5-HT1A and 5-HT2A receptors in post-mortem human brain tissue from the hippocampus and frontal cortex, respectively, was determined using the technique of radioligand binding.

Human brain samples were obtained from the Edinburgh Sudden Death Brain Bank. All donors were sudden deaths with no prior history of coma, psychiatric or neurological disorders and under the age of 65 with a post-mortem interval of less than or equal to 72 hours.

Binding to 5-HT1A Receptors in Post-Mortem Human Hippocampus

Hippocampus was homogenised in ice-cold 0.25 M sucrose (1:30 w/v) using a motor driven Teflon pestle (12 strokes at 120 rpm). Myelin and cell debris were removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold membrane preparation buffer, (1:100 w/v) using a tight-fitting glass/Teflon homogeniser (12 strokes, 800 rpm) and centrifuged at 20,500 g for 10 minutes. The pellet was resuspended in ice-cold membrane preparation buffer and incubated at 37° C. for 10 minutes before being centrifuged at 20,500 g for 10 minutes. The pellet was resuspended and centrifuged a final time to wash the tissue (20,500×g for 10 mins). The resulting pellet was then resuspended in ice-cold assay buffer at a tissue concentration equivalent to 3.125 mg wet weight of tissue/ml. All centrifugations were carried out at 4° C. The membrane preparation buffer consisted of 50 mM Tris-HCl, pH 7.7, 4 mM CaCl₂ and 0.1% ascorbic acid. The assay buffer consisted of 50 mM Tris, pH 7.7, 4 mM CaCl₂, 0.1% ascorbic acid and 10 μM Pargyline.

For saturation binding analysis, hippocampal membranes (400 μl; equivalent 1.25 mg wet weight tissue/tube) was incubated with 50 μl of 0.075-9.6 nM [³H]8-OH-DPAT and either 50 μl of assay buffer (total binding) or 50 μl of 1 μM WAY 100635 (non-specific binding) at 25° C. for 30 minutes. The wash buffer consisted of 50 mM Tris, pH 7.7.

In a displacement assay, hippocampal membranes (400 μl; equivalent 1.25 mg wet weight tissue/tube) were incubated with 50 μl of 0.6 nM [³H]8-OH-DPAT and either 50 μl of assay buffer (total binding) or 50 μl of 1 μM WAY 100635 (non-specific binding) or 50 μl of one of the test compounds in one of ten concentrations between 1 and 10000 nM at 25° C. for 30 minutes.

Membrane bound radioactivity was recovered by filtration under vacuum through Skatron 11731 filters, pre-soaked in 0.5% polyethylenimine (PEI) using a Skatron cell harvester.

Filters were rapidly washed with ice-cold wash buffer (wash setting 0,9,9) and radioactivity determined by liquid scintillation counting (1 ml Packard MV Gold scintillator).

The concentration of compound required to inhibit 50% of specific binding (IC₅₀) and the Hill Slope were calculated by using non-linear regression. The Kᵢ was calculated using the one-site binding model allowing for ligand depletion.

Binding to 5-HT2A Receptors in Post-Mortem Human Frontal Cortex

Frontal cortex was homogenised in ice-cold 0.25 M sucrose (1:30 w/v) using a motor driven Teflon pestle (12 strokes at 120 rpm). Myelin and cell debris was removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold 50 mM Tris-HCl assay buffer, pH 7.4 (1:100 w/v), homogenised using a tight-fitting glass/Teflon homogeniser (12 strokes, 800 rpm) and centrifuged at 20,500 g for 10 minutes. The pellet was centrifuged a further two times to wash the tissue (20,500×g for 10 mins). The resulting pellet was then resuspended in ice-cold 50 mM Tris-HCl assay buffer, pH 7.4 at a tissue concentration equivalent to 10 mg wet weight of tissue/ml. All centrifugations were carried out at 4° C.

For saturation binding analysis, frontal cortical membranes (400 μl; equivalent to 4 mg wet weight of tissue/tube) were incubated with 50 μl of 0.00625-0.8 nM [³H]MDL100,907 and either 50 μl of assay buffer or 50 μl of 10 μM ketanserin (non-specific binding) at 25° C. for 60 minutes. The assay and wash buffer consisted of 50 mM Tris-HCl buffer pH 7.4.

In a displacement assay, frontal cortical membranes (400 μl; equivalent 4 mg wet weight tissue/tube) was incubated with 50 μl of 0.1 nM [3H]MDL-100,907 and either 50 μl of assay buffer (total binding) or 50 μl of 10 μM ketanserin (non-specific binding) or 50 μl of one of the test compounds in one of ten concentrations between 1 and 10000 nM at 25° C. for 60 minutes.

Membrane bound radioactivity was recovered and determined as above. Data analysis was also as above.

Results

The dissociation constant (K_{d} value) of [³H]8-OH-DPAT for 5-HT1A receptors in hippocampal membranes from post-mortem human brain tissue was determined for each of the three donors. The dissociation constants (K_{d} values) obtained were 0.51, 0.28 and 0.52 nM, respectively.

Mean inhibition constants (Kᵢ values) for psilocin, DMT and 5-MeO-DMT were 48, 38 and 1.80 nM (mean n=3), respectively. All compounds gave Hill slopes approximating to unity, suggesting a one-site binding model.

The dissociation constant (K_{d} values) of [³H]MDL-100,907 for 5-HT2A receptors in frontal cortical membranes from post-mortem human brain tissue was determined for each of the three donors. The dissociation constants (K_{d} values) obtained were 0.11, 0.08 and 0.08 nM, respectively.

Mean inhibition constants (Kᵢ values) for psilocin, DMT and 5-MeO-DMT were 37, 117 and 122 nM (mean n=3), respectively. All compounds gave Hill slopes approximating to unity, suggesting a one-site binding model.

The selectivity ratio of psilocin, DMT and 5-MeO-DMT for 5-HT2A over 5-HT1A receptors was 0.78, 3.1 and 68, respectively.

### Example 5—Clinical Trial in Patients Suffering from TRD

A Phase 1/2 clinical trial of 5-MeO-DMT, administered via inhalation as described herein, in patients with treatment-resistant major depressive disorder (TRD) has been completed. This trial was designed in two parts. Part A was an open-label, single-arm, single-dose Phase 1 trial with two dose levels (12 mg (n=4) and 18 mg (n=4)). Part B was an open-label, single-arm Phase 2 trial applying an individualized dosing regimen with intra-patient dose escalation with 5-MeO-DMT. Patients (n=8) received at least one and up to three doses of 5-MeO-DMT (6 mg, 12 mg and 18 mg) in a single day, with higher doses only being administered if a peak experience was not achieved at the previously administered dose. The primary endpoint of Part A was to assess the safety and tolerability of single dosing of 5-MeO-DMT in patients with TRD. The primary endpoint of Part B was to assess the effects on the severity of depression, as assessed by the proportion of patients in remission on day seven after dosing, defined as a MADRS total score of less than or equal to 10.

In Part A, 3 of 4 patients in both groups (12 mg and 18 mg) experienced at least one ADR, all of which were mild and resolved spontaneously. No SAEs were reported.

Two of four patients (50%) in the 12 mg group and one of four patients (25%) in the 18 mg group had a MADRS remission on day seven after dosing, and one further patient (25%) in the 18 mg group had a MADRS clinical response on day seven after dosing. The mean MADRS change from baseline at day seven was −21.0 (−65%) in the 12 mg group and −12.8 (−41%) in the 18 mg group.

In Part B, 7 of 8 patients (87.5%) experienced at least one ADR. All ADRs resolved spontaneously. No SAEs were reported.

The primary endpoint was met with seven of eight patients (87.5%) achieving a MADRS remission on day seven (p<0.0001). The mean MADRS change from baseline at day seven was 24.4 (76%).

No clinically significant changes were observed in either Part A or Part B in any of the safety laboratory analyses, vital signs, psychiatric safety assessments or measures of cognitive function.

Results are summarized in the tables below.

TABLE 2-A
Scores recorded against relevant MADRS and BPRS items for patients assigned to
intra-day individualised dosing regimen (IDR). The item scores represent the sum
of the individual patient scores for all patients (n = 8) in the IDR group.
Assessment 2 hours (MADRS) or 3 hours (BPRS) after administration of the last dose.

| | | Baseline | 2/3 | Improvement | Percentage |
|---|---|---|---|---|---|
| Scale item | Scale | IDR | hours | 2/3 hours | improvement |
| Concentration Difficulties | MADRS | 30 | 11 | 19 | 63.33% |
| Lassitude | MADRS | 27 | 10 | 17 | 62.96% |
| Inability to Feel | MADRS | 36 | 12 | 24 | 66.67% |
| Inner Tension | MADRS | 26 | 11 | 15 | 57.69% |
| Pessimistic Thoughts | MADRS | 28 | 7 | 21 | 75.00% |
| Suicidal Thoughts | MADRS | 11 | 3 | 8 | 72.72% |
| Guilt Feelings | BPRS | 34 | 14 | 20 | 58.82% |
| Tension | BPRS | 16 | 11 | 5 | 31.25% |
| Anxiety | BPRS | 37 | 19 | 18 | 48.65% |
| Emotional Withdrawal | BPRS | 13 | 8 | 5 | 38.46% |
| Blunted Affect | BPRS | 15 | 11 | 4 | 26.67% |

TABLE 2-B
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 1.

| | | Baseline | | Improvement | Percentage |
|---|---|---|---|---|---|
| Scale item | Scale | IDR | Day 1 | at Day 1 | improvement |
| Reduced Sleep | MADRS | 25 | 12 | 13 | 52.00% |
| Concentration Difficulties | MADRS | 30 | 1 | 29 | 96.67% |
| Lassitude | MADRS | 27 | 5 | 22 | 81.48% |
| Inability to Feel | MADRS | 36 | 2 | 34 | 94.44% |
| Inner Tension | MADRS | 26 | 6 | 20 | 76.92% |
| Pessimistic Thoughts | MADRS | 28 | 4 | 24 | 85.71% |
| Suicidal Thoughts | MADRS | 11 | 1 | 10 | 90.91% |
| Guilt Feelings | BPRS | 34 | 11 | 23 | 67.65% |
| Tension | BPRS | 16 | 11 | 5 | 31.25% |
| Anxiety | BPRS | 37 | 16 | 21 | 56.76% |
| Emotional Withdrawal | BPRS | 13 | 8 | 5 | 38.46% |
| Blunted Affect | BPRS | 15 | 8 | 7 | 46.67% |

TABLE 2-C
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 7.

| | | Baseline | | Improvement | Percentage |
|---|---|---|---|---|---|
| Scale item | Scale | IDR | Day 7 | at Day 7 | improvement |
| Reduced Sleep | MADRS | 25 | 9 | 16 | 64.00% |
| Concentration Difficulties | MADRS | 30 | 9 | 21 | 70.00% |
| Lassitude | MADRS | 27 | 3 | 24 | 88.89% |
| Inability to Feel | MADRS | 36 | 6 | 30 | 83.33% |
| Inner Tension | MADRS | 26 | 12 | 14 | 53.85% |
| Pessimistic Thoughts | MADRS | 28 | 3 | 25 | 89.29% |
| Suicidal Thoughts | MADRS | 11 | 3 | 8 | 72.73% |
| Guilt feelings | BPRS | 34 | 10 | 24 | 70.59% |
| Tension | BPRS | 16 | 10 | 6 | 37.50% |
| Anxiety | BPRS | 37 | 17 | 20 | 54.05% |
| Emotional withdrawal | BPRS | 13 | 8 | 5 | 38.46% |
| Blunted affect | BPRS | 15 | 8 | 7 | 46.67% |

TABLE 3-A
Scores recorded against relevant MADRS and BPRS items for patients assigned
to 12 mg dosing regimen. The item scores represent the sum of the individual
patient scores for all patients (n = 4) in the 12 mg group. Assessment
2 hours (MADRS) or 3 hours (BPRS) after administration of the dose.

| | | | | Improvement | |
|---|---|---|---|---|---|
| | | Baseline | 2/3 | after | Percentage |
| Scale item | Scale | 12 mg | hours | 2/3 hours | improvement |
| Concentration Difficulties | MADRS | 16 | 7 | 9 | 56.25% |
| Lassitude | MADRS | 16 | 10 | 6 | 37.50% |
| Inability to Feel | MADRS | 16 | 9 | 7 | 43.75% |
| Inner Tension | MADRS | 13 | 2 | 11 | 84.62% |
| Pessimistic Thoughts | MADRS | 16 | 8 | 8 | 50.00% |
| Suicidal Thoughts | MADRS | 8 | 3 | 5 | 62.50% |
| Guilt Feelings | BPRS | 18 | 9 | 9 | 50.00% |
| Tension | BPRS | 14 | 9 | 5 | 35.71% |
| Anxiety | BPRS | 25 | 11 | 14 | 56.00% |
| Emotional Withdrawal | BPRS | 13 | 11 | 2 | 15.38% |
| Blunted Affect | BPRS | 11 | 8 | 3 | 27.27% |

TABLE 3-B
Scores recorded against relevant MADRS and BPRS items for patients assigned to
12 mg dosing regimen. The item scores represent the sum of the individual patient
scores for all patients (n = 4) in the 12 mg group. Assessment on day 1.

| | | Baseline | | Improvement | Percentage |
|---|---|---|---|---|---|
| Scale item | Scale | 12 mg | Day 1 | at Day 1 | improvement |
| Reduced Sleep | MADRS | 12 | 10 | 2 | 16.67% |
| Concentration Difficulties | MADRS | 16 | 2 | 14 | 87.50% |
| Lassitude | MADRS | 16 | 0 | 16 | 100.00% |
| Inability to Feel | MADRS | 16 | 1 | 15 | 93.75% |
| Inner Tension | MADRS | 13 | 3 | 10 | 76.92% |
| Pessimistic Thoughts | MADRS | 16 | 7 | 9 | 56.25% |
| Suicidal Thoughts | MADRS | 8 | 5 | 3 | 37.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 8 | 5 | 38.46% |
| Blunted Affect | BPRS | 11 | 6 | 5 | 45.45% |

TABLE 3-C
Scores recorded against relevant MADRS and BPRS items for patients assigned to
12 mg dosing regimen. The item scores represent the sum of the individual patient
scores for all patients (n = 4) in the 12 mg group. Assessment on day 7.

| | | Baseline | | Improvement | Percentage |
|---|---|---|---|---|---|
| Scale item | Scale | 12 mg | Day 7 | at Day 7 | improvement |
| Reduced Sleep | MADRS | 12 | 6 | 6 | 50.00% |
| Concentration Difficulties | MADRS | 16 | 3 | 13 | 81.25% |
| Lassitude | MADRS | 16 | 3 | 13 | 81.25% |
| Inability to Feel | MADRS | 16 | 1 | 15 | 93.75% |
| Inner Tension | MADRS | 13 | 5 | 8 | 61.54% |
| Pessimistic Thoughts | MADRS | 16 | 8 | 8 | 50.00% |
| Suicidal Thoughts | MADRS | 8 | 7 | 1 | 12.50% |
| Guilt feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional withdrawal | BPRS | 13 | 6 | 7 | 53.85% |
| Blunted affect | BPRS | 11 | 5 | 6 | 54.55% |

### Example 6—Assessment of the Pharmacokinetics of 5-MeO-DMT and Bufotenine

In order to investigate the pharmacokinetic properties of 5-MeO-DMT, three groups of 8 subjects each were formed. Subjects were administered a single dose of 6 mg; 12 mg or 18 mg 5-MeO-DMT via inhalation. Blood samples were obtained at 1; 2; 4; 7; 10; 15; 20; 30; 45 min and 1; 1.5; 2; 3; 4 hours after administration.

5-MeO-DMT concentrations were determined using LC-MS/MS. PK parameters were generated by algebraic analysis of the concentration versus time plots for each individual. The analysis was carried out using the software Phoenix WinNonlin 6.3.

Median Cmax values obtained for the three groups were 11.85 ng/ml (6 mg group), 22.90 ng/ml (12 mg group) and 38.45 ng/ml (18 mg group).

Table 4 below shows median percentage plasma concentrations relative to Cmax as determined for the time points indicated.

Median percentage plasma concentration relative to Cmax

| time | | | | | | | | | | | | | | |
|---|---|---|---|---|---|---|---|---|---|---|---|---|---|---|
| (min) | 1 | 2 | 4 | 7 | 10 | 15 | 20 | 30 | 45 | 60 | 90 | 120 | 180 | 240 |
| 6 mg | 96 | 98 | 83 | 56 | 40 | 22 | 16 | 10 | 8 | 4 | 2 | 1 | 0 | N.A. |
| 12 mg | 100 | 79 | 59 | 30 | 22 | 12 | 8 | 5 | 3 | 2 | 0 | 0 | 0 | 0 |
| 18 mg | 100 | 79 | 59 | 30 | 22 | 12 | 8 | 5 | 3 | 2 | 0 | 0 | 0 | 0 |

Pharmacokinetic measurements were also carried out for dosing schemes relying on uptitration. Substantially similar results were obtained.

Blood concentrations were also determined for the 5-MeO-DMT metabolite bufotenine. Only in few samples, concentrations were above the lower level of quantification (LLOQ) (25 μg/ml). From 15 min onwards, the bufotenine concentration was always below the LLOQ.

Substantially similar observations were made when subjects receiving an uptitration scheme were included.

### Example 7—Toxicological Testing of 5-MeO-DMT

5-MeO-DMT did not induce mutation in four histidine-requiring bacterial strains (TA98, TA100, TA1535 and TA1537) of Salmonella typhimurium, and one tryptophan-requiring strain (WP2 uvrA pKM101) of Escherichia coli. These conditions included treatments at concentrations up to 5000 μg/plate (the maximum recommended concentration according to current regulatory guidelines), in the absence and in the presence of a rat liver metabolic activation system (S-9).

### Example 8-5-MeO-DMT Binding to Human Plasma Proteins

The in vitro binding of 5-MeO-DMT to plasma proteins was determined using high throughput dialysis. Equilibration time and non-specific binding were determined at a nominal 5-MeO-DMT concentration of 1 μM using human plasma. Following evaluation of the equilibration data, plasma protein binding was investigated at nominal concentrations of 0.1, 1 and 10 μM using a 4-hour dialysis time. The concentration of 5-MeO-DMT in the samples from the plasma and buffer compartments was determined by LC-MS/MS. The protein binding results are presented below:

TABLE 5
Percentage of free fractions of 5-MeO-DMT in human plasma

| 5-MeO-DMT (μM) | Free Fraction, % |
|---|---|
| 0.1 | 78.7 |
| 1 | 77.3 |
| 10 | 87.0 |

### Example 9—Human Metabolism of 5-MeO-DMT

5-MeO-DMT was incubated at a nominal concentration of 1 μM and 10 μM with human hepatocytes in suspension in Leibovitz L-15 medium (1×106 cells/mL).

A standard stock solution of 5-MeO-DMT was prepared in ethanol at 20 mM and was further diluted with Leibovitz L-15 medium to a concentration of 2 mM. For incubations with cryopreserved hepatocytes, the 2 mM stock solution was diluted with Leibovitz L-15 medium to a concentration of 20 μM or 2 μM. An aliquot (250 μL) of the 20 μM and 2 μM test substance formulations was added to each hepatocyte incubation sample (250 μL), as appropriate, so that the final test substance concentration in the incubations was 10 μM or 1 μM, respectively, and incubations contained less than 1% (v/v) solvent.

Incubations were performed at ca. 37° C. in a shaking water bath (total incubation volume 0.5 mL). For 1 μM, incubations were terminated after 0, 5, 10, 20, 30, 60 and 120 minutes, by the addition of ice-cold acetonitrile (0.5 mL). For 10 μM, incubations were terminated after 0, 10, 30, 60 and 120 minutes, by the addition of ice-cold acetonitrile containing internal standard (1 μg/mL Psilocin-d10).

The samples were vortex mixed and centrifuged at ca 13,000 rpm for 10 minutes at room temperature. Following centrifugation, the protein-free supernatants were removed for analysis.

Blank control incubations were carried out with Leibovitz L-15 medium in place of the test substance. No cells control samples were performed with Leibovitz L-15 medium in place of hepatocytes. Aliquots of the blank control samples were taken at 120 minutes, while no cells control samples were taken at 0, 30 and 120 for 1 μM incubations and at 0 and 120 minutes for 10 μM incubations.

All 1 μM incubations were performed in duplicate, while all 10 μM incubations were performed in singlet. All samples were stored at −80° C. (nominal) prior to analysis.

Suitable chromatographic conditions were developed to retain the parent compound and give a suitable chromatographic response. The 0, 30 and 120-minute incubation samples generated following incubation of 5-MeO-DMT at 10 μM were analysed using reverse phase LC-MS analysis to generate high and low energy mass spectra (MSE). Prior to sample analysis a 100 μL aliquot of each sample was evaporated to near dryness under a steady stream of nitrogen at room temperature, and subsequently reconstituted in 50 μL of mobile phase A (0.1% formic acid in water). Each sample (0, 30 and 120-minute, 10 μM) was analysed using accurate mass LC-MS to determine relative levels of parent compound at each time-point, and determine the profile of metabolites formed. Appropriate blank and control samples were also analysed. The 10 and 60-minute, 10 μM incubation samples were not analysed and were stored at −80° C. (nominal).

Data were interrogated for the presence of metabolites by comparison of retention times with the test substance reference standard and based on the accurate masses of potential metabolites using screening software (UNIFI version 1.9.4), and user defined search parameters. To confirm a suspected metabolite, the measured accurate mass of the peak detected in the sample used for structural elucidation had to be within 5 ppm of the theoretical mass in order to confirm the molecular formula.

Results obtained are summarized in the above table 1.

### Example 10—Metabolic Stability for 5-Methoxy Indole-3-Acetic Acid (5-MIAA) and 5-Methoxytryptophol in a Human Hepatocyte Co-Culture Model

The metabolic stability of 5-MIAA and 5-methoxytryptophol was investigated in a Hμrel co-culture assay with human hepatocytes (Hμrel HumanPool™, primary hepatic co-culture model from Visikol Inc.).

The incubations were performed using 1 and 10 μM initial concentrations and sampling at 0, 1, 2, 4, 8, 24, 48, and 72 hours (h) time points. The samples were analysed using UPLC/QE-orbitrap-MS.

The remaining LC/MS peak areas detected for test compounds after each incubation time point with Hμrel co-culture assay, relative to corresponding 0 min incubation samples, are shown in the tables below. Results (disappearance half-lives) for the assay control diazepam indicated that enzyme activities were within the normal level.

TABLE 6
The relative LC/MS peak areas for 5-MIAA after 0-72 h incubations. The initial
substrate concentrations were 1 and 10 μM (n = 2 for 1 μM and n = 1 for 10 μM).

| Incubation | 0 h, % | 1 h, % | 2 h, % | 4 h, % | 8 h, % | 24 h, % | 48 h, % | 72 h, % |
|---|---|---|---|---|---|---|---|---|
| 1 μM Hepatocytes | 100 | 98 | 100 | 99 | 95 | 99 | 88 | 82 |
| 1 μM Stromal cells | 100 | — | — | 103 | 107 | 108 | 111 | 135 |
| 10 μM Hepatocytes | 100 | 97 | 100 | 103 | 96 | 96 | 86 | 75 |
| 10 μM Stromal | 100 | — | — | 101 | 106 | 97 | 103 | 115 |
| cells | | | | | | | | |

TABLE 7
The relative LC/MS peak areas for 5-methoxytryptophol after 0-72 h incubations.
The initial substrate concentrations were 1 and 10 μM (n = 2 for 1 μM and n = 1
for 10 μM).

| Incubation | 0 h, % | 1 h, % | 2 h, % | 4 h, % | 8 h, % | 24 h, % | 48 h, % | 72 h, % |
|---|---|---|---|---|---|---|---|---|
| 1 μM Hepatocytes | 100 | 79 | 57 | 33 | 10 | 0 | 0 | 0 |
| 1 μM Stromal cells | 100 | — | — | 97 | 107 | 94 | 102 | 104 |
| 10 μM Hepatocytes | 100 | 87 | 81 | 68 | 42 | 1 | 0 | 0 |
| 10 μM Stromal cells | 100 | — | — | 99 | 105 | 86 | 98 | 104 |

A low metabolic turnover was observed for 5-MIAA, the remaining abundances after 72h period being 75-82% in the presence of hepatocytes, while no disappearance was observed with stromal cell controls.

For 5-methoxytryptophol, a high metabolic turnover was observed, with complete disappearance in 24 h in the presence of hepatocytes, and no disappearance with stromal cell controls.

With human hepatocytes and 1 μM test concentration, in vitro intrinsic clearance of 0.15 μl/min/million cells (half-life 15 400 min) was obtained for 5-MIAA, while the corresponding value for 5-methoxytryptophol was 16.2 μl/min/million cells (half-life 142 min).

The predicted hepatic extraction ratios were 2% for 5-MIAA and 67% 5-methoxytryptophol.

### Example 11—Plasma Binding of 5-MIAA

Binding to human plasma protein was determined. Reported are the unbound fraction (fu) for three replicates as well as the mean unbound fraction, the standard deviation and the mean % recovery (Table 8).

| Com- | Repli- | Repli- | Repli- | Mean fu | SD | Mean % |
|---|---|---|---|---|---|---|
| pound | cate 1 | cate 2 | cate 3 | | | Recovery |
| 5-MIAA | 0.499 | 0.462 | 0.544 | 0.502 | 0.0411 | 81.4 |
| Warfarin | 0.0418 | 0.0356 | 0.0358 | 0.0377 | 0.00352 | 78.1 |
| (control) | | | | | | |

### Example 12—Clinical Trial of 5-MeO-DMT Administered Via Inhalation to Patients with Postpartum Depression

The single-arm, open-label clinical trial will involve 15 adult female patients with clinically diagnosed postpartum depression (PPD).

The patients will receive a single-day individualized 5-MeO-DMT dosing regimen via inhalation after vaporization.

More in particular, the patients will receive up to three doses of 5-MeO-DMT on Day 0: 6 mg, 12 mg, and 18 mg.
1. All patients will receive an initial dose of 6 mg 5-MeO-DMT.
2. The second dose (12 mg) will only be administered if:
   a. A peak experience (total score of ≥75) has not been achieved following the 6 mg dose, and
   b. The 6 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (18 mg) will only be administered if:
   a. A peak experience (total score of ≥75) has not been achieved following the 12 mg dose, and
   b. The 12 mg dose was safe and well-tolerated according to the investigator, and
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing. Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate <1%), medically accepted contraceptive method for 30 days prior to dosing and for 90 days after 5-MeO-DMT dosing. Patients must have a negative pregnancy test at screening and on the pre-test day (Day −1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-MeO-DMT.
8. Has any current or past clinically significant condition (e.g., severe infection, pulmonary disease, uncontrolled hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day −1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 6 mg, 12 mg and 18 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 6 mg, 12 mg and 18 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day IDR of 6 mg, 12 mg and 18 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS:10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (≥50% reduction from baseline in MADRS total score) at 2 hours after the final study drug dosing on Day 0, and at Day 1 and Day 7;
   Change from baseline in Clinical Global Impression-Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day −1), 1 hour after last study drug dosing, at discharge, in the evening of Day 0, and on Day 1 and on Day 7;
Exposure of 5-MeO-DMT and bufotenine in blood obtained at the pre-test day (Day −1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the
   achievement of a PE (PE scale total score ≥75);
   Challenging Experience Questionnaire (CEQ);
   Mystical Experience Questionnaire (MEQ-30);
Duration of the PsE defined as the time from study drug dosing to the time when the PsE have subsided (investigator- and patient-scored), completed 30 to 60 minutes after each dosing;

One patient with postpartum depression diagnosed by a psychiatrist has, so far, been recruited into the clinical trial. Diagnosis was Major Depressive Disorder without psychotic features, confirmed by the Mini-International Neuropsychiatric Interview (MINI) (v7.0.2), with peri-partum onset that began no earlier than gestation and no later than the first 4 weeks postpartum. The patient was diagnosed with postpartum depression after giving birth to her third child. The patient completed all planned visit days. The inhalation procedure was adequately performed by the patient and was well tolerated with no inhalation-related adverse events.

Results

Except for a temporary, clinically non-relevant increase in heart rate and blood pressure shortly after administration of 5-MeO-DMT, no other noteworthy changes in vital parameters occurred. Assessments of ECG (at 3 hours after administration) and safety laboratory analyses (at 7 days), CADSS (at 3 hours, 1 day and 7 days) were unremarkable. The few reported adverse events (cramping left abdominal pain and headache, both on Day 0) were mild, short-lasting and resolved spontaneously by the end of the study.

With regard to the intensity of the psychedelic experience, the recorded PES score achieved upon exposure to a nominal dose of 6 mg was 17.3. This score indicated the need to proceed to the administration of a subsequent, higher dose of 12 mg, per the design of the individualised dosing regimen. The PES score achieved for this dose was 85.7 and, being ≥75, indicated the occurrence of a peak psychedelic experience and the completion of the IDR for this patient.

Significantly, the patient reported a major improvement in her depressive symptoms as assessed by MADRS at the earliest assessment timepoint of 2 hours after drug administration, with the effect being maintained over time (Table 9). The patient also fulfilled standard criteria for MADRS response (at least 50% improvement from baseline) and MADRS remission (MADRS total score equal or less than 10).

TABLE 9
MADRS/BPRS scores table

| | | 2 hr | | | | | |
|---|---|---|---|---|---|---|---|
| | | (MADRS); | | | Improvement | | |
| | | Dis- | | | 2 hr; | | |
| | Base- | charge | | | Dis- | Day | Day |
| Scale item | line | (BPRS) | Day 1 | Day 7 | charge | 1 | 7 |
| MADRS | | | | | | | |
| Reported sadness | 4 | 0 | 0 | 0 | 4 | 4 | 4 |
| Apparent sadness | 5 | 0 | 0 | 0 | 5 | 5 | 5 |
| Inner Tension | 5 | 0 | 0 | 1 | 5 | 5 | 4 |
| Reduced Sleep | 5 | 5 | 0 | 0 | 0 | 5 | 5 |
| Reduced appetite | 0 | 0 | 0 | 0 | 0 | 0 | 0 |
| Concentration | 0 | 0 | 0 | 0 | 0 | 0 | 0 |
| Difficulties | | | | | | | |
| Lassitude | 3 | 0 | 0 | 0 | 3 | 3 | 3 |
| Inability to Feel | 3 | 0 | 0 | 0 | 3 | 3 | 3 |
| Pessimistic | 4 | 0 | 0 | 0 | 4 | 4 | 4 |
| Thoughts | | | | | | | |
| Suicidal Thoughts | 0 | 0 | 0 | 0 | 0 | 0 | 0 |
| Total MADRS | 29 | 5 | 0 | 1 | 24 | 29 | 28 |
| BPRS | | | | | | | |
| Somatic Concern | 6 | 1 | 1 | 1 | 5 | 5 | 5 |
| Anxiety | 7 | 1 | 1 | 1 | 6 | 6 | 6 |
| Emotional | 5 | 1 | 1 | 1 | 4 | 4 | 4 |
| withdrawal | | | | | | | |
| Conceptual | 1 | 1 | 1 | 1 | 0 | 0 | 0 |
| disorganization | | | | | | | |
| Guilt feelings | 6 | 1 | 1 | 1 | 5 | 5 | 5 |
| Tension | 6 | 1 | 1 | 1 | 5 | 5 | 5 |
| Mannerisms and | 1 | 1 | 1 | 1 | 0 | 0 | 0 |
| Posturing | | | | | | | |
| Grandiosity | 1 | 1 | 1 | 1 | 0 | 0 | 0 |
| Depressive Mood | 6 | 1 | 1 | 1 | 5 | 5 | 5 |
| Hostility | 1 | 1 | 1 | 1 | 0 | 0 | 0 |
| Suspiciousness | 1 | 1 | 1 | 1 | 0 | 0 | 0 |
| Hallucinatory | 1 | 1 | 1 | 1 | 0 | 0 | 0 |
| behaviour | | | | | | | |
| Motor retardation | 1 | 1 | 1 | 1 | 0 | 0 | 0 |
| Uncooperativeness | 1 | 1 | 1 | 1 | 0 | 0 | 0 |
| Unusual Thought | 1 | 1 | 1 | 1 | 0 | 0 | 0 |
| Content | | | | | | | |
| Blunted affect | 1 | 1 | 1 | 1 | 0 | 0 | 0 |
| Excitement | 2 | 1 | 1 | 1 | 1 | 1 | 1 |
| Disorientation | 1 | 1 | 1 | 1 | 0 | 0 | 0 |

Significant improvements were noted for several MADRS items in particular. The items are outlined in Table 9. While the patient's baseline scores for some items reflected absence of the symptom (reduced appetite, concentration difficulties, suicidal thoughts), items with scores reflecting severe symptoms (e.g., reduced sleep, inner tension) saw remarkable improvement.

Similarly, improvements were seen in several BPRS items, including Somatic Concerns, Anxiety, Emotional withdrawal, Guilt feelings and Tension.

Additionally, improvements in maternal functioning were evidenced by improvements in the BIMF score recorded at Day 7, as outlined in Table 10, with the total score improving by 14% from 92 to 105 (out of a possible total of 120).

Several functional domains of maternal function were also assessed, as defined by Barkin et al. The improvements in each functional domain are outlined in more detail in Table 11.

Here, noteworthy improvements in self-care, psychological well-being and management were achieved, with percentage improvements ranging from 18% (management) to (44%),% (self-care). These improvements reinforce the relationship between improvement in depressive items, as assessed by the MADRS, and improvements in maternal functioning.

It is noted that the patient scored comparatively high already before treatment. In some functional domains, the score was at the maximum value, or close to it (see Table 11), so that the scope for improvement by therapy was limited.

TABLE 10
BIMF scores table

| No. | BIMF item | Day 0 | Day 7 | Δ (Day 7) |
|---|---|---|---|---|
| 1 | I am a good mother | 6 | 6 | 0 |
| 2 | I feel rested | 0 | 2 | 2 |
| 3 | I am comfortable with the way I've chosen to feed my | 6 | 6 | 0 |
| | baby (either bottle or breast, or both) | | | |
| 4 | My baby and I understand each other | 5 | 5 | 0 |
| 5 | I am able to relax and enjoy time with my baby | 5 | 6 | 1 |
| 6 | There are people in my life that I can trust to care for | 6 | 6 | 0 |
| | my baby when I need a break | | | |
| 7 | I am comfortable allowing a trusted friend or relative | 6 | 6 | 0 |
| | to care for my baby (can include baby's father or partner) | | | |
| 8 | I am getting enough adult interaction | 6 | 6 | 0 |
| 9 | I am getting enough encouragement from other people | 5 | 6 | 1 |
| 10 | I trust my own feelings (instincts) when it comes to | 5 | 6 | 1 |
| | taking care of my baby | | | |
| 11 | I take a little time each week to do something for myself | 5 | 6 | 1 |
| 12 | I am taking good care of my baby's physical needs | 6 | 6 | 0 |
| | (feedings, changing diapers, doctor's appointments) | | | |
| 13 | I am taking good care of my physical needs (eating, | 4 | 5 | 1 |
| | showering, etc) | | | |
| 14 | I make good decisions about my baby's health and | 6 | 6 | 0 |
| | well being | | | |
| 15 | My baby and I are getting into a routine | 5 | 6 | 1 |
| 16 | I worry about how other people judge me (as a | 3 | 5 | 2 |
| | mother) | | | |
| 17 | I am able to take care of my baby and my other | 5 | 5 | 0 |
| | responsibilities | | | |
| 18 | Anxiety or worry often interferes with my mothering | 2 | 5 | 3 |
| | ability | | | |
| 19 | As time goes on, I am getting better at taking care of | 0 | 1 | 1 |
| | my baby | | | |
| 20 | I am satisfied with the job I am doing as a new mother | 6 | 5 | −1 |
| | TOTAL | 92 | 105 | 13 |

TABLE 11
BIMF functional domain scores table

| | | Score | | | | |
|---|---|---|---|---|---|---|
| | | Maxi- | | | | |
| | | mum | | | Δ | % Im- |
| Functional | Related BIMF | Possi- | Day | Day | (Day | prove- |
| Domain | Items | ble | 0 | 7 | 7) | ment |
| Self-Care | 2, 11, 13 | 18 | 9 | 13 | 4 | 44.44% |
| Infant Care | 12, 14 | 12 | 12 | 12 | 0 | 0.00% |
| Mother-Child | 4, 5,15 | 18 | 15 | 17 | 2 | 13.33% |
| Interaction | | | | | | |
| Psychological | 1, 2, 3, 5, 7, 10, | 60 | 44 | 53 | 9 | 20.45% |
| Well-being | 11, 16, 18, 20 | | | | | |
| Social Support | 6,8, 9 | 18 | 17 | 18 | 1 | 5.88% |
| Management | 7, 11, 13, 14, 17, | 36 | 28 | 33 | 5 | 17.86% |
| Adjustment | 18 17, 19 | 12 | 5 | 6 | 1 | 20.00% |

### Summary and Conclusions

A. An individualised dosing regimen of 6 mg 5-MeO-DMT, followed by 12 mg 5-MeO-DMT administered via inhalation was well tolerated and induced an astonishing and very significant clinical response in a patient formally diagnosed with postpartum depression.
B. The clinical response occurs rapidly within 2 hours after 5-MeO-DMT administration. Such rapid onset is unusual and has not been seen with conventional classes of antidepressants, including tricyclic antidepressants, monoamine oxidase inhibitors, selective serotonin-reuptake inhibitors (SSRIs), serotonin-norepinephrine reuptake inhibitors (SNRIs), and others, which generally take 4 to 6 weeks to show their effect.
C. The patient experienced a clinical remission within 2 hours after 5-MeO-DMT administration according to the IDR. This is highly superior to any approved therapy for postpartum depression, and also to all previously tested psychedelic agents.
D. A significant clinical response was sustained over the 7-day follow-up period, although 5-MeO-DMT was only given once and is no longer efficaciously present in the body during this time frame (see pharmacokinetic data in Example 6 above). This observation supports the superior clinical profile of 5-MeO-DMT and allows for convenient administration intervals.
E. In addition to anti-depressive effects, endpoints assessing other symptoms (such as somatic concerns, emotional withdrawal, anxiety, guilt and tension) were positively impacted, supporting the use of 5-MeO-DMT in patients with other mental diseases.
F. In addition to anti-depressive effects, endpoints assessing maternal functioning, as assessed using the BIMF, such as self-care, psychological well-being and management, were positively impacted. This supports additional benefits of 5-MeO-DMT to patients suffering from PPD beyond improvement in their core depressive symptoms.

The highlighted aspects show that 5-MeO-DMT has a significantly improved efficacy profile compared to approved pharmacological therapies for postpartum depression and to all previously tested psychedelic agents, when used according to the present invention.

Together with the short duration of the acute psychedelic effects and the favourable safety profile, these data show that the technical problem to provide an improved psychoactive therapy in a patient with a postpartum depression is solved by the present invention.

A second patient with postpartum depression diagnosed by a psychiatrist has been recruited into the clinical trial. Diagnosis was Major Depressive Disorder without psychotic features, confirmed by the Mini-International Neuropsychiatric Interview (MINI) (v7.0.2), with peri-partum onset that began no earlier than gestation and no later than the first 4 weeks postpartum. The patient completed all planned visit days (here on day 5 instead of day 7). The inhalation procedure was adequately performed by the patient and was well tolerated with no inhalation-related adverse events.

Except for a temporary, clinically non-relevant increase in heart rate shortly after administration of 5-MeO-DMT, no other noteworthy changes in vital parameters occurred. Assessments of ECG (at 3 hours after administration) and safety laboratory analyses (at 3 hours and 7 days), CADSS (at 3 hours, 1 day and 7 days) were unremarkable. The few reported adverse events (vomiting on Day 0 and headache on Day 5) were mild, short-lasting and resolved spontaneously by the end of the study.

With regard to the intensity of the psychedelic experience, the recorded PES score achieved upon exposure to a nominal dose of 6 mg was 1. This score indicated the need to proceed to the administration of a subsequent, higher dose of 12 mg, one hour after the first dose, per the design of the individualised dosing regimen. The PES score achieved for this dose was 94.7 and, being ≥75, indicated the occurrence of a peak psychedelic experience and the completion of the DR for this patient.

Significantly, the patient reported a major improvement in her depressive symptoms as assessed by MADRS at the earliest assessment timepoint of 2 hours after drug administration, with the effect being maintained over time. The patient also fulfilled standard criteria for MADRS response (at least 50% improvement from baseline) and MADRS remission (MADRS total score equal or less than 10).

The second patients was lactating, and, per the clinical trial protocol, breastmilk samples were collected pre-dosing and at multiple timepoints after administration of the last dose. These samples were subsequently analysed via LC-MS/MS assay for the detection of 5-MeO-DMT, bufotenine (a primary metabolite of 5-MeO-DMT) and 5-MIAA (a final metabolite of 5-MeO-DMT). Similar analyses were carried out on patient serum and urine samples. The data are summarised in the table below.

| Timepoint [hrs] | Urine [pg/ml] | Serum [pg/ml] | Breastmilk [pg/ml] |
|---|---|---|---|
| 5-MeO-DMT | | | |
| −24.0 | BLQ | BLQ | BLQ |
| 1.0 | ND | 132.9 | 2167.0 |
| 2.5 | 566.7 | BLQ | 560.6 |
| 8.5 | BLQ | ND | 42.1 |
| 24.0 | BLQ | BLQ | BLG |
| 120.0 | BLQ | BLQ | BLQ |
| 5-MIAA | | | |
| −24.0 | 8113 | 51.0 | BLQ |
| 1.0 | ND | 263092.0 | 13945.2 |
| 2.5 | 12980501 | 101994.7 | 13240.9 |
| 8.5 | 291651 | ND | 359.4 |
| 24.0 | 18649 | 270.6 | 35.5 |
| 120.0 | 9444 | 67.1 | BLQ |
| Bufotenine | | | |
| −24.0 | BLQ | BLQ | BLQ |
| 1.0 | ND | BLQ | BLQ |
| 2.5 | 32.3 | BLQ | BLQ |
| 8.5 | BLQ | ND | BLQ |
| 24.0 | BLQ | BLQ | BLQ |
| 120.0 | BLQ | BLQ | BLQ |

ND: Not determined.
BLQ: Below limit of quantification. The lower limit of quantification is 25 pg/ml for both 5-MeO-DMT and 5-MIAA in both serum and breastmilk; it is 25 pg/ml in urine for 5-MeO-DMT and 250 pg/ml for 5-MIAA in urine.

The data indicate the rapid clearance of 5-MeO-DMT from serum by the first measurement timepoint of 1 hour post last dose, with a concentration of 132.9 μg/ml detected at this point and all subsequent timepoints resulting in concentrations below the lower limit of quantification of the assay. Additionally, endogenous levels of 5-MIAA were confirmed in serum prior to dosing, with highest concentrations detected at the first measurement timepoint (1 hour after dosing) and returning to baseline levels within 1 day. The urine assay data demonstrate the presence of 5-MeO-DMT at concentrations approx. four-fold higher than those detected in serum at 1 hour (indicating already significant excretion by this point), falling off rapidly to undetectable levels after this timepoint, while 5-MIAA levels peak at concentrations approx. 127 times higher than those detected in serum at 2.5 hours and reducing rapidly to endogenous levels within 7 days. Bufotenine was not detected at any timepoint with the exception of 2.5 hours post-dosing in urine. Taken together, these data confirm the rapid metabolism and clearance of 5-MeO-DMT in vivo.

The analysis of breastmilk samples indicated the absence of bufotenine, while the 5-MeO-DMT concentration was 2167 μg/ml at one hour, falling rapidly to levels at 8.5 hours following administration similar to those seen endogenously in serum pre-dosing. A similar trend was observed for 5-MIAA.

### Example 13—Clinical Trial of 5-MeO-DMT Administered Via Intravenous Injection to Patients with Bipolar II Disorder—Prophetic Example

The clinical trial will involve adult patients with bipolar II disorder and a current major depressive episode.

Patients who are currently taking anti-depressive medication need to discontinue or taper overtime such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 2 mg, 5 mg, and 8 mg.
1. All patients will receive an initial dose of 2 mg 5-MeO-DMT.
2. The second dose (5 mg) will only be administered if:
   a. A peak experience (PES total score of ≥75) has not been achieved following the 2 mg dose, and
   b. The 2 mg dose was safe and well-tolerated.
3. Similarly, a third dose (8 mg) will only be administered if:
   a. A peak experience (PES total score of ≥75) has not been achieved following the 5 mg dose, and
   b. The 5 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviors within the past year; or (c) clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, uncontrolled hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 2 mg, 5 mg and 8 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 2 mg, 5 mg and 8 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS 510) at 2 hours after the final study drug dosing on Day 0, and at Day 1 and Day 7;
   Change from baseline in MADRS assessed at 2 hours after the final study drug dosing on Day 0, and at Day 1;
   The proportion of responders (≥50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
The safety and tolerability of 5-MeO-DMT administered via intravenous injection evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the
   achievement of a PE (PES total score ≥75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 14—Clinical Trial of 5-MeO-DMT Administered Via Intravenous Injection to Patients with Bipolar II Disorder—Prophetic Example

The clinical trial will involve adult patients with bipolar II disorder and a current major depressive episode.

Patients who are currently taking anti-depressive medication need to discontinue or taper overtime such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 1 mg, 2 mg, and 3 mg.
1. All patients will receive an initial dose of 1 mg 5-MeO-DMT.
2. The second dose (2 mg) will only be administered if:
   a. A peak experience (PES total score of ≥75) has not been achieved following the 1 mg dose, and
   b. The 1 mg dose was safe and well-tolerated.
3. Similarly, a third dose (3 mg) will only be administered if:
   a. A peak experience (PES total score of ≥75) has not been achieved following the 2 mg dose, and
   b. The 2 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviors within the past year; or (c) clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, uncontrolled hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 1 mg, 2 mg and 3 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 1 mg, 2 mg and 3 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS 510) at 2 hours after the final study drug dosing on Day 0, and at Day 1 and Day 7;
   Change from baseline in MADRS assessed at 2 hours after the final study drug dosing on Day 0, and at Day 1;
   The proportion of responders (≥50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
The safety and tolerability of 5-MeO-DMT administered via intravenous injection evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score ≥75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 15—Clinical Trial of 5-MeO-DMT Administered Via Intravenous Injection to Patients with Postpartum Depression—Prophetic Example

The clinical trial will involve adult female patients with clinically diagnosed postpartum depression (PPD).

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on Day 0: 2 mg, 5 mg, and 8 mg.
1. All patients will receive an initial dose of 2 mg 5-MeO-DMT.
2. The second dose (5 mg) will only be administered if:
   a. A peak experience (total score of ≥75) has not been achieved following the 2 mg dose, and
   b. The 2 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (8 mg) will only be administered if:
   a. A peak experience (total score of ≥75) has not been achieved following the 5 mg dose, and
   b. The 5 mg dose was safe and well-tolerated according to the investigator,
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing.

Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate <1%), medically accepted contraceptive method for 30 days prior to dosing and for 90 days after 5-MeO-DMT dosing. Patients must have a negative pregnancy test at screening and on the pre-test day (Day −1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-MeO-DMT.
8. Has any current or past clinically significant condition (e.g., severe infection, pulmonary disease, uncontrolled hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day −1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 2 mg, 5 mg and 8 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 2 mg, 5 mg and 8 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day DR of 2 mg, 5 mg and 8 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS:10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (≥50% reduction from baseline in MADRS total score) at 2 hours after the final study drug dosing on Day 0, and at Day 1 and Day 7;
   Change from baseline in Clinical Global Impression-Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day −1), 1 hour after last study drug dosing, at discharge, in the evening of Day 0, and on Day 1 and on Day 7;
Exposure of 5-MeO-DMT and bufotenine in blood obtained at the pre-test day (Day −1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the achievement of a PE (PE scale total score ≥75);
   Challenging Experience Questionnaire (CEQ);
   Mystical Experience Questionnaire (MEQ-30);
Duration of the PsE defined as the time from study drug dosing to the time when the PsE have subsided (investigator- and patient-scored), completed 30 to 60 minutes after each dosing;

### Example 16—Clinical Trial of 5-MeO-DMT Administered Via Intravenous Injection to Patients with Postpartum Depression—Prophetic Example

The clinical trial will involve adult female patients with clinically diagnosed postpartum depression (PPD).

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on Day 0: 1 mg, 2 mg, and 3 mg.
1. All patients will receive an initial dose of 1 mg 5-MeO-DMT.
2. The second dose (2 mg) will only be administered if:
   a. A peak experience (total score of ≥75) has not been achieved following the 1 mg dose, and
   b. The 1 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (3 mg) will only be administered if:
   a. A peak experience (total score of ≥75) has not been achieved following the 2 mg dose, and
   b. The 2 mg dose was safe and well-tolerated according to the investigator,
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing.

Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate <1%), medically accepted contraceptive method for 30 days prior to dosing and for 90 days after 5-MeO-DMT dosing. Patients must have a negative pregnancy test at screening and on the pre-test day (Day −1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-MeO-DMT.
8. Has any current or past clinically significant condition (e.g., severe infection, pulmonary disease, uncontrolled hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day −1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 1 mg, 2 mg and 3 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 1 mg, 2 mg and 3 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day DR of 1 mg, 2 mg and 3 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS:10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (≥50% reduction from baseline in MADRS total score) at 2 hours after the final study drug dosing on Day 0, and at Day 1 and Day 7;
   Change from baseline in Clinical Global Impression-Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day −1), 1 hour after last study drug dosing, at discharge, in the evening of Day 0, and on Day 1 and on Day 7;
Exposure of 5-MeO-DMT and bufotenine in blood obtained at the pre-test day (Day −1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the achievement of a PE (PE scale total score ≥75);
   Challenging Experience Questionnaire (CEQ);
   Mystical Experience Questionnaire (MEQ-30);

Duration of the PsE defined as the time from study drug dosing to the time when the PsE have subsided (investigator- and patient-scored), completed 30 to 60 minutes after each dosing.

1. A method of treating a mental or nervous system disorder in a breastfeeding mother, comprising administering to the breastfeeding mother suffering from a mental or nervous system disorder, an effective amount of 5-Methoxy-N,N-dimethyltryptamine (5-MeO-DMT) or a pharmaceutically acceptable salt thereof,
wherein a dosage of about 1 mg to about 10 mg 5-MeO-DMT or of an equimolar amount of the pharmaceutically acceptable salt is administered as a single dose or as the highest dose in an uptitration scheme involving intervals between administrations of at least about 1 hour,
wherein the 5-MeO-DMT or the pharmaceutically acceptable salt thereof is administered via the intravenous, intramuscular, or subcutaneous route, and
wherein the patient is advised to temporarily cease breastfeeding.

2. The method according to claim 1, wherein the patient is advised to temporarily cease breastfeeding from 1 hour or more prior to receiving the first dose and not to resume breastfeeding until at least 24 hours post last dose.

3. The method according to claim 1, wherein the patient is advised to temporarily cease breastfeeding from 1 hour or more prior to receiving the first dose and not to resume breastfeeding until at least 12 hours post last dose.

4. The method according to claim 1, wherein the patient is advised to temporarily cease breastfeeding from 1 hour or more prior to receiving the first dose and not to resume breastfeeding until at least 6 hours post last dose.

5. The method according to claim 1, wherein the patient is advised to temporarily cease breastfeeding from 1 hour or more prior to receiving the first dose and not to resume breastfeeding until at least 2 hours post last dose.

6. The method according to claim 1, wherein the patient is advised to temporarily cease breastfeeding from 1 hour or more prior to receiving the first dose and not to resume breastfeeding until at least 1 hours post last dose.

7. The method according to claim 1, wherein the patient is advised to temporarily cease breastfeeding from just prior to receiving the first dose and not to resume breastfeeding until at least 24 hours post last dose.

8. The method according to claim 1, wherein the patient is advised to temporarily cease breastfeeding from just prior to receiving the first dose and not to resume breastfeeding until at least 12 hours post last dose.

9. The method according to claim 1, wherein the patient is advised to temporarily cease breastfeeding from just prior to receiving the first dose and not to resume breastfeeding until at least 6 hours post last dose.

10. The method according to claim 1, wherein the patient is advised to temporarily cease breastfeeding from just prior to receiving the first dose and not to resume breastfeeding until at least 2 hours post last dose.

11. The method according to claim 1, wherein the patient is advised to temporarily cease breastfeeding from just prior to receiving the first dose and not to resume breastfeeding until at least 1 hours post last dose.

12. The method according to claim 1, wherein the patient is advised to temporarily cease breastfeeding only for the period of the actual treatment.

13. The method according to claim 1, wherein the patient is advised to temporarily cease breastfeeding from just prior to receiving the first dose until discharge readiness.

14. The method according to claim 13, wherein the determination of discharge readiness occurs at about 1 hour after the last dose.

15. The method according to claim 13, wherein the patient is advised not to recommence breastfeeding before the later of discharge and 6 hours after the last dose.

16. The method according to claim 1, wherein the patient is advised not to recommence breastfeeding before the later of discharge and 3 hours after the last dose.

17. The method according to claim 1, wherein the patient is advised not to recommence breastfeeding before the later of discharge and 2 hours after the last dose.

18. The method according to claim 1, wherein the patient is advised not to recommence breastfeeding before the later of discharge and 1 hours after the last dose.

19. The method according to claim 1, wherein the patient is advised to temporarily cease breastfeeding from just prior to receiving the first dose until at least 24 hours post last dose and to discard all breast milk expressed during the 24 hour period.

20. The method according to claim 1, wherein the patient is advised to temporarily cease breastfeeding from just prior to receiving the first dose until at least 2.5 hours post last dose and to pump and discard breast milk at 2.5 hours post last dose.

21. The method according to claim 1, wherein the patient is advised to pump and discard breast milk at 24 hours post last dose prior to reinitiating breastfeeding.

22. The method according to claim 1, wherein the patient is advised to breastfeed the child not more than 2 times during the first 12 hours post last dose.

23. The method according to claim 1, wherein any expressed breast milk is discarded as long as the concentrations of 5-MeO-DMT and/or 5-MIAA in breast milk exceed predetermined thresholds.

24. The method according to claim 23, wherein the threshold for 5-MeO-DMT is 2000 pg.

25. The method according to claim 23, wherein the threshold for 5-MeO-DMT is 500 pg.

26. The method according to claim 23, wherein the threshold for 5-MeO-DMT is 75 pg.

27. The method according to claim 23, wherein the threshold for 5-MIAA is 14000 pg.

28. The method according to claim 23, wherein the threshold for 5-MIAA is 2000 pg.

29. The method according to claim 23, wherein the threshold for 5-MIAA is 75 pg.

30. The method according to claim 1, breastfeeding is resumed when the 5-MeO-DMT concentration and/or the 5-MIAA concentration in breast milk is below a predetermined threshold.

31. The method according to claim 30, wherein the threshold for 5-MeO-DMT is 2000 pg.

32. The method according to claim 30, wherein the threshold for 5-MeO-DMT is 500 pg.

33. The method according to claim 30, wherein the threshold for 5-MeO-DMT is 75 pg.

34. The method according to claim 30, wherein the threshold for 5-MIAA is 14000 pg.

35. The method according to claim 30, wherein the threshold for 5-MIAA is 2000 pg.

36. The method according to claim 30, wherein the threshold for 5-MIAA is 75 pg.

37. The method according to claim 1, wherein breastfeeding is adapted such that the DID of 5-MeO-DMT per kg infant weight is 1 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

38. The method according to claim 1, wherein breastfeeding is adapted such that the DID of 5-MeO-DMT per kg infant weight is 0.4 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

39. The method according to claim 1, wherein breastfeeding is adapted such that the DID of 5-MeO-DMT per kg infant weight is 0.2 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

40. The method according to claim 1, wherein breastfeeding is adapted such that the RID of 5-MeO-DMT is 10% or below, for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

41. The method according to claim 1, wherein breastfeeding is adapted such that the RID of 5-MeO-DMT is 5% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

42. The method according to claim 1, wherein breastfeeding is adapted such that the RID of 5-MeO-DMT is 1% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

43. The method according to claim 1, wherein breastfeeding is adapted such that the DID of the terminal metabolite 5-MIAA per kg infant weight is 4 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

44. The method according to claim 1, wherein breastfeeding is adapted such that the DID of the terminal metabolite 5-MIAA per kg infant weight is 2 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

45. The method according to claim 1, wherein breastfeeding is adapted such that the DID of the terminal metabolite 5-MIAA per kg infant weight is 1 μg/kg/day or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

46. The method according to claim 1, wherein breastfeeding is adapted such that the RID of the terminal metabolite 5-MIAA is 4% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

47. The method according to claim 1, wherein breastfeeding is adapted such that the RID of the terminal metabolite 5-MIAA is 2% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

48. The method according to claim 1, wherein breastfeeding is adapted such that the RID of the terminal metabolite 5-MIAA is 1% or below for the first 24 hours of feeding after resumption of breastfeeding, wherein adaptation is by selecting an appropriate time point for resumption of breastfeeding, an appropriate number of feeds during the first 24 hours after resumption of breastfeeding, by pumping and discarding breastmilk for an appropriate period of time or by a combination of these measures.

49. The method according to claim 1, wherein the patient suffers from PPD.

50. The method according to claim 49, wherein a remission of depressive symptoms, as assessed by a MADRS score equal to or less than 10 occurs not later than about 2 hours after the last administration of 5-MeO-DMT or a pharmaceutically acceptable salt thereof.

51. The method according to claim 50, wherein a remission of depressive symptoms, as assessed by a HAM-D score equal to or less than 7, occurs not later than about 2 hours after the last administration of 5-MeO-DMT or a pharmaceutically acceptable salt thereof.

52. The method according to claim 1, wherein maternal functioning is improved which is reflected in improvements in the functional domains according to the Barkin Index of Maternal Functioning (BIMF) of mother-child interaction and psychological well-being.

53. The method according to claim 52, wherein the improvement of the cumulative score of the BIMF scale items reflecting psychological well-being is at least 25% and the improvement of the cumulative score of the BIMF scale items reflecting mother-child interaction is at least 5%.

54. The method according to claim 52, wherein improvements in the MADRS items lassitude, pessimistic thoughts, inability to feel, inner tension and/or reduced sleep lead to an increase in the BIMF scale scores reflecting psychological well-being and improvements in the MADRS items inability to feel and inner tension lead to an increase in the BIMF scale scores reflecting mother-child interaction.

55. The method according to claim 1, wherein the patient suffers from a mental or nervous system disorders involving one or more symptoms selected from sleep disturbance and anxiety, wherein each of these symptoms compromises maternal functioning.

56. The method according to claim 55, wherein the patient suffers from a mental or nervous system disorders involving anxiety symptoms and wherein a treatment reduces or eliminates symptoms of anxiety, in particular of inner tension.

57. The method according to claim 56, wherein a treatment leads to a clinical response which is reflected by an at least 50% decrease of the HAM-A score, compared to the respective score prior to treatment, about 2 hours after the last administration of 5-MeO-DMT or a pharmaceutically acceptable salt thereof after the last administration of 5-MeO-DMT or a pharmaceutically acceptable salt thereof.

58. The method according to claim 56, wherein a treatment leads to a clinical response which is reflected by an at least 50% decrease of the HAM-A score, compared to the respective score prior to treatment, on day 1, for instance after about 24 hours after the last administration of 5-MeO-DMT or a pharmaceutically acceptable salt thereof.

59. The method according to claim 56, wherein a treatment leads to a clinical response which is reflected by an at least 50% decrease of the HAM-A score, compared to the respective score prior to treatment, on day 7 after the last administration of 5-MeO-DMT or a pharmaceutically acceptable salt thereof.

60. The method according to claim 56, wherein a treatment leads to a clinical response which is reflected by an at least 50% decrease of the HAM-A score, compared to the respective score prior to treatment, on day 14 after the last administration of 5-MeO-DMT or a pharmaceutically acceptable salt thereof.

61. The method according to claim 56, wherein a treatment leads to a clinical response which is reflected by an at least 50% decrease of the HAM-A score, compared to the respective score prior to treatment, on day 28 after the last administration of 5-MeO-DMT or a pharmaceutically acceptable salt thereof.

62. The method according to claim 55, wherein the patient suffers from a mental or nervous system disorders involving sleep disturbance symptoms and wherein a treatment reduces or eliminates symptoms of sleep disturbance.

63. The method according to claim 62, wherein the patient suffers from sleep disturbance as reflected by a Pittsburgh Sleep Quality Index (PSQI) global score of >5.

64. The method according to claim 62, wherein the treatment reduces or eliminates sleep disturbance and the reduction or elimination of sleep disturbance is reflected by an improvement in the Pittsburgh Sleep Quality Index (PSQI) global score on day 1, for instance, about 24 hours after the last administration of 5-MeO-DMT or a pharmaceutically acceptable salt thereof, wherein the recall period spans from the time point when acute psychedelic experiences have subsided after the last administration to the assessment time point.

65. The method according to claim 62, wherein the treatment success is indicated by a decrease in the PSQI global score.

66. The method according to claim 62, wherein the treatment success is indicated by a decrease in at least four of the seven component scores of the PSQI.

67. The method according to claim 62, wherein the treatment success is indicated by a decrease in the PSQI global score to 5 or below.

68. The method according to claim 62, wherein the sleep disturbance is insomnia.

69. The method according to claim 1, wherein the 5-MeO-DMT or salt thereof is administered at a dose or in a dosage regimen that causes the patient to experience a peak psychedelic experience.

70. The method according to claim 1, wherein a dosage of about 2 mg; or of about 5 mg; or of about 8 mg is administered, or wherein equimolar amounts of the pharmaceutically acceptable salt are administered instead of 5-MeO-DMT.

71. The method according to claim 1, wherein a dosage of about 1 mg; or of about 2 mg; or of about 3 mg is administered, or wherein equimolar amounts of the pharmaceutically acceptable salt are administered instead of 5-MeO-DMT.

72. The method according to claim 1, wherein a dosage of about 2 mg; or of about 4 mg; or of about 6 mg is administered, or wherein equimolar amounts of the pharmaceutically acceptable salt are administered instead of 5-MeO-DMT.

73. The method according to claim 1, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

74. The method according to claim 1 or 73, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.

75. The method according to claim 74, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.

76. The method according to claim 1, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.

77. The method according to claim 76, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.

78. The method according to claim 1, wherein the 5-MeO-DMT is administered in a dosage from about 2 mg to about 3 mg for a first administration, and then increased, unless the patient has already experienced a peak psychedelic experience, to a dosage from about 4 mg to about 5 mg for a second administration, and then increased, unless the patient has already experienced a peak psychedelic experience, to a dosage from about 6 mg to about 7.5 mg for a third administration, or wherein equimolar amounts of the pharmaceutically acceptable salt are administered instead of 5-MeO-DMT.

79. The method according to claim 78, wherein the first dosage of 5-MeO-DMT is about 2 mg, the second dosage of 5-MeO-DMT is about 4 mg, and the third dosage of 5-MeO-DMT is about 6 mg; or wherein equimolar amounts of the pharmaceutically acceptable salt are administered instead of 5-MeO-DMT.

80. The method according to claim 73, wherein the interval between two administrations is not less than 1 hour and not more than 24 hours.

81. The method according to claim 1,
wherein a dosage of about 1 mg to about 5 mg 5-MeO-DMT or of an equimolar amount of the pharmaceutically acceptable salt is administered as a single dose or as the highest dose in an uptitration scheme involving intervals between administrations of at least about 1 hour,
wherein the 5-MeO-DMT or the pharmaceutically acceptable salt thereof is administered via the intravenous, intramuscular, or subcutaneous route, and
wherein the patient is advised to temporarily cease breastfeeding.

82. The method according to claim 81, wherein a dosage of about 1 mg; or of about 5 mg is administered, or wherein equimolar amounts of the pharmaceutically acceptable salt are administered instead of 5-MeO-DMT.

83. The method according to claim 82, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

84. The method according to claim 83, wherein the interval between two administrations is not less than 1 hour and not more than 24 hours.

85. The method according to claim 69, wherein the occurrence of a peak psychedelic experience is identified through achievement of at least 60% of the maximum possible score in each of the four subscales including mystical, positive mood, transcendence of time and space, and ineffability, of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.

86. The method according to claim 85, wherein the occurrence of a peak psychedelic experience is identified through achievement of a Peak Experience Scale (PES) Total Score of at least 75.

87. The method according to claim 1, wherein the 5-MeO-DMT or a pharmaceutically acceptable salt thereof is administered via intravenous injection.
